(19) United States
(12) Patent Application Publication
(10) Pub. No.: US 2008/0312208 A1
(43) Pub. Date: Dec. 18, 2008

(54) Pyridine Analogues

(75) Inventors: Soren Andersen, Molndal (SE); Peter Bach, Molndal (SE); Kay Brickmann, Molndal (SE); Fabrizio Giordanetto, Molndal (SE); Fredrik Zetterberg, Molndal (SE); Krister Osterlund, Molndal (SE)

Correspondence Address: Pepper Hamilton LLP, 400 Berwyn Park, 899 Cassatt Road, Berwyn, PA, 19312-1183, US

(73) Assignee: ASTRAZENECA AB, Sodertalje (SE)

(21) Appl. No.: 11/995,326

(22) PCT Filed: Jul. 4, 2006
(86) PCT No.: PCT/SE2006/000832

§ 371 Date: Jan. 11, 2008

(30) Foreign Application Priority Data
Jul. 13, 2005 (SE) 0501663-9
Oct. 24, 2005 (SE) 0502354-4

(51) Int. Cl. A61K 31/4427; A61K 31/496; C07D 401/04; A61K 31/4545; A61P 7/02

(52) U.S. Cl. 514/210.18; 544/360; 514/253.01; 514/318; 546/193; 546/268.1

(57) ABSTRACT

The present invention relates to certain new pyridin analogues of Formula (I) Chemical formula should be inserted here. Please see paper copy Formula (I) to processes for preparing such compounds, to their utility as P2Y₁₂ inhibitors and as anti-thrombotic agents etc, their use as medicaments in cardiovascular diseases as well as pharmaceutical compositions containing them.

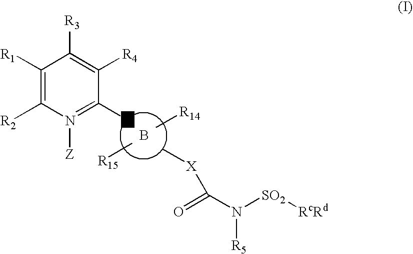

### FIELD OF THE INVENTION

The present invention provides novel pyridine compounds, their use as medicaments, compositions containing them and processes for their preparation.

### BACKGROUND OF THE INVENTION

Platelet adhesion and aggregation are initiating events in arterial thrombosis. Although the process of platelet adhesion to the sub-endothelial surface may have an important role to play in the repair of damaged vessel walls, the platelet aggregation that this initiates can precipitate acute thrombotic occlusion of vital vascular beds, leading to events with high morbidity such as myocardial infarction and unstable angina. The success of interventions used to prevent or alleviate these conditions, such as thrombolysis and angioplasty is also compromised by platelet mediated occlusion or re-occlusion.

Haemostasis is controlled via a tight balance between platelet aggregation, coagulation and fibrinolysis. Thrombus formation under pathological conditions, like e.g. arteriosclerotic plaque rupture, is firstly initiated by platelet adhesion, activation and aggregation. This results not only in the formation of a platelet plug but also in the exposure of negatively charged phospholipids on the outer platelet membrane promoting blood coagulation. Inhibition of the build-up of the initial platelet plug would be expected to reduce thrombus formation and reduce the number of cardiovascular events as was demonstrated by the anti-thrombotic effect of e.g. Aspirin (BMJ 1994; 308: 81-106 Antiplatelet Trialists' Collaboration. Collaborative overview of randomised trials of antiplatelet therapy, I: Prevention of death, myocardial infarction, and stroke by prolonged antiplatelet therapy in various categories of patients). Platelet activation/aggregation can be induced by a variety of different agonists. However, distinct intracellular signalling pathways have to be activated to obtain full platelet aggregation, mediated via G-proteins G_{q}, G_{12/13} and Gᵢ (Platelets, A D Michelson ed., Elsevier Science 2002, ISBN 0-12-493951-1; 197-213: D Woulfe, et al. Signal transduction during the initiation, extension, and perpetuation of platelet plug formation) In platelets, the G-protein coupled receptor P2Y₁₂ (previously also known as the platelet P_{2T}, P2T_{ac}, or P2Y_{cyc} receptor) signals via Gi, resulting in a lowering of intra-cellular cAMP and full aggregation (Nature 2001; 409: 202-207 G Hollopeter, et al. Identification of the platelet ADP receptor targeted by antithrombotic drugs.). Released ADP from dense-granules will positively feedback on the P2Y12 receptor to allow full aggregation.

Clinical evidence for the key-role of the ADP-P2Y₁₂ feedback mechanism is provided by the clinical use of clopidogrel, an thienopyridine prodrug which active metabolite selectively and irreversibly binds to the P2Y₁₂ receptor, that has shown in several clinical trials to be effective in reducing the risk for cardiovascular events in patients at risk (Lancet 1996; 348: 1329-39: CAPRIE Steering committee, A randomised, blinded, trial of clopidogrel versus aspirin in patients at risk of ischaemic events (CAPRIE); N Engl J Med 2001; 345 (7): 494-502): The Clopidogrel in Unstable Angina to prevent Recurrent Events Trial Investigators. Effects of clopidogrel in addition to aspirin in patients with acute coronary syndromes without ST-segment elevation.). In these studies, the clinical benefit with a reduced bleeding risk as compared to thienopyridines (Sem Thromb Haemostas 2005; 31(2): 195-204 J J J van Giezen & R G Humphries. Preclinical and clinical studies with selective reversible direct P2Y₁₂ antagonists.

Accordingly it is an object of the present invention to provide potent, reversible and selective P2Y₁₂-antagonists as anti-trombotic agents.

### SUMMARY OF THE INVENTION

We have now surprisingly found that certain pyridine compounds of Formula (I) or a pharmaceutically acceptable salt thereof are reversible and selective P2Y₁₂ antagonists, hereinafter referred to as the compounds of the invention. The compounds of the invention unexpectedly exhibit beneficial properties that render them particularly suitable for use in the treatment of diseases/conditions as described below (See p. 69-70). Examples of such beneficial properties are high potency, high selectivity, and an advantageous therapeutic window.

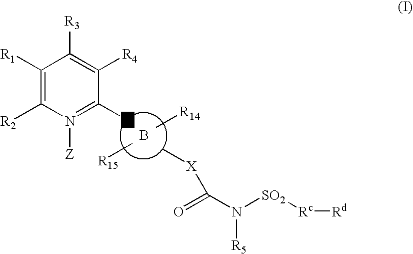

### DETAILED DESCRIPTION OF THE INVENTION

According to the present invention there is provided a novel compound of formula (I) or a pharmaceutically acceptable salt thereof:

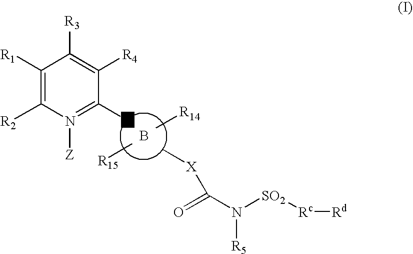

wherein

R₁ represents R₆OC(O), R₇C(O), R₁₆SC(O), R₁₇S, R₁₈C(S) or a group gII

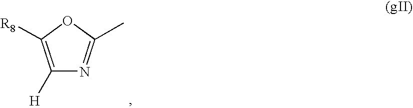

preferably R₁ represents R₆OC(O), R₁₆SC(O) or the group gII;

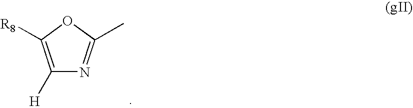

R₂ represents H, CN, halogen (P, Cl, Br, I), NO₂, (C₁-C₁₂)alkyl optionally interrupted by oxygen and/or optionally substituted by OH, aryl, cycloalkyl, heterocyclyl or one or more halogen (F, Cl, Br, I) atoms; further R₂ represents (C₁-C₁₂)alkoxy optionally substituted by one or more halogen (F, Cl, Br, I) atoms; further R₂ represents (C₃-C₆)cycloalkyl, hydroxy(C₁-C₁₂)alkyl, (C₁-C₁₂)alkylC(O), (C₁-C₂)alkylthioC(O), (C₁-C₁₂)alkylC(S), (C₁-C₁₂)alkoxyC(O), (C₃-C₆)cycloalkoxy, aryl, arylC(O), aryl(C₁-C₁₂)alkylC(O), heterocyclyl, heterocyclylC(O), heterocyclyl(C₁-C₂)alkylC(O), (C₁-C₁₂)alkylsulfinyl, (C₁-C₁₂)alkylsulfonyl, (C₁-C₂)alkylthio, (C₃-C₆)cycloalkylthio, arylsulfinyl, arylsulfonyl, arylthio, aryl(C₁-C₁₂)alkylthio, aryl(C₁-C₁₂)alkylsulfinyl, aryl(C₁-C₁₂)alkylsulfonyl, heterocyclyl(C₁-C₁₂)alklthio, heterocyclyl(C₁-C₁₂)alkylsulfinyl, heterocyclyl(C₁-C₁₂)alkylsulfonyl, (C₃-C₆)cycloalkyl(C₁-C₁₂)alkylthio, (C₃-C₆)cycloalkyl(C₁-C₁₂)alkylsulfinyl, (C₃-C₆)cycloalkyl(C₁-C₁₂)alkylsulfonyl or a group of formula NR^{a(2)}R^{b(2)} in which R^{a(2)} and R^{b(2)} independently represent H, (C₁-C₁₂)alkyl, (C₁-C₁₂)alkylC(O) or R^{a(2)} and R^{b(2)} together with the nitrogen atom represent piperidine, pyrrolidine, azetidine or aziridine;

Further, R₁+R₂ together (with two carbon atoms of the pyridine ring) may form a 5-membered or 6-membered cyclic lactone;

R₃ represents H, CN, NO₂, halogen (F, Cl, Br, I), (C₁-C₁₂)alkyl optionally interrupted by oxygen and/or optionally substituted by OH, aryl, cycloalkyl, heterocyclyl or one or more halogen (F, Cl, Br, I) atoms; further R₃ represents (C₁-C₁₂)alkoxy optionally substituted by one or more halogen (F, Cl, Br, I) atoms; further R₃ represents (C₃-C₆)cycloalkyl, hydroxy(C₁-C₁₂)alkyl, (C₁-C₁₂)alkylC(O), (C₁-C₁₂)alkylthioC(O), (C₁-C₁₂)alkylC(S), (C₁-C₁₂)alkoxyC(O), (C₃-C₆)cycloalkoxy, aryl, arylC(O), aryl(C₁-C₁₂)alkylC(O), heterocyclyl, heterocyclylC(O), heterocyclyl(C₁-C₁₂)alkylC(O), (C₁-C₁₂)alkylsulfinyl, (C₁-C₁₂)alkylsulfonyl, (C₁-C₁₂)alkylthio, (C₃-C₆)cycloalkylthio, arylsulfinyl, arylsulfonyl, arylthio, aryl(C₁-C₁₂)alkylthio, aryl(C₁-C₁₂)alkylsulfinyl, aryl(C₁-C₁₂)alkylsulfonyl, heterocyclyl(C₁-C₁₂)alkylthio, heterocyclyl(C₁-C₁₂)alkylsulfinyl, heterocyclyl(C₁-C₁₂)alkylsulfonyl, (C₃-C₆)cycloalkyl(C₁-C₁₂)alkylthio, (C₃-C₆)cycloalkyl(C₁-C₁₂)alkylsulfinyl, (C₃-C₆)Cycloalkyl(C₁-C₂)alkylsulfonyl or a group of formula NR^{a(3)}R^{b(3)} in which R^{a(3)} and R^{b(3)} independently represent H, (C₁-C₁₂)alkyl, (C₁-C₁₂)alkylC(O) or R^{a(3)} and R^{b(3)} together with the nitrogen atom represent piperidine, pyrrolidine, azetidine or aziridine;

R₄ represents H, CN, NO₂, halogen (F, Cl, Br, I), (C₁-C₁₂)alkyl optionally interrupted by oxygen and/or optionally substituted by OH, COOH, (C₁-C₆)alkoxycarbonyl, aryl, cycloalkyl, heterocyclyl or one or more halogen (F, Cl, Br, I) atoms; further R₄ represents (C₃-C₆)cycloalkyl, hydroxy(C₁-C₁₂)alkyl, (C₁-C₁₂)alkylC(O), (C₁-C₁₂)arylcycloalkyl, (C₁-C₁₂)alkoxy wherein the alkoxy group may optionally be substituted by one or more halogen (F, Cl, Br, I) atoms, OH and/or COOH and/or (C₁-C₆)alkoxycarbonyl; further R₄ represents (C₁-C₂)alkylthioC(O), (C₁-C₁₂)alkylC(S), (C₁-C₁₂)alkoxyC(O), (C₃-C₆)cycloalkoxy, aryl, arylC(O), aryl(C₁-C₁₂)alkylC(O), heterocyclyl, heterocyclylC(O), heterocyclyl(C₁-C₁₂)alkylC(O), (C₁-C₁₂)alkylsulfinyl, (C₁-C₁₂)alkylsulfonyl, (C₁-C₁₂)alkylthio, (C₃-C₆)cycloalkylthio, arylsulfinyl, arylsulfonyl, arylthio, aryl(C₁-C₁₂)acylthio, aryl(C₁-C₁₂)alkylsulfinyl, aryl(C₁-C₁₂)alkylsulfonyl, heterocyclyl(C₁-C₁₂)alkylthio, heterocyclyl(C₁-C₁₂)alkylsulfinyl, heterocyclyl(C₁-C₁₂)alkylsulfonyl, (C₃-C₆)cycloalkyl(C₁-C₁₂)alkylthio, (C₃-C₆)cycloalkyl(C₁-C₁₂)alkylsulfinyl, (C₃-C₆)cycloalkyl(C₁-C₁₂)alkylsulfonyl or a group of formula NR^{a(4)}R^{b(4)} in which R^{a(4)} and R^{b(4)} independently represent H, (C₁-C₁₂)alkyl, (C₁-C₁₂)alkylC(O) or R^{a(4)} and R^{b(4)} together with the nitrogen atom represent piperidine, pyrrolidine, azetidine or aziridine;

Z represents O or is absent;

R₅ represents H or (C₁-C₁₂)alkyl;

R₆ represents (C₁-C₁₂)alkyl optionally interrupted by oxygen, (with the proviso that any such oxygen must be at least 2 carbon atoms away from the ester-oxygen connecting the R₆ group) and/or optionally substituted by OH, aryl, cycloalkyl, heterocyclyl or one or more halogen (F, Cl, Br, I) atoms; further R₆ represents (C₃-C₆)cycloalkyl, hydroxy(C₂-C₁₂)alkyl, aryl or heterocyclyl;

R₇ represents (C₁-C₁₂)alkyl optionally interrupted by oxygen, and/or optionally substituted by OH, aryl, cycloalkyl, heterocyclyl or one or more halogen (F, Cl, Br, I) atoms; further R₇ represents (C₃-C₆)cycloalkyl, hydroxy(C₁-C₁₂)alkyl, aryl or heterocyclyl;

R₈ represents H, (C₁-C₁₂)alkyl optionally interrupted by oxygen, and/or optionally substituted by aryl, cycloalkyl, heterocyclyl or one or more halogen (F, Cl, Br, I) atoms; further R₈ represents (C₃-C₆)cycloalkyl, hydroxy(C₁-C₁₂)alkyl, (C₁-C₁₂)alkoxy, (C₃-C₆)cycloalkoxy, aryl, heterocyclyl, (C₁-C₁₂)alkylsulfinyl, (C₁-C₁₂)alkylsulfonyl, (C₁-C₁₂)alkylthio, (C₃-C₆)cycloalkylthio, arylsulfinyl, arylsulfonyl, arylthio, aryl(C₁-C₁₂)alkylthio, aryl(C₁-C₁₂)alkylsulfinyl, aryl(C₁-C₁₂)alkylsulfonyl, heterocyclyl(C₁-C₁₂)alkylthio, heterocyclyl(C₁-C₁₂)alkylsulfinyl, heterocyclyl(C₁-C₁₂)alkylsulfonyl, (C₃-C₆)cycloalkyl(C₁-C₁₂)alkylthio, (C₃-C₆)cycloalkyl(C₁-C₁₂)alkylsulfinyl or (C₃-C₆)cycloalkyl(C₁-C₁₂)alkylsulfonyl;

R₁₄ represents H, OH with the proviso that the OH group must be at least 2 carbon atoms away from any heteroatom in the B ring/ring system, (C₁-C₁₂)alkyl optionally interrupted by oxygen and/or optionally substituted by one or more of OH, COOH and COOR^{e}; wherein R^{e} represents aryl, cycloalkyl, heterocyclyl or (C₁-C₁₂)alkyl optionally substituted by one or more of halogen (F, Cl, Br, I) atoms, OH, aryl, cycloalkyl and heterocyclyl; further R₁₄ represents aryl, heterocyclyl, one or more halogen (F, Cl, Br, I) atoms, (C₃-C₆)cycloalkyl, hydroxy(C₁-C₁₂)alkyl, (C₁-C₁₂)alkoxy, (C₃-C₆)cycloalkoxy, aryl, heterocyclyl, (C₁-C₁₂)alkylsulfinyl, (C₁-C₁₂)alkylsulfonyl, (C₁-C₁₂)alkylthio, (C₃-C₆)cycloalkylthio, arylsulfinyl, arylsulfonyl, arylthio, aryl(C₁-C₁₂)alkylthio, aryl(C₁-C₁₂)alkylsulfinyl, aryl(C₁-C₁₂)alkylsulfonyl, heterocyclyl(C₁-C₁₂)alkylthio, heterocyclyl(C₁-C₁₂)alkylsulfinyl, heterocyclyl(C₁-C₁₂)alkylsulfonyl, (C₃-C₆)cycloalkyl(C₁-C₁₂)alkylthio, (C₃-C₆)cycloalkyl(C₁-C₁₂)alkylsulfinyl or (C₃-C₆)cycloalkyl(C₁-C₁₂)alkylsulfonyl, a group of formula NR^{a(14)}R^{b(14)} in which R^{a(14)} and R^{b(14)} independently represent H, (C₁-C₁₂)alkyl, (C₁-C₁₂)alkylC(O), (C₁-C₁₂)alkoxyC(O) or R^{a(14)} and R^{b(14)} together with the nitrogen atom represent piperidine, pyrrolidine, azetidine or aziridine;

R₁₅ represents H, OH with the proviso that the OH group must be at least 2 carbon atoms away from any heteroatom in the B ring/ring system, (C₁-C₁₂)alkyl optionally interrupted by oxygen and/or optionally substituted by one or more of OH, COOH and COOR^{e}; wherein R^{e} represents aryl, cycloalkyl, heterocyclyl or (C₁-C₁₂)alkyl optionally substituted by one or more of halogen (F, Cl, Br, I) atoms, OH, aryl, cycloalkyl and heterocyclyl; further R₁₅ represents aryl, heterocyclyl, one or more halogen (F, Cl, Br, I) atoms, (C₃-C₆)cycloalkyl, hydroxy(C₁-C₁₂)alkyl, (C₁-C₁₂)alkoxy, (C₃-C₆)cycloalkoxy, aryl, heterocyclyl, (C₁-C₁₂)alkylsulfinyl, (C₁-C₁₂)alkylsulfonyl, (C₁-C₁₂)alkylthio, (C₃-C₆)cycloalkylthio, arylsulfinyl, arylsulfonyl, arylthio, aryl(C₁-C₁₂)alkylthio, aryl(C₁-C₁₂)alkylsulfinyl, aryl(C₁-C₁₂)alkylsulfonyl, heterocyclyl(C₁-C₁₂)alkylthio, heterocyclyl(C₁-C₁₂)alkylsulfinyl, heterocyclyl(C₁-C₁₂)alkylsulfonyl, (C₃-C₆)cycloalkyl(C₁-C₁₂)alkylthio, (C₃-C₆)cycloalkyl(C₁-C₁₂)alkylsulfinyl, (C₃-C₆)cycloalkyl(C₁-C₁₂)alkylsulfonyl or a group of formula NR^{a(15)}R^{b(15)} in which R^{a(15)} and R^{b(15)} independently represent H, (C₁-C₁₂)ally, (C₁-C₁₂)alkylC(O)), (C₁-C₁₂)alkoxyC(O) or R^{a(15)} and R^{b(15)} together with the nitrogen atom represent piperidine, pyrrolidine, azetidine or aziridine;

R₁₆ represents (C₁-C₁₂)alkyl optionally interrupted by oxygen and/or optionally substituted by OH, aryl, cycloalkyl, heterocyclyl or one or more halogen (F, Cl, Br, I) atoms; further R₁₆ represents (C₃-C₆)cycloalkyl, hydroxy(C₂-C₁₂)alkyl, (C₁-C₁₂)alkoxy, (C₃-C₆)cycloalkoxy, aryl or heterocyclyl;

R₁₇ represents (C₁-C₁₂)alkyl optionally interrupted by oxygen and/or optionally substituted by OH, aryl, cycloalkyl, heterocyclyl or one or more halogen (F, Cl, Br, I) atoms; further R₁₇ represents (C₃-C₆)cycloalkyl, hydroxy(C₁-C₁₂)alkyl, (C₁-C₁₂)alkoxy, (C₃-C₆)cycloalkoxy, aryl or heterocyclyl;

R₁₈ represents (C₁-C₁₂)alkyl optionally interrupted by oxygen and/or optionally substituted by OH, aryl, cycloalkyl, heterocyclyl or one or more halogen P, Cl, Br, I) atoms; further R₁₈ represents (C₃-C₆)cycloalkyl, hydroxy(C₁-C₁₂)alkyl, (C₁-C₁₂)alkoxy, (C₃-C₆)cycloalkoxy, aryl or heterocyclyl;

R^{c} represents an unsubstituted or monosubstituted or polysubstituted (C₁-C₄)alkylene group, (C₁-C₄)oxoalkylene group, (C₁-C₄)alkyleneoxy or oxy-(C₁-C₄)alkylene group, wherein any substituents each individually and independently are selected from (C₁-C₄)alkyl, (C₁-C₄)alkoxyl, oxy-(C₁-C₄)alkyl, (C₂-C₄)alkenyl, (C₂-C₄)alkynyl, (C₃-C₆)cycloalkyl, carboxyl, carboxy-(C₁-C₄)alkyl, aryl, heterocyclyl, nitro, cyano, halogeno p, Cl; Br, I), hydroxyl, NR^{a(Rc)}R^{b(Rc)} in which R^{a(Rc)} and R^{b(Rc)} individually and independently from each other represents hydrogen, (C₁-C₄)alkyl or R^{s(Rc)} and R^{b(Rc)} together with the nitrogen atom represent piperidine, pyrrolidine, azetidine or aziridine; Further R^{c} represents imino (—NH—), N-substituted imino (—NR₁₉—), (C₁-C₄)alkyleneimino or N-substituted (C₁-C₄)alkyleneimino (—N(R₁₉)—((C₁-C₄)alkylene) wherein the mentioned alkylene groups are unsubstituted or monosubstituted or polysubstituted with any substituents according to above; preferably R^{c} represents imino or (C₁-C₄)alkyleneimino or an unsubstituted or monosubstituted or polysubstituted (C₁-C₄)alkylene group or (C₁-C₄)oxoalkylene group with any substituents according to above;

R₁₉ represents H or (C₁-C₄)alkyl;

R^{d} represents (C₃-C₈)cycloalkyl, aryl or heterocyclyl, and anyone of these groups optionally substituted with one or more halogen (E, Cl, Br, I) atoms and/or one or more of the following groups, OH, CN, NO₂, (C₁-C₁₂)alkyl, (C₁-C₁₂)alkoxyC(O), (C₁-C₁₂)alkoxy, halogen substituted (C₁-C₁₂)alkyl, (C₃-C₆)cycloalkyl, aryl, heterocyclyl, (C₁-C₁₂)alkylsulfinyl, (C₁-C₁₂)alkylsulfonyl, (C₁-C₁₂)alkylthio, (C₃-C₆)cycloalkylthio, arylsulfinyl, arylsulfonyl, arylthio, aryl(C₁-C₁₂)alkylthio, aryl(C₁-C₁₂)alkylsulfinyl, aryl(C₁-C₁₂)alkylsulfonyl, heterocyclyl(C₁-C₁₂)alkylthio, heterocyclyl(C₁-C₁₂)alkylsulfinyl, heterocyclyl(C₁-C₁₂)alkylsulfonyl, (C₃-C₆)cycloalkyl(C₁-C₁₂)alkylthio, (C₃-C₆)cycloalkyl(C₁-C₁₂)alkylsulfinyl, (C₃-C₆)cycloalkyl(C₁-C₁₂)alkylsulfonyl or a group of formula NR^{a(Rd)}R^{b(Rd)} in which R^{a(Rd)} and R^{b(Rd)} independently represent H, (C₁-C₁₂)alkyl, (C₁-C₁₂)alkylC(O) or R^{a(Rd)} and R^{b(Rd)} together with the nitrogen atom represent piperidine, pyrrolidine, azetidine or aziridine;

X represents a single bond, imino (—NH—), methylene (—CH₂—), iminomethylene (—CH₂—NH—) wherein the carbon is connected to the B-ring/ring system, methyleneimino (—NH—CH₂—) wherein the nitrogen is connected to the B-ring/ring system and any carbon and/or nitrogen in these groups may optionally be substituted with (C₁-C₆) alkyl; further X may represent a group (—CH₂—)ₙ wherein n=2-6, which optionally is unsaturated and/or substituted by one or more substituent chosen among halogen, hydroxyl or (C₁-C₆)alkyl;

B is a monocyclic or bicyclic, 4 to 11-membered heterocyclic ring/ring system comprising one or more nitrogen and optionally one or more atoms selected from oxygen or sulphur, which nitrogen is connected to the pyridine-ring (according to formula I) and further the B-ring/ring system is connected to X in another of its positions. The substituents R₁₄ and R₁₅ are connected to the B ring/ring system in such a way that no quarternary ammonium compounds are formed (by these connections).

Preferred values of each variable group are as follows. Such values may be used where appropriate with any of the values, definitions, claims, aspects or embodiments defined hereinbefore or hereinafter. In particular, each may be used as an individual limitation on the broadest definition of formula (I).

For the avoidance of doubt it is to be understood that where in this specification a group is qualified by ‘hereinbefore defined’, ‘defined hereinbefore’ or ‘defined above’ the said group encompasses the first occurring and broadest definition as well as each and all of the particular definitions for that group.

It will be understood that when formula I compounds contain a chiral centre, the compounds of the invention may exist in, and be isolated in, optically active or racemic form. The invention includes any optically active or racemic form of a compound of formula I which act as P2Y₁₂ receptor antagonists. The synthesis of optically active forms may be carried out by standard techniques of organic chemistry well known in the art, for example by, resolution of a racemic mixture, by chiral chromatography, synthesis from optically active starting materials or by asymmetric synthesis.

It will also be understood that the compounds of the formula I may exhibit the phenomenon of tautomerism, the present invention includes any tautomeric form of a compound of formula I which is a P2Y₁₂ receptor antagonist.

It will also be understood that in so far as compounds of the present invention exist as solvates, and in particular hydrates, these are included as part of the present invention.

It is also to be understood that generic terms such as “alkyl” include both the straight chain and branched chain groups such as butyl and tert-butyl. However, when a specific term such as “butyl” is used, it is specific for the straight chain or “normal” butyl group, branched chain isomers such as “t-butyl” being referred to specifically when intended.

In one embodiment allyl is unsubstituted or substituted by one or more halogen (F, Cl, Br, I) atoms and/or one or more of the following groups, OH, CN, NO₂, (C₁-C₁₂)alkyl, (C₁-C₁₂)alkoxyC(O), (C₁-C₁₂)alkoxy, halogen substituted (C₁-C₁₂)alkyl, (C₃-C₆)cycloalkyl, aryl, heterocyclyl, (C₁-C₁₂)alkylsulfinyl, (C₁-C₁₂)alkylsulfonyl, (C₁-C₁₂)alkylthio, (C₃-C₆)cycloalkylthio, arylsulfinyl, arylsulfonyl, arylthio, aryl(C₁-C₁₂)alkylthio, aryl(C₁-C₁₂)alkylsulfinyl, aryl(C₁-C₁₂)alkylsulfonyl, heterocyclyl(C₁-C₁₂)alkylthio, heterocyclyl(C₁-C₁₂)alkylsulfinyl, heterocyclyl(C₁-C₁₂)alkylsulfonyl, (C₃-C₆)cycloalkyl(C₁-C₁₂)alkylthio, (C₃-C₆)cycloalkyl(C₁-C₁₂)alkylsulfinyl, (C₃-C₆)cycloalkyl(C₁-C₁₂)alkylsulfonyl or a group of formula NR^{a}R^{b} in which R^{a} and R^{b} independently represent H, (C₁-C₁₂)alkyl, (C₁-C₁₂)alkylC(O) or R^{a} and R^{b} together with the nitrogen atom represent piperidine, pyrrolidine, azetidine or aziridine.

The term “alkyl” includes both linear or branched chain groups, optionally substituted by one or more halogens (F, Cl, Br, I) or mixed halogenatoms.

One embodiment of alkyl when substituted by one or more halogen atoms (F, Cl, Br, I) is, for example, alkyl substituted by one or more fluorine atoms. Another embodiment of halogen substituted alkyl includes perfluoroalkyl groups such as trifluoromethyl.

The term “cycloalkyl” generally denotes a substituted or unsubstituted (C₃-C₆), unless other chain length specified, cyclic hydrocarbon.

In one embodiment cycloalkyl is substituted by one or more halogen (F, Cl, Br, I) atoms and/or one or more of the following groups, OH, CN, NO₂, (C₁-C₁₂)alkyl, (C₁-C₁₂)alkoxyC(O), (C₁-C₁₂)alkoxy, halogen substituted (C₁-C₁₂)alkyl, (C₃-C₆)cycloalkyl, aryl, heterocyclyl, (C₁-C₁₂)alkylsulfinyl, (C₁-C₁₂)alkylsulfonyl, (C₁-C₁₂)alkylthio, (C₃-C₆)cycloalkylthio, arylsulfinyl, arylsulfonyl, arylthio, aryl(C₁-C₁₂)alkylthio, aryl(C₁-C₁₂)alkylsulfinyl, aryl(C₁-C₁₂)alkylsulfonyl, heterocyclyl(C₁-C₁₂)alkylthio, heterocyclyl(C₁-C₁₂)alkylsulfinyl, heterocyclyl(C₁-C₁₂)alkylsulfonyl, (C₃-C₆)cycloalkyl(C₁-C₁₂)alkylthio, (C₃-C₆)cycloalkyl(C₁-C₁₂)alkylsulfinyl, (C₃-C₆)cycloalkyl(C₁-C₁₂)alkylsulfonyl or a group of formula NR^{a}R^{b} in which R^{a} and R^{b} independently represent H, (C₁-C₁₂)alkyl, (C₁-C₁₂)alkylC(O) or R^{a} and R^{b} together with the nitrogen atom represent piperidine, pyrrolidine, azetidine or aziridine.

The term “alkoxy” includes both linear or branched chain groups, optionally substituted by one or more halogens (, Cl, Br, I) or mixed halogenatoms.

The term aryl denotes a substituted or unsubstituted (C₆-C₁₄) aromatic hydrocarbon and includes, but is not limited to, phenyl, naphthyl, tetrahydronaphthyl, indenyl, indanyl, antracenyl, fenantrenyl, and fluorenyl.

In one embodiment aryl is substituted by one or more halogen (F, Cl, Br, I) atoms and/or one or more of the following groups, OH, CN, NO₂, (C₁-C₁₂)alkyl, (C₁-C₁₂)alkoxyC(O), (C₁-C₁₂)alkoxy, halogen substituted (C₁-C₁₂)alkyl, (C₃-C₆)cycloalkyl, aryl, heterocyclyl, (C₁-C₁₂)alkylsulfinyl, (C₁-C₁₂)alkylsulfonyl, (C₁-C₁₂)alkylthio, (C₃-C₆)cycloalkylthio, arylsulfinyl, arylsulfonyl, arylthio, aryl(C₁-C₁₂)alkylthio, aryl(C₁-C₁₂)alkylsulfinyl, aryl(C₁-C₁₂)alkylsulfonyl, heterocyclyl(C₁-C₂)alkylthio, heterocyclyl(C₁-C₁₂)alkylsulfinyl, heterocyclyl(C₁-C₁₂)alkylsulfonyl, (C₃-C₆)cycloalkyl(C₁-C₁₂)alkylthio, (C₃-C₆)cycloalkyl(C₁-C₁₂)alkylsulfinyl, (C₃-C₆)cycloalkyl(C₁-C₁₂)alkylsulfonyl or a group of formula NR^{a}R^{b} in which R^{a} and R^{b} independently represent H, (C₁-C₁₂)alkyl, (C₁-C₁₂)alkylC(O) or R^{a} and R^{b} together with the nitrogen atom represent piperidine, pyrrolidine, azetidine or aziridine.

The term “heterocyclyl” denotes a substituted or unsubstituted, 4- to 10-membered monocyclic or multicyclic ring system in which one or more of the atoms in the ring or rings is an element other than carbon, for example nitrogen, oxygen or sulfur, especially 4-, 5- or 6-membered aromatic or aliphatic heterocyclic groups, and includes, but is not limited to azetidine, furan, thiophene, pyrrole, pyrroline, pyrrolidine, dioxolane, oxathiolane, oxazolane, oxazole, thiazole, imidazole, imidazoline, imidazolidine, pyrazole, pyrazoline, pyrazolidine, isothiazole, oxadiazole, furazan, triazole, thiadiazole, pyran, pyridine as well as pyridine-N-oxide, piperidine, dioxane, morpholine, dithiane, oxathiane, thiomorpholine, pyridazine, pyrimidine, pyrazine, piperazine, triazine, thiadiazine, dithiazine, azaindole, azaindoline, indole, indoline, naphthyridine, benzoxadiazole, dihydrobenzodioxin, benzothiophene, benzothiadiazole, imidazothiazole, 2,3-dihydrobenzofuran, isoxazole, 3-benzisoxazole, 1,2-benzisoxazole, dihydropyrazole groups, and shall be understood to include all isomers of the above identified groups. For the above groups, e.g. azetidinyl, the term “azetidinyl” as well as “azetidinylene”, etc., shall be understood to include all possible regio isomers. It is further to be understood that the term heterocyclyl may be embodified by one selection among the given possible embodiments for a variable and embodified by another (or the same) selection for another variable, e.g. R₄ when selected as heterocyclyl may be a furan, when R^{d} (also when selected as heterocyclyl) may be a pyrrole.

In one embodiment heterocyclyl is substituted by one or more halogen (F, Cl, Br, I) atoms and/or one or more of the following groups, OH, CN, NO₂, (C₁-C₁₂)allyl, (C₁-C₁₂)alkoxyC(O), (C₁-C₁₂)alkoxy, halogen substituted (C₁-C₁₂)alkyl, (C₃-C₆)cycloalkyl, aryl, heterocyclyl, (C₁-C₁₂)alkylsulfinyl, (C₁-C₁₂)alkylsulfonyl, (C₁-C₁₂)alkylthio, (C₃-C₆)cycloalkylthio, arylsulfinyl, arylsulfonyl, arylthio, aryl(C₁-C₁₂)alkylthio, aryl(C₁-C₁₂)alkylsulfinyl, aryl(C₁-C₁₂)alkylsulfonyl, heterocyclyl(C₁-C₁₂)alkylthio, heterocyclyl(C₁-C₁₂)alkylsulfinyl, heterocyclyl(C₁-C₁₂)alkylsulfonyl, (C₃-C₆)cycloalkyl(C₁-C₁₂)alkylthio, (C₃-C₆)cycloalkyl(C₁-C₁₂)alkylsulfinyl, (C₃-C₆)cycloalkyl(C₁-C₁₂)alkylsulfonyl or a group of formula NR^{a}R^{b} in which R^{a} and R^{b} independently represent H, (C₁-C₁₂)alkyl, (C₁-C₁₂)alkylC(O) or R^{a} and R^{b} together with the nitrogen atom represent piperidine, pyrrolidine, azetidine or aziridine.

In another embodiment of the invention the heterocyclyl group comprises an aromatic 5-membered or 6-membered heterocyclic ring containing one, two or three heteroatoms selected from nitrogen, oxygen and sulphur, and an aromatic 5-membered or 6-membered heterocyclic ring containing one, two or three heteroatoms selected from nitrogen, oxygen and sulphur which is fused to a benzene ring;

In an alternative embodiment of the invention the heterocyclyl group is a nor-aromatic 5-membered or 6-membered heterocyclic ring containing one, two or three heteroatoms selected from nitrogen, oxygen and sulphur, fused to a benzene ring.

In a further embodiment of the invention the heterocyclyl group is a group chosen among furyl, pyrrolyl, thienyl, pyridyl, N-oxido-pyridyl, pyrazinyl, pyrimidinyl, pyridazinyl, imidazolyl, oxazolyl, isooxazolyl, thiazolyl, isothiazolyl, oxadiazolyl, 1,2,3-triazolyl, 1,2,4-triazolyl, benzfuranyl, quinolyl, isoquinolyl, benzimidazolyl, indolyl, benzdihydrofuranyl, benzodioxolyl (such as 1,3-benzodioxolyl), benzoxadiazole, dihydrobenzodioxin, benzothiophene, benzothiadiazole, imidazothiazole, 2,3-dihydrobenzofuran, isoxazole, dihydropyrazole and benzdioxanyl (such as 1,4-benzdioxanyl). More particular values include, for example, furyl, pyrrolyl, thienyl, pyridyl, pyrazinyl, pyrimidinyl, pyridazinyl, benzoxadiazole, dihydrobenzodioxin, benzothiophene, benzothiadiazole, imidazothiazole, 2,3-dihydrobenzofuran, isoxazole, 1,2-benzisoxazole, dihydropyrazole and benzdioxanyl (such as 1,4-benzdioxanyl).

In an even further embodiment of the invention the heterocyclyl group is a group chosen among furyl, pyrrolyl, thienyl, pyridyl, N-oxido-pyridyl, pyrazinyl, pyrimidinyl, pyridazinyl, benzoxadiazole, dihydrobenzodioxin, benzothiophene, benzothiadiazole, imidazothiazole, 2,3-dihydrobenzofuran, isoxazole, 1,2-benzisoxazole or dihydropyrazole.

In one embodiment of the invention R₁ represents R₆OC(O).

In another embodiment of the invention R₁ represents R₁₆SC(O).

In yet another embodiment R₁ represents a group (gII),

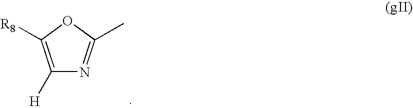

In a further embodiment of the invention R₁ is selected among R₆OC(O) and R₁₆SC(O) wherein R₆ can be methyl, ethyl, 2-hydroxyethyl, 2,2,2-trifluoroethyl, isopropyl, cyclo-propyl, iso-butyl, n-butyl, cyclo-butyl, n-propyl, tertbutyl, cyclo-pentyl, 2,2-dimethylpropyl, benzyl and 4-fluorobenzyl and wherein R₁₆ is ethyl.

R₁ may also be embodified by the group gII,

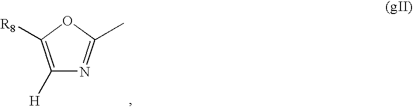

in which R₈ is selected from H, (C₁-C₆)alkyl, such as methyl or ethyl.

In another embodiment for the group R₈ this group can be chosen among hydrogen, methyl, ethyl, n-propyl and n-butyl.

Embodiments for R₂ include, for example, H and (C₁-C₄)alkyl. Other embodiments for R₂ are methyl, ethyl, iso-propyl, phenyl, methoxy, or amino unsubstituted or optionally substituted with methyl.

Embodiments for R₃ include, for example, H, methyl, methylsulfinyl, hydroxymethyl, methoxy or amino unsubstituted or optionally substituted with one or two methyl groups.

Other embodiments for R₃ include H or amino unsubstituted or optionally substituted with one or two methyl groups.

Embodiments for R₄ include H, halogen such as chloro, methyl, cyano, nitro, amino unsubstituted or optionally substituted with one or two methyl groups and further includes 4-methoxy-4-oxobutoxy, 3-carboxy-propoxy and methylcarbonyl.

In one embodiment of the invention Z is absent.

In another embodiment of the invention Z represents O.

In one embodiment R₅ represents hydrogen or methyl. In another embodiment R₅ is hydrogen.

Further embodiments for R₈ include, hydrogen, methyl and ethyl.

Further embodiments for R₁₄ include, for example, hydrogen, methyl, amino, tert-butyloxycarbonyl, tert-butyloxycarbonyl-imino, 2-carboxyethyl and 3-tert-butoxy-3-oxopropyl.

Other further embodiments for R₁₄ include, for example, hydrogen, methyl, tert-butyloxycarbonyl imino, and amino.

In one embodiment of the invention R₁₅ represents H.

Further embodiments for R^{d} includes aryl or heterocyclyl, more particularly, aryl or aromatic heterocyclyl.

Another embodiment for R^{d} include, aryl such as phenyl and aromatic heterocyclyl such as thienyl.

Other embodiments of R^{d} include phenyl which optionally may be substituted.

In a special embodiment R^{d} represents aryl, heterocyclyl or (C₃-C₆)cycloalkyl, and anyone of these groups are optionally substituted with one or more halogen (F, Cl, Br, I) atoms or mixed halogen atoms, and/or one or more of the following groups, OH, CN, NO₂, (C₁-C₁₂)alkyl, (C₁-C₁₂)alkoxyC(O), (C₁-C₁₂)alkoxy, halogen substituted (C₁-C₁₂)alkyl, (C₃-C₆)cycloalkyl, aryl, heterocyclyl, (C₁-C₁₂)alkylsulfinyl, (C₁-C₁₂)alkylsulfonyl, (C₁-C₁₂)alkylthio, (C₃-C₆)cycloalkylthio, arylsulfinyl, arylsulfonyl, arylthio, aryl(C₁-C₁₂)alkylthio, aryl(C₁-C₁₂)alkylsulfinyl, aryl(C₁-C₁₂)alkylsulfonyl, heterocyclyl(C₁-C₁₂)alklthio, heterocyclyl(C₁-C₁₂)alkylsulfinyl, heterocyclyl(C₁-C₁₂)alkylsulfonyl, (C₃-C₆)cycloalkyl(C₁-C₁₂)alkylthio, (C₃-C₆)cycloalkyl(C₁-C₁₂)alkylsulfinyl, (C₃-C₆)cycloalkyl(C₁-C₁₂)alkylsulfonyl or a group of formula NR^{a(Rd)}R^{b(Rd)} in which R^{a(Rd)} and R^{b(Rd)} independently represent H, (C₁-C₁₂)alkyl, (C₁-C₁₂)alkylC(O) or R^{a(Rd)} and R^{b(Rd)} together with the nitrogen atom represent piperidine, pyrrolidine, azetidine or aziridine;

Even further embodiments for R^{d} include phenyl optionally substituted at the 2, 3, 4 or 5-positions as well as any combination thereof. Example of substituents are cyano, tetrazol-5-yl, methoxy, trifluoromethoxy, methyl, trifluoromethyl, fluoro, chloro, bromo, methylsulfonyl, nitro, 3-methyl-5-oxo-4,5-dihydro-1H-pyrazol-1-yl. Two adjacent positions (e.g. 2,3) may also be connected to form a ring. Example of such a substituent is 2-naphtyl. Further more specific values for heteroaryls are 2-chloro-5-thienyl, 3-bromo-5-chloro-2-thienyl, 2,1,3-benzoxadiazol-4-yl, 2,4-dimethyl-1,3-thiazol-5-yl 2,3-dihydro-1,4-benzodioxin-6-yl, 5-chloro-3-methyl-1-benzothien-2-yl, 2,1,3-benzothiadiazol-4-yl, 2,5-dimethyl-3-furyl, 6-chloroimidazo[2,1-b][1,3]thiazol-5-yl, 2,3-dihydro-1-benzofuran-5-yl, 5-chloro-3-thienyl, 5-isoxazol-5-yl-2-thienyl, 5-isoxazol-3-yl-2-thienyl, 4-bromo-5-chloro-2-thienyl, 5-bromo-6-chloropyridin-3-yl, 5-bromo-2-thienyl, 5-pyridin-2-yl-2-thienyl, 2,5-dichloro-3-thienyl, 4,5-dichloro-2-thienyl, benzothien-3-yl, 2,5-dimethyl-3-thienyl, 3-thienyl, 2-thienyl, 5-methylisoxazol-4-yl, pyridin-3-yl, [1-methyl-5-(trifluoromethyl)-1H-pyrazol-3-yl]-2-thienyl, 5-chloro-1,3-dimethyl-1H-pyrazol-4-yl, 4-[(4-chlorophenyl)sulfonyl]-3-methyl-2-thienyl, 5-(methoxycarbonyl)-2-furyl and 4-(methoxycarbonyl)-5-methyl-2-furyl.

In one embodiment of the invention R^{c} represents an unsubstituted or monosubstituted or disubstituted (C₁-C₄)alkylene group wherein any substituents each individually and independently are selected from (C₁-C₄)alkylene, (C₁-C₄)alkoxyl, oxy-(C₁-C₄)allyl, (C₂-C₄)alkenyl, (C₂-C₄)alkynyl, (C₃-C₆)cycloalkyl, carboxyl, carboxy-(C₁-C₄)alkyl, aryl, heterocyclyl, nitro, cyano, halogeno (F, Cl, Br, I), hydroxyl, NR^{a(Rc)}R^{b(Rc)} in which R^{a(Rc)} and R^{b(Rc)} individually and independently from each other represents hydrogen, (C₁-C₄)alkyl or R^{a(Rc)} and R^{b(Rc)} together with the nitrogen atom represent piperidine, pyrrolidine, azetidine or aziridine, and R^{d} represents aryl, i.e. R^{c}R^{d} represents an aryl-(C₁-C₄)alkylene group with any substituents according to above.

In a preferred embodiment of the invention R^{c} represents an unsubstituted or monosubstituted or disubstituted (C₁-C₃)alkylene group wherein any substituents each individually and independently are selected from (C₁-C₄)alkyl, (C₁-C₄)alkoxyl, oxy-(C₁-C₄)allyl, (C₂-C₄)alkenyl, (C₂-C₄)alkynyl, (C₃-C₆)cycloalkyl, carboxyl, carboxy-(C₁-C₄)alkyl, aryl, heterocyclyl, nitro, cyano, halogeno (F, Cl, Br, I), hydroxyl, NR^{a(Rc)}R^{b(Rc)} in which R^{a(Rc)} and R^{b(Rc)} individually and independently from each other represents hydrogen, (C₁-C₄)alkyl or R^{a(Rc)} and R^{b(Rc)} together with the nitrogen atom represent piperidine, pyrrolidine, azetidine or aziridine, and R^{d} represents aryl, i.e. R^{c}R^{d} represents an aryl-(C₁-C₃)alkylene group with any substituents according to above.

In a further embodiment of the invention R^{c} represents an unsubstituted or monosubstituted or disubstituted (C₁-C₄)alkylene group wherein any substituents each individually and independently are selected from (C₁-C₄)alkyl, (C₁-C₄)alkoxyl, oxy-(C₁-C₄)alkyl, (C₂-C₄)alkenyl, (C₂-C₄)alkynyl, (C₃-C₆)cycloalkyl, carboxyl, carboxy-(C₁-C₄)alkyl, aryl, heterocyclyl, nitro, cyano, halogeno (F, Cl, Br, I), hydroxyl, NR^{a(Rc)}R^{b(Rc)} in which R^{a(Rc)} and R^{b(Rc)} individually and independently from each other represents hydrogen, (C₁-C₄)alkyl or R^{a(Rc)} and R^{b(Rc)} together with the nitrogen atom represent piperidine, pyrrolidine, azetidine or aziridine, and R^{d} represents heterocyclyl, i.e. R^{c}R^{d} represents a heterocyclyl-(C₁-C₄)alkylene group with any substituents according to above.

In a further preferred embodiment of the invention R^{c} represents an unsubstituted or monosubstituted or disubstituted (C₁-C₃)alkylene group wherein any substituents each individually and independently are selected from (C₁-C₄)allyl, (C₁-C₄)alkoxy, oxy-(C₁-C₄)alkyl, (C₂-C₄)alkenyl, (C₂-C₄)aryl, (C₃-C₆)cycloalkyl, carboxyl, carboxy-(C₁-C₄)allyl, aryl, heterocyclyl, nitro, cyano, halogeno (F, Cl, Br, I), hydroxyl, NR^{a(Rc)}R^{b(Rc)} in which R^{a(Rc)} and R^{b(Rc)} individually and independently from each other represents hydrogen, (C₁-C₄)alkyl or R^{a(Rc)} and R^{b(Rc)} together with the nitrogen atom represent piperidine, pyrrolidine, azetidine or aziridine, and R^{d} represents heterocyclyl, i.e. R^{c}R^{d} represents a heterocyclyl-(C₁-C₃)alkylene group with any substituents according to above.

In a particular embodiment of the invention R^{c} represents a C₁-alkylene group wherein any substituents each individually and independently are selected from (C₁-C₄)alkyl, (C₁-C₄)alkoxy, oxy-(C₁-C₄)alkyl, (C₂-C₄)alkenyl, (C₂-C₄)alkynyl, (C₃-C₆)cycloalkyl, carboxyl, carboxy-(C₁-C₄)alkyl, aryl, heterocyclyl, nitro, cyano, halogeno (F, Cl, Br, I), hydroxyl, NR^{a(Rc)}R^{b(Rc)} in which R^{a(Rc)} and R^{b(Rc)} individually and independently from each other represents hydrogen, (C₁-C₄)alkyl or R^{a(Rc)} and R^{b(Rc)} together with the nitrogen atom represent piperidine, pyrrolidine, azetidine or aziridine, and R^{d} represents aryl, i.e. R^{c}R^{d} represents an aryl-C₁-alkylene group with any substituents according to above.

In one embodiment of the invention R₁₉ represents hydrogen.

In another embodiment of the invention R₁₉ represents methyl.

In a most particular embodiment of the invention R^{c}R^{d} represents a benzyl group, or a benzyl group which is substituted according to what is described in connection to substitution of the aryl group.

In one embodiment of the invention X represents a single bond.

In another embodiment of the invention X represents imino (—NH—) or methylene (—CH₂—). In yet another embodiment X represents imino (—NH—). In a further embodiment X represents methylene (—CH₂—).

Suitable values for the B ring/ring system include, for example, diazepanylene, piperazinylene, piperidinylene, pyrrolidinylene and azetidinylene, wherein anyone of them may be presents in any of their isomeric forms (e.g. piperazin-tetrahydropyridazin-tetrahydropyrimidin).

Embodiments for the B ring/ring system include, for example, diazepanylene, piperazinylene, piperidinylene, pyrrolidinylene and azetidinylene. Further embodiments include these groups which are substituted with R₁₄ having a (C₁-C₆)alkyl group, wherein the (C₁-C₆)alkyl group optionally is substituted with OH, COOH or COOR^{e} group(s), e.g. a 2-carboxyethyl group, and wherein R^{e} represents H, aryl, cycloalkyl, heterocyclyl or (C₁-C₁₂)alkyl optionally substituted by one or more of halogen (F, Cl, Br, I) or mixed halogen atoms, OH, aryl, cycloalkyl and heterocyclyl.

In an alternative to the embodiment for the B ring/ring system above, the embodiment include, for example, diazepanylene, piperazinylene, piperidinylene, pyrrolidinylene or azetidinylene groups which are substituted with R₁₄ having a (C₁-C₆)alkyl group, wherein the (C₁-C₆)alkyl group optionally is substituted with OH, COOH or COOR^{e} group(s), e.g. a 2-carboxyethyl group, and wherein R^{e} represents H, aryl, cycloalkyl, heterocyclyl or (C₁-C₆)alkyl optionally substituted by one or more of halogen (F, Cl, Br, I) or mixed halogen atoms, OH, aryl, cycloalkyl and heterocyclyl.

A 2nd embodiment of formula I is defined by;

R₁ represents R₆OC(O), R₇C(O), R₁₆SC(O), R₁₇S, R₁₈C(S) or a group gII,

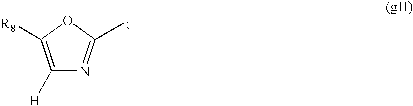

R₂ represents H, CN, NO₂, (C₁-C₆)alkyl optionally interrupted by oxygen and/or optionally substituted by OH, aryl, cycloalkyl, heterocyclyl or one or more halogen (F, Cl, Br, I) atoms; further R₂ represents (C₁-C₆)alkoxy optionally substituted by one or more halogen (F, Cl, Br, I) atoms; further R₂ represents (C₃-C₆)cycloalkyl, hydroxy(C₁-C₆)alkyl, (C₁-C₆)alkylC(O), (C₁-C₆)alkylthioC(O), (C₁-C₆)alkylC(S), (C₁-C₆)alkoxyC(O), (C₃-C₆)cycloalkoxy, aryl, arylC(O), aryl(C₁-C₆)alkylC(O), heterocyclyl, heterocyclylC(O), heterocyclyl(C₁-C₆)alkylC(O), (C₁-C₆)alkylsufinyl, (C₁-C₆)alkylsulfonyl, (C₁-C_{6)alkylthio, (C}₃-C₆)cycloalkylthio, arylsulfinyl, arylsulfonyl, arylthio, aryl(C₁-C₆)alkylthio, aryl(C₁-C₆)alkylsulfinyl, aryl(C₁-C₆)alkylsulfonyl, heterocyclyl(C₁-C₆)alkylthio, heterocyclyl(C₁-C₆)alkylsulfinyl, heterocyclyl(C₁-C₆)alkylsulfonyl, (C₃-C₆)cycloalkyl(C₁-C₆)alkylthio, (C₃-C₆)cycloalkyl(C₁-C₆)alkylsulfinyl, (C₃-C₆)cycloalkyl(C₁-C₆)alkylsulfonyl or a group of formula NR^{a(2)}R^{b(2)} in which R^{a(2)} and R^{b(2)} independently represent H, (C₁-C₆)alkyl, (C₁-C₆)alkylC(O) or R^{a(2)} and R^{b(2)} together with the nitrogen atom represent piperidine, pyrrolidine, azetidine or aziridine;

Further, R₁+R₂ together (with two carbons from the pyridine ring) may form a 5-membered or 6-membered cyclic lactone;

R₃ represents H, CN, NO₂, halogen (F, Cl, Br, I), (C₁-C₆)alkyl optionally interrupted by oxygen and/or optionally substituted by OH, aryl, cycloalkyl, heterocyclyl or one or more halogen atoms; further R₃ represents (C₁-C₆)alkoxy optionally substituted by one or more halogen (F, Cl, Br, I) atoms; further R₃ represents (C₃-C₆)cycloalkyl, hydroxy(C₁-C₆)alkyl, (C₁-C₆)alkylC(O), (C₁-C₆)alkylthioC(O), (C₁-C₆)alkylC(S), (C₁-C₆)alkoxyC(O), (C₃-C₆)cycloalkoxy, aryl, arylC(O), aryl(C₁-C₆)alkylC(O), heterocyclyl, heterocyclylC(O), heterocyclyl(C₁-C₆)alkylC(O), (C₁-C₆)alkylsulfinyl, (C₁-C₆)alkylsulfonyl, (C₁-C₆)alkylthio, (C₃-C₆)cycloalkylthio, arylsulfinyl, arylsulfonyl, arylthio, aryl(C₁-C₆)alkylthio, aryl(C₁-C₆)alkylsulfinyl, aryl(C₁-C₆)alkylsulfonyl, heterocyclyl(C₁-C₆)alkylthio, heterocyclyl(C₁-C₆)alkylsulfinyl, heterocyclyl(C₁-C₆)alkylsulfonyl, (C₃-C₆)cycloalkyl(C₁-C₆)alkylthio, (C₃-C₆)cycloalkyl(C₁-C₆)alkylsulfinyl, (C₃-C₆)cycloalkyl(C₁-C₆)alkylsulfonyl or a group of formula NR^{a(3)}R^{b(3)} in which R^{a(3)} and R^{b(3)} independently represent H, (C₁-C₆)alkyl, (C₁-C₆)alkylC(O) or R^{a(3)} and R^{b(3)} together with the nitrogen atom represent piperidine, pyrrolidine, azetidine or aziridine;

R₄ represents H, CN, NO₂, halogen (F, Cl, Br, I), (C₁-C₆)alkyl optionally interrupted by oxygen and/or optionally substituted by OH, COOH, (C₁-C₆)alkoxycarbonyl, aryl, cycloalkyl, heterocyclyl or one or more halogen atoms; further R₄ represents (C₃-C₆)cycloalkyl, hydroxy(C₁-C₆)alkyl, (C₁-C₆)alkylC(O), (C₁-C₆)alkoxy wherein the alkoxy group may optionally be substituted by one or more halogen (F, Cl, Br, I) atoms, OH and/or COOH and/or (C₁-C₃)alkoxycarbonyl; further R₄ represents (C₁-C₆)alkylthioC(O), (C₁-C₆)alkylC(S), (C₁-C₆)alkoxyC(O), (C₃-C₆)cycloalkoxy, aryl, arylC(O), aryl(C₁-C₆)alkylC(O), heterocyclyl, heterocyclylC(O), heterocyclyl(C₁-C₆)alkylC(O), (C₁-C₆)alkylsulfinyl(C₁-C₆) alkylsulfonyl, (C₁-C₆)alkylthio, (C₃-C₆)cycloalkylthio, arylsulfinyl, arylsulfonyl, arylthio, aryl(C₁-C₆)alkylthio, aryl(C₁-C₆)alkylsulfinyl, aryl(C₁-C₆)alkylsulfonyl, heterocyclyl(C₁-C₆)alkylthio, heterocyclyl(C₁-C₆)alkylsulfinyl, heterocyclyl(C₁-C₆)alkylsulfonyl, (C₃-C₆)cycloalkyl(C₁-C₆)alkylthio, (C₃-C₆)cycloalkyl(C₁-C₆)alkylsufinyl, (C₃-C₆)cycloalkyl(C₁-C₆)alkylsulfonyl or a group of formula NR^{a(4)}R^{b(4)} in which R^{a(4)} and R^{b(4)} independently represent H, (C₁-C₆)alkyl, (C₁-C₆)alkylC(O) or R^{a(4)} and R^{b(4)} together with the nitrogen atom represent piperidine, pyrrolidine, azetidine or aziridine;

Z represents O or is absent;

R₅ represents H or (C₁-C₆)allyl;

R₆ represents (C₁-C₆)alkyl optionally interrupted by oxygen, (with the proviso that any such oxygen must be at least 1 carbon atom away from the ester-oxygen connecting the R₆ group) and/or optionally substituted by OH, aryl, cycloalkyl, heterocyclyl or one or more halogen (F, Cl, Br, I) atoms; further R₆ represents (C₃-C₆)cycloalkyl, hydroxy(C₂-C₆)alkyl, aryl or heterocyclyl;

R₇ represents (C₁-C₆)alkyl optionally interrupted by oxygen, and/or optionally substituted by OH, aryl, cycloalkyl, heterocyclyl or one or more halogen (F, Cl, Br, I) atoms; further R₇ represents (C₃-C₆)cycloalkyl, hydroxy(C₁-C₆)alkyl, aryl or heterocyclyl;

R₈ represents H, (C₁-C₆)alkyl optionally interrupted by oxygen, and/or optionally substituted by aryl, cycloalkyl, heterocyclyl or one or more halogen (F, Cl, Br, I) atoms; further R₈, represents (C₃-C₆)cycloalkyl, hydroxy(C₁-C₆)alkyl, (C₁-C₆)alkoxy, (C₃-C₆)cycloalkoxy, aryl, heterocyclyl, (C₁-C₆)alkylsulfinyl, (C₁-C₆)alkylsulfonyl, (C₁-C₆)alkylthio, (C₃-C₆)cycloalkylthio, arylsulfinyl, arylsulfonyl, arylthio, aryl(C₁-C₆)alkylthio, aryl(C₁-C₆)alkylsulfinyl, aryl(C₁-C₆)alkylsulfonyl, heterocyclyl(C₁-C₆)alkylthio, heterocyclyl(C₁-C₆)alkylsulfinyl, heterocyclyl(C₁-C₆)alkylsulfonyl, (C₃-C₆)cycloalkyl(C₁-C₆)alkylthio, (C₃-C₆)cycloalkyl(C₁-C₆)alkylsulfinyl or (C₃-C₆)cycloalkyl(C₁-C₆)alkylsulfonyl;

R₁₄ represents H, OH with the proviso that the OH group must be at least 2 carbon atoms away from any heteroatom in the B ring/ring system, (C₁-C₆)alkyl optionally interrupted by oxygen and/or optionally substituted by one or more of OH, COOH and COOR^{e}; wherein R^{e} represents aryl, cycloalkyl, heterocyclyl or (C₁-C₆)alkyl optionally substituted by one or more of halogen (F, Cl, Br, I) atoms, OH, aryl, cycloalkyl and heterocyclyl; further R₁₄ represents aryl, heterocyclyl, one or more halogen F, Cl, Br, I) atoms, (C₃-C₆)cycloalkyl, hydroxy(C₁-C₆)alkyl, (C₁-C₆)alkoxy, (C₃-C₆)cycloalkoxy, aryl, heterocyclyl, (C₁-C₆)alkylsulfinyl, (C₁-C₆)alkylsulfonyl, (C₁-C₆)alkylthio, (C₃-C₆)cycloalkylthio, arylsulfinyl, arylsulfonyl, arylthio, aryl(C₁-C₆)alkylthio, aryl(C₁-C₆)alkylsulfinyl, aryl(C₁-C₆)alkylsulfonyl, heterocyclyl(C₁-C₆)alkylthio, heterocyclyl(C₁-C₆)alkylsulfinyl, heterocyclyl(C₁-C₆)alkylsulfonyl, (C₃-C₆)cycloalkyl(C₁-C₆)alkylthio, (C₃-C₆)cycloalkyl(C₁-C₆)alkylsulfinyl, (C₃-C₆)cycloalkyl(C₁-C₆)alkylsulfonyl or a group of formula NR^{a(14)}R^{b(14)} in which R^{a(14)} and R^{b(14)} independently represent H, (C₁-C₆)alkyl, (C₁-C₆)alkylC(O), (C₁-C₆)alkoxyC(O) or R^{a(14)} and R^{b(14)} together with the nitrogen atom represent piperidine, pyrrolidine, azetidine or aziridine;

R₁₅ represents H, OH with the proviso that the OH group must be at least 2 carbon atoms away from any heteroatom in the B ring/ring system, (C₁-C₆)alkyl optionally interrupted by oxygen and/or optionally substituted by one or more of OH, COOH and COOR^{e}; wherein R^{e} represents aryl, cycloalkyl, heterocyclyl or (C₁-C₆)alkyl optionally substituted by one or more of halogen (F, Cl, Br, I) atoms, OH, aryl, cycloalkyl and heterocyclyl; further R₁₅ represents aryl, heterocyclyl, one or more halogen (F, Cl, Br, I) atoms, (C₃-C₆)cycloalkyl, hydroxy(C₁-C₆)alkyl, (C₁-C₆)alkoxy, (C₃-C₆)cycloalkoxy, aryl, heterocyclyl, (C₁-C₆)alkylsulfinyl, (C₁-C₆)alkylsulfonyl, (C₁-C₆)alkylthio, (C₃-C₆)cycloalkylthio, arylsulfinyl, arylsulfonyl, arylthio, aryl(C₁-C₆)alkylthio, aryl(C₁-C₆)alkylsulfinyl, aryl(C₁-C₆)alkylsulfonyl, heterocyclyl(C₁-C₆)alkylthio, heterocyclyl(C₁-C₆)alkylsulfinyl, heterocyclyl(C₁-C₆)alkylsulfonyl, (C₃-C₆)cycloalkyl(C₁-C₆)alkylthio, (C₃-C₆)cycloalkyl(C₁-C₆)alkylsulfinyl, (C₃-C₆)cycloalkyl(C₁-C₆)alkylsulfonyl or a group of formula NR^{a(15)}R^{b(15)} in which R^{a(15)} and R^{b(15)} independently represent H, (C₁-C₆)alkyl, (C₁-C₆)alkylC(O), (C₁-C₆)alkoxyC(O) or R^{a(15)} and R^{b(15)} together with the nitrogen atom represent piperidine, pyrrolidine, azetidine or aziridine;

R₁₆ represents (C₁-C₆)alkyl optionally interrupted by oxygen and/or optionally substituted by OH, aryl, cycloalkyl, heterocyclyl or one or more halogen (F, Cl, Br, I) atoms; further R₁₆ represents (C₃-C₆)cycloalkyl, hydroxy(C₂-C₆)alkyl, (C₁-C₆)alkoxy, (C₃-C₆)cycloalkoxy, aryl, or heterocyclyl;

R₁₇ represents (C₁-C₆)alkyl optionally interrupted by oxygen and/or optionally substituted by OH, aryl, cycloalkyl, heterocyclyl or one or more halogen (F, Cl, Br, I) atoms; further R₁₇ represents (C₃-C₆)cycloalkyl, hydroxy(C₁-C₆)alkyl, (C₁-C₆)alkoxy, (C₃-C₆)cycloalkoxy, aryl or heterocyclyl;

R₁₈ represents (C₁-C₆)alkyl optionally interrupted by oxygen and/or optionally substituted by OH, aryl, cycloalkyl, heterocyclyl or one or more halogen (F, Cl, Br, I) atoms; further R₁₈ represents (C₃-C₆)cycloalkyl, hydroxy(C₁-C₆)allyl, (C₁-C₆)alkoxy, (C₃-C₆)cycloalkoxy, aryl or heterocyclyl;

R^{c} represents an unsubstituted or monosubstituted or polysubstituted (C₁-C₄)alkylene group, (C₁-C₄)oxoalkylene group, (C₁-C₄)alkyleneoxy or oxy-(C₁-C₄)alkylene group, wherein any substituents each individually and independently are selected from (C₁-C₄)alkyl, (C₁-C₄)alkoxyl, oxy-(C₁-C₄)alkyl, (C₂-C₄)alkenyl, (C₂-C₄)alkynyl, (C₃-C₆)cycloalkyl, carboxyl, carboxy-(C₁-C₄)alkyl, aryl, heterocyclyl, nitro, cyano, halogeno (F, Cl, Br, I), hydroxyl, NR^{a(Rc)}R^{b(Rc)} in which R^{a(Rc)} and R^{b(Rc)} individually and independently from each other represents hydrogen, (C₁-C₄)alkyl or R^{a(Rc)} and R^{b(Rc)} together with the nitrogen atom represent piperidine, pyrrolidine, azetidine or aziridine; Further R^{k} represents imino (—NH—), N-substituted imino (—NR₁₉—), (C₁-C₄)alkyleneimino or N-substituted (C₁-C₄)alkyleneimino (—N(R₁₉)—((C₁-C₄)alkylene) wherein the mentioned alkylene groups are unsubstituted or monosubstituted or polysubstituted with any substituents according to above; preferably R^{c} represents imino or (C₁-C₄)alkyleneimino or an unsubstituted or monosubstituted or polysubstituted (C₁-C₄)alkylene group or (C₁-C₄)oxoalkylene group with any substituents according to above;

R₁₉ represents H or (C₁-C₄)alkyl;

R^{d} represents (C₃-C₈)cycloalkyl, aryl or heterocyclyl, and anyone of these groups optionally substituted with one or more halogen (F, Cl, Br, I) atoms and/or one or more of the following groups, OH, CN, NO₂, (C₁-C₆)alkyl, (C₁-C₆)alkoxyC(O), (C₁-C₆)alkoxy, halogen substituted (C₁-C₆)alkyl, (C₃-C₆)cycloalkyl, aryl, heterocyclyl, (C₁-C₆)alkylsulfinyl, (C₁-C₆)alkylsulfonyl, (C₁-C₆)alkylthio, (C₃-C₆)cycloalkylthio, arylsulfinyl, arylsulfonyl, arylthio, aryl(C₁-C₆)alkylthio, aryl(C₁-C₆)alkylsulfinyl, aryl(C₁-C₆)alkylsulfonyl, heterocyclyl(C₁-C₆)alkylthio, heterocyclyl(C₁-C₆)alkylsulfinyl, heterocyclyl(C₁-C₆)alkylsulfonyl, (C₃-C₆)cycloalkyl(C₁-C₆)alkylthio, (C₃-C₆)cycloalkyl(C₁-C₆)alkylsulfinyl, (C₃-C₆)cycloalkyl(C₁-C₆)alkylsulfonyl or a group of formula NR^{a(Rd)}R^{b(Rd)} in which R^{a(Rd)} and R^{b(Rd)} independently represent H, (C₁-C₆)alkyl, (C₁-C₆)alkylC(O) or R^{a(Rd)} and R^{b(Rd)} together with the nitrogen atom represent piperidine, pyrrolidine, azetidine or aziridine;

X represents a single bond, imino (—NH—), methylene (—CH₂—), iminomethylene (—CH₂—NH—) wherein the carbon is connected to the B-ring/ring system, methyleneimino (—NH—CH₂—) wherein the nitrogen is connected to the B-ring/ring system and any carbon and/or nitrogen in these groups may optionally be substituted with (C₁-C₆) alkyl; further X may represent a group (—CH₂—)ₙ wherein n=2-6, which optionally is unsaturated and/or substituted by one or more substituent chosen among halogen, hydroxyl or (C₁-C₆)alkyl;

B is a monocyclic or bicyclic, 4 to 11-membered heterocyclic ring/ring system comprising one or more nitrogen and optionally one or more atoms selected from oxygen or sulphur, which nitrogen is connected to the pyridine-ring (according to formula I) and further the B-ring/ring system is connected to X in another of its positions. The substituents R₁₄ and R₁₅ are connected to the B ring/ring system in such a way that no quarternary ammonium compounds are formed (by these connections).

A 3rd embodiment of formula I is defined by;

R₁ represents R₆OC(O), R₁₆SC(O), or a group gII,

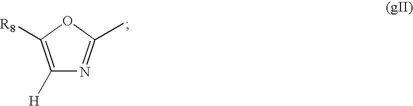

R₂ represents H, CN, NO₂, (C₁-C₆)alkyl optionally interrupted by oxygen and/or optionally substituted by OH, aryl, cycloalkyl, heterocyclyl or one or more halogen (F, Cl, Br, I) atoms; further R₂ represents (C₁-C₆)alkoxy optionally substituted by one or more halogen (F, Cl, Br, I) atoms; further P2 represents (C₃-C₆)cycloalkyl, hydroxy(C₁-C₆)alkyl, (C₁-C₆)alkylC(O), (C₁-C₆)alkylthioC(O), (C₁-C₆)alkylC(S), (C₁-C₆)alkoxyC(O), (C₃-C₆)cycloalkoxy, aryl, arylC(O), aryl(C₁-C₆)alkylC(O), heterocyclyl, heterocyclylC(O), heterocyclyl(C₁-C₆)alkylC(O) or a group of formula NR^{a(2)}R^{b(2)} in which R^{a(2)} and R^{b(2)} independently represent H, (C₁-C₆)alkyl, (C₁-C₆)alkylC(O) or R^{a(2)} and R^{b(2)} together with the nitrogen atom represent piperidine, pyrrolidine, azetidine or aziridine;

R₃ represents H, CN, NO₂, halogen (F, Cl, Br, I), (C₁-C₆)alkyl optionally interrupted by oxygen and/or optionally substituted by OH, aryl, cycloalkyl, heterocyclyl or one or more halogen atoms; further R₃ represents (C₁-C₆)alkoxy optionally substituted by one or more halogen (F, Cl, Br, I) atoms; further R₃ represents (C₃-C₆)cycloalkyl, hydroxy(C₁-C₆)alkyl, (C₁-C₆)alkylC(O), (C₁-C₆)alkylthioC(O), (C₁-C₆)alkylC(S), (C₁-C₆)alkoxyC(O), (C₃-C₆)cycloalkoxy, aryl, arylC(O), aryl(C₁-C₆)alkylC(O), heterocyclyl, heterocyclylC(O), heterocyclyl(C₁-C₆)alkylC(O), (C₁-C₆)alkylsulfinyl, or a group of formula NR^{a(3)}R^{b(3)} in which R^{a(3)} and R^{b(3)} independently represent H, (C₁-C₆)allyl, (C₁-C₆)alkylC(O) or R^{a(3)} and R^{b(3)} together with the nitrogen atom represent piperidine, pyrrolidine, azetidine or aziridine;

R₄ represents H, CN, NO₂, halogen (F, Cl, Br, I), (C₁-C₆)alkyl optionally interrupted by oxygen and/or optionally substituted by OH, COOH, aryl, cycloalkyl, heterocyclyl or one or more halogen atoms; further R₄ represents (C₃-C₆)cycloalkyl, hydroxy(C₁-C₆)alkyl, (C₁-C₆)alkylC(O), (C₁-C₆)alkoxy wherein the alkoxy group may optionally be substituted by one or more halogen (F, Cl, Br, I) atoms, OH and/or COOH and/or methoxycarbonyl; further R₄ represents (C₁-C₆)alkylthioC(O), (C₁-C₆)alkylC(S), (C₁-C₆)alkoxyC(O), (C₃-C₆)cycloalkoxy, aryl, arylC(O), aryl(C₁-C₆)alkylC(O), heterocyclyl, heterocyclylC(O), heterocyclyl(C₁-C₆)alkylC(O) or a group of formula NR^{a(4)}R^{b(4)} in which R^{a(4)} and R^{b(4)} independently represent H, (C₁-C₆)alkyl, (C₁-C₆)alkylC(O) or R^{a(4)} and R^{b(4)} together with the nitrogen atom represent piperidine, pyrrolidine, azetidine or aziridine;

Z represents O or is absent;

R₅ represents H or (C₁-C₆)alkyl;

R₆ represents (C₁-C₆)alkyl optionally interrupted by oxygen, (with the proviso that any such oxygen must be at least 1 carbon atom away from the ester-oxygen connecting the R₆ group) and/or optionally substituted by OH, aryl, cycloalkyl, heterocyclyl or one or more halogen (F, Cl, Br, I) atoms; further R₆ represents (C₃-C₆)cycloalkyl, hydroxy(C₂-C₆)alkyl, aryl or heterocyclyl;

R₈ represents H, (C₁-C₆)alkyl optionally interrupted by oxygen, and/or optionally substituted by aryl, cycloalkyl, heterocyclyl or one or more halogen (F, Cl, Br, I) atoms; further R₈ represents (C₃-C₆)cycloalkyl, hydroxy(C₁-C₆)alkyl, (C₁-C₆)alkoxy, (C₃-C₆)cycloalkoxy, aryl or heterocyclyl;

R₁₄ represents H, OH with the proviso that the OH group must be at least 2 carbon atoms away from any heteroatom in the B ring/ring system, (C₁-C₆)alkyl optionally interrupted by oxygen and/or optionally substituted by one or more of OH, COOH and COOR^{e}; wherein R^{e} represents aryl, cycloalkyl, heterocyclyl or (C₁-C₆)alkyl optionally substituted by one or more of halogen (F, Cl, Br, I) atoms, OH, aryl, cycloalkyl and heterocyclyl; ether R₁₄ represents aryl, heterocyclyl, one or more halogen (F, Cl, Br, I) atoms, (C₃-C₆)cycloalkyl, hydroxy(C₁-C₆)alkyl, (C₁-C₆)alkoxy, (C₃-C₆)cycloalkoxy, aryl, heterocyclyl or a group of formula NR^{a(14)}R^{b(14)} in which R^{a(14)} and R^{b(14)} independently represent H, (C₁-C₆)alkyl, (C₁-C₆)alkylC(O), (C₁-C₆)alkoxyC(O) or R^{a(14)} and R^{b(14)} together with the nitrogen atom represent piperidine, pyrrolidine, azetidine or aziridine;

R₁₅ represents H, OH with the proviso that the OH group must be at least 2 carbon atoms away from any heteroatom in the B ring/ring system, (C₁-C₆)alkyl optionally interrupted by oxygen and/or optionally substituted by one or more of OH, COOH and COOR^{e}; wherein R^{e} represents aryl, cycloalkyl, heterocyclyl or (C₁-C₆)alkyl optionally substituted by one or more of halogen (F, Cl, Br, I) atoms, OH, aryl, cycloalkyl and heterocyclyl; further R₁₅ represents aryl, heterocyclyl, one or more halogen (F, Cl, Br, I) atoms, (C₃-C₆)cycloalkyl, hydroxy(C₁-C₆)alkyl, (C₁-C₆)alkoxy, (C₃-C₆)cycloalkoxy, aryl, heterocyclyl or a group of formula NR^{a(15)}R^{b(15)} in which R^{a(15)} and R^{b(15)} independently represent H, (C₁-C₆)alkyl, (C₁-C₆)alkylC(O), (C₁-C₆)alkoxyC(O) or R^{a(15)} and R^{b(15)} together with the nitrogen atom represent piperidine, pyrrolidine, azetidine or aziridine;

R₁₆ is ethyl;

R^{c} represents an unsubstituted or monosubstituted or polysubstituted (C₁-C₄)alkylene group, (C₁-C₄)oxoalkylene group, (C₁-C₄)alkyleneoxy or oxy-(C₁-C₄)alkylene group, wherein any substituents each individually and independently are selected from (C₁-C₄)alkyl, (C₁-C₄)alkoxyl, oxy-(C₁-C₄)aryl, (C₂-C₄)alkenyl, (C₂-C₄)alkynyl, (C₃-C₆)cycloalkyl, carboxyl, carboxy-(C₁-C₄)alkyl, aryl, heterocyclyl, nitro, cyano, halogeno (F, Cl, Br, I), hydroxyl, NR^{a(Rc)}R^{b(Rc)} in which R^{a(Rc)} and R^{b(Rc)} individually and independently from each other represents hydrogen, (C₁-C₄)alkyl or R^{a(Rc)} and R^{b(Rc)} together with the nitrogen atom represent piperidine, pyrrolidine, azetidine or aziridine; Further R^{c} represents imino (—NH—), N-substituted imino (—NR₁₉—), (C₁-C₄)alkyleneimino or N-substituted (C₁-C₄)alkyleneimino (—N(R₁₉)—((C₁-C₄)alkylene) wherein the mentioned alkylene groups are unsubstituted or monosubstituted or polysubstituted with any substituents according to above; preferably R^{c} represents imino or (C₁-C₄)alkyleneimino or an unsubstituted or monosubstituted or polysubstituted (C₁-C₄)allylene group or (C₁-C₄)oxoalkylene group with any substituents according to above;

R₁₉ represents H or (C₁-C₄)alkyl;

R^{d} represents (C₃-C₈)cycloalkyl, aryl or heterocyclyl, and anyone of these groups optionally substituted with one or more halogen (F, Cl, Br, I) atoms and/or one or more of the following groups, CN, NO₂, (C₁-C₆)alkyl, (C₁-C₆)alkoxy, halosubstituted (C₁-C₆)alkyl, (C₃-C₆)cycloalkyl, aryl, heterocyclyl, (C₁-C₆)alkylsulfinyl, (C₁-C₆)alkylsulfonyl, (C₁-C₆)alkylthio, (C₃-C₆)cycloalkylthio, arylsulfinyl, arylsulfonyl, arylthio, aryl(C₁-C₆)alkylthio, aryl(C₁-C₆)alkylsulfinyl, aryl(C₁-C₆)alkylsulfonyl, heterocyclyl(C₁-C₆)alkylthio, heterocyclyl(C₁-C₆)alkylsulfinyl, heterocyclyl(C₁-C₆)alkylsulfonyl, (C₃-C₆)cycloalkyl(C₁-C₆)alkylthio, (C₃-C₆)cycloalkyl(C₁-C₆)alkylsulfinyl or (C₃-C₆)cycloalkyl(C₁-C₆)alkylsulfonyl;

X represents a single bond, imino (—NH—), methylene (—CH₂—), iminomethylene (—CH₂—NH—) wherein the carbon is connected to the B-ring/ring system, methyleneimino (—NH—CH₂—) wherein the nitrogen is connected to the B-ring/ring system and any carbon and/or nitrogen in these groups may optionally be substituted with (C₁-C₆) alkyl; further X may represent a group (—CH₂—)ₙ wherein n=2-6, which optionally is unsaturated and/or substituted by one or more substituent chosen among halogen, hydroxyl or (C₁-C₆)alkyl;

B is a monocyclic or bicyclic, 4 to 11-membered heterocyclic ring/ring system comprising one or more nitrogen and optionally one or more atoms selected from oxygen or sulphur, which nitrogen is connected to the pyridine-ring (according to formula I) and ether the B-ring/ring system is connected to X in another of its positions. The substituents R₁₄ and R₁₅ are connected to the B ring/ring system in such a way that no quarternary ammonium compounds are formed (by these connections).

A 4th embodiment of formula I is defined by;

R₁ represents R₆OC(O), R₁₆SC(O) or a group gII

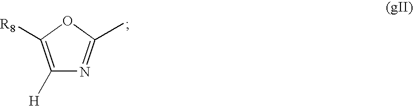

R₂ represents H or (C₁-C₆)alkyl optionally interrupted by oxygen and/or optionally substituted by OH, aryl, cycloalkyl, heterocyclyl or one or more halogen (F, Cl, Br, I) atoms; further R₂ represents a group of formula NR^{a(2)}R^{b(2)} in which R^{a(2)} and R^{b(2)} independently represent H, (C₁-C₆)alkyl, (C₁-C₆)alkylC(O) or R^{a(2)} and R^{b(2)} together with the nitrogen atom represent piperidine, pyrrolidine, azetidine or aziridine;

R₃ represents H or a group of formula NR^{a(3)}R^{b(3)} in which R^{a(3)} and R^{b(3)} independently represent H, (C₁-C₆)alkyl, (C₁-C₆)alkylC(O) or R^{a(3)} and R^{b(3)} together with the nitrogen atom represent piperidine, pyrrolidine, azetidine or aziridine;

R₄ represents CN, halogen (F, Cl, Br, I), further R₄ represents (C₁-C₆)alkylC(O), (C₁-C₆)alkoxy wherein the alkoxy group may optionally be substituted by one or more halogen F, Cl, Br, I) atoms, OH and/or COOH and/or methoxycarbonyl;

Z represents O or is absent;

R₅ represents H;

R₆ represents (C₁-C₁₂)alkyl optionally interrupted by oxygen, (with the proviso that any such oxygen must be at least 2 carbon atoms away from the ester-oxygen connecting the R₆ group) and/or optionally substituted by OH, aryl, cycloalkyl, heterocyclyl or one or more halogen (F, Cl, Br, I) atoms; further R₆ represents (C₃-C₆)cycloalkyl or hydroxy(C₂-C₁₂)alkyl;

R₈ represents H, (C₁-C₆)alkyl optionally interrupted by oxygen, and/or optionally substituted by aryl, cycloalkyl, heterocyclyl or one or more halogen (F, Cl, Br, I) atoms;

R₁₄ represents H, OH with the proviso that the OH group must be at least 2 carbon atoms away from any heteroatom in the B ring/ring system, (C₁-C₆)alkyl optionally interrupted by oxygen and/or optionally substituted by one or more of OH, COOH and COOR^{e}; wherein R^{e} represents aryl, cycloalkyl, heterocyclyl or (C₁-C₆)alkyl optionally substituted by one or more of halogen (F, Cl, Br, I) atoms, OH, aryl, cycloalkyl and heterocyclyl; further R₁₄ represents or a group of formula NR^{a(14)}R^{b(14)} in which R^{a(14)} and R^{b(14)} independently represent H, (C₁-C₆)alkyl, (C₁-C₆)alkylC(O), (C₁-C₆)alkoxyC(O) or R^{a(14)} and R^{b(14)} together with the nitrogen atom represent piperidine, pyrrolidine, azetidine or aziridine;

R₁₅ represents H;

R₁₆ is ethyl;

R^{c} represents an unsubstituted or monosubstituted (C₁-C₄)alkylene group, (C₁-C₄)alkyleneoxy or oxy-(C₁-C₄)alkylene group, wherein any substituents each individually and independently are selected from (C₁-C₄)alkyl; Further R^{c} represents imino (—NH—), N-substituted imino (—NR₁₉—);

R₁₉ represents H or methyl;

R_{d} represents (C₃-C₈)cycloalkyl, aryl or heterocyclyl, and anyone of these groups optionally substituted with one or more halogen (F, Cl, Br, I) atoms and/or one or more of the following groups, CN, NO₂, (C₁-C₆)alkyl, (C₁-C₆)alkoxy, halosubstituted (C₁-C₆)alkyl;

X represents a single bond, imino (—NH—) or methylene (—CH₂—); and

B is a monocyclic or bicyclic, 4 to 11-membered heterocyclic ring/ring system comprising one or more nitrogen and optionally one or more atoms selected from oxygen or sulphur, which nitrogen is connected to the pyridine-ring (according to formula I) and further the B-ring/ring system is connected to X in another of its positions. The substituents R₁₄ and R₁₅ are connected to the B ring/ring system in such a way that no quarternary ammonium compounds are formed (by these connections).

A 5th embodiment of formula I is defined by that;

R₁ is chosen from a group consisting of methoxycarbonyl, ethoxycarbonyl, (n-propyl)-oxycarbonyl, (iso-propyl)oxycarbonyl, (iso-butyl)oxycarbonyl, (tert-butyl)oxycarbonyl, (2,2-dimethylpropyl)-oxycarbonyl, (cyclo-propyl)oxycarbonyl, (cyclo-butyl)-oxycarbonyl, (cyclo-pentyl)-oxycarbonyl, (2-hydroxyethyl)-oxycarbonyl), (2,2,2-trifluoroethyl)-oxycarbonyl, benzyl oxycarbonyl, 4-fluorobenzyl-oxycarbonyl, ethylthiocarbonyl, and 5-ethyl-1,3-oxazol-2-yl;

R₂ is chosen from a group consisting of H, methyl, ethyl, isopropyl, and dimethylamino;

R₃ is chosen from a group consisting of H and amino;

R₄ is chosen from a group consisting of methoxy, chloro, cyano, (4-methoxy-4-oxobutoxy), (3-carboxy-propoxy) and methylcarbonyl;

Z represents O or is absent;

R₅ is H;

R₆ is chosen from a group consisting of methyl, ethyl, 2-hydroxyethyl, (2,2,2-trifluoroethyl), n-propyl, iso-propyl, cyclo-propyl, iso-butyl, tert-butyl, cyclo-butyl, 2,2-dimethylpropyl, cyclo-pentyl, benzyl and 4-fluorobenzyl;

R₈ is ethyl;

R₁₄ is chosen from a group consisting of H, methyl, tert-butyloxycarbonyl-imino and amino;

R₁₅ is H;

R₁ is ethyl;

R^{c} is chosen from a group consisting of methylene (—CH₂—), methylmethylene (—CH(CH3)-), ethylene (—CH₂CH₂—), oxypropylene (—OCH₂CH₂CH₂—), imino (—NH—) and methylimino (—N(CH₃)—;

R₁₉ is chosen from a group consisting of H and methyl;

R^{d} is chosen from a group consisting of cyclopentyl, cyclohexyl, 4-methylcyclohexyl, phenyl, 2-methylphenyl, 3-methylphenyl, 4-methylphenyl, 4-ethylphenyl, 2-methoxycarbonyl-phenyl, 3-(trifluoromethyl)phenyl, 4-(trifluoromethyl)phenyl, 2-(trifluoromethyl)phenyl, 2-fluorophenyl, 3-fluorophenyl, 4-fluorophenyl, 2-chlorophenyl, 3-chlorophenyl, 4-chlorophenyl, 3-bromophenyl, 4-cyanophenyl, 4-methoxyphenyl, 2-nitrophenyl, 3-nitrophenyl, 4-nitrophenyl, 3,4-dichlorophenyl, 3,5-dichlorophenyl, 3,4-difluorophenyl, 2,5-dimethylphenyl, 3,5-dimethylphenyl, 4-isopropylphenyl, 3-fluoro-4-methyl-phenyl, 2-pyridyl 3-pyridyl, 4-pyridyl, N-oxido-2-pyridyl, 6-[3-benzo[d]isoxazol-3-yl] and N-[(1,2-benzisoxazol-3-yl)];

X represents a single bond, imino (—NH—) or methylene (—CH₂—);

B is chosen from the group consisting of 4-piperazin-1-ylene, 4-piperidin-1-ylene, 3-piperidin-1-ylene, 3-azetidin-1-ylene, and the substituents R₁₄ and R₁₅ are connected to the B ring/ring system, in such a way that no quarternary ammonium compounds are formed (by these connections).

In a 6th embodiment of formula (I), formula (I) is defined as being any compound(s) of formula (Ia)-(Ii):

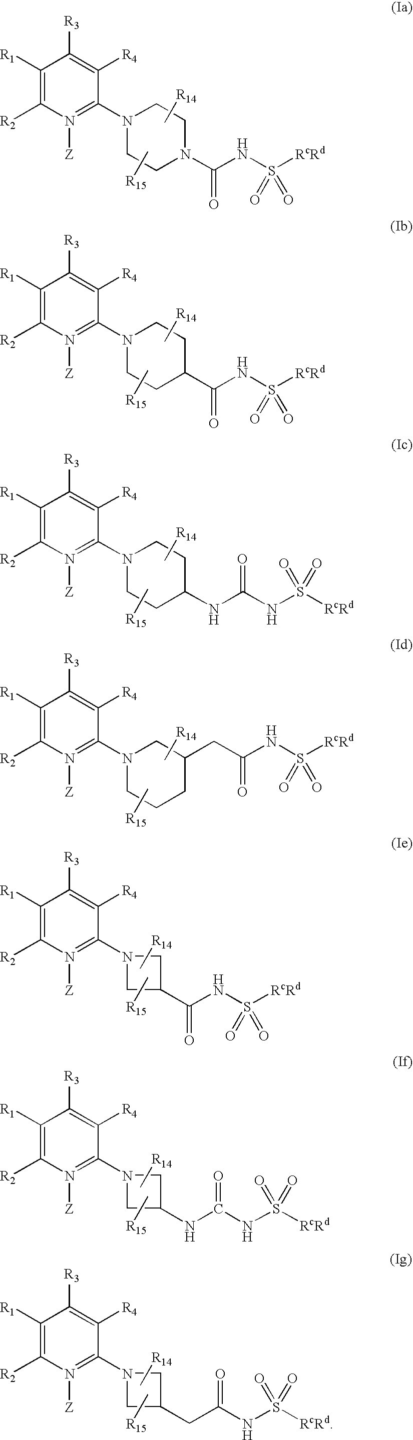

In the above Ia to Ig the various values of Z and R (except R₅ being H) are as defined above and include the previously mentioned embodiments.

In a 7^{th} embodiment formula (I) is defined as being any compound(s) of formula (Iaa)-(Ijj);

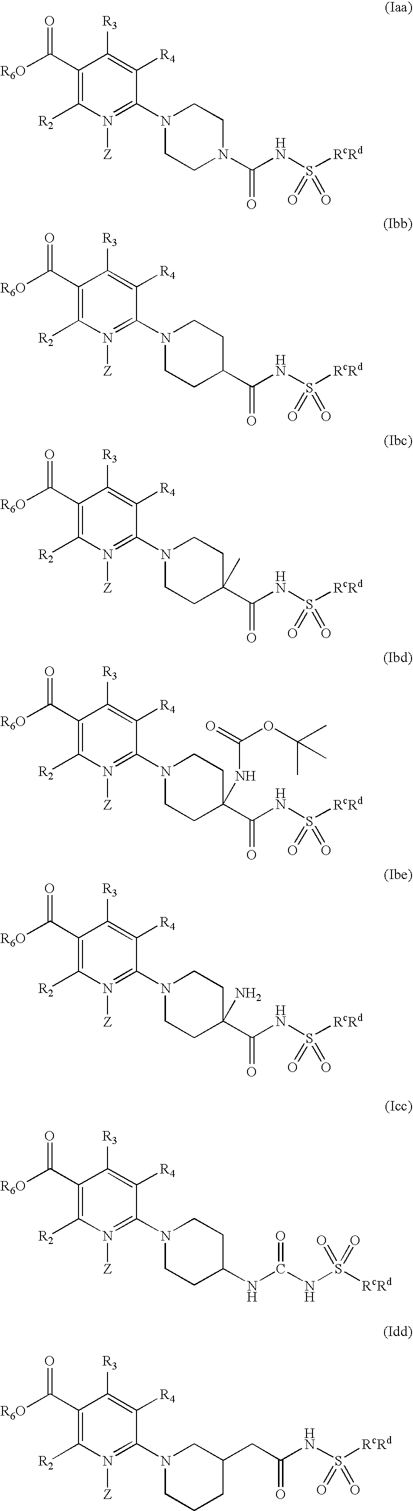

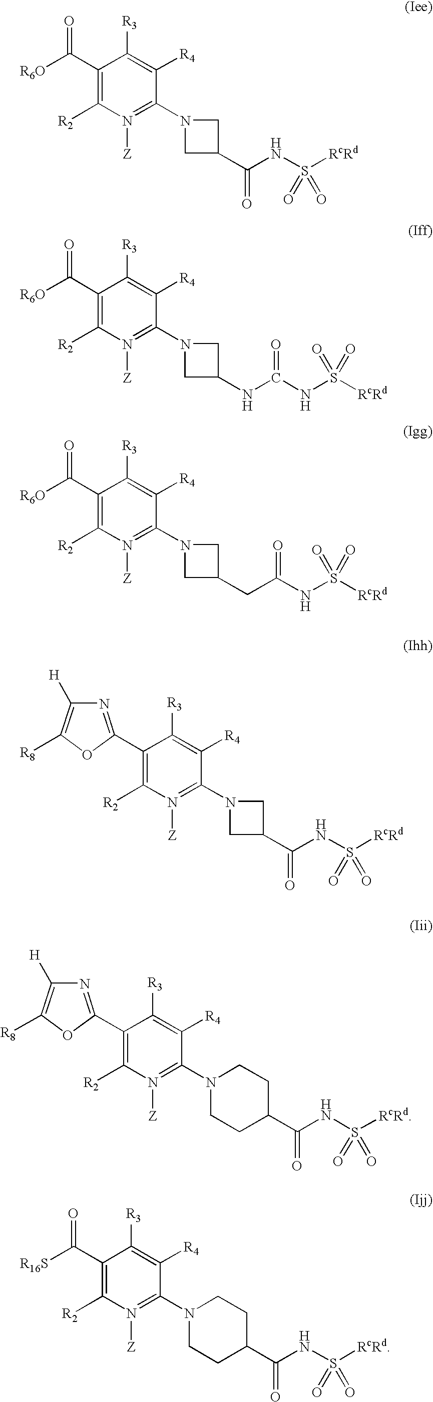

In the above Iaa to Ijj the various values of Z and R (except R₅, R₁₄ and R₁₅, all being H) are as defined above and include the previously mentioned embodiments.

Examples of specific compounds according to the invention can be selected from;
5-Cyano-6-[3-(2-methoxycarbonyl-phenylmethanesulfonylaminocarbonyl)-azetidin-1-yl]-2-methylnicotinic acid ethyl ester
6-[3-({[(3-Bromobenzyl)sulfonyl]amino}carbonyl)azetidin-1-yl]-5-cyano-2-methylnicotinic acid ethyl ester
5-Cyano-2-methyl-6-[3-(2-nitro-phenylmethanesulfonylaminocarbonyl)-azetidin-1-yl]-nicotinic acid ethyl ester
6-[3-(2-Chloro-phenylmethanesulfonylaminocarbonyl)-azetidin-1-yl]-5-cyano-2-methyl-nicotinic acid ethyl ester
6-[3-(4-Chloro-phenylmethanesulfonylaminocarbonyl)-azetidin-1-yl]-5-cyano-2-methyl-nicotinic acid ethyl ester
5-Cyano-2-methyl-6-[3-(4-trifluoromethyl-phenylmethanesulfonylaminocarbonyl)-azetidin-1-yl]-nicotinic acid ethyl ester
5-Cyano-6-[3-(3-fluoro-phenylmethanesulfonylaminocarbonyl)-azetidin-1-yl]-2-methyl-nicotinic acid ethyl ester
5-Cyano-2-methyl-6-[3-(3-trifluoromethyl-phenylmethanesulfonylaminocarbonyl)-azetidin-1-yl]-nicotinic acid ethyl ester
6-[3-(3-Chloro-phenylmethanesulfonylaminocarbonyl)-azetidin-1-yl]-5-cyano-2-methyl nicotinic acid ethyl ester
6-{3-[2-(3-Chloro-phenyl)-ethanesulfonylaminocarbonyl]-azetidin-1-yl}-5-cyano-2-methyl-nicotinic acid ethyl ester
5-Cyano-2-methyl-6-[3-(4-nitro-phenylmethanesulfonylaminocarbonyl)-azetidin-1-yl]-nicotinic acid ethyl ester
5-Cyano-2-methyl-6-[3-(2-phenyl-ethanesulfonylaminocarbonyl)-azetidin-1-yl]-nicotinic acid ethyl ester
5-Cyano-2-methyl-6-(3-o-toylynethanesulfonylaminocarbonyl-azetidin-1-yl)-nicotinic acid ethyl ester
5-Cyano-2-methyl-6-[3-(3-nitro-phenylmethanesulfonylaminocarbonyl)-azetidin-1-yl]-nicotinic acid ethyl ester
5-Cyano-6-{3-[2-(4-fluoro-phenyl)-ethanesulfonylaminocarbonyl]-azetidin-1-yl}-2-methyl-nicotinic acid ethyl ester
5-Cyano-2-methyl-6-[3-(2-trifluoromethyl-phenylmethanesulfonylaminocarbonyl)-azetidin-1-yl]-nicotinic acid ethyl ester
5-Cyano-6-[3-(4-fluoro-phenylmethanesulfonylaminocabonyl)-azetidin-1-yl]-2-methyl nicotinic acid ethyl ester
5-Cyano-6-(3-cyclopentylmethanesulfonylaminocarbonyl-azetidin 1-yl)-2-methyl-nicotinic acid ethyl ester
5-Cyano-6-{3-[2-(2-fluoro-phenyl)-ethanesulfonylaminocarbonyl]-azetidin-1-yl}-2-methyl nicotinic acid ethyl ester
5-Cyano-6-[3-(3,5-dichloro-phenylmethanesulfonylaminocarbonyl)-azetidin-1-yl]-2-methyl-nicotinic acid ethyl ester
5-Cyano-6-(3-cyclohexylmethanesulfonylaminocarbonyl-azetidin-1-yl)-2-methyl-nicotinic acid ethyl ester
5-Cyano-6-{3-[2-(3-fluoro-phenyl)-ethanesulfonylaminocarbonyl]-azetidin-1-yl}-2-methyl-nicotinic acid ethyl ester
6-[3-(Benzo[d]isoxazol-3-ylmethanesulfonylaminocarbonyl)-azetidin-1-yl]-5-cyano-2-methyl-nicotinic acid ethyl ester
1-[4-Amino-3-chloro-5-(5-ethyl-1,3-oxazol-2-yl)pyridin-2-yl]-N-(benzylsulfonyl)piperidine-4-carboxamide
4-Amino-6-(4-{[(benzylsulfonyl)amino]carbonyl}piperidin-1-yl)-5-chloronicotic acid ethyl ester
6-[3-({[(Benzylsulfonyl)amino]carbonyl}amino)azetidin-1-yl]-5-cyano-2-methylnicotinic acid isopropyl ester
6-[3-({[(Benzylsulfonyl)amino]carbonyl}amino)azetidin 1-yl]-5-cyano-2-methylnicotinic acid tert-butyl ester
6-[3-({[(Benzylsulfonyl)amino]carbonyl}amino)azetidin-1-yl]-5-cyano-2-methylnicotinic acid ethyl ester
6-(3-{2-[(Benzylsulfonyl)amino]-2-oxoethyl}piperidin-1-yl)-5-cyano-2-methylnicotinic acid ethyl ester
6-(4 {[(Benzylsulfonyl)amino]carbonyl}-4-methylpiperidin-1-yl)-5-cyano-2-methylnicotinic acid ethyl ester
N-(Benzylsulfonyl)-1-[3-chloro-5-(5-ethyl-1,3-oxazol-2-yl)pyridin-2-yl]piperidine-4-carboxamide
6-(3-{[(Benzylsulfonyl)amino]carbonyl}azetidin-1-yl)-5-cyano-2-methylnicotinic acid cyclopentyl ester
6-(4-{[(Benzylsulfonyl)amino]carbonyl}piperidin-1-yl)-5-cyano-2-methylnicotinic acid propyl ester
6-(4-{[(Benzylsulfonyl)amino]carbonyl}piperidin-1-yl)-5-cyano-2-isopropylnicotinic acid ethyl ester
6-(4-{[(Benzylsulfonyl)amino]carbonyl}piperidin-1-yl)-5-cyano-2-ethylnicotinic acid ethyl ester
6-(3-{[(Benzylsulfonyl)amino]carbonyl}azetidin-1-yl)-5-cyano-2-methylnicotinic acid 2,2-dimethylpropyl ester
N-(Benzylsulfonyl)-1-[3-cyano-5-(5-ethyl 1,3-oxazol-2-yl)-6-methylpyridin-2-yl]piperidine-4-carboxamide
6-(3-{[(Benzylsulfonyl)amino]carbonyl}azetidin 1-yl)-5-cyano-2-methylnicotinic acid isopropyl ester
6-(4-{[(Benzylsulfonyl)amino]carbonyl}piperidin-1-yl)-5-cyano-2-methylnicotinic acid isopropyl ester
5-Cyano-6-[4-({[(4-cyanobenzyl)sulfonyl]amino}carbonyl)piperidin-1-yl]-2-methylnicotinic acid ethyl ester
6-[4-({[(4-Chlorobenzyl)sulfonyl]amino}carbonyl)piperidin-1-yl]-5-cyano-2-methylnicotinic acid ethyl ester
6-(4-{[(Benzylsulfonyl)amino]carbonyl}piperidin-1-yl)-5-cyano-2-methylnicotinic acid ethyl ester
N-[(1,2-Benzisoxazol-3-ylmethyl)sulfonyl]-1-[3-cyano-5-(5-ethyl-1,3-oxazol-2-yl)-6-methylpyridin-2-yl]piperidine-4-carboxamide
N-(Benzylsulfonyl)-1-[3-cyano-5-(5-ethyl-1,3-oxazol-2-yl)-6-methylpyridin-2-yl]azetidine-3-carboxamide
N-[(4-Chlorobenzyl)sulfonyl]-1-[3-cyano-5-(5-ethyl-1,3-oxazol-2-yl)-6-methylpyridin-2-yl]piperidine-4-carboxamide
5-Cyano-2-methyl-6-(3-phenylmethanesulfonylaminocarbonyl-azetidin-1-yl)-nicotinic acid ethyl ester
ethyl 5-cyano-6-{3-[({[3-(4-methoxyphenoxy)propyl]sulfonyl}amino)carbonyl]azetidin-1-yl}-2-methylnicotinate
ethyl 4-amino-6-(3-{[(benzylsulfonyl)amino]carbonyl}azetidin-1-yl)-5-chloronicotinate
ethyl 5-cyano-2-methyl-6-[3-({[(3-methylbenzyl)sulfonyl]amino}carbonyl)azetidin-1-yl]nicotinate
2,2-dimethylpropyl 6-(4-{[(benzylsulfonyl)amino]carbonyl}piperidin-1-yl)-5-cyano-2-methylnicotinate
ethyl 5-cyano-2-methyl-6-[3-({[(4-methylbenzyl)sulfonyl]amino}carbonyl)azetidin 1-yl]nicotinate
ethyl 5-cyano-6-[4-({[(4-fluorobenzyl)sulfonyl]amino) carbonyl)piperidin-1-yl]-2-methylnicotinate
ethyl 6-[4-{([(3-bromobenzyl)sulfonyl]amino}carbonyl)piperidin-1-yl]-5-cyano-2-methylnicotinate
cyclopropyl 6-(3-{[(benzylsulfonyl)amino]carbonyl}azetidin-1-yl)-5-cyano-2-methylnicotinate
2,2,2-trifluoroethyl 6-(4-{[(benzylsulfonyl)amino]carbonyl}piperidin-1-yl)-5-cyano-2-methylnicotinate
2,2,2-trifluoroethyl 6-(3-{[(benzylsulfonyl)amino]carbonyl}azetidin-1-yl)-5-cyano-2-methylnicotinate
2,2,2-trifluoroethyl 6-[3-({[(4-chlorobenzyl)sulfonyl]amino}carbonyl)azetidin-1-yl]-5-cyano-2-methylnicotinate
cyclopropyl 6-(4-{[(benzylsulfonyl)amino]carbonyl}piperidin 1-yl)-5-cyano-2-methylnicotinate
cyclobutyl 6-(3-{[(benzylsulfonyl)amino]carbonyl}azetidin-1-yl)-5-cyano-2-methylnicotinate
2-hydroxyethyl 6-(3-{[(benzylsulfonyl)amino]carbonyl}azetidin 1-yl)-5-cyano-2-methylnicotinate
benzyl 6-(3-{[(benzylsulfonyl)amino]carbonyl}azetidin-1-yl)-5-cyano-2-methylnicotinate
isopropyl 5-cyano-6-[4-({[(3,4-dichlorobenzyl)sulfonyl]amino}carbonyl)piperidin-1-yl]-2-methylnicotinate
ethyl 5-cyano-6-[3-({[(3,4-dichlorobenzyl)sulfonyl]amino}carbonyl)azetidin-1-yl]-2-methylnicotinate
ethyl 5-cyano-6-[4-({[(3,4-dichlorobenzyl)sulfonyl]amino}carbonyl)piperidin 1-yl]-2-methylnicotinate
isopropyl 5-cyano-6-[4-({[(4-cyanobenzyl)sulfonyl]amino}carbonyl)piperidin-1-yl]-2-methylnicotinate
ethyl 5-cyano-6-[3-({[(4-cyanobenzyl)sulfonyl]amino}carbonyl)azetidin-1-yl]-2-methylnicotinate
isopropyl 5-cyano-6-[4-({[(4-fluorobenzyl)sulfonyl]amino}carbonyl)piperidin-1-yl]-2-methylnicotinate
isopropyl 6-[4-({[(4-chlorobenzyl)sulfonyl]amino}carbonyl)piperidin 1-yl]-5-cyano-2-methylnicotinate
ethyl 6-(3-{[(benzylsulfonyl)amino]carbonyl}azetidin-1-yl) 5-cyano-2-isopropylnicotinate
ethyl 6-(3-{[(benzylsulfonyl)amino]carbonyl}azetidin 1-yl)-5-cyano-2-ethylnicotinate
ethyl 5-cyano-2-methyl-6-[3-({[(1-phenylethyl)sulfonyl]amino}carbonyl)azetidin-1-yl]nicotinate
propyl 6-(3-{[(benzylsulfonyl)amino]carbonyl}azetidin-1-yl)-5-cyano-2-methylnicotinate
isobutyl 6-(3-{[(benzylsulfonyl)amino]carbonyl}azetidin-1-yl)-5-cyano-2-methylnicotinate
isopropyl 5-cyano-2-methyl-6-{4-[({[4-(trifluoromethyl)benzyl]sulfonyl}amino)carbonyl]piperidin-1-yl}nicotinate
isopropyl 5-cyano-2-methyl-6-[4-({[(4-methylbenzyl)sulfonyl]amino}carbonyl)piperidin-1-yl]nicotinate
isopropyl 5-cyano-2-methyl-6-[4-({[(3-methylbenzyl)sulfonyl]amino}carbonyl)piperidin-1-yl]nicotinate
isopropyl 5-cyano-6-[4-({[(3-fluorobenzyl)sulfonyl]amino}carbonyl)piperidin 1-yl]-2-methylnicotinate
isopropyl 5-cyano-6-[4-({([(2-fluorobenzyl)sulfonyl]amino}carbonyl)piperidin-1-yl]-2-methylnicotinate
isopropyl 6-[4-({[(3-chlorobenzyl)sulfonyl]amino}carbonyl)piperidin-1-yl]-5-cyano-2-methylnicotinate
isopropyl 6-[4-({[(2-chlorobenzyl)sulfonyl]amino}carbonyl)piperidin-1-yl]-5-cyano-2-methylnicotinate
ethyl 5-cyano-2-methyl-6-[4-({[(4-methylbenzyl)sulfonyl]amino}carbonyl)piperidin 1-yl]nicotinate
ethyl 5-cyano-6-{4-[({[2-(methoxycarbonyl)benzyl]sulfonyl}amino)carbonyl]piperidin-1-yl}-2-methylnicotinate
ethyl 5-cyano-6-[4-({[(3-fluorobenzyl)sulfonyl]amino}carbonyl)piperidin-1-yl]-2-methylnicotinate
isopropyl 5-cyano-2-methyl-6-(4-[({[2-(2-methylphenyl)ethyl]sulfonyl}amino)carbonyl]piperidin-1-yl)nicotinate
ethyl 6-(4 {[(benzylsulfonyl)amino]carbonyl}piperidin-1-yl)-5-(4-methoxy-4-oxobutoxy)-2-methylnicotinate
4-{[2-(4-{[(benzylsulfonyl)amino]carbonyl}piperidin-1-yl)-5-(ethoxycarbonyl)-6-methylpyridin-3-yl]oxy}butanoic acid
ethyl 6-(3-{[(benzylsulfonyl)amino]carbonyl}azetidin-1-yl)-5-(4-methoxy-4-oxobutoxy)-2-methylnicotinate
ethyl 6-(4-{[(aminosulfonyl)amino]carbonyl}piperidin-1-yl)-5-cyano-2-methylnicotinate
ethyl 5-cyano-2-methyl-6-{4-[({[methyl(phenyl)amino]sulfonyl}amino)carbonyl]piperidin-1-yl}nicotinate
isopropyl 5-cyano-2-methyl-6-[3-({[(4-methylbenzyl)sulfonyl]amino}carbonyl)azetidin-1-yl]nicotinate
isopropyl 5-cyano-6-[3-({[(3-fluorobenzyl)sulfonyl]amino}carbonyl)azetidin-1-yl]-2-methylnicotinate
isopropyl 5-cyano-2-methyl-6-[3-({[(2-phenylethyl)sulfonyl]amino}carbonyl)azetidin-1-yl]nicotinate
isopropyl 5-cyano-6-[3-({[(cyclopentylmethyl)sulfonyl]amino) carbonyl)azetidin-1-yl]-2-methylnicotinate
isopropyl 5-cyano-6-{3-[({[2-(methoxycarbonyl)benzyl]sulfonyl}amino)carbonyl]azetidin-1-yl}-2-methylnicotinate
isopropyl 5-cyano-6-[3-({[(2-fluorobenzyl)sulfonyl]amino}carbonyl)azetidin-1-yl]-2-methylnicotinate
isopropyl 6-[3-({[(4-chlorobenzyl)sulfonyl]amino}carbonyl)azetidin-1-yl]-5-cyano-2-methylnicotinate
isopropyl 5-cyano-6-[3-({[(4-fluorobenzyl)sulfonyl]amino) carbonyl)azetidin-1-yl]-2-methylnicotinate
isopropyl 5-cyano-6-[3-([[(4-cyanobenzyl)sulfonyl]amino}carbonyl)azetidin-1-yl]-2-methylnicotinate
methyl 6-(4-{[(benzylsulfonyl)amino]carbonyl}piperidin 1-yl)-5-cyano-2-methylnicotinate
methyl 5-cyano-2-methyl-6 [4-({[(4-methylbenzyl)sulfonyl]amino}carbonyl)piperidin-1-yl]nicotinate
S-ethyl 6-(4 {[(benzylsulfonyl)amino]carbonyl}piperidin-1-yl)-5-cyano-2-methylpyridine-3-carbothioate
S-ethyl 5-cyano-2-methyl-6-[4-({[(4-methylbenzyl)sulfonyl]amino}carbonyl)piperidin-1-yl]pyridine-3-carbothioate
S-ethyl 6-[4-({[(4-chlorobenzyl)sulfonyl]amino}carbonyl)piperidin 1-yl]-5-cyano-2-methylpyridine-3-carbothioate
S-ethyl 5-cyano-6-[4-({[(4-fluorobenzyl)sulfonyl]amino}carbonyl)piperidin-1-yl]-2-methylpyridine-3-carbothioate
ethyl 6-(3-{[(benzylsulfonyl)amino]carbonyl}azetidin-1-yl)-5-methoxy-2-methylnicotinate
ethyl 6-[4-({[(benzylsulfonyl)amino]carbonyl}amino)piperidin-1-yl]-5-cyano-2-methylnicotinate
ethyl 6-({[(benzylsulfonyl)amino]carbonyl}piperazin 1-yl)-5-cyano-2-methylnicotinate
4-{[2-(3-{[(benzylsulfonyl)amino]carbonyl}azetidin-1-yl)-5-(ethoxycarbonyl)-6-methylpyridin-3-yl]oxy}butanoic acid
ethyl 5-cyano-2-methyl-6-{3-[({[(1-oxidopyridin-2-yl)methyl]sulfonyl}amino)carbonyl]azetidin-1-yl}nicotinate
ethyl 5-cyano-2-methyl-6-[3-({[(pyridin-3-ylmethyl)sulfonyl]amino}carbonyl)azetidin-1-yl]nicotinate
ethyl 5-cyano-2-methyl-6-{4-[({[(1-oxidopyridin-2-yl)methyl]sulfonyl}amino)carbonyl]piperidin 1-yl}nicotinate
ethyl 5-cyano-2-methyl-6-[4-({[(pyridin-3-ylmethyl)sulfonyl]amino}carbonyl)piperidin-1-yl]nicotinate
ethyl 6-(4-{[(benzylsulfonyl)amino]carbonyl}piperidin 1-yl)-5-cyano-2-(dimethylamino)nicotinate
ethyl 5-cyano-2-methyl-6-[4-({[(pyridin-4-ylmethyl)sulfonyl]amino}carbonyl)piperidin-1-yl]nicotinate
ethyl 5-cyano-2-methyl-6-[3-({[(pyridin-2-ylmethyl)sulfonyl]amino}carbonyl)azetidin-1-yl]nicotinate
ethyl 5-cyano-6-[3-({[(3,5-dimethylbenzyl)sulfonyl]amino}carbonyl)azetidin-1-yl]-2-methylnicotinate
isopropyl 5-cyano-6-[4-({[(cyclopentylmethyl)sulfonyl]amino}carbonyl)piperidin-1-yl]-2-methylnicotinate
ethyl 5-cyano-6-[4-({[(2,5-dimethylbenzyl)sulfonyl]amino}carbonyl)piperidin-1-yl]-2-methylnicotinate
ethyl 5-cyano-6-[4-({[(4-isopropylbenzyl)sulfonyl]amino}carbonyl)piperidin-1-yl]-2-methylnicotinate
benzyl 6-(4-{1-[(benzylsulfonyl)amino]carbonyl}piperidin-1-yl)-5-cyano-2-methylnicotinate
ethyl 5-cyano-2-methyl-6-{4-[({[(4-methylcyclohexyl)methyl]sulfonyl}amino)carbonyl]piperidin-1-yl}nicotinate
ethyl 5-cyano-6-[3-({[(4-isopropylbenzyl)sulfonyl]amino}carbonyl)azetidin 1-yl]-2-methylnicotinate
ethyl 5-cyano-2-methyl-6-[4-({[(2-phenylethyl)sulfonyl]amino}carbonyl)piperidin-1-yl]nicotinate
ethyl 5-cyano-2-methyl-6-[4-({[(pyridin-2-ylmethyl)sulfonyl]amino}carbonyl)piperidin-1-yl]nicotinate
ethyl 5-cyano-6-[3-({[(2,5-dimethylbenzyl)sulfonyl]amino}carbonyl)azetidin-1-yl]-2-methylnicotinate
ethyl 6-(3-{[(benzylsulfonyl)amino]carbonyl}azetidin-1-yl)-5-chloro-2-methylnicotinate
ethyl 6-(3-{2-[(benzylsulfonyl)amino]-2-oxoethyl}azetidin-1-yl)-S-cyano-2-methylnicotinate
ethyl 5-cyano-6-[4-({[(cyclopentylmethyl)sulfonyl]amino}carbonyl)piperidin 1-yl]-2-methylnicotinate
ethyl 5-cyano-6-[3-(2-{[(4-fluorobenzyl)sulfonyl]amino}-2-oxoethyl)azetidin-1-yl]-2-methylnicotinate
ethyl 5-cyano-6-[4-({[(3-fluoro-4-methylbenzyl)sulfonyl]amino}carbonyl)piperidin-1-yl]-2-methylnicotinate
ethyl 6-(4-{[(benzylsulfonyl)amino]carbonyl}piperidin-1-yl)-5-chloro-2-methylnicotinate
4-fluorobenzyl 6-(4-{[(benzylsulfonyl)amino]carbonyl}piperidin 1-yl)-5-cyano-2-methylnicotinate
ethyl 5-cyano-6-[4-({[(4-ethylbenzyl)sulfonyl]amino}carbonyl)piperidin-1-yl]-2-methylnicotinate
ethyl 5-cyano-6-[3-({[(3,4-difluorobenzyl)sulfonyl]amino}carbonyl)azetidin-1-yl]-2-methylnicotinate
ethyl 5-cyano-6-[4-({[(4-methoxybenzyl)sulfonyl]amino}carbonyl)piperidin-1-yl]-2-methylnicotinate
ethyl 5-cyano-2-methyl-6-[4-({[(3-methylbenzyl)sulfonyl]amino}carbonyl)piperidin-1-yl]nicotinate
ethyl 5-cyano-6-[3-({[(4-ethylbenzyl)sulfonyl]amino}carbonyl)azetidin 1-yl]-2-methylnicotinate
ethyl 5-chloro-2-methyl-6-[3-({[(4-methylbenzyl)sulfonyl]amino}carbonyl)azetidin-1-yl]nicotinate
ethyl 5-cyano-6-[4-({[(3,4-difluorobenzyl)sulfonyl]amino}carbonyl)piperidin-1-yl]-2-methylnicotinate
ethyl 5-cyano-6-[3-({[(4-methoxybenzyl)sulfonyl]amino}carbonyl)azetidin-1-yl]-2-methylnicotinate
cyclopropyl 5-cyano-2-methyl-[4-({[(4-methylbenzyl)sulfonyl]amino}carbonyl)piperidin 1-yl]nicotinate
ethyl 5-cyano-2-methyl-6-[3-({[(pyridin-4-ylmethyl)sulfonyl]amino}carbonyl)azetidin-1-yl]nicotinate
ethyl 6-(3-{[(benzylsulfonyl)amino]carbonyl}azetidin-1-yl) 5-cyano-2-(dimethylamino)nicotinate
ethyl 6-(4-{[(benzylsulfonyl)amino]carbonyl}piperidin 1-yl) 5-cyano-2-methylnicotinate 1-oxide
ethyl 5-acetyl-6-(4-{[(benzylsulfonyl)amino]carbonyl)piperidin-1-yl)-2-methylnicotinate
ethyl 6-{4-{[(benzylsulfonyl)amino]carbonyl}-4-[(tert-butoxycarbonyl)amino]piperidin-1-yl}-5-cyano-2-methylnicotinate
ethyl 6-(4-amino-4-{[(benzylsulfonyl)amino]carbonyl}piperidin-1-yl)-5-cyano-2-methylnicotinate
ethyl 6-(4-{[(benzylsulfonyl)amino]carbonyl}piperidin 1-yl)-5-chloro-2-(difluoromethyl)nicotinate
ethyl 6-(4-{[(benzylsulfonyl)amino]carbonyl}piperidin-1-yl) 5-cyano-2-(difluoromethyl)nicotinate
ethyl 6-(4-{[(benzylsulfonyl)amino]carbonyl}piperidin-1-yl)-5-cyano-2-(trifluoromethyl)nicotinate
ethyl 6-(3-{[(benzylsulfonyl)amino]carbonyl}azetidin-1-yl)-5-cyano-2-(difluoromethyl)nicotinate
ethyl 6-(3-{[(benzylsulfonyl)amino]carbonyl}azetidin-1-yl)-5-cyano-2-(trifluoromethyl)nicotinate
ethyl 6-(4-{[(benzylsulfonyl)amino]carbonyl}piperidin-1-yl)-5-cyano-2-(fluoromethyl)nicotinate
ethyl 6-(3-{[(benzylsulfonyl)amino]carbonyl}azetidin 1-yl)-5-cyano-2-(fluoromethyl)nicotinate
ethyl 5-cyano-2-(difluoromethyl)-6-{4-[({[(4-methylcyclohexyl)methyl]sulfonyl}amino)carbonyl]piperidin-1-yl}nicotinate
ethyl 5-cyano-2-(difluoromethyl)-6-[3-({[(2-fluorobenzyl)sulfonyl]amino}carbonyl)azetidin-1-yl]nicotinate
ethyl 5-cyano-2-(difluoromethyl)-6-[4-({[(2-fluorobenzyl)sulfonyl]amino}carbonyl)piperidin-1-yl]nicotinate
ethyl 5-cyano-2-(difluoromethyl)-6-[4-({[(3-fluorobenzyl)sulfonyl]amino}carbonyl)piperidin-1-yl]nicotinate
ethyl 5-cyano-2-(difluoromethyl)-6-[4-({[(4-fluorobenzyl)sulfonyl]amino}carbonyl)piperidin-1-yl]nicotinate
ethyl 6-[4-({[(2-chlorobenzyl)sulfonyl]amino}carbonyl)piperidin-1-yl]-5-cyano-2-(difluoromethyl)nicotinate
ethyl 6-[4-({[(3-chlorobenzyl)sulfonyl]amino}carbonyl)piperidin-1-yl]-5-cyano-2-(difluoromethyl)nicotinate
ethyl 6-[4-({[(4-chlorobenzyl)sulfonyl]amino}carbonyl)piperidin-1-yl]-5-cyano-2-(difluoromethyl)nicotinate
ethyl 5-cyano-2-(difluoromethyl) 6-[4-({[(3-methylbenzyl)sulfonyl]amino}carbonyl)piperidin-1-yl]nicotinate
ethyl 5-cyano-2-(difluoromethyl)-6-[4-({[(4-methylbenzyl)sulfonyl]amino}carbonyl)piperidin-1-yl]nicotinate
ethyl 5-cyano-6-[4-({[(2,4-dichlorobenzyl)sulfonyl]amino}carbonyl)piperidin-1-yl]-2-(difluoromethyl)nicotinate
ethyl 5-cyano-2-(difluoromethyl)-6-[3-({[(3-fluorobenzyl)sulfonyl]amino}carbonyl)azetidin-1-yl]nicotinate
ethyl 5-cyano-2-(difluoromethyl)-6-[3-({[(4-fluorobenzyl)sulfonyl]amino}carbonyl)azetidin 1-yl]nicotinate
ethyl 6-[3-({[(2-chlorobenzyl)sulfonyl]amino}carbonyl)azetidin-1-yl]-5-cyano-2-(difluoromethyl)nicotinate
ethyl 6-[3-({[(3-chlorobenzyl)sulfonyl]amino}carbonyl)azetidin-1-yl]-5-cyano-2-(difluoromethyl)nicotinate
ethyl 6-[3-({[(4-chlorobenzyl)sulfonyl]amino}carbonyl)azetidin-1-yl]-5-cyano-2-(difluoromethyl)nicotinate
ethyl 5-cyano-2-(difluoromethyl)-6-[3-({[(3-methylbenzyl)sulfonyl]amino}carbonyl)azetidin-1-yl]nicotinate
ethyl 5-cyano-2-(difluoromethyl)-6-[3-({[(4-methylbenzyl)sulfonyl]amino}carbonyl)azetidin-1-yl]nicotinate
ethyl 5-cyano-6-[3-({[(2,4-dichlorobenzyl)sulfonyl]amino}carbonyl)azetidin-1-yl]-2-(difluoromethyl)nicotinate
ethyl 5-cyano-2-(difluoromethyl)-6-{3-[({[(4-methylcyclohexyl)methyl]sulfonyl}amino)carbonyl]azetidin-1-yl}nicotinate
ethyl 5-cyano-6-[3-({[(3-cyanophenyl)sulfonyl]amino}carbonyl)azetidin 1-yl]-2-(difluoromethyl)nicotinate
ethyl 5-cyano-6-[3-({[(4-cyanophenyl)sulfonyl]amino}carbonyl)azetidin-1-yl]-2-(difluoromethyl)nicotinate
ethyl 5-cyano-2-(difluoromethyl)-6-{3-[({[4-(trifluoromethoxy)phenyl]sulfonyl}amino)carbonyl]azetidin-1-yl}nicotinate
ethyl 5-cyano-2-(difluoromethyl-6-{3-[({[2-(trifluoromethoxy)phenyl]sulfonyl}amino)carbonyl]azetidin-1-yl}nicotinate
ethyl 5-cyano-6-[3-({[(2-cyanobenzyl)sulfonyl]amino}carbonyl)azetidin 1-yl]-2-(difluoromethyl)nicotinate
ethyl 5-cyano-2-(difluoromethyl)-6-(3-{[(2-naphthylsulfonyl)amino]carbonyl}azetidin-1-yl)nicotinate
ethyl 6-(3-{[(butylsulfonyl)amino]carbonyl}azetidin-1-yl)-5-cyano-2-(difluoromethyl)nicotinate
ethyl 5-cyano-6-[4-({[(3-cyanophenyl)sulfonyl]amino}carbonyl)piperidin-1-yl]-2-(difluoromethyl)nicotinate
ethyl 5-cyano-6-[4-({[(4-cyanophenyl)sulfonyl]amino}carbonyl)piperidin-1-yl]-2-(difluoromethyl)nicotinate
ethyl 5-cyano-2-(difluoromethyl)-6-{4-[({[4-(trifluoromethoxy)phenyl]sulfonyl}amino)carbonyl]piperidin-1-yl}nicotinate
ethyl 5-cyano-2-(difluoromethyl)-6-{4-[({[2-(trifluoromethoxy)phenyl]sulfonyl}amino)carbonyl]piperidin-1-yl}nicotinate
ethyl 5-cyano-6-[4-({[(2-cyanobenzyl)sulfonyl]amino}carbonyl)piperidin-1-yl]-2-(difluoromethyl)nicotinate
ethyl 5-cyano-2-(difluoromethyl)-6-(4-{[(2-naphthylsulfonyl)amino]carbonyl}piperidin-1-yl)nicotinate
ethyl 6-(4-{[(butylsulfonyl)amino]carbonyl}piperidin-1-yl)-5-cyano-2-(difluoromethyl)nicotinate
ethyl 6-(3-{2-[(benzylsulfonyl)amino]-2-oxoethyl}pyrrolidin-1-yl)-5-cyano-2-(trifluoromethyl)nicotinate
ethyl 5-cyano-6-[3-(2-oxo-2-{[(2-phenylethyl)sulfonyl]amino}ethyl)pyrrolidin 1-yl]-2-(trifluoromethyl)nicotinate
ethyl 6-[3-(2-{[(5-chloro-2-thienyl)sulfonyl]amino}-2-oxoethyl)pyrrolidin-1-yl]-5-cyano-2-(trifluoromethyl)nicotinate
ethyl 5-cyano-6-[3-({[(4-fluorobenzyl)sulfonyl]amino}carbonyl)azetidin-1-yl]-2-(trifluoromethyl)nicotinate
ethyl 5-cyano-6-[3-({[(3-fluorobenzyl)sulfonyl]amino}carbonyl)azetidin-1-yl]-2-(trifluoromethyl)nicotinate
ethyl 5-cyano-6-[3-({[(2-fluorobenzyl)sulfonyl]amino}carbonyl)azetidin-1-yl]-2-(trifluoromethyl)nicotinate
ethyl 5-cyano-6-[3-({[(4-methylbenzyl)sulfonyl]amino}carbonyl)azetidin-1-yl]-2-(trifluoromethyl)nicotinate
ethyl 5-cyano-6-[3-({[(3-methylbenzyl)sulfonyl]amino}carbonyl)azetidin 1-yl]-2-(trifluoromethyl)nicotinate
ethyl 6-[3-({[(4-chlorobenzyl)sulfonyl]amino}carbonyl)azetidin-1-yl]-5-cyano-2-(trifluoromethyl)nicotinate
ethyl 6-[3-({[(2-chlorobenzyl)sulfonyl]amino}cabonyl)azetidin-1-yl]-5-cyano-2-(trifluoromethyl)nicotinate
ethyl 6-[3-({[(3-chlorobenzyl)sulfonyl]amino}carbonyl)azetidin 1-yl]-5-cyano-2-(trifluoromethyl)nicotinate
ethyl 5-cyano-6-[3-({[(2,4-dichlorobenzyl)sulfonyl]amino}carbonyl)azetidin-1-yl]-2-(trifluoromethyl)nicotinate
ethyl 6-[3-({[(5-chloro-2-thienyl)sulfonyl]amino}carbonyl)azetidin-1-yl]-5-cyano-2-(trifluoromethyl)nicotinate
ethyl 5-cyano-6-[4-({[(4-fluorobenzyl)sulfonyl]amino}carbonyl)piperidin-1-yl]-2-(trifluoromethyl)nicotinate
ethyl 5-cyano-6-[4-({[(3-fluorobenzyl)sulfonyl]amino}carbonyl)piperidin-1-yl]-2-(trifluoromethyl)nicotinate
ethyl 5-cyano-6-[4-({[(2-fluorobenzyl)sulfonyl]amino}carbonyl)piperidin-1-yl]-2-(trifluoromethyl)nicotinate
ethyl 5-cyano-6-[4-({[(4-methylbenzyl)sulfonyl]amino}carbonyl)piperidin-1-yl]-2-(trifluoromethyl)nicotinate
ethyl 5-cyano-6-[4-({[(3-methylbenzyl)sulfonyl]amino}carbonyl)piperidin-1-yl]-2-(trifluoromethyl)nicotinate
ethyl 6-[4-({[(4-chlorobenzyl)sulfonyl]amino}carbonyl)piperidin-1-yl]-5-cyano-2-(trifluoromethyl)nicotinate
ethyl 6-[4-({[(2-chlorobenzyl)sulfonyl]amino}carbonyl)piperidin 1-yl]-5-cyano-2-(trifluoromethyl)nicotinate
ethyl 6-[4-({[(3-chlorobenzyl)sulfonyl]amino}carbonyl)piperidin-1-yl]-5-cyano-2-(trifluoromethyl)nicotinate
ethyl 5-cyano-6-[4-({[(2,4-dichlorobenzyl)sulfonyl]amino}carbonyl)piperidin-1-yl]-2-(trifluoromethyl)nicotinate
ethyl 6-[4-({[(5-chloro-2-thienyl)sulfonyl]amino)}carbonyl)piperidin-1-yl]-5-cyano-2-(trifluoromethyl)nicotinate
ethyl 5-cyano-6-[3-({[(2-fluorobenzyl)sulfonyl]amino}carbonyl)azetidin-1-yl]-2-(fluoromethyl)nicotinate
ethyl 5-cyano-6-[3-({[(3-fluorobenzyl)sulfonyl]amino}carbonyl)azetidin 1-yl]-2-(fluoromethyl)nicotinate
ethyl 5-cyano-6-[3-({[(4-fluorobenzyl)sulfonyl]amino}carbonyl)azetidin-1-yl]-2-(fluoromethyl)nicotinate
ethyl 6-[3-({[(2-chlorobenzyl)sulfonyl]amino}carbonyl)azetidin-1-yl]-5-cyano-2-(fluoromethyl)nicotinate
ethyl 6-[3-({[(3-chlorobenzyl)sulfonyl]amino}carbonyl)azetidin 1-yl]-5-cyano-2-(fluoromethyl)nicotinate
ethyl 6-[3-({[(4-chlorobenzyl)sulfonyl]amino}carbonyl)azetidin-1-yl]-5-cyano-2-(fluoromethyl)nicotinate
ethyl 5-cyano-2-(fluoromethyl)-6-[3-({[(3-methylbenzyl)sulfonyl]amino}carbonyl)azetidin-1-yl]nicotinate
ethyl 5-cyano-2-(fluoromethyl)-6-[3-({[(4-methylbenzyl)sulfonyl]amino}carbonyl)azetidin-1-yl]nicotinate
ethyl 5-cyano-6-[3-({[(2,4-dichlorobenzyl)sulfonyl]amino}carbonyl)azetidin-1-yl]-2-(fluoromethyl)nicotinate
ethyl 5-cyano-2-(fluoromethyl)-6-{3-[({[(4-methylcyclohexyl)methyl]sulfonyl}amino)carbonyl]azetidin-1-yl}nicotinate
ethyl 5-cyano-6-[4-({[(2-fluorobenzyl)sulfonyl]amino}carbonyl)piperidin-1-yl]-2-(fluoromethyl)nicotinate
ethyl 5-cyano-6-[4-({[(3-fluorobenzyl)sulfonyl]amino}carbonyl)piperidin-1-yl]-2-(fluoromethyl)nicotinate
ethyl 5-cyano-6-[4-({[(4-fluorobenzyl)sulfonyl]amino}carbonyl)piperidin-1-yl]-2-(fluoromethyl)nicotinate
ethyl 6-[4-({[(2-chlorobenzyl)sulfonyl]amino}carbonyl)piperidin-1-yl]-5-cyano-2-(fluoromethyl)nicotinate
ethyl 6-[4-({[(3-chlorobenzyl)sulfonyl]amino}cabonyl)piperidin-1-yl]-5-cyano-2-(fluoromethyl)nicotinate
ethyl 6-[4-({[(4-chlorobenzyl)sulfonyl]amino}carbonyl)piperidin-1-yl]-5-cyano-2-(fluoromethyl)nicotinate
ethyl 5-cyano-2-(fluoromethyl)-6-[4-({[(3-methylbenzyl)sulfonyl]amino}carbonyl)piperidin 1-yl]nicotinate
ethyl 5-cyano-2-(fluoromethyl)-6-[4-({[(4-methylbenzyl)sulfonyl]amino}carbonyl)piperidin 1-yl]nicotinate
ethyl 5-cyano-6-[4-({[(2,4-dichlorobenzyl)sulfonyl]amino}carbonyl)piperidin-1-yl]-2-(fluoromethyl)nicotinate
ethyl 5-cyano-2-(fluoromethyl)-6-{4-[({[(4-methylcyclohexyl)methyl]sulfonyl}amino)carbonyl]piperidin-1-yl}nicotinate
ethyl 6-(3-{2-[(benzylsulfonyl)amino]2-oxoethyl}azetidin-1-yl)-5-cyano-2-(difluoromethyl)nicotinate;
   and pharmaceutically acceptable salts thereof.

Processes

The following processes together with the intermediates are provided as a further feature of the present invention.

Compounds of formula (I) may be prepared by the following processes a1-a8;

a1) Compounds of formula (I) in which R₁, R₂, R₃, R₄, B, R₅, R₁₄, R₁₅, Z, R^{c} and R^{d} are defined as above, X is a single bond or a carbon, can be formed by reacting a compound of formula (II), in which R₁, R₂, R₃, R₄, B, Z, R₁₄, and R₅ are defined

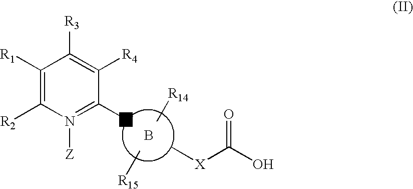

as above, X is a single bond or a carbon, with a compound of formula (III) in which R₅, R^{c} and R^{d} are defined as above.

R₅—NHSO₂—R^{c}R^{d} (III)

The reaction is generally carried out in an inert organic solvent such as dichloromethane at ambient temperature. The reaction may be carried out using standard conditions or in the presence of TBTU, EDCI or the combination of EDCI and HOBT. Optionally, the reaction may be carried out in the presence of an organic base such as triethylamine or DIPEA.

a2) Compounds of formula (I) in which R₁, R₂, R₃, R^{d}, B, R₅, R₁₄, R₁₅, Z, R^{c} and R^{d} are defined as above, X is a nitrogen or a single bond connected to a nitrogen which is a member of the B ring, can be formed by reacting a compound of formula (IV), in which R₁, R₂, R₃, R₄, R₁₄, and R₁₅ are defined as above and X is a nitrogen or a hydrogen, with a compound of the general

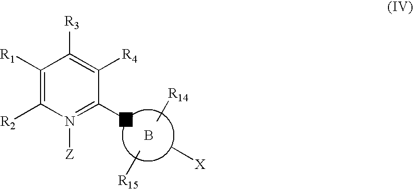

formula (III) which is defined as above.
The reaction is generally carried out in an inert solvent such as DCM. The reaction may be carried out in the presence of CDI. Optionally, the reaction may be carried out in the presence of an organic base such as triethylamine or DIPEA.

a3) Compounds of formula (I) in which R₁, R₂, R₃, R₄, B, R₁₄, R₁₅, Z, R^{c} and R^{d} are defined as above, R₅ is a hydrogen, X is a nitrogen or a single bond connected to a nitrogen which is a member of the B ring, can be formed by reacting a compound of formula (IV) which is defined in a2) above, with a compound of formula (V)

O═C═N—SO₂—R^{c}R^{d} (V)

in which R^{c} and R^{d} is as defined above.

The reaction is generally carried out in an inert solvent such as THF. Optionally, the reaction may be carried out in the presence of an organic base such as triethylamine or DIPEA.

a4) Compounds of formula (I) in which R₁, R₂, R₃, R₄, B, R₅, R₁₄, R₁₅, Z, R^{c} and R^{d} are defined as above, X is a nitrogen or a single bond connected to a nitrogen which is a member of the B ring, can be formed by reacting a compound of formula (IV) which is defined in above, with a compound of formula (VI),

R^{d}R^{c}—SONR₅—COOCH₂CCl₃ (VI)

in which R₅, R^{c} and R^{d} are as defined above. The reaction is generally carried out in a solvent such as DMA. Optionally, the reaction may be carried out in the presence of an organic base such as triethylamine or DIPEA.

a5) Compounds of formula (I) may also be prepared by reacting a compound of formula (VII) in which R₁, R₂, R₃, R₄ and Z are defined as above and L is a suitable leaving group, such as chloro, bromo, iodo, fluoro, triflate or tosyl,

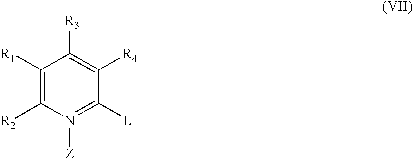

with a compound of the general formula (VIII) in which B, R₅, R₁₄, R₁₅, R^{c} and R^{d} are defined as in formula (I).

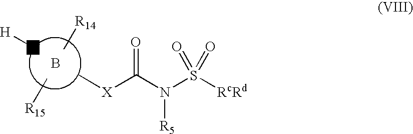

The reaction is generally carried out in an inert solvent such as DMA. Optionally, the reaction may be carried out in the presence of an organic base such as triethylamine or DIPEA.

The reaction is generally carried out at elevated temperatures using standard equipment or in a single-node microwave oven.

Generally, using the zwitterion of (VIII) when R₅ is H, leads to shorter reaction times than when using the corresponding salt of the B-ring amine, e.g. HCl salt.

For some compounds, it is advantageous to carry out the reaction in ethanol in the presence of an organic base such as triethylamine.

a6) Compounds of formula (I) where R₁ represents R₆OC(O) and R₂, R₃, R₄, B, R₅, R₁₄, R₁₅, X, Z, R^{c} and R^{d} are defined as for formula (I), can be transesterified using standard procedures or by reacting with R_{6′}—O⁻Li⁺ reagent, to become another compound of the general formula (I) wherein R₁ becomes R_{6′}OC(O).

a7) A compound of formula (I) in which R₁, R₂, R₃, R₄, B, R₅, R₁₄, R₁₅, Z and R^{d} are defined as above and R^{c} represents imino (—NH—) or (C₁-C₄)alkylimino in which the imino group could be substituted using standard conditions or using an alkylating agent like L-R₁₉, in which R₁₉ is defined as above and L is a leaving group exemplified by chloro, bromo, iodo, triflate or tosyl, to give compounds of formula (I) in which R₁, R₂, R₃, R₄, B, R₅, R₁₄, R₁₅, Z and R^{d} are defined as above and R^{c} represents N-substituted imino (—NR₁₉—) or N-substituted (C₁-C₄)alkylimino (—N(R₁₉)—((C₁-C₄)alkyl), optionally in the presence of a strong base such as NaH.

a8) Certain compounds of formula (I) in which R₂, R₃, R₄, B, R₁₄, R₁₅, R^{c} and R^{d} are defined as above, R₁ is R₆OC(O) wherein R₆ is defined as above, X is a single bond, Z is absent and R₅ is hydrogen, are advantageously prepared by the following steps (a8:1-a8:5);

a8:1) Reacting a compound of the formula R₁CH₂C(O)R₂, with dimethoxy-N,N-dimethylmethaneamine to form a compound of the formula

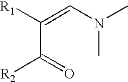

a8:2) This compound is further reacted with a compound of the general formula R₄—CH₂C(O)NH₂, in which R₄ is defined as for formula (I) to give a compound of the general formula

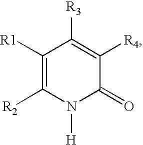

in which R₂, R₃, R₄ are defined as for formula (I), R₁ is R₆OC(O), wherein R₆ is defined as above and Z is absent The reaction is generally performed in an inert solvent such as ethanol. This reaction is performed in the presence of a strong base such as sodium ethoxide. The process is further advantageously performed by washing the final product with an alkaline water solution, e.g. a sodium bicarbonate solution.

a8:3) The compound from a8:2) is reacted with a chlorinating agent such as thionyl chloride to give a compound of formula (VII) wherein L is a chlorine. A further improvement of this reaction is to add dimethylformamide. Advantageously the reaction is performed in an inert solvent such as toluene.

a8:4) Compounds of the general formula (VIII) in which B, R₁₄, R₁₅, R^{c} and R^{d} are defined as above, X is a single bond and R₅ is a hydrogen, are formed by reacting a compound of formula (X) with a compound of formula (III), in which the ring nitrogen is protected, for example by t-butyloxycarbonyl. The reaction is generally carried out in an inert organic solvent such as THF. The reaction is carried out using a coupling reagent such as TBTU. Optionally, the reaction is carried out in the presence of an organic base such as triethylamine or DIPEA. A further improvement of this reaction is to add LiCl. When the product contains a t-butyloxycarbonyl this group is removed using standard procedures or in the presence of formic acid. In one advantageous embodiment of the process (a8) the product is isolated as a zwitterion by adjusting the pH of the reaction mixture to between approximately 5-9 with ammonia dissolved in water.

a8:5) The product from a8:3 is reacted with the product from a8:4, preferentially the zwitterion, to give a compound of formula (I) in which R₂, R₃, R₄, B, R₁₄, R₁₅, R^{c} and R^{d} are defined as above, R₁ is R₆OC(O) wherein R₆ is defined as above, X is a single bond, Z is absent and R₅ are hydrogen. The reaction is generally carried out in an inert solvent such as ethanol at elevated temperatures. Optionally, the reaction is carried out in the presence of an organic base such as triethylamine. In one advantageous embodiment of the process (a8) the final product is purified and isolated by recrystallisation from ethyl acetate.

Thus, in one embodiment of the invention, an advantageous process for manufacturing a compound of formula (I) in which R₂, R₃, R₄, R₆, B, R₁₄, R₁₅, R^{c} and R^{d} are defined according to above, R₁ is R₆OC(O) wherein R₆ is defined as above, X is a single bond, Z is absent and R₅ is hydrogen exists, characterised in that the process comprises the following steps (i-vi);

i.) Reacting a compound of the formula R₁CH₂C(O)R₂, with dimethoxy-N,N-dimethylmethaneamine to form a compound of the formula

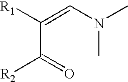

ii.) Reacting the compound from step i.) with a compound of the general formula R₄—CH₂C(O)NH₂ in an inert solvent such as ethanol in the presence of a strong base such as sodium ethoxide, to give a compound of the general formula

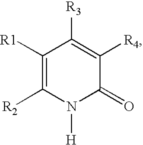

in which R₂, R₃, R^{a}, are defined according to above, R₁ is R₆OC(O) wherein R₆ is defined according to above, and Z is absent.

iii) The product from step ii) is first washed with an alkaline water solution, e.g. a sodium bicarbonate solution and then washed with water whereafter the washed product is collected.

iv.) The compound from step iii) is reacted with a chlorinating agent such as thionyl chloride in an inert solvent, to give a compound of formula (VII) wherein L is a chlorine.

v.) reacting a compound of formula (X) with a compound of formula (III), in which B, R₁₄, R₁₅, R^{c} and R^{d} are defined according to above, X is a single bond and R₅ is a hydrogen, while the compound of formula (III) is having the ring nitrogen protected by t-butyloxycarbonyl, in an inert organic solvent, in the presence of a coupling reagent and optionally an organic base such as triethylamine or DIPEA, to give a compound of the general formula (VIII) after standard deprotection of the t-butyloxycarbonyl.

vi) The product from step v.) is reacted with the product from step iv.) in an inert solvent, optionally in the presence of an organic base such as triethylamine, to give a compound of formula (I) in which R₂, R₃, R₄, B, R₁₄, R₁₅, R^{c} and R^{d} are defined according to above, R₁ is R₆OC(O) and R₆ is defined according to above, X is a single bond, Z is absent and R₅ is hydrogen.

In a separate embodiment of the process step iv.) comprises adding dimethylformamide to the reaction mixture.

In another separate embodiment of the advantageous process the process step iv.) comprises adding dimethylformamide to the reaction mixture and the inert solvent in step iv.) is toluene.

In another separate embodiment of the advantageous process it is possible to combine one or more of the previous process embodiments with selecting the inert organic solvent in step v.) to be THF.

In a further separate embodiment of the process it is possible to combine one or more of the previous process embodiments with selecting that the coupling reagent in step v.) is TBTU.

In a further separate embodiment of the advantageous process it is possible to combine one or more of the previous process embodiments with adding LiCl to the reaction mixture in step v.).

In an even further separate embodiment of the advantageous process it is possible to combine one or more of the previous process embodiments with isolating the product obtained in step v.) by adding ammonia dissolved in water.

In an even further separate embodiment of the advantageous process it is possible to combine any of the previous process embodiments with purifying and isolating the product from step vi) by recrystallisation from ethyl acetate.

The intermediates referred to above may be prepared by, for example, the methods/processes outlined below.

b) The compounds of formula (II) in which R₁, R₂, R₃, R₄, B, Z, R₁₄, and R₁₅ are defined as above, X is a single bond or a carbon, may be prepared by reacting a compound of formula (IX)

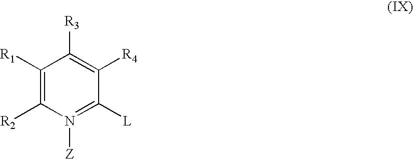

in which R₁, R₂, R₃, R₄ and Z are defined as for formula (I) above and L is a suitable leaving group (such as fluoro, chloro, bromo, iodo, triflate or tosyl), with a compound of the general formula (X),

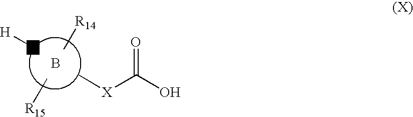

in which B, R₁₄, R₁₅ are defined as above and X is a single bond or a carbon.

The reaction is generally carried out at elevated temperatures using standard equipment or in a single-node microwave oven. The reaction can be carried out in an inert solvent such as ethanol, DMA or a mixture of solvents such as ethanol water. Optionally the reaction may be carried out in the presence of an organic base such as TEA or DIPEA.

c) Compounds of formula (IV) which are defined as above may be prepared by reacting the corresponding compound of formula (IX) which is defined above, with a compound of formula (XI) in which B, R₁₄, R₁₅ are defined as above, X is a nitrogen or a single bond connected to a nitrogen which is a member of the B ring.

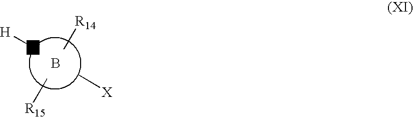

The reaction is generally carried out at elevated temperatures using standard equipment or in a single-node microwave oven. The reaction can be carried out in an inert solvent such as ethanol, DMA or a mixture of solvents such as ethanol water. Optionally the reaction may be carried out in the presence of an organic base such as TEA or DIPEA.

d) Synthesis of compounds of the general formula (XXX),

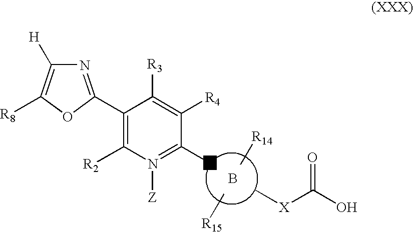

in which R₂, R₃, R₄, B, R₈, R₁₄ and R₁₅ are defined as above and X is a carbon or a single bond comprises the below steps. (d1-d5)

d1) Reacting the corresponding compounds of the general formula (X) which is defined as above with a compound of the general formula (XXI)

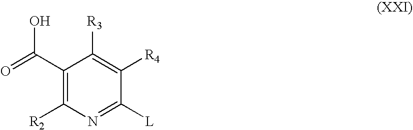

in which R₂, R₃ and R₄ are defined as for formula I, and L is a suitable leaving group, such as chloro, bromo, iodo, triflate or tosyl, to give a compound of formula (XXII).

The reactions are carried out at elevated temperatures using standard equipment or a single-node microwave oven. Optionally the reaction may be carried out in the presence of an organic base such as TEA or DIPEA.

d2) The compounds of formula (XXII) can then be reacted

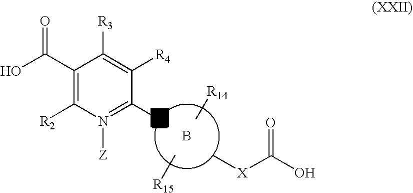

with a compound of the general formula (XXIII),

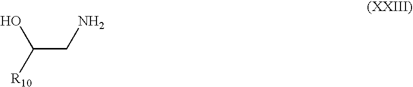

in which R₁₀ is defined as above, to give compounds of the general formula (XXIV). The reactions are carried out using standard conditions or in the presence of EDCI or the combination of EDCI and HOBT. Optionally the reaction may be carried out in the presence of an organic base such as TEA or DIPEA.

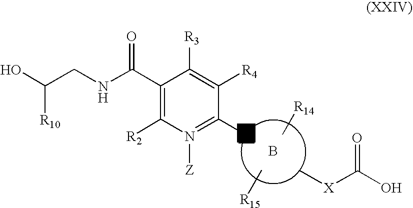

d3) This compound (XXIV) can then be transformed to a compound of the general formula (XX)

d4) The preparation of compounds with the general formula (XX),

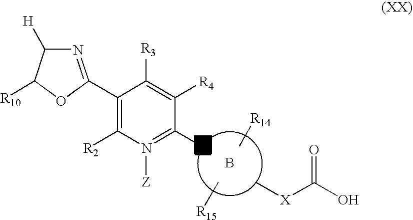

in which R₂, R₃, R₄, B, R₁₀, R₁₄ and R₁₅ are defined as above and X is a carbon or a single bond using known methods or a known reagent such as methanesulfonyl chloride. Optionally the reaction may be carried out in the presence of an organic base such as TEA.

d5) can be made by oxidising the corresponding compound of the general formula (XX) wherein R₁₀ is the same substituent as to R₈ using a known oxidation reagent such as DDQ.

e) The preparation of compounds of the general formula (XXX) also comprises the steps (e1-e4) below;

e1) Reacting a compound the general formula (XXXI),

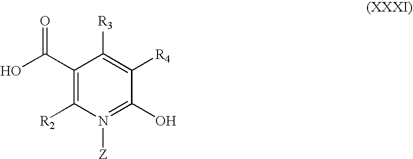

in which R₂, R₃ and R₄ are defined as for compound (I) above, with a compound of the general formula (XXXII), in which R₈ is defined as above,

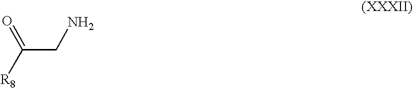

using standard conditions or in the presence of EDCI or the combination of EDCI and HOBT. Optionally the reaction may be carried out in the presence of an organic base such as TEA. This reaction gives a compound of the general formula (XXXIII).

e2) The compound of the general formula (XXXIII) obtained

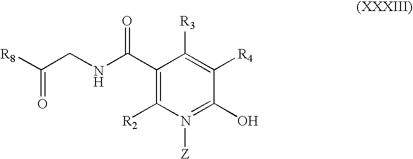

can then be transformed to a compound of the general formula (XXXIV), in which R₂, R₃, R₄ and R₅ are defined as above, using known techniques or using a known reagent such as POCl₃.

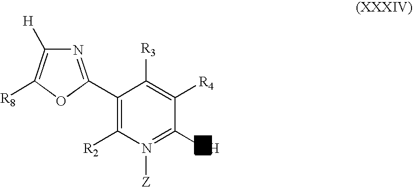

e3) A compound of the general formula (XXXIV) can then be transformed to a compound of the general formula (XXXV),

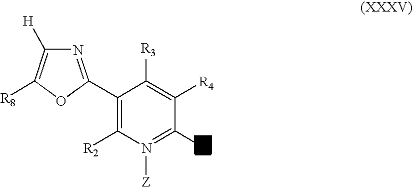

in which R₂, R₃, R₄, R₈ are defined as above and L is a sufficient leaving group, such as chloro, bromo, iodo, triflate or tosyl, using a known techniques or a reagent such as oxalyl chloride or thionyl chloride.

e4) The compound of formula (XXXV) can then be reacted with a compound of the general formula (X), which is defined as above, to give a compound of the general formula (XXX), defined as above. The reactions are carried out at elevated temperatures using standard equipment or a single-node microwave oven. Optionally the reactions may be carried out in the presence of an organic base such as TEA or DIPEA.

f) Preparation of Compounds of the general formula (XXVI),

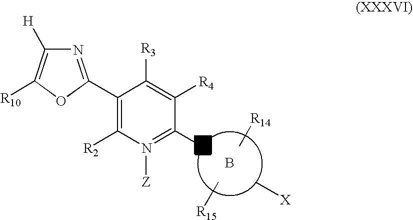

in which R₂, R₃, R₄, B, R₁₀, R₁₄ and R₁₅ are defined as above, X is a nitrogen or a single bond connected to a nitrogen which is a member of the B ring, comprises the below steps. (f1-f4)

f1) Reacting a compound of the general formula (XI) which is defined as above with a compound of the general formula (XXI) which is defined as above, to give a compound of the general formula (XXVIII).

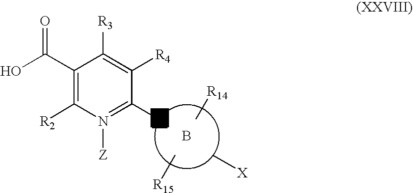

The reactions are carried out at elevated temperatures using standard equipment or a single-node microwave oven. Optionally the reaction may be carried out in the presence of an organic base such as TEA or DIPEA.

f2) The compound of formula (XXVIII) can be reacted with a compound of formula (XXIII), which is defined as above, to give compounds of the general formula (XXIX). The reactions are carried out using standard conditions or in the presence of EDCI or the combination of EDCI and HOBT. Optionally the reactions may be carried out in the presence of an organic base such as TEA or DIPEA.

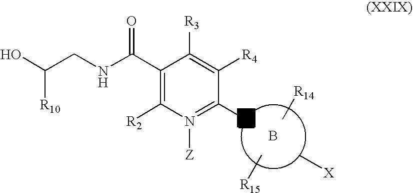

f3 This compound can then be transformed to a compound of the general formula (XXVI) in which R₂, R₃, R₄, B, R₁₀, R₁₄ and R₁₅, are defined as above,

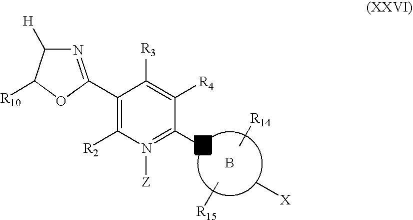

X is a nitrogen or a hydrogen connected to a nitrogen which is a member of the B ring, using known methods or a sufficient reagent such as methanesulfonyl chloride. Optionally the reaction may be carried out in the presence of an organic base such as TEA.

f4) (XXXVI) can then prepared by oxidising a compound of the general formula (XXVI), which is defined as above. The reaction can be performed using standard conditions or a reagent like DDQ.

Compounds of the general formula (II), in which R₁ is R₇C(O), R₂, R₃, R₄, B, R₁₄ and R₁₅ are defined as above, X is a single bond comprises the following steps (g1-g2):

g1) Reacting a compound of the general formula (XXII), described above, with N,O-dimethylhydroxylamine. The reaction can be performed using known reagents like CDI to give a compound of the general formula (XXXVIII).

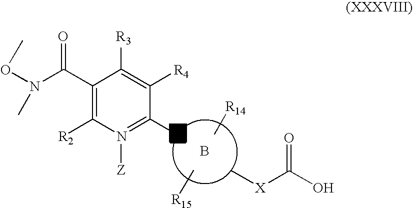

g2) Reacting compounds of the general formula (XXVIII), defined as above, with a reagent of the general formula R₇—MgX, in which R₁ is defined as above and X is a halogen, or a reagent of the formula R₇-M, in which M is a metal exemplified by Zn and Li.

Compounds of the general formula (IV), in which R₁ is R₇C(O), R₂, R₃, R₄, B, R₁₄ and R₁₅ are defined as above, X is a nitrogen or a single bond connected to a nitrogen which is a member of the B ring, comprises the following steps (h1-h2).

h1) Reacting a compound of the general formula (XXVIII), defined as above, with N,O-dimethylhydroxylamine. The reaction can be performed using known reagents like CDI to give a compound of the general formula (XLI).

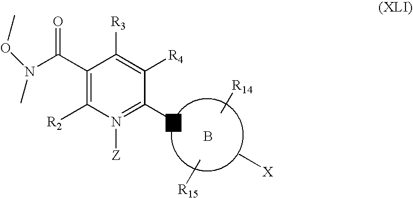

h2) A compound of the general formula (XLI), which is defined as above can be reacted with a reagent of the general formula R₇—MgX, in which R₇ is defined as above and X is a halogen, or a reagent of the formula R₇-M, in which M is a metal exemplified by Zn and Li.

Compounds of the general formula (VIII) can be formed in one of the processes (i1-i5). The compounds of formula (VIII) in which R₅ is a hydrogen are advantageously isolated as a zwitterion. A ring nitrogen of compounds of formula (X) and (XI) used in the below steps may be protected by a protective group such as t-butyloxycarbonyl.

i1) Compounds of the general formula (VIII) in which B, R₅, R₁₄, R₁₅, R^{c} and R^{d} are defined as above, X is a single bond or a carbon, may be formed by reacting a compound of formula (X) with a compound of formula (III). The reaction is generally carried out in an inert organic solvent such as dichloromethane at ambient temperature. The reaction may be carried out using standard conditions or in the presence of EDCI or the combination of EDCI and HOBT. Optionally, the reaction may be carried out in the presence of an organic base such as triethylamine or DIPEA.

i2) Compounds of the general formula (VIII) in which B, R₅, R₁₄, R₁₅, R^{c} and R^{d} are defined as above, X is a single bond or a carbon, may also be formed by reacting a compound of formula (X) with a compound of formula (III), in which the nitrogen in the B-ring is protected, for example by t-butyloxycarbonyl. The reaction is generally carried out in an inert organic solvent such as THF. The reaction may be carried out using standard conditions or in the presence of TBTU. Optionally, the reaction may be carried out in the presence of an organic base such as triethylamine or DIPEA. Advantageously a reagent such as LiCl may be used. When the product contains a t-butyloxycarbonyl this may be removed using standard procedures or in the presence of formic acid. When R₅ is a hydrogen, compound (VIII) can be isolated as a zwitterion.

i3) Compounds of the general formula (VIII) in which R₅ is hydrogen, B, R₁₄, R₁₅, R^{c} and R^{d} are defined as above, X is a nitrogen or a single bond connected to a nitrogen which is a member of the B ring, can be formed by reacting a compound of formula (XI) defined as above with a compound of formula (V), defined as above. The reaction is generally carried out in an inert solvent such as THF. The reaction may also be carried out in the presence of an organic base such as triethylamine or DIPEA.

i4) Compounds of the general formula (VIII) in which B, R₅, R₁₄, R₁₅, R^{c} and R^{d} defined as above, X is a nitrogen or a single bond connected to a nitrogen which is a member of the B ring, can also be formed by reacting a compound of formula (XI) with a compound of formula (VI) which is defined as above. The reaction is generally carried out in a solvent such as DMA. This reaction may also be carried out in the presence of an organic base such as triethylamine or DIPEA

i5) A compound of formula (VIII) which is protected with t-butoxy carbonyl may be transformed into a compound without the protective group using standard procedures or a reagent such as formic acid.

(j) Compounds of the general formula (VII) which are defined as above can be formed by reacting a compound of formula (XLVI) using standard conditions or with a chlorinating reagent such as thionyl chloride or POCl₃. Advantageously dimethylformamide may be used. The reaction may be performed in an inert solvent. Advantageously the inert solvent is toluene.

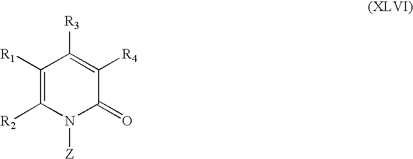

The preparation of compounds of the general formula (XLVII) which is defined as above comprises the steps (k1-k3) below;

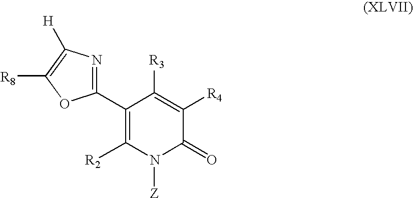

k1) Reacting a compound of the general formula (XLVIII)

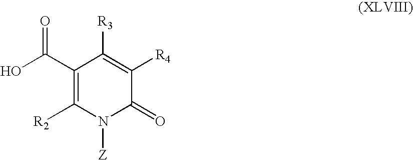

with a compound of the general formula (XXIII), which is having R₈ instead of R₁₀, otherwise defined as above, to give a compound of the formula (IL). The reaction is generally carried out in DCM at ambient temperature. The reaction may be carried out using standard conditions or in the presence of EDCI or the combination of EDCI and HOBT. Optionally the reaction may be carried out in the presence of an organic base such as TEA or DIPEA.

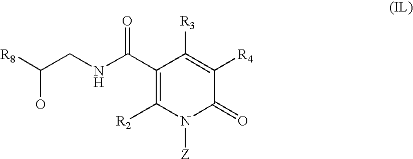

k2) The compound of formula (IL) can be transformed to a compound (L) using standard conditions or an oxidising agent such as the mixture of oxalylchloride and DMSO.

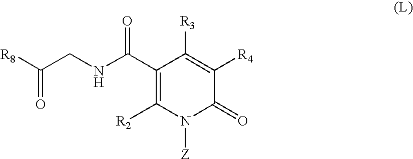

k3) The compound of formula (L) can then be transformed into a compound of the general formula (XLVII), using standard conditions or in the presence of (Methoxycarbonylsulfamoyl)triethylammonium hydroxide (Burgess reagent). The reaction is generally performed in an inert solvent such as TIP. The reaction is carried out at elevated temperatures using standard equipment or a single-node microwave oven.

Compounds of the general formula (III) can be formed by reacting the corresponding sulfonyl chloride using known methods with ammonia in an inert solvent such as methanol.

l) Preparation of compounds of the general formula (XLVIII) which is defined as above except for R₃ which is hydrogen, comprises the following steps (l₁-l₃);
l1) Reacting a compound of the formula (LI), in which R₂ and R₆ are defined as for formula (I) with dimethoxy-N,N-dimethylmethaneamine to form a

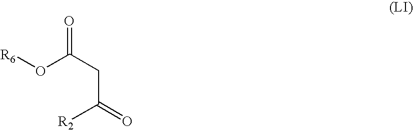

compound of formula (LII).

l2) This compound (LII) can then be reacted further with a compound of the

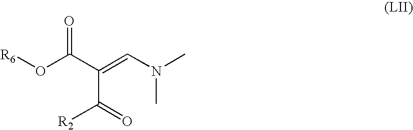

general formula R₄CH₂C(O)NH₂, in which R₄ is defined as for formula (I) to give a compound of the general formula (LIII). The reaction is generally performed in an inert solvent such as ethanol, optionally in the presence of a strong base such as sodium ethoxide.

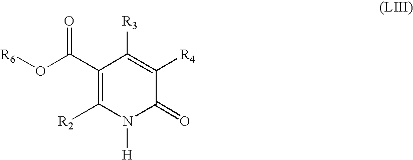

(l3) A compound of the general formula (LIII) can then be transformed to a compound of the general formula (XLVIII). The reaction is generally performed in a protic solvent such as water together with a co-solvent such as THE or methanol. The reaction can be performed using standard reagents or in the presence of LiOH, NaOH or KOH.

(m) The formation of a compound of the general formula (XXX), which is defined as above can be made the below synthesis;

m1) A compound of the general formula (LIV) where R₈ is defined as formula (I) above can be

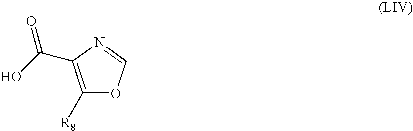

transformed in to a compound of the formula (LV)

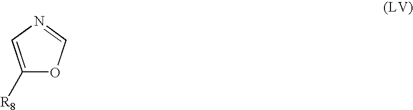

using standard conditions or using Cu(II)O and quinoline.

m2) The compound of the general formula (LV) can be reacted with a compound of the general formula (LVI) in

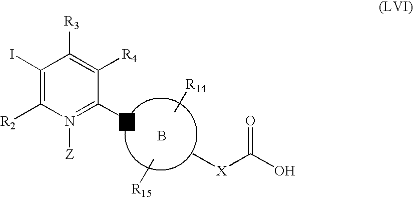

which R₂, R₃, R₄, B, R₁₄ and R₁₅ are defined as for formula (I) and X is a carbon or a single bond, to give compounds of the general formula (XXX). The reaction is generally performed in an inert solvent such as THF under inert atmosphere. The reaction can be performed using standard conditions or in the presence of AlkylLi such as BuLi, ZnCl₂, Pd(Ph₃)₄.

(n) Compounds of the general formula (XXXVI) can also be made by the step below;

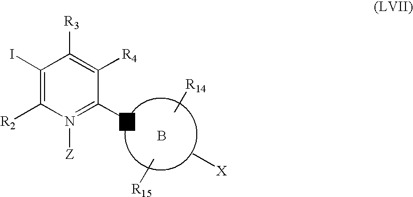

n1) Reacting a compound of the general formula (LV), which is defined as above, with a compound of the general formula (LVI), in which R₂, R₃, R₄, B, R₁₄ and R₁₅ are defied as in formula (I) above, X is a nitrogen or a single bond connected to a nitrogen which is a member of the B ring.

o) The preparation of compounds of the general formula (LVIII), in which R₁₄ and R₁₅ are defined as for formula (I) with the exception that R₁₄ is connected to the same atom as X, and X is defined as a single bond, comprises the below step;

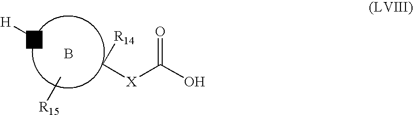

o1) Reacting the corresponding (LIX) with R₁₄-L, wherein L is a suitable leaving group, such as chloro, bromo, iodo,

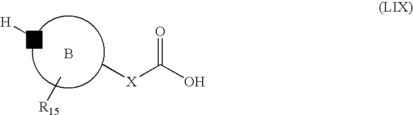

triflate or tosyl to form compounds of the general formula (LVIII), using standard conditions or in the presence of with BuLi and diisopropylamine mixture.

The preparation of compounds of the formula (III) comprises the below processes. (p1-p3)

p1) A compound of the formula LR^{c}R^{d} wherein L is a suitable leaving group, such as chloro, bromo, iodo could be transformed to the corresponding compound (III) using a sequence of reactions using first SMOPS* (*Baskin and Wang. Tetrahedron Letters, 2002, 43, 8479-83. See esp. page 8480, left hand column.) followed by hydrolysis using a base like NaOMe in an inert solvent like DMSO at room temperature. Followed by treatment by NH₂OSO₃H and NaOAc to give a compound of formula (III).

p2) A compound of the formula LSO₂R^{c}R^{d} wherein L is a suitable leaving group, such as chloro, bromo, iodo could be reacted with ammonium hydroxide or H₂NR₅ in an inert solvent such as DCM to give a compound of formula (III).

p3) A compound of the formula LR^{c}R^{d} wherein L is a suitable leaving group, such as chloro, bromo, iodo could be transformed to the corresponding compound (III) using a sequence of reactions first NaSO₃, followed by a using a reagent such as PCl₃, POCl₃ or SOCl₂, followed by ammonium hydroxide or H₂NR₅ to give a compound of formula (III).

At any stage in the synthesis of amine substituted pyridines, a chlorine substituent in the 2, 4 or 6 position of the pyridine can be substituted with azide using known techniques. The azide can be reduced to the corresponding amine. These amines can subsequently be alkylated or acylated using known methods or with an alkylhalide or acylhalide, respectively.

Persons skilled in the art will appreciate that an acid can be transformed to the corresponding activated ester such as an acid chloride, followed by reaction with a thiol, R₁₆SH to give thioesters, R₁₆SC(O).

Persons skilled in the art will appreciate that an acid can be transformed to the corresponding activated ester such as an acid chloride, followed by reaction with a alcohol, R₆OH to give esters, R₆OC(O).

Persons skilled in the art will appreciate that a compound of formula (III) could be alkylated at the carbon atom in the alpha position to the sulfonamide using an alkylhalide. Preferably under basic conditions using a strong base such as sodium hydride.

Persons skilled in the art will appreciate that a nitrogen substituent at the 3 position of a pyridine could be replaced by a thioether chain, R₁₇S—, using known techniques or R₁₇SSR₁₇ and tert-Butylnitrite.

Persons skilled in the art will appreciate that a thioketone could be made from the corresponding ketone using known techniques or using Lawessons reagent.

Persons skilled in the art will appreciate that a pyridine N-oxide could be formed by from a pyridine using an oxidising agent such as Urea hydrogen peroxide or hydrogen peroxide, with or without the presence of trifluoroaceticanhydride.

The compounds of the invention may be isolated from their reaction mixtures using conventional techniques.

Persons skilled in the art will appreciate that, in order to obtain compounds of the invention in an alternative and in some occasions, more convenient manner, the individual process steps mentioned hereinbefore may be performed in different order, and/or the individual reactions may be performed at different stage in the overall route (i.e. chemical transformations may be performed upon different intermediates to those associated hereinbefore with a particular reaction).

It will be appreciated that by those skilled in the art that the processes described above and hereinafter the functional groups of intermediate compounds may need to be protected by protecting groups.

Functional groups that it is desirable to protect include hydroxy, amino and carboxylic acid. Suitable protecting groups for hydroxy include optionally substituted and/or unsaturated alkyl groups (e.g. methyl, allyl, benzyl or tert-butyl), trialkyl silyl or diarylalkylsilyl groups (e.g. t-butyldimethylsilyl, t-butyldiphenylsilyl or trimethylsilyl) and tetrahydropyranyl. Suitable protecting groups for carboxylic acids include (C₁-C₆)allyl or benzyl esters. Suitable protecting groups for amino include t-butyloxycarbonyl, benzyloxycarbonyl, 2-(trimethylsilyl)ethoxymethyl or 2-trimethylsilylethoxycarbonyl (Teoc).

The protection and deprotection of functional groups may take place before or after any reaction in the above mentioned processes.

Persons skilled in the art will appreciate that, in order to obtain compounds of the invention in an alternative, and on some occasions, more convenient, manner, the individual process steps mentioned hereinbefore may be performed in different order, and/or the individual reactions may be performed at a different stage in the overall route (i.e. substituents may be added to and/or chemical transformations performed upon, different intermediates to hose mentioned hereinbefore in conjunction with a particular reaction). This may negate, or render necessary, the need for protecting groups.

Persons skilled in the art will appreciate that starting materials for any of the above processes can in some cases be commercially available.

Persons skilled in the art will appreciate that processes above could for some starting materials above be found in the general common knowledge.

The type of chemistry involved will dictate the need for protecting groups as well as sequence for accomplishing the synthesis.

The use of protecting groups is fully described in “Protective groups in Organic Chemistry”, edited by J W F McOmie, Plenum Press (1973), and “Protective Groups in Organic Synthesis”, 3^{rd} edition, T. W. Greene & P. G. M Wutz, Wiley-Interscience (1999).

Protected derivatives of the invention may be converted chemically to compounds of the invention using standard deprotection techniques (e.g. under alkaline or acidic conditions). The skilled person will also appreciate that certain compounds of Formula (II)-(LIX) may also be referred to as being “protected derivatives”

Compounds of the invention may also contain one or more asymmetric carbon atoms and may therefore exhibit optical and/or diastereoisomerism. Diastereoisomers may be separated using conventional techniques, e.g. chromatography or crystallization. The various stereoisomers may be isolated by separation of a racemic or other mixture of the compounds using conventional, e.g. HPLC techniques. Alternatively the desired optical isomers may be made by reaction of the appropriate optically active starting materials under conditions which will not cause racemisation or epimerisation, or by derivatisation, for example with a homochiral acid followed by separation of the diasteromeric derivatives by conventionals means (e.g. HPLC, chromatography over silica or crystallization). Stereocenters may also be introduced by asymmetric synthesis, (e.g. metalloorganic reactions using chiral ligands). All stereoisomers are included within the scope of the invention.

All novel intermediates form a further aspect of the invention.

Salts of the compounds of formula (I) may be formed by reacting the free acid, or a salt thereof, or the free base, or a salt or a derivative thereof, with one or more equivalents of the appropriate base (for example ammonium hydroxide optionally substituted by C₁-C₆-alkyl or an alkali metal or alkaline earth metal hydroxide) or acid (for example a hydrohalic (especially HCl), sulphuric, oxalic or phosphoric acid). The reaction may be carried out in a solvent or medium in which the salt is insoluble or in a solvent in which the salt is soluble, e.g. water, ethanol, tetrahydrofuran or diethyl ether, which may be removed in vacuo, or by freeze drying. The reaction may also carried out on an ion exchange resin. The nontoxic physiologically acceptable salts are preferred, although other salts may be useful, e.g. in isolating or purifying the product.

### Pharmacological Data

Functional inhibition of the P2Y₁₂ receptor can be measured by in vitro assays using cell membranes from P2Y₁₂ transfected CHO-cells, the methodology is indicated below.

Functional inhibition of 2-Me-S-ADP induced P2Y₁₂ signalling: 5 μg of membranes were diluted in 200 μl of 200 mM NaCl, 1 mM MgCl₂, 50 mM HEPES (pH 7.4), 0.01% BSA, 30 μg/ml saponin and 10 μM GDP. To this was added an EC₈₀ concentration of agonist (2-methyl-thio-adenosine diphosphate), the required concentration of test compound and 0.1 μCi ³⁵S-GTPγS. The reaction was allowed to proceed at 30° C. for 45 min. Samples were then transferred on to GF/B filters using a cell harvester and washed with wash buffer (50 mM Tris (pH 7.4), 5 mM MgCl₂, 50 mM NaCl). Filters were then covered with scintillant and counted for the amount of ³⁵S-GTPγS retained by the filter. Maximum activity was that determined in the presence of the agonist and minimum activity in the absence of the agonist following subtraction of the value determined for nonspecific activity. The effect of compounds at various concentrations was plotted according to the equation

y=A+((B−A)/(1+((C/x)̂D)))

and IC₅₀ estimated where
A is the bottom plateau of the curve i.e. the final minimum y value
B is the top of the plateau of the curve i.e. the final maximum y value
C is the x value at the middle of the curve. This represents the log EC₅₀ value when A+B=100
D is the slope factor.
x is the original known x values.
Y is the original known y values.
Most of the compounds of the invention have an activity, when tested in the functional inhibition of 2-Me-S-ADPinduced P2Y₁₂ signalling assay described, at a concentration of around 4 μM or below.

For example the compounds described in Examples 91 and 119 gave the following test result in the functional inhibition of 2-Me-S-ADPinduced P2Y₁₂ signalling assay described.

IC₅₀(μM)

| | |
|---|---|
| Example 91 | 0.46 |
| Example 119 | 0.25 |

The compounds of the invention act as P2Y₁₂ receptor antagonists and are therefore useful in therapy. Thus, according to a further aspect of the invention there is provided a compound of formula (I), or a pharmaceutically acceptable salt thereof, for use in therapy.

In a further aspect there is provided the use of a compound of formula (I), or a pharmaceutically acceptable salt thereof, for the manufacture of a medicament for treatment of a platelet aggregation disorder. In another aspect of the invention there is provided the use of a compound of formula (I), or a pharmaceutically acceptable salt thereof, for the manufacture of a medicament for the inhibition of the P2Y₁₂ receptor.

The compounds are useful in therapy, especially adjunctive therapy, particularly they are indicated for use as: inhibitors of platelet activation, aggregation and degranulation, promoters of platelet disaggregation, anti-thrombotic agents or in the treatment or prophylaxis of unstable angina, coronary angioplasty (PTCA), myocardial infarction, perithrombolysis, primary arterial thrombotic complications of atherosclerosis such as thrombotic or embolic stroke, transient ischaemic attacks, peripheral vascular disease, myocardial infarction with or without thrombolysis, arterial complications due to interventions in atherosclerotic disease such as angioplasty, endarterectomy, stent placement, coronary and other vascular graft surgery, thrombotic complications of surgical or mechanical damage such as tissue salvage following accidental or surgical trauma, reconstructive surgery including skin and muscle flaps, conditions with a diffuse thrombotic/platelet consumption component such as disseminated intravascular coagulation, thrombotic thrombocytopaenic purpura, haemolytic uraemic syndrome, thrombotic complications of septicaemia, adult respiratory distress syndrome, anti-phospholipid syndrome, heparin-induced thrombocytopaenia and pre-eclampsia/eclampsia, or venous thrombosis such as deep vein thrombosis, venoocclusive disease, haematological conditions such as myeloproliferative disease, including thrombocythaemia, sickle cell disease; or in the prevention of mechanically-induced platelet activation in vivo, such as cardiopulmonary bypass and extracorporeal membrane oxygenation (prevention of microthromboembolism), mechanically-induced platelet activation in vitro, such as use in the preservation of blood products, e.g. platelet concentrates, or shunt occlusion such as in renal dialysis and plasmapheresis, thrombosis secondary to vascular damage/inflammation such as vasculitis, arteritis, glomerulonephritis, inflammatory bowel disease and organ graft rejection, conditions such as migraine, Raynaud's phenomenon, conditions in which platelets can contribute to the underlying inflammatory disease process in the vascular wall such as atheromatous plaque formation/progression, stenosis/restenosis and in other inflammatory conditions such as asthma, in which platelets and platelet-derived factors are implicated in the immunological disease process.

According to the invention there is further provided the use of a compound according to the invention in the manufacture of a medicament for the treatment of the above disorders. In particular the compounds of the invention are useful for treating myocardial infarction, thrombotic stroke, transient ischaemic attacks, peripheral vascular disease and angina, especially unstable angina. The invention also provides a method of treatment of the above disorders which comprises administering to a patient suffering from such a disorder a therapeutically effective amount of a compound according to the invention.

In a further aspect the invention provides a pharmaceutical composition comprising a compound of formula (I), or a pharmaceutically acceptable salt thereof, together with a pharmaceutically acceptable diluent, adjuvant and/or carrier.

The compounds may be administered topically, e.g. to the lung and/or the airways, in the form of solutions, suspensions, HFA aerosols and dry powder formulations; or systemically, e.g. by oral administration in the form of tablets, pills, capsules, syrups, powders or granules, or by parenteral administration in the form of sterile parenteral solutions or suspensions, by subcutaneous administration, or by rectal administration in the form of suppositories or transdermally.

The compounds of the invention may be administered on their own or as a pharmaceutical composition comprising the compound of the invention in combination with a pharmaceutically acceptable diluent, adjuvant or carrier. Particularly preferred are compositions not containing material capable of causing an adverse, e.g. an allergic, reaction.

Dry powder formulations and pressurised HFA aerosols of the compounds of the invention may be administered by oral or nasal inhalation. For inhalation the compound is desirably finely divided. The compounds of the invention may also be administered by means of a dry powder inhaler. The inhaler may be a single or a multi dose inhaler, and may be a breath actuated dry powder inhaler.

One possibility is to mix the finely divided compound with a carrier substance, e.g. a mono-, di- or polysaccharide, a sugar alcohol or another polyol. Suitable carriers include sugars and starch. Alternatively the finely divided compound may be coated by another substance. The powder mixture may also be dispensed into hard gelatine capsules, each containing the desired dose of the active compound.

Another possibility is to process the finely divided powder into spheres, which break up during the inhalation procedure. This spheronized powder may be filled into the drug reservoir of a multidose inhaler, e.g. that known as the Turbuhaler® in which a dosing unit meters the desired dose which is then inhaled by the patient. With this system the active compound with or without a carrier substance is delivered to the patient.

The pharmaceutical composition comprising the compound of the invention may conveniently be tablets, pills, capsules, syrups, powders or granules for oral administration; sterile parenteral or subcutaneous solutions, suspensions for parenteral administration or suppositories for rectal administration.

For oral administration the active compound may be admixed with an adjuvant or a carrier, e.g. lactose, saccharose, sorbitol, mannitol, starches such as potato starch, corn starch or amylopectin, cellulose derivatives, a binder such as gelatine or polyvinylpyrrolidone, and a lubricant such as magnesium stearate, calcium stearate, polyethylene glycol, waxes, paraffin, and the like, and then compressed into tablets. If coated tablets are required, the cores, prepared as described above, may be coated with a concentrated sugar solution which may contain e.g. gum arabic, gelatine, talcum, titanium dioxide, and the like. Alternatively, the tablet may be coated with a suitable polymer dissolved either in a readily volatile organic solvent or an aqueous solvent.

For the preparation of soft gelatine capsules, the compound may be admixed with e.g. a vegetable oil or polyethylene glycol. Hard gelatine capsules may contain granules of the compound using either the above mentioned excipients for tablets, e.g. lactose, saccharose, sorbitol, mannitol, starches, cellulose derivatives or gelatine. Also liquid or semisolid formulations of the drug may be filled into hard gelatine capsules.

Liquid preparations for oral application may be in the form of syrups or suspensions, for example solutions containing the compound, the balance being sugar and a mixture of ethanol, water, glycerol and propylene glycol. Optionally such liquid preparations may contain colouring agents, flavouring agents, saccharine and carboxymethylcellulose as a thickening agent or other excipients known to those skilled in art.

The invention will be her illustrated with the following non-limiting examples:

### EXAMPLES

### General Experimental Procedure

Mass spectra was recorded on a Finnigan LCQ Duo ion trap mass spectrometer equipped with an electrospray interface (LC-ms) or LC-ms system consisting of a Waters ZQ using a LC-Agilent 1100 LC system.

1H NMR measurements were performed on a Varian Mercury VX

400 spectrometer, operating at a 1H frequency of 400 and Varian UNITY plus 400, 500 and 600 spectrometers, operating at 1H frequencies of 400, 500 and 600 respectively. Chemical shifts are given in ppm with the solvent as internal standard. Chromatography was performed using Biotage silica gel 40S, 40M, 12i or Merck silica gel 60 (0.063-0.200 mm). Flash chromatography was performed using either standard glass- or plastic-columns column or on a Biotage Horizon system. HPLC separations were performed on a Waters YMC-ODS AQS-3 120 Angstrom 3×500 mm or on a Waters Delta Prep Systems using Kromasil C8, 10 μm columns. Reactions performed in a microwave reactor were performed in a Personal Chemistry Smith Creator, Smith synthesizer or an Emrys Optimizer.

### LIST OF USED ABBREVIATIONS

| Abbreviation | Explanation |
|---|---|
| AcOH | Acetic acid |
| Aq | Aqueous |
| br | Broad |
| Brine | A saturated solution of sodium chloride in water |
| BSA | Bovine Serum Albumine |
| CDI | Carbonyldiimidazole |
| d | Doublet |
| DCE | 1,2-Dichloroethane |
| DCM | Dichloromethane |
| DDQ | 2,3-Dichloro-5,6-dicyano-1,4-benzoquinone |
| DIEA | N,N-Diisopropylethylamine |
| DIPEA | N,N-Diisopropylethylamine |
| DMA | N,N-Dimethylacetamide |
| DMAP | N,N-dimethylpyridin-4-amine |
| DMF | N,N-dimethylformamide |
| DMSO | Dimethylsulphoxide |
| EDCI | N-[3-(dimethylamino)propyl]-N′-ethylcarbodiimide |
| | hydrochloride |
| EtOAc | Ethyl acetate |
| EtOH | Ethanol |
| HATU | O-(7-Azabenzotriazol-1-yl)-1,1,3,3- |
| | tetramethyluromium hexafluorophosphate |
| HEPES | [4-(2-hydroxyethyl)-1-piperazineethanesulfonic acid |
| HFA | Hydrofluoroalkanes |
| HOAc | Acetic acid |
| HOBT | 1-Hydroxybenzotriazole |
| HPLC | High-performance liquid chromatography |
| Hz | Hertz |
| J | Coupling constant |
| LDA | Litiumdiisopropyl amide |
| M | Multiplet |
| m-CPBA | 3-chlorobenzenecarboperoxoic acid |
| MeOH | Methanol |
| MHz | Megahertz |
| mL | Millilitre |
| MS | Mass spectra |
| NBS | 1-Bromopyrrolidine-2,5-dione(N-bromo |
| | succinimide) |
| NCS | 1-chloropyrrolidine-2,5-dione |
| q | Quartet |
| r.t. | Room temperature |
| s | Singlet |
| SMOPS | sodium 3-methoxy-3-oxopropane-1-sulfinate |
| t | triplet |
| TB | Tyrodes Buffer |
| TBTU | N-[(1H-1,2,3-benzotriazol-1- |
| | yloxy)(dimethylamino)methylene]-N- |
| | methylmethanaminium tetrafluoroborate |
| TEA | Triethylamine |
| TFA | Trifluoroacetic acid |
| THF | Tetrahydrofurane |

### Sulphone Amides

### Synthesis of Sulfone Amides

Each of the following substances was made by reacting the corresponding sulfonyl chloride (0.75 mmol) with a saturated solution of ammonia in MeOH (5 mL). After evaporation of the ammonia and MeOH the residues were dissolved in MeOH (5 mL) and to a few samples DMF (2 mL) was also added to dissolve the reaction mixtures. The solutions where then separately filtered through ISOLUTE SCX-2, (25 mL cartridge) containing acidic ion exchange resin (propylsulphonic acid type, 5 g). MeOH (16 mL) was used to rinse the product from the resin. After removal of the solvent each of the products were used without further purification as described in Method A below.

The sulfonamides made by this procedure are listed in table 1. Other sulphone amides were made via methods described in the examples or methods similar to those described.

TABLE 1

| | | | Crude yield | |
|---|---|---|---|---|
| Products | Compound name | Mw | mg | % |
| 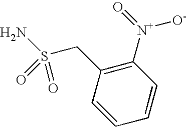 | 1-(2-nitrophenyl)methanesulfonamide | 216.22 | 222 | 134% |
| 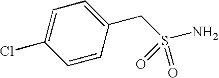 | 1-(4-chlorophenyl)methanesulfonamide | 205.66 | 212 | 129% |
| 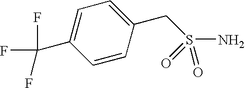 | 1-[4-(trifluoromethyl)phenyl]methanesulfonamide | 239.22 | 114 | 123% |
| 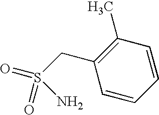 | 1-(2-methylphenyl)methanesulfonamide | 185.25 | 10 | 7% |
| 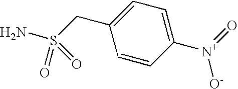 | 1-(4-nitrophenyl)methanesulfonamide | 216.22 | 198 | 119% |
| 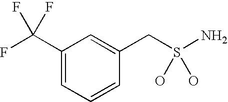 | 1-[3-(trifluoromethyl)phenyl]methanesulfonamide | 239.22 | 210 | 114% |
| 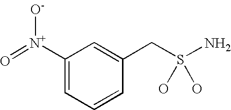 | 1-(3-nitrophenyl)methanesulfonamide | 216.22 | 194 | 118% |
| 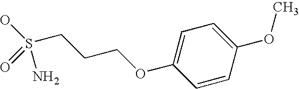 | 3-(4-methoxyphenoxy)propane-1-sulfonamide | 245.30 | 210 | 113% |
| 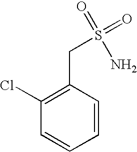 | 1-(2-chlorophenyl)methanesulfonamide | 205.66 | 200 | 122% |
| 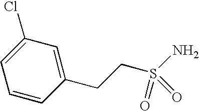 | 2-(3-chlorophenyl)ethanesulfonamide | 219.69 | 204 | 119% |
| 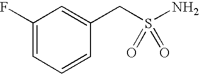 | 1-(3-fluorophenyl)methanesulfonamide | 189.21 | 192 | 130% |
| 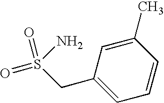 | 1-(3-methylphenyl)methanesulfonamide | 185.25 | 284 | 196% |
| 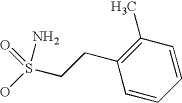 | 2-(2-methylphenyl)ethanesulfonamide | 199.27 | 324 | 185% |
| 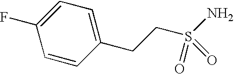 | 2-(4-fluorophenyl)ethanesulfonamide | 203.24 | 164 | 106% |
| 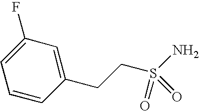 | 2-(3-fluorophenyl)ethanesulfonamide | 203.24 | 176 | 111% |
| 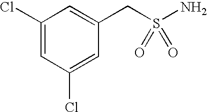 | 1-(3,5-dichlorophenyl)methanesulfonamide | 240.11 | 192 | 109% |
| 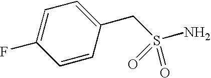 | 1-(4-fluorophenyl)methanesulfonamide | 189.21 | 156 | 108% |
| 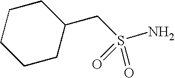 | 1-cyclohexylmethanesulfonamide | 177.27 | 134 | 95% |
| 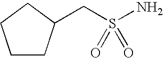 | 1-cyclopentylmethanesulfonamide | 163.24 | 148 | 108% |

### Synthesis of Examples

### Method A:

1-[3-Cyano-5-(ethoxycarbonyl)-6-methylpyridine-2-yl]azetidine-3-carboxylic acid (See Example 1(d)) (1 Eq), sulfone amide (1.48 Eq, the amount and structure of the sulfonamide used is specified in each of the examples below) and DIPEA (5 Eq) was stirred in DMF (8 mL/mmol of the acid used). HATU (1.05 Eq) was dissolved in DMF (4 mL/mmol of the acid used) added and the reaction was stirred at r.t over night. The solvent was removed in vacuo and the crude reaction mixture was dissolved in DMSO (1 mL) and purified by preparative HPLC (Kromasil C8, 5 μm particles, 100×21.2 nm column, Eluent A: 100% acetonitrile, Eluent B: 0.1M ammonium acetate in water containing 5% acetonitrile, flow 30 mL/min, using a increasing gradient of acetonitrile over 8 minutes to afford the products after evaporation of the solvents).

### Method B

To a solution of 1-[3-cyano-5-(ethoxycarbonyl)-6-methylpyridin-2-yl]piperidine-4-carboxylic acid (0.21 mmol) or 1-[3-cyano-5-(ethoxycarbonyl)-6-methylpyridin-2-yl]azetidine-3-carboxylic acid (0.21 mmol) DCM (2 mL) was added TBTU 0.25 mmol) and DIPEA (1.05) mmol. The reaction mixture was stirred for 10 minutes followed by addition of sulfonamide (0.25 mmol) e.g. 5-chlorothiophene-3-sulfonamide. The reaction mixture was stirred over night followed by addition of 0.1 M KHSO₄ (2 mL), the organic phase was isolated and the crude reaction mixture was submitted to preparative HPLC (see below for details) in order to isolate the wanted product, e.g. ethyl 6-[4-({[(5-chloro-3-thienyl)sulfonyl]amino}carbonyl)piperidin-1-yl]-5-cyano-2-methylnicotinate.

The preparative HPLC system used was a Waters Fraction Lynx Purification System with Kromasil C8 5 mm 20×100 nm u columns. The mobile phase used was varying gradients of CH₃CN and 0.1 M NH₄OAc (aq) buffer. The flow was 30 mL/minute. MS triggered fraction collection was used. Mass spectra were recorded on either a Micromass ZQ single quadrupole or a Micromass Quattro micro, both equipped with a pneumatically assisted electro spray interface.

### Method C

A solution of 1-[3-cyano-5-(isopropoxycarbonyl)-6-methylpyridin-2-yl]azetidine-3-carboxylic acid (0.091 g, 0.3 mmol), DIPEA 0.074 g, 0.6 mmol) and TBTU (0.039 g, 0.3 mmol) in 1 eq. DCM/1 eq. DMF (2 mL) was added to sulfonamide (0.4 mmol), e.g. 4-(trifluoromethyl)benzenesulfonamide. The reaction mixture was stirred for 48 h followed by addition of TBTU (0.013 g, 0.1 mmol). After 20 h the solvents were removed in vacuo. The crude reaction mixture was added NaHSO₄ (2 mL, 1M) and due to differences in solubility between products DCM and DCM/ethyl acetate was used for extraction. The organic phase was isolated and the solvents were removed in vacuo. The crude material was purified using preparative HPLC (see below for details) in order to isolate the desired product, e.g. isopropyl 5-cyano-2-methyl-6-{3-[({[4-(trifluoromethyl)phenyl]sulfonyl}amino)carbonyl]azetidin-1-yl}nicotinate.

### Example 1

### 5-Cyano-6-[3-(2-methoxycarbonyl-phenylmethanesulfonylaminocarbonyl)-azetidin-1-yl]-2-methyl-nicotinic acid ethyl ester

### (a) Ethyl 2-((dimethylamino)methylene)-3-oxobutanoate

1,1-dimethoxy-N,N-dimethylmethanamine (500 g, 4195 mmol) was added to ethyl 3-oxobutanoate (461.6 g, 3547 mmol) under an atmosphere of nitrogen at r.t during 13 minutes (weak exotherm). The orange red solution was stirred for 22 hours and concentrated in vacuo. The residue was co-evaporated with toluene (3 times 200 ml each) and used without no need for further purification in the next step.

MS ^{m}/_{z}: 186 (M+1).

### (b) Ethyl 5-cyano-2-methyl-6-oxo-1,6-dihydropyridine-3-carboxylate

Sodium ethoxide (1240.7 g of a 21 wt % solution in EtOH, 3829 mmol) was added to a stirred suspension of 2-cyanoacetamide (298 g, 3544 mmol) in EtOH (3000 mL) during 8 minutes under an atmosphere of nitrogen at r.t. The crude condensation product from step (a) above dissolved in 950 ml EtOH was added slowly (slightly exothermic reaction) and after about one third had been added further EtOH (1000 mL) was added to allow efficient stirring (suspension) followed by the addition of the rest of the condensation product (total addition time 30 min). After stirring over night at r.t. HOAc (526 g, 8759 mmol) was added to the reaction and the mixture was concentrated in vacuo leaving a thick orange slurry (volume about 3000 mL), 1 M HCl (4628 mL, 4628 mmol) was added during 10 min followed by water (500 mL). The stirring was stopped and the precipitate was filtered off and washed with water (200 mL). NMR showed the presence of about 5-10% of the corresponding acid and the solid was washed by stirring with further water (1500 mL+3×1000 mL), a solution of saturated NaHCO₃ (400 mL) in water (600 mL) and finally water (1000 mL). Filtration of the solid and drying in vacuo at 80° C. gave pure ethyl 5-cyano-2-methyl-6-oxo-1,6-dihydropyridine-3-carboxylate. Yield: 493 g (67%).

¹H NMR (400 MHz, DMSO-d₆): δ 1.36 (3H, t, J=7.1 Hz), 2.62 (3H, s), 4.25 (2H, q, J=7.1 Hz), 8.71 (1H, s), 12.79 (1H, br s).

### (c) Ethyl 6-chloro-5-cyano-2-methylnicotinate

Toluene (4000 mL) and thionylchloride (507 g, 4262 mmol) were added to ethyl 5-cyano-2-methyl-6-oxo-1,6-dihydropyridine-3-carboxylate (293 g, 1421 mmol) under an atmosphere of nitrogen and the mixture was heated to 50° C. (oil bath temperature) and DMF (100 g, 1368 mmol) was added during 2 minutes. The temperature was raised to 80° C. (oil bath temperature) and the stirring was continued for 2 hours. The mixture was concentrated in vacuo (about 3500 ml was evaporated off) leaving a red oil. EtOH (1000 mL, 99%) was added and then evaporated off. Dichloromethane (4000 mL) was added followed by 4% NaHCO₃ solution (4000 mL) and the mixture was stirred for 15 minutes. The organic phase was separated and evaporated to give ethyl 6-chloro-5-cyano-2-methylnicotinate as a dark red crude solid which was used without further purification. Yield 301 g (75%/O).

¹H NMR (400 MHz, CDCl₃): δ 1.42 (3H, t, J=7.1 Hz), 2.91 (3H, s), 4.40 (21 q, J=7.1 Hz), 8.49 (1H, s).

### (d) 1-[3-Cyano-5-(ethoxycarbonyl)-6-methylpyridine-2-yl]azetidine-3-carboxylic acid

Ethyl 6-chloro-5-cyano-2-methylnicotinate (50.98 g, 227 mmol), azetidine-3-carboxylic acid (24.09 g, 238 mmol) and DIPEA (118.9 mL, 681 mmol) were suspended in EtOH (250 mL) and heated at reflux for 1 h. The reaction mixture was cooled to r.t and added drop-wise to KHSO₄ (154.5 g, 1135 mmol) in water (3000 mL). The solids were collected by filtration and dried under vacuum to afford 1-[3-Cyano-5-(ethoxycarbonyl)-6-methylpyridine-2-yl]azetidine-3-carboxylic acid as a solid, which was used without further purification. Yield: 65.33 g (100%).

¹H NMR (400 MHz, CDCl₃): δ 1.37 (3H, t, J=7.1 Hz), 2.72 (3H, s), 3.59-3.68 (1H, m), 4.31 (2H, q, J=7.1 Hz), 4.55-4.68 (4H, m), 8.28 (1H, s).

MS ^{m}/_{z}: 290 (M+1).

### (e) 5-Cyano-6-[3-(2-methoxycarbonyl-phenylmethanesulfonylaminocarbonyl)azetidin-1-yl]-2-methyl-nicotinic acid ethyl ester

1-[3-Cyano-5-(ethoxycarbonyl)-6-methylpyridine-2-yl]azetidine-3-carboxylic acid (0.072 g, 0.25 mmol), methyl 2-[(aminosulfonyl)methyl]benzoate (0.085 g, 0.375 mmol) and triethyl amine (0.55 mL, 4 mmol) was stirred in DMF (2 mL). HATU (0.100 g, 0.263 mmol) dissolved in DMF (1 mL) was added and the reaction was stirred at r.t over night. The reaction mixture was purified by preparative HPLC using Kromasil C8, 5μ particles, 100×21.2 mm column, Eluent A: 100% acetonitrile, Eluent B: 95% 0.1M ammonium acetate, 5% acetonitrile flow 30 mL/min, gradient 25% A to 75% A in 8 minutes to afford 5-cyano-6-[3-(2-methoxycarbonyl-phenylmethanesulfonylaminocarbonyl)-azetidin 1-yl]-2-methyl nicotinic acid ethyl ester as a solid. Yield: 0.063 g (50%).

¹H NMR (400 MD, DMSO-d₆) 1.26 (t, J=7.2 Hz, 311), 2.59 (s, 3H), 3.30 (m, 1H overlapped by water), 3.76 (s, 3H), 4.20 (q, J=7.1 Hz, 2H), 4.27 (t, J=2.6 Hz, 21), 4.35 (t, J=4.3 Hz, 2H), 5.12 (s, 21), 7.40 (d, J=7.3 Hz, 1H), 7.48 (t, J=3.9 Hz, 1H), 7.55 (t, J=7.1 Hz, 1H), 7.78 (d, J=7.7 Hz, 1H), 8.26 (s, 1H)

MS ^{m}/_{z}: 501 (M+1)

### Example 2

### 6-[3-({[(3-Bromobenzyl)sulfonyl]amino}carbonyl)azetidin-1-yl]-5-cyano-2-methylnicotinic acid ethyl ester

1-[3-cyano-5-(ethoxycarbonyl)-6-methylpyridin-2-yl]azetidine-3-carboxylic acid (0.257 g, 0.89 mmol), 1-(3-bromophenyl)methanesulfonamide (0.223 g, 0.89 mmol) and TEA (0.360 g, 3.6 mmol) was stirred in DMF (5 mL). HATU (0.405 g, 1.07 mmol) was added and reaction mixture was stirred at r.t for 3.5 h. An additional amount of HATU (0.073 g, 0.18 mmol) was added and the stirring was continued for 18 h. The DMF was evaporated and the residue was dissolved in EtOAc (80 mL). The organic phase was washed with NH₄Cl (sat, aq) (2×8 mL), water (5 mL), dried (MgSO₄), filtered and evaporated to afford 0.658 g of the crude product as a solid. Purification by flash chromatography gave 0.429 g of the product which was about 90% pure according to LC-MS. Further purification of this material was done by preparative HPLC (Kromasil C8 10 μm 250 mm×50 id. Eluent A: 100% acetonitrile, Eluent B: 95% 0.1M aq. ammonium acetate and 5% acetonitrile. Conditions used: Flow 50 mL/minutes, a linear gradient from 0% A to 100% A during 42 minutes was used). The product precipitated during the evaporation of the solvent and was filtered off and washed with water. This afforded the pure product as a white solid. Yield: 0.181 g (39%).

¹H NMR (400 MHz, DMSO-d₆) δ 1.29 (t, J=7.0 Hz, 311), 2.62 (s, 31), 3.56 (m, 1M), 4.23 (q, J=7.0 Hz, 21), 4.25-4.31 (m, 2H), 4.41 (m, 2H), 4.78 (s, 2H), 7.34 (m, 2H), 7.53 (s, 1H), 7.56-7.62 (m, 1H), 8.30 (s, 1H), 11.88 (s, 1H).

MS ^{m}/_{z}: 522 (M+1).

### Example 3

### 5-Cyano-2-methyl-6-[3-(2-nitro-phenylmethanesulfonylaminocarbonyl)-azetidin-1-yl]-nicotinic acid ethyl ester

Prepared according to method A using 1-(2-nitrophenyl)methanesulfonamide (0.11 g crude, 0.37 mmol). Yield: 0.031 g (25%).

¹H NMR (400 MHz, DMSO-d₆) d 1.24 (t, J=7.1 Hz, 3H), 2.57 (s, 3H), 3.5 (m, 1H, overlapped by water), 4.18 (q, J=7.1 Hz, 2H), 4.26 (t, J=3.1 Hz, 2H), 4.34 (t, J=4.2 Hz, 2H), 5.04 (s, 2H), 7.51 (d, J=7.5 Hz, 1H), 7.59 (t, J=7.3 Hz, 1H), 7.66 (t, J=6.9 Hz, 1H), 7.94 (d, J=8.1 Hz, 1H), 8.24 (s, 1H)

MS ^{m}/_{z}: 488 (M+1)

### Example 4

### 6-[3-(2-Chloro-phenylmethanesulfonylaminocarbonyl)azetidin-1-yl]-5-cyano-2-methyl-nicotinic acid ethyl ester

Prepared according to method A using 1-(2-chlorophenyl)methanesulfonamide (0.100 g crude, 0.37 mmol). Yield: 0.031 g (25%).

¹H NMR (400 MHz, DMSO-d₆) δ 1.23 (t, J=7.1 Hz, 3H), 2.57 (s, 3H), 3.52 (m, 1H, overlapped by water), 4.18 (q, J=7.1 Hz, 2H), 4.30 (t, J=7.4 Hz, 2H), 4.40 (t, J=9.4 Hz, 2H), 4.81 (s, 2H), 7.31-7.38 (m, 2H), 7.44 (m, 2H), 8.25 (s, 1H)

MS ^{m}/_{z}: 477 (M+1)

### Example 5

### 6-[3-(4-Chloro-phenylmethanesulfonylaminocarbonyl)-azetidin-1-yl]-5-cyano-2-methyl-nicotinic acid ethyl ester

Prepared according to method A using 1-(4-chlorophenyl)methanesulfonamide (0.106 g crude, 0.37 mmol). Yield: 0.057 g (48%).

¹H NMR (400 MHz, DMSO-d₆) δ 1.24 (t, J=7.1 Hz, 3H), 2.58 (s, 3H), 3.45 (m, 1H, overlapped by water), 4.18 (q, J=7.1 Hz, 2H), 4.23 (m, 2H), 4.36 (t, J=8.6 Hz, 2H), 4.65 (s, 2H), 7.29 (d, J=8.5 Hz, 2H), 7.37 (d, J=8.5 Hz, 2M, 8.25 (s, 1H)

MS ^{m}/_{z}: 477 (M+1)

### Example 6

### 5-Cyano-2-methyl-6-[3-(4-trifluoromethyl-phenylmethanesulfonylaminocarbonyl)-azetidin-1-yl]-nicotinic acid ethyl ester

Prepared according to method A using 1-[4-(trifluoromethyl)phenyl]methanesulfonamide (0.057 g crude, 0.23 mmol). Yield: 0.050 g (390%).

¹H NMR (400 MHz, DMSO-d₆) δ 1.24 (t, J=7.1 Hz, 3H), 2.57 (s, 3H), 3.49 (m, 1H, overlapped by water), 4.2 (q, J=7.1 Hz, 2H), 4.24 (m, 2H), 4.36 (t, J=8.8 Hz, 2H), 4.75 (s, 2H), 7.51 (d, J=7.9 Hz, 2H), 7.68 (d, J=8.1 Hz, 2H), 8.24 (s, 1H)

MS ^{m}/_{z}: 511 (M+1)

### Example 7

### 5-Cyano-6-[3-(3-fluoro-phenylmethanesulfonylaminocarbonyl)-azetidin-1-yl]-2-methyl-nicotinic acid ethyl ester

Prepared according to method A using 1-(3-fluorophenyl)methanesulfonamide (0.095 g crude, 0.37 mmol). Yield: 0.065 g (56%).

¹H NMR (400 MHz, DMSO-d₆) δ 1.24 (t, J=7.1 Hz, 3H), 2.57 (s, 3H), 3.48 (m, 1H, overlapped by water), 4.23-4.15 (m, 4H), 4.36 (t, J=9.1 Hz, 2H), 4.69 (s, 2H), 7.18-7.09 (m, 3H), 7.36 (q, J=7.5 Hz, 1H), 8.24 (s, 1H)

MS ^{m}/_{z}: 461 (M+1)

### Example 8

### 5-Cyano-2-methyl-6-[3-(3-trifluoromethyl-phenylmethanesulfonylaminocarbonyl)azetidin-4-yl]-nicotinic acid ethyl ester

Prepared according to method A using 1-[3-(trifluoromethyl)phenyl]methanesulfonamide (0.105 g crude, 0.37 mmol). Yield: 0.050 g (39%).

¹H NMR (400 MHz, DMSO-d₆) d 1.24 (t, J=7.1 Hz, 3H), 2.57 (s, 3H), 3.48 (m, 1H), 4.18 (m, 4H), 4.35 (t, J=8.8 Hz, 2H), 4.78 (s, 2H), 7.57 (m, 3H), 7.69 (d, J=6.6 Hz, 1H), 8.24 (s, MS ^{m}/_{z}: 511 (M+1)

### Example 9

### 6-[3-(3-Chloro-phenylmethanesulfonylaminocarbonyl)-azetidin-1-yl]-5-cyano-2-methyl-nicotinic acid ethyl ester

Prepared according to method A using 1-(3-chlorophenyl)methanesulfonamide (0.096 g crude, 0.37 mmol). Yield: 0.050 g (42%).

¹H NMR (400 MHz, DMSO-d₆) d 1.24 (t, J=7.1 Hz, 3H), 2.57 (s, 3H), 3.46 (m, 1H overlapped by water), 4.23-4.15 (m, 4H), 4.35 (d, J=9.3 Hz, 21H), 4.68 (s, 2H), 7.24 (d, J=7.3 Hz, 1H), 7.38-7.31 (m, 3H), 8.24 (s, 1H)

MS ^{m}/_{z}: 477 (M+1)

### Example 10

### 6-{3-[2-(3-Chloro-phenyl)-ethanesulfonylaminocarbonyl]-azetidin-1-yl}-5-cyano-2-methyl-nicotinic acid ethyl ester

Prepared according to method A using 2-(3-chlorophenyl)ethanesulfonamide (0.102 g crude, 0.37 mmol). Yield: 0.055 g (45%).

¹H NMR (400 MHz, DMSO-d₆) d 1.23 (t, J=7.1 Hz, 3H), 2.56 (s, 3H), 2.98 (t, J=7.5 Hz, 2H), 3.45 (m, 2H), 3.8-3.5 (m, 2H overlapped by water), 4.17 (m, 3H), 4.34 (t, J=8.5 Hz, 2H), 7.17 (d, J=7.1 Hz, 2H), 7.29-7.24 (m, 2H), 8.23 (s, 1H)

MS ^{m}/_{z}: 491 (M+1)

### Example 11

### 5-Cyano-2-methyl-6-[3-(4-nitro-phenylmethanesulfonylaminocarbonyl)-azetidin-1-yl]-nicotinic acid ethyl ester

Prepared according to method A using 1-(4-nitrophenyl)methanesulfonamide (0.099 g crude, 0.37 mmol). Yield: 0.032 g (26%).

1H NMR (400 MHz, DMSO-d₆) δ 1.24 (t, J=7.2 Hz, 3H), 2.56 (s, 3H), 3.44 (m, 1H), 4.18 (m, 4H), 4.33 (t, J=8.4 Hz, 2H), 4.79 (s, 2H), 7.56 (d, J=8.7. Hz, 2H), 8.15 (d, J=8.7 Hz, 2H), 8.23 (s, 1H)

MS ^{m}/_{z}: 488 (M+1)

### Example 12

### 5-Cyano-2-methyl-6-[3-(2-phenyl-ethanesulfonylaminocarbonyl)-azetidin-1-yl]-nicotinic acid ethyl ester

Prepared according to method A using 2-phenylethanesulfonamide (0.078 g crude, 0.37). Yield: 0.044-g (39%).

¹H NMR (400 M, DMSO-d₆) d 1.23 (t, J=7.2 Hz, 3H), 2.55 (s, 3H), 2.95 (t, J=7.7 Hz, 2H), 3.45 (m, 1H), 4.17 (q, J=7.1 Hz, 4H), 4.34 (t, J=8.6 Hz, 2H), 7.25-7.12 (m, 5H), 8.23 (s, 1H) (The two protons next to the sulfone are overlapping with the DMSO signal)

MS ^{m}/_{z}: 457 (M+1)

### Example 13

### 5-Cyano-2-methyl-&(3-o-tolylmethanesulfonylaminocarbonyl-azetidin-1-yl)-nicotinic acid ethyl ester

Prepared according to method A using 1-(2-methylphenyl)methanesulfonamide (0.010 g crude, 0.05 mmol). Yield: 0.002 g (2%)ₒ.

¹H N (400 MHz, DMSO-d₆) δ 1.24 (t, J=7.2 Hz, 3M), 2.32 (s, 3H), 2.57 (s, 3H), 3.4 (m, 1H overlapped by water), 4.18 (m, 2H), 4.30 (m, 2H), 4.39 (m, 2H), 4.63 (s, 21), 7.15 (m, 4H), 8.24 (s, 1H)

MS ^{m}/_{z}: 457 (M+1)

### Example 14

### 5-Cyano-2-methyl-6-[3-(3-nitro-phenylmethanesulfonylaminocarbonyl)-azetidin-1-yl]-nicotinic acid ethyl ester

Prepared according to method A using 1-(3-nitrophenyl)methanesulfonamide (0.097 g crude, 0.37 mmol). Yield: 0.055 g (45%).

¹H NMR (400 MHz, DMSO-d₆) d 1.24 (t, J=7.2 Hz, 3H), 2.57 (s, 3H), 3.46 (m, 1H), 4.18 (m, 4H), 4.32 (t, J=9.2 Hz, 2H), 4.82 (s, 2H), 7.63 (t, J=8.0 Hz, 1H), 7.75 (d, J=7.7 Hz, 1H), 8.10 (s, 1H), 8.17 (d, J=8.1 Hz, 1H), 8.23 (s, 1H)

MS ^{m}/_{z}: 488 (M+1)

### Example 15

### 5-Cyano-6-{3-[2-(4-fluoro-phenyl)-ethanesulfonylaminocarbonyl]-azetidin-1-yl}-2-methyl-nicotinic acid ethyl ester

Prepared according to method A using 2-(4-fluorophenyl)ethanesulfonamide (0.082 g crude, 0.37 mmol). Yield: 0.051 g (43%).

¹H NMR (400 MHz, DMSO-d₆) d 1.23 (t, J=7.2 Hz, 3H), 2.55 (s, 3H), 2.95 (t, J=7.6 Hz, 2H), 3.48 (m, 1H), 3.70-3.50 (m, 2H), 4.17 (q, J=7.2 Hz, 4H), 4.36 (t, J=9.0 Hz, 2H), 7.04 (t, J=8.9 Hz, 2H), 7.24 (dd, J=8.6, 5.5 Hz, 2H), 8.23 (s, 1H)

MS ^{m}/_{z}: 475 (M+1)

### Example 16

### 5-Cyano-2-methyl-6-[3-(2-trifluoromethyl-phenylmethanesulfonylaminocarbonyl)-azetidin-1-yl]-nicotinic acid ethyl ester

Prepared according to method A using 1-[2-(trifluoromethyl)phenyl]methanesulfonamide (0.100 g crude, 0.37 mmol). Yield: 0.045 g (35%).

¹H N (400 MHz, DMSO-d₆) d 1.23 (t, J=7.1 Hz, 3H), 2.57 (s, 3H), 3.58 (m, 1H), 4.18 (q, J=7.1 Hz, 2H), 4.31 (t, J=6.9 Hz, 2H), 4.41 (t, J=8.8 Hz, 2H), 4.83 (s, 2H), 7.68-7.54 (m, 3H), 7.74 (d, J=8.1 Hz, 1H), 8.24 (s, 1H)

MS ^{m}/_{z}: 511 (M+1)

### Example 17

### 5-Cyano-6-[3-(4-fluoro-phenylmethanesulfonylaminocarbonyl)-azetidin-1-yl]-2-methyl-nicotinic acid ethyl ester

Prepared according to method A using 1-(4-fluorophenyl)methanesulfonamide (0.078 g crude, 0.37 mmol). Yield: 0.050 g (43%).

¹H NMR (400 MHz, DMSO-d₆) d 1.24 (t, J=7.1 Hz, 3H), 2.57 (s, 3H), 3.49 (m, 1H), 4.20 (m, 4H), 4.36 (t, J=9.0 Hz, 2H), 4.65 (s, 2H), 7.13 (t, J=8.8 Hz, 2H), 7.32 (dd, J=8.6, 5.5 Hz, 2H), 8.25 (s, 1H)

MS ^{m}/_{z}: 461 (M+1)

### Example 18

### 5-Cyano-6-(3-cyclopentylmethanesulfonylaminocarbonyl-azetidin-1-yl)-2-methyl-nicotinic acid ethyl ester

Prepared according to method A using 1-cyclopentylmethanesulfonamide (0.074 g crude, 0.37 mmol). Yield: 0.013 g (12%).

¹H NMR (400 MHz, DMSO-d₆) d 1.21 (m, 2H), 1.23 (t, J=7.1 Hz, 3H), 1.44 (m, 2H), 1.52 (m, 2H), 1.79 (m, 2H), 2.13 (m, 1H), 2.56 (s, 3H), 3.38 (d, J=6.8 Hz, 2H), 3.51 (m, 1H overlapped by water), 4.17 (q, J=7.1 Hz, 2H), 4.29 (m, 2H), 4.41 (t, J=9.0 Hz, 2H), 8.23 (s, 1H)

MS ^{m}/_{z}: 435.0 (M+1)

### Example 19

### 5-Cyano-6-{3-[2-(2-fluoro-phenyl)-ethanesulfonylaminocarbonyl]-azetidin-1-yl}-2-methyl-nicotinic acid ethyl ester

Prepared according to method A using 2-(2-fluorophenyl)ethanesulfonamide (0.084 g crude, 0.37 mmol). Yield: 0.060 g (51%).

¹H NMR (400 MHz, DMSO-d) d 1.23 (t, J=7.1 Hz, 3H), 2.56 (s, 3H), 2.99 (t, J=7.7 Hz, 2H), 3.46 (m, 1H overlapped by water), 3.67-3.54 (m, 2H overlapped by water), 4.17 (m, 4H), 4.36 (t, J=8.8 Hz, 2H), 7.08 (t, J=8.0 Hz, 2H), 7.21 (m, 1H), 7.30 (t, J=7.7 Hz, 1H), 8.23 (s, 1H)

MS ^{m}/_{z}: 475 (M+1)

### Example 20

### 5-Cyano-6-[3-(3,5-dichloro-phenylmethanesulfonylaminocarbonyl)-azetidin-1-yl]-2-methyl-nicotinic acid ethyl ester

Prepared according to method A using 1-(3,5-dichlorophenyl)methanesulfonamide (0.181 g crude, 0.37 mmol). Yield: 0.053 g (41%).

¹H NMR (400 MHz, DMSO-d₆) d 1.23 (t, J=7.2 Hz, 3H), 2.56 (s, 3H), 3.47 (m, 1H), 4.18 (m, 2H), 4.18 (q, J=7.1 Hz, 2H), 4.35 (t, J=9 Hz, 21H), 4.69 (s, 2H), 7.30 (s, 2H), 7.55 (s, 1H), 8.23 (s, 1H)

MS ^{m}/_{z}: 511 (M+1)

### Example 21

### 5-Cyano-6-(3-cyclohexylmethanesulfonylaminocarbonyl-azetidin-1-yl)-2-methyl-nicotinic acid ethyl ester

Prepared according to method A using 1-cyclohexylmethanesulfonamide (0.065 g crude, 0.37 mmol). Yield: 0.012 g (11%).

¹H NMR (400 MHz, DMSO-d₆) d 0.98-1.25 (m, 8H), 1.60-1.50 (m, 3H), 1.74 (m, 3H), 2.55 (s, 3H), 3.26 (d, J=6.0 Hz, 2H), 3.58 (m, 1H), 4.17 (q, J=7.1 Hz, 2H), 4.28 (t, J=7.1 Hz, 2H), 4.41 (t, J=8.7 Hz, 2H), 8.23 (s, 1H)

MS ^{m}/_{z}: 449 (M+1)

### Example 22

### 5-Cyano-6-{3-[2-(3-fluoro-phenyl)-ethanesulfonylaminocarbonyl]-azetidin-1-yl}-2-methyl-nicotinic acid ethyl ester

Prepared according to method A using 2-(3-fluorophenyl)ethanesulfonamide (0.088 g crude, 0.37 mmol). Yield: 0.044 g (37%).

¹H N (400 MHz, DMSO-d₆) d 1.23 (t, J=7.1 Hz, 31), 2.56 (s, 3H), 2.98 (t, J=7.7 Hz, 2H), 3.45 (m, 1H overlapped by water), 3.69-3.56 (m, 2H overlapped by water), 4.17 (m, 2H), 4.17 (q, J=7.1 Hz, 2H), 4.35 (t, J=8.9 Hz, 2H), 6.95 (m, 1H), 7.06 (m, 2H), 7.27 (dd, J=14.4, 8.0 Hz, 1H), 8.23 (s, 1H)

MS ^{m}/_{z}: 475 (M+1)

### Example 23

### 6-[3-(Benzo[d]isoxazol-3-ylmethanesulfonylaminocarbonyl)-azetidin-1-yl]-5-cyano-2-methyl-nicotinic acid ethyl ester

Prepared according to method A using 1-(1,2-benzisoxazol-3-yl)methanesulfonamide (0.080 g, 0.37 mmol). Yield: 0.035 g (28%).

¹H NMR (400 MHz, DMSO-d₆) δ 1.26 (t, J=7.1 Hz, 3H), 2.58 (s, 3H), 3.27 (m, 1H overlapped by water), 4.20 (q, J=7.1 Hz, 2H), 4.84 (s, 2H), 4.27 (m, 4H), 7.28 (t, J=7.5 Hz, 1H), 7.56 (t, J=7.6 Hz, 1H), 7.66 (d, J=8.3 Hz, 1H), 7.91 (d, J=7.9 Hz, 1H), 8.24 (s, 1H)

MS ^{m}/_{z}: 484 (M+1)

### Example 24

### 1-[4-Amino-3-chloro-5-(5-ethyl-1,3-oxazol-2-yl)pyridin-2-yl]-N-(benzylsulfonyl)piperidine 4-carboxamide

### (a) 5,6-Dichloro-N-(2-hydroxybutyl)nicotinamide

5,6-Dichloronicotinic acid (20.0 g, 104 mmol), EDCI (26.0 g, 135 mmol) and HOBt (18.3 g, 135 mmol) were dissolved in DCM (500 mL) at r.t. The reaction mixture was stirred at r.t for 90 minutes and then 1-aminobutan-2-ol (15.0 g, 156 mmol) and DIPEA (54.4 mL, 313 mmol) were added. The reaction mixture was stirred at r.t for 18 h. The reaction mixture was diluted with DCM (400 mL) and the combined organics were washed with saturated NH₄Cl (2×100 mL), saturated NaHCO₃ (2×100 mL), dried (MgSO₄) and concentrated under reduced pressure to afford 5,6-dichloro-N-(2-hydroxybutyl)nicotinamide as a solid, which was used crude assuming a 100% conversion

### (b) 5,6-Dichloro-N-(2-oxobutyl)nicotinamide

Oxalyl Chloride (16.3 mL, 187 mmol) was dissolved in DCM (500 mL) and cooled to −78° C. DMSO (26.3 mL, 374 mmol) was added drop-wise and stirred at −78° C. for 10 minutes. 5,6-Dichloro-N-(2-hydroxybutyl)nicotinamide (30 g, 94 mmol) was dissolved in DCM/DMSO (3:1) and added slowly to the solution. The solution was stirred at −78° C. for 30 minutes. TEA (65.2 μL, 467 mmol) was added to the solution and stirred for 30 minutes. The solution was warmed to r.t and stirred for 3 h. The reaction mixture was diluted with DCM (200 mL) and the combined organics were washed with water (2×200 mL), brine (2×200 mL), dried (MgSO₄) and concentrated under reduced pressure to afford 5,6-dichloro-N-(2-oxobutyl)nicotinamide as a solid, which was used crude assuming a 100% conversion

### (c) 2,3-dichloro-5-(5-ethyl-1,3-oxazol-2-yl)pyridine

5,6-Dichloro-N-(2-oxobutyl)nicotinamide (26.7 g, 78 mmol) and POCl₃ (59.6 g, 389 mmol) were dissolved in DMF (500 mL) and heated at 90 DC for 30 minutes. The reaction mixture was poured onto ice. Solid NaHCO₃ was added in portions until the pH was raised to pH>8. The reaction mixture was diluted with water (500 mL) and the combined aqueous were washed with EtOAc (3×400 mL), dried (MgSO₄) and concentrated under reduced pressure to afford the crude product as a solid. Flash chromatography (EtOAc/hexanes, 1/9) gave 2,3-dichloro-5-(5-ethyl-1,3-oxazol-2-yl)pyridine as a solid. Yield: 7.08 g (37.5%).

¹H NMR (400 MHz, CDCl₃): δ 1.33 (2H, t, J=7.5 Hz), 2.78 (2H, q, J=7.5 Hz), 6.91 (1H, s), 8.35 (1H, d, J=1.9 Hz) 8.29 (1H, d, J=1.9 Hz).

MS ^{m}/_{z}: 244 (M+1).

### (d) 2,3-dichloro-5-(5-ethyl-1,3-oxazol-2-yl)-4-(methylthio)pyridine

n-Butyllithium (2.5 M in hexanes, 7.14 mL, 17 mmol) was added drop-wise to diisopropylamine (2.62 mL, 19 mmol) in THF (5 mL) at 0° C. The solution was stirred at 0° C. for 30 minutes and then cooled to −78° C. 2,3-dichloro-5-(5-ethyl-1,3-oxazol-2-yl)pyridine (3.50 g, 14 mmol) in THF (30 mL) was added to the solution and the reaction was stirred at −78° C. for 1 h. S-methyl methanesulfonothioate (1.77 mL, 19 mmol) was added and the solution warmed to r.t. The reaction mixture was stirred for 16 h. The reaction mixture was diluted with saturated NH₄Cl (100 mL). The solution was washed with EtOAc (3×50 mL). The combined organics were washed with brine (1×50 mL), dried (MgSO₄) and concentrated under reduced pressure to afford the crude product as a solid. Flash chromatography (15% EtOAc/hexanes to 20% EtOAc/hexanes) gave 2,3-dichloro-5-(5-ethyl-1,3-oxazol-2-yl)-4-(methylthio)pyridine as a solid. Yield: 2.71 g (65.1%).

¹H NMR (400 MHz, CDCl₃): δ 1.33 (2H, t, J=7.6 Hz), 2.35 (3H, s), 2.79 (2H, q, J=7.6 Hz), 6.98 (1H, s), 8.58 (1H, s).

MS ^{m}/_{z}: 290 (M+1).

### (e) Methyl 1-[3-chloro-5-(5-ethyl-1,3-oxazol-2-yl)-4-(methylthio)pyridin-2-yl]piperidine-4-carboxylate

2,3-dichloro-5-(5-ethyl-1,3-oxazol-2-yl)-4-(methylthio)pyridine (3.11 g, 11 mmol), methyl piperidine-4-carboxylate (2.00 g, 14 mmol) and DIPEA (3.75 mL, 22 mmol) were dissolved in DMA (50 mL) and heated to 120° C. for 2 h. The reaction mixture was cooled to r.t and concentrated under reduced pressure. The crude material was dissolved in EtOAc (100 mL), washed with NH₄Cl (2×60 mL), dried (MgSO₄) and concentrated under reduced pressure to afford the crude product as a solid. Flash chromatography (1:5 EtOAc/hexanes to 1:3 EtOAc/hexanes) gave methyl 1-[3-chloro-5-(5-ethyl-1,3-oxazol-2-yl)-4-(methylthio)pyridin-2-yl]piperidine-4-carboxylate as a solid. Yield: 4.26 g (87.6%).

¹H NMR (400 MHz, CDCl₃): δ 1.33 (2H, t, J=7.6 Hz), 1.88-2.06 (4H, m), 2.32 (3H, s), 2.51-2.58 (1H, m), 2.76 (3H, q, J=7.6 Hz), 2.93-2.99 (2H, m), 3.72 (3H, s), 3.81-3.92 (2H, n), 6.91 (1H, s), 8.43 (1H, s).

MS ^{m}/_{z}: 396 (M+1).

### (f) Methyl 1-[3-chloro-5-(5-ethyl-1,3-oxazol-2-yl)-4-(methylsulfinyl)pyridin-2-yl]piperidine-4-carboxylate

Methyl 1-[3-chloro-5-(5-ethyl 1,3-oxazol-2-yl)-4-(methylthio)pyridin-2-yl]piperidine-4-carboxylate (2.12 g, 5.4 mmol) was dissolved in DMF (500 mL) and 3-chlorobenzenecarboperoxoic acid (2.64 g, 10.7 mmol) was slowly added at r.t. The solution was stirred at r.t for 4 h. 3-chlorobenzenecarboperoxoic acid (1.32 g, 5.35 mmol) was slowly added at r.t for 3 h. Saturated Na₂S₂O₃ (30 mL) was added and the solution was stirred for 5 minutes. The reaction mixture was diluted with CH₂Cl₂ (40 mL) and the combined organics were separated and washed with NaOH (1M, 2×40 mL), brine (1×30 mL), dried (MgSO₄) and concentrated under reduced pressure to afford the crude product. Flash chromatography (1:2 EtOAc/hexanes) gave methyl 1-[3-chloro-5-(5-ethyl-1,3-oxazol-2-yl)-4-(methylsulfinyl)pyridin-2-yl]piperidine-4-carboxylate as a solid. Yield: 2.71 g (65.1%).

¹H NMR (400 MHz, CDCl₃): δ 1.30 (1H, t, J=7.5 Hz), 1.83-2.08 (4H, m) 2.52-2.61 (1H, m), 2.75 (2H, q, J=7.5 Hz), 2.93-3.00 (1H, m), 3.04-3.13 (1H, m), 3.23 (3H, s), 3.72 (3H, s), 3.86-4.01 (2-H, m), 6.87 (1H, s), 8.51 (1H, s).

MS ^{m}/_{z}: 412 (M+1).

### (g) Methyl 1-[4-azido-3-chloro-5-(5-ethyl-1,3-oxazol-2-yl)pyridin-2-yl]piperidine-4-carboxylate

Methyl 1-[3-chloro-5-(5-ethyl-1,3-oxazol-2-yl)-4-(methylsulfinyl)pyridin-2-yl]piperidine-4-carboxylate (0.150 g, 0.36 mmol) and sodium azide (0.026 g, 0.40 mmol) were dissolved in DMA (1 mL) and stirred at r.t for 48 h The reaction mixture was diluted with EtOAc (40 mL) and the combined organics were separated and washed with water (2×40 mL), brine (1×30 mL), dried (MgSO₄) and concentrated under reduced pressure to afford methyl 1-[4-azido-3-chloro-5-(5-ethyl-1,3-oxazol 2-yl)pyridin-2-yl]piperidine-4-carboxylate as a solid, which was used crude assuming a 100% conversion

### (h) Methyl 1-[4-amino-3-chloro-5-(5-ethyl-1,3-oxazol-2-yl)pyridin-2-yl]piperidine-4-carboxylate

Methyl 1-[azido-3-chloro-5-(5-ethyl-1,3-oxazol-2-yl)pyridin-2-yl]piperidine-4-carboxylate (0.150 g, 0.36 mmol) was dissolved in THF (0.90 mL) and cooled to 0° C. Zinc dust (0.109 g, 1.66 mmol) was added. NH₄Cl (0.900 mL) was added slowly to the solution. The solution was warmed to r.t for 1.5 h. The reaction mixture was filtered (celite) and diluted with EtOAc (40 mL) and the combined organics were washed with saturated with NH₄OAc (2×30 mL), brine (1×30 mL), dried (MgSO₄) and concentrated under reduced pressure to afford methyl 1-[4-amino-3-chloro-5-(5-ethyl-1,3-oxazol-2-yl)pyridin-2-yl]piperidine-4-carboxylate as a solid, which was used crude assuming a 100% conversion.

### (i) 1-[4-Amino-3-chloro-5-(5-ethyl-1,3-oxazol-2-yl)pyridin-2-yl]piperidine-4-carboxylic acid

Methyl 1-[4-amino-3-chloro-5-(5-ethyl-1,3-oxazol-2-yl)pyridin-2-yl]piperidine-4-carboxylate (0.045 g, 0.123 mmol), and lithium hydroxide (2 M, 1.23 mL, 2.46 mmol) were suspended in THF (1 mL) and stirred at r.t for 16 h HCl (conc.) was added drop-wise to the mixture until the pH was lowered to pH 2. The solution was washed with EtOAc (3×40 nm), dried (MgSO₄), and concentrated under reduced pressure to afford 1-[4-amino-3-chloro-5-(5-ethyl-1,3-oxazol-2-yl)pyridin-2-yl]piperidine-4-carboxylic acid as a solid, which was used crude assuming a 100% yield.

### (j) 1-[4-Amino-3-chloro-5-(5-ethyl-1,3-oxazol-2-yl)pyridin-2-yl]-N-(benzylsulfonyl)piperidine-4-carboxamide

1-[4-amino-3-chloro-5-(5-ethyl 1,3-oxazol-2-yl)pyridin-2-yl]piperidine-4-carboxylic acid (0.040 g, 0.11 mmol), EDCI (0.026 g, 0.14 mmol) and HOBt (0.019 g, 0.14 mmol) were dissolved in DCM (2 mL) at r.t The reaction mixture was stirred at r.t for 30 minutes and then 1-phenylmethanesulfonamide (0.023 g, 0.14 mmol) and DIPEA (0.099 mL, 0.57 mmol) were added. The reaction mixture was stirred at r.t for 48 h The reaction mixture was diluted with EtOAc (50 mL). The combined organics were washed with saturated NH₄Cl (2×30 mL), dried (MgSO₄) and concentrated under reduced pressure to afford the crude product Flash chromatography (EtOAc/hexanes 5/1 to EtOAc/hexanes 5/1 with 0.5% AcOH) gave 1-[4-amino-3-chloro-5-(5-ethyl 1,3-oxazol-2-yl)pyridin 2-yl]-1-(benzylsulfonyl)piperidine-4-carboxamide as a solid. Yield: 0.018 g (30.50%).

¹H NMR (400 MHz, CDCl₃): δ 1.24-1.33 (6H, m), 1.85-1.93 (4H, m), 2.26-2.35 (1H, m), 2.71-2.88 (4H, m), 3.80-3.89 (2H, m), 4.69 (2H, s), 6.81 (1H, s), 7.35-7.44 (5H, m), 8.52 (1H, s).

MS ^{m}/_{z}: 505 (M+1).

### Example 25

### 4-Amino-6-(4-{[(benzylsulfonyl)amino]carbonyl}piperidin-1-yl)-5-chloronicotic acid ethyl ester

### (a) Ethyl 4-azido-5,6-dichloronicotinate

4,5,6-Trichloronicotinic acid (1.28 g, 5.65 mmol) and sodium azide (0.370 g, 5.69 mmol) were dissolved in DMA (10 mL) and stirred at r.t for 16 h. Iodoethane (0.670 mL, 6.60 mmol) and potassium carbonate (3.90 g, 28.25 mmol) were added to the reaction mixture and stirred at r.t for 16 h. The reaction mixture was diluted with EtOAc (40 mL) and the combined organics were washed with water (2×40 mL), brine (1×30 mL), dried (MgSO₄) and concentrated under reduced pressure to yield ethyl 4-azido-5,6-dichloronicotinate as a solid, which was used crude assuming 100% conversion

### (b) Ethyl 4-amino-5,6-dichloronicotinate

Ethyl 4-azido-5,6-dichloronicotinate (0.700 g, 2.68 mmol) was dissolved in 1:1 THF/MeOH (10 mL). Zinc dust (0.109 g, 1.66 mmol) was added and the solution was cooled to 5° C. NH₄Cl (2 mL) was added slowly to the solution. The solution was warmed to r.t for 2 h. The reaction mixture was filtered (celite), washed with MeOH (50 mL) and concentrated to yield ethyl 4-amino-5,6-dichloronicotinate as a solid, which was used crude assuming a 100% conversion

### (c) 1-[4-Amino-3-chloro-5-(ethoxycarbonyl)pyridin-2-yl]piperidine-4-carboxylic acid

Ethyl 4-amino-5,6-dichloronic (0.320 g, 1.36 mmol), piperidine 4-carboxylic acid (0.352 g, 2.72 mmol) and DIPEA (11.9 mL, 68.2 mmol) were dissolved in DMA (2.5 mL) and heated at 120° C. for 2 h. The reaction mixture was cooled to r.t and concentrated under reduced pressure. The crude material was dissolved in EtOAc (40 mL), washed with NH₄Cl (1×40 mL), dried (MgSO₄) and concentrated under reduced pressure to afford the crude product. Flash chromatography (EtOAc/hexanes 1/3 to EtOAc/hexanes 2/3 with 0.5% AcOH) gave 1-[4-amino-3-chloro-5-(ethoxycarbonyl)pyridin-2-yl]piperidine-4-carboxylic acid as a solid.

Yield: 0.154 g (34.5%).

¹H NMR (400 MHz, CDCl₃): δ 1.37 (2H, t, J=7.1 Hz), 1.88-2.07 (4H, m), 2.55-2.62 (1H, m), 2.92-3.01 (2H, m), 3.87-3.90 (2H, m), 4.33 (3H, q, J=7.1 Hz), 8.60 (1H, s).

MS ^{m}/_{z}: 328 (M+1).

### (d) 4-Amino-6-(4-{[(benzylsulfonyl)amino]carbonyl}piperidin-1-yl)-5-chloronicotic acid ethyl ester

1-[4-Amino-3-chloro-5-(ethoxycarbonyl)pyridin-2-yl]piperidine-4-carboxylic acid (0.070 g, 0.21 mmol), EDCI (0.053 g, 0.28 mmol) and HOBt (0.038 g, 0.28 mmol) were dissolved in DCM (5 mL) at r.t. The reaction mixture was stirred at r.t for 30 minutes and then 1-phenylmethanesulfonamide (0.051 g, 0.30 mmol) and DIPEA (0.22 mL, 1.3 mmol) were added. The reaction mixture was stirred at r.t until complete consumption of starting material was observed by HPLC analysis. The reaction mixture was diluted with DCM (30 mL) and washed with saturated NH₄Cl (2×30 mL). The combined organics were dried (MgSO₄) and concentrated under reduced pressure to afford the crude product. Flash chromatography (3:7 EtOAc/hexanes then 3:7 EtOAc/hexanes with 0.5% AcOH) 4-amino-6-(4-{[(benzylsulfonyl)amino]carbonyl}piperidin-1-yl)-5-chloronicotic acid ethyl ester as a solid. Yield: 0.079 g (77%).

¹H NMR (400 MHz, CDCl₃): δ 1.37 (3H, t, J=7.4 Hz), 1.83-1.88 (4H, m), 2.28-2.36 (1H, m), 2.81-2.88 (2H, m), 3.92-3.95 (2H, m), 4.33 (2H, q, J=7.4 Hz), 4.69 (4H, s), 7.35-7.41 (5H, m), 8.59 (1H, s).

MS ^{m}/_{z}: 481 (M+1),

### Example 26

### 6-[3-({[(Benzylsulfonyl)amino]carbonyl}amino)azetidin-1-yl]-S-cyano-2-methylnicotinic acid isopropyl ester

### (a) 6-{3-[(tert-butoxycarbonyl)amino]azetidin-1-yl}-5-cyano-2-methylnicotinic acid

Ethyl 6-(3-(tert-butoxycarbonyl)azetidin-1-yl)-5-cyano-2-methylnicotinate (1.50 g, 4.16 mmol), and lithium hydroxide (3.00 g, 8.32 mmol) were suspended in MeOH (40 mL) and heated at 90° C. for 1 h. HCl (conc.) was added dropwise to the mixture until the pH was lowered to pH 2. The precipitate was filtered and collected. The mother liquor was washed with EtOAc (1×60 mL), dried (MgSO₄), concentrated under reduced pressure and combined with the solid to afford 6-{3-[(tert-butoxycarbonyl)amino]azetidin-1-yl}-5-cyano-2-methylnicotinic acid as a solid, which was used crude.

### (b) Isopropyl 6-{3-[(tert-butoxycarbonyl)amino]azetidin-1-yl}-5-cyano-2-methylnicotinate

6-{3-[(tert-Butoxycarbonyl)amino]azetidin 1-yl}-5-cyano-2-methylnicotinic acid (0.400 g, 1.20 mmol), 2-iodopropane (0.181 mL, 1.81 mmol), and potassium carbonate (0.216 g, 1.56 mmol) were dissolved in DMA (5 mL). The reaction mixture was stirred at r.t for 16 h. 2-Iodopropane (0.154 g, 0.91 mmol) was added to the solution and stirring continued for an additional 8 h. The reaction mixture was diluted with EtOAc (40 mL). The combined organics were washed with saturated NaHCO₃ (2×40 mL), dried (MgSO₄) and concentrated under reduced pressure to afford isopropyl 6-{3-[(tert-butoxycarbonyl)amino]azetidin-1-yl}-5-cyano-2-methylnicotinate as a solid, which was used crude.

### (c) Isopropyl 6-(3-aminoazetidin-1-yl)-5-cyano-2-methylnicotinate bis(trifluoroacetate)

Isopropyl 6-{3-[(tert-butoxycarbonyl)amino]azetidin 1-yl}-5-cyano-2-methylnicotinate (0.376 g, 1.00 mmol) was dissolved in DCM (1 mL). TFA (1.16 mL, 15.1 mmol) was added slowly. The reaction mixture was stirred at r.t for 16 h. The mixture was concentrated under reduced pressure to afford isopropyl 6-(3-aminoazetidin-1-yl)-5-cyano-2-methylnicotinate bis(trifluoroacetate) as a solid, which was used crude assuming a 100% conversion.

### (d) 6-[3-({[(Benzylsulfonyl)amino]carbonyl}amino)azetidin-1-yl]-5-cyano-2-methylnicotinic acid isopropyl ester

1,1′-carbonylbis(1H-imidazole) (0.035 g, 0.216 mmol) and 1-phenylmethanesulfonamide (0.037 g, 0.216 mmol) were dissolved in DCE (2 mL) and stirred for 16 h at r.t. Isopropyl 6-(3-aminoazetidin-1-yl)-5-cyano-2-methylnicotinate bis(trifluoroacetate) (0.102 g, 0.216 mmol) in DCE (2 mL) and DIPEA (0.564 mL, 0.740 mmol) were added to the reaction mixture and stirring continued at r.t for 16 h. The reaction mixture was heated at 70° C. for 16 h. 1,1′-carbonylbis(1H-imidazole) (0.035 g, 0.216 mmol) and 1-phenylmethnesulfonamide (0.037 g, 0.216 mmol) were added to the solution and the reaction mixture was heated at 70° C. for 16 h. The reaction mixture was concentrated under reduced pressure and diluted with EtOAc (40 mL). The combined organics were washed with saturated NaHCO₃ (2×30 mL), dried (MgSO₄) and concentrated under reduced pressure to afford the crude product. Trituration (1:1 EtOAc/hexanes) gave 6-[3-({[(benzylsulfonyl)amino]carbonyl}amino)azetidin 1-yl]-5-cyano-2-methylnicotinic acid isopropyl ester as a solid. Yield: 0.017 g (16.2%).

¹H NMR (400 MHz, DMSO-d₆): δ 1.29 (6H, d, J=6.2 Hz), 2.62 (3H, s), 4.18 (2H, m), 4.55 (3H, m), 4.69 (2H, m), 5.03-5.09 (1H, m), 7.10 (1H, s), 7.32-7.40 (5H, m), 8.31 (1H, s), 10.5 (s, 1H).

MS ^{m}/_{z}: 472 (M+1).

### Example 27

### 6-[3-({[(Benzylsulfonyl)amino]carbonyl}amino)azetidin-1-yl]-5-cyano-2-methylnicotinic acid tert-butyl ester

### (a) tert-Butyl 6-{3-[(tert-butoxycarbonyl)amino]azetidin-1-yl}-5-cyano-2-methylnicotinic acid

6-{3-[(tert-Butoxycarbonyl)amino]azetidin 1-yl}-5-cyano-2-methylnicotinic acid (0.400 g, 1.20 mmol), and tert-butyl imidocarbamate (0.964 g, 4.80 mmol) were dissolved in THF (5 mL) and heated at 80° C. for 27 h. The reaction mixture was concentrated under reduced pressure. The reaction mixture was diluted with DCM (40 mL) and filtered through a silica plug with EtOAc. The filtrate was concentrated under reduced pressure to afford the crude product Flash chromatography (1:6 EtOAc/hexanes) gave tert-butyl 6-{3-[(tert-butoxycarbonyl)amino]azetidin-1-yl}-5-cyano-2-methylnicotinic acid as a solid. Yield: 0.342 g (73.2%).

### (b) tert-Butyl 6-(3-aminoazetidin-1-yl)-5-cyano-2-methylnicotinate dihydrochloride

tert-Butyl 6-{3-[(tert-butoxycarbonyl)amino]azetidin-1-yl}-5-cyano-2-methylnicotinic acid (0.342 g. 0.880 mmol) was dissolved HCl (1 M in dioxane, 4.40 mL, 4.40 mmol). The reaction mixture was stirred at r.t for 16 h and concentrated under reduced pressure to yield tert-butyl 6-(3-aminoazetidin-1-yl)-5-cyano-2-methylnicotinate dihydrochloride as a solid, which was used crude assuming 100% conversion.

### (c) 6-[3-({[(Benzylsulfonyl)amino]carbonyl}amino)azetidin-1-yl]-5-cyano-2-methylnicotinic acid tert-butyl ester

1,1′-carbonylbis(1H-imidazole) (0.034 g, 0.208 mmol) and 1-phenylmethanesulfonamide (0.034 g, 0.208 mmol) were dissolved in DCE (2 mL) and stirred for 16 h at r.t. tert-Butyl 6-(3-aminoazetidin-1-yl)-5-cyano-2-methylnicotinate dihydrochloride (0.130 g, 0.208 mmol) in DCE (2 mL) and DIPEA (2.08 mL, 0.362 mmol) were added to this solution and sired at r.t for 48 h. The reaction mixture was heated to 70° C. for 16 h The reaction mixture was concentrated under reduced pressure and diluted with EtOAc (40 mL). The combined organics were washed with saturated NaHCO₃ (2×30 mL), dried (MgSO₄) and concentrated under reduced pressure to afford the crude product. Flash chromatography (1:6 EtOAc/hexanes) followed by trituration (1:1 EtOAc/hexanes) gave 6-[3-({[(benzylsulfonyl)amino]carbonyl}amino)azetidin-1-yl]-5-cyano-2-methylnicotinic acid tert-butyl ester as a solid. Yield: 0.031 g (30%)

¹H NMR (400 z, DMSO-d₆): δ 1.52 (9H, s), 2.60 (3H, s), 4.16 (2H, m), 4.55 (3H, m), 4.69 (2H, m), 7.10 (1H, s), 7.33-7.40 (5H, m), 8.23 (1H, s), 10.5 (1H, s).

MS ^{m}/_{z}: 486 (M+1).

### Example 28

### 6-[3-({[(Benzylsulfonyl)amino]carbonyl}amino)azetidin-1-yl]-S-cyano-2-methylnicotic acid ethyl ester

### (a) Ethyl 6-{3-[(tert-butoxycarbonyl)amino]azetidin-1-yl}-5-cyano-2-methylnicotinate

Ethyl 6-chloro-5-cyano-2-methylnicotinate (6.20 g, 29.4 mmol), tert-butyl azetidin-3-ylcarbamate (5.07 g, 29.4 mmol), and DIPEA (5.13 mL, 29.4 mmol) were dissolved in DCE (40 mL) and stirred at r.t for 1 h The reaction mixture was concentrated under reduced pressure and diluted with EtOAc (40 mL). The combined organics were washed with saturated NaHCO₃ (2×30 mL), dried (MgSO₄) and concentrated under reduced pressure to afford the crude product. Flash chromatography (1:6 EtOAc/hexanes) gave ethyl 6-{3-[(tertbutoxycarbonyl)amino]azetidin-1-yl}-5-cyano-2-methylnicotinate as a solid Yield: 7.00 g (66.0%)

¹H NMR (400 MHz, CDCl₃): δ 1.37 (3H, t, J=7.2 Hz), 1.46 (9H, s), 2.70 (1H, s), 4.18-4.22 (2H, m), 430 (2H, q, J-7.2 Hz), 4.59 (1H, s), 4.67-4.72 (2H, m), 5.00 (1H, s), 8.26 (1H, s).

MS ^{m}/_{z}: 361 (M+1).

### (b) Ethyl 6-(3-aminoazetidin-1-yl)-5-cyano-2-methylnicotinate bis(trifluoroacetate)

Ethyl 6-{3-[(tert-butoxycarbonyl)amino]azetidin-1-yl}-5-cyano-2-methylnicotinate (1.00 g, 2.77 mmol) was dissolved in DCM (10 mL). TFA (6.40 mL, 83.2 mmol) was added slowly. The reaction mixture was stirred at r.t for 30 minutes. The mixture was concentrated under reduced pressure to afford ethyl 6-(3-aminoazetidin-1-yl)-5-cyano-2-methylnicotinate bis(trifluoroacetate) as a solid, which was used crude assuming a 100% conversion.

### (c) 6-[3-({[(Benzylsulfonyl)amino]carbonyl}amino)azetidin-1-yl]-5-cyano-2-methylnicotic acid ethyl ester

1,1′-carbonylbis(1H-imidazole) (0.054 g, 0.333 mmol) and 1-phenylmethanesulfonamide (0.057 g, 0.333 mmol) were dissolved in DCE (2 mL) and stirred for 16 h at r.t. 6-(3-Aminoazetidin-1-yl)-5-cyano-2-methylnicotinate bis(trifluoroacetate) (0.210 g, 0.333 mmol) in DCE (2 mL) and DIPEA (0.580 mL, 3.33 mmol) Were added to this solution and stirred at r.t for 2 h. The reaction mixture was concentrated under reduced pressure and diluted with EtOAc (40 mL). The combined organics were washed with saturated NaHCO₃ (2×30 mL), dried (MgSO₄) and concentrated under reduced pressure to afford the crude product. Trituration (1:1 EtOAc/hexanes) gave 6-[3-({[(benzylsulfonyl)amino]carbonyl}amino)azetidin-1-yl]-5-cyano-2-methylnicotic acid ethyl ester as a solid Yield: 0.073 g (47.9%)

1H NMR (400 MHz, DMSO-d): δ 1.30 (3H, t, J=7.1 Hz), 2.63 (3H, s), 2.70 (1H, s), 4.18-4.19 (2H, m), 4.24 (2H q, J=7.1 Hz), 4.56 (3H, m), 4.70 (2H, m), 7.1 (1H, s), 7.32-7.43 (51H, m), 8.31 (1H, s).

MS ^{m}/_{z}: 458 (M+1).

### Example 29

### 6-(3-{2-[(Benzylsulfonyl)amino]-2-oxoethyl}piperidin-1-yl)-5-cyano-2-methylnicotinic acid ethyl ester

### (a) Piperidin-3-ylacetic acid potassium salt

Potassium trimethylsilanoate (0.89 g, 5.2 mmol) and ethyl piperidin-3-ylacetate (0.87 g, 6.8 mmol) were stirred in DCM (50 mL) at r.t for 2 days. Concentration of the reaction mixture afforded solid piperidin-3-ylacetic acid as the potassium salt, which was used crude assuming complete conversion. Yield. 0.74 g (100%).

### (b) {1-[3-Cyano-5-(ethoxycarbonyl)-6-methylpyridin-2-yl]piperidin-3-yl}acetic acid

Ethyl 6-chloro-5-cyano-2-methylnicotinate (1.00 g, 4.45 mmol), piperidin-3-ylacetic acid (0.701 g, 4.90 mmol) and DIPEA (2.33 mL, 13:4 mmol) were dissolved in DM (30 mL) and stirred at r.t for 3 days. The reaction mixture was diluted with EtOAc (100 mL), washed with saturated NH₄Cl (2×25 mL), saturated NaHCO₃ (2×25 mL), brine (25 m), dried (MgSO₄) and concentrated under reduced pressure to afford crude material. Flash chromatography (9:1 EtOAc/hexanes with 1% HOAc) gave {1-[3-cyano-5-(ethoxycarbonyl)-6-methylpyridin-2-yl]piperidin-3-yl}acetic acid as a solid. Yield: 0.791 g (54%).

¹H NMR (400 MHz, CDCl₃): δ 1.37 (3H, t, J=7.1 Hz), 1.39-1.44 (1H, m), 1.63-1.73 (1H, m), 1.78-1.85 (1H, m), 1.98-2.03 (1H, m), 2.16-2.24 (1H, m), 2.29-2.34 (1H, m), 2.40-2.46 (1H, m), 2.71 (3H, s), 3.08-3.13 (1H, m), 3.26-3.32 (1H, m), 4.31 (2H, q, J=7.1 Hz), 4.44-4.50 (1H, m), 4.52-4.56 (1H, m), 8.33 (1H, s).

MS ^{m}/_{z}: 330 (M−1).

### (c) 6-(3-{2-[(Benzylsulfonyl)amino]-2-oxoethyl}piperidin-1-yl)-5-cyano-2-methylnicotinic acid ethyl ester

{1-[3-Cyano-5-(ethoxycarbonyl)-6-methylpyridin-2-yl]piperidin-3-yl}acetic acid (0.152 g, 0.459 mmol), EDCI (0.114 g, 0.596 mmol), HOBt (0.081 g, 0.596 mmol), 1-phenylmethanesulfonamide (0.102 g, 0.596 mmol) and DIPEA (0.160 mL, 917 mmol) were dissolved in DCM (6 mL) and stirred at r.t for 18 h. The reaction mixture was diluted with EtOAc (50 mL) and washed with saturated NH₄Cl (2×40 mL) and brine (40 mL). The organics were dried (MgSO₄) and concentrated under reduced pressure to afford crude product. Flash chromatography (1:4 EtOAc/hexanes, 1.0% AcOH gave 6-(3-{2-[(benzylsulfonyl)amino]-2-oxoethyl}piperidin-1-yl)-5-cyano-2-methylnicotinic acid ethyl ester as a solid. Yield: 0.065 g (28%).

¹H NMR (400 MHz, CDCl₃): δ 1.35-1.40 (3H, m), 1.53-1.58 (1H, m), 1.64-1.71 (1H, m), 1.73-1.80 (1H, m), 1.93-2.00 (1H, m), 2.11-2.22 (2H, m), 2.27-2.34 (1H, m), 2.68 (3H, s), 3.09-3.16 (1H, m), 3.30-3.38 (1H, m), 4.29-4.40 (4H, m), 4.68 (2H, s), 7.37-7.38 (5H, m), 7.70 (1H, br s), 8.33 (1H, s).

MS ^{m}/_{z}: 485 (M+1).

### Example 30

### 6-(4-{[(Benzylsulfonyl)amino]carbonyl}-4-methylpiperidin-1-yl)-5-cyano-2-methylnicotinic acid ethyl ester

### (a) 1-tert-Butyl 4-methyl piperidine-1,4-dicarboxylate

1-(tert-Butoxycarbonyl)piperidine-4-carboxylic acid (3.00 g, 13 mmol) was dissolved in MeOH (50 mL) and TMSCHN₂ (32.7 nm of a 2 M solution in hexanes, 65 mmol) was added drop-wise at r.t. TMSCHN₂ was added until a persistent yellow color was produced indicating excess reagent. AcOH was added drop-wise to quench the excess TMSCHN₂ and the reaction mixture was concentrated under reduced pressure and azeotroped with Toluene (3×30 mL) to remove any trace MeOH or AcOH. The crude 1-tert-Butyl 4-methyl piperidine-1,4-dicarboxylate was used without further purification

### (b) 1-tert-Butyl 4-methyl 4-methylpiperidine-1,4-dicarboxylate

DIPA (2.40 mL, 17 mmol) was dissolved in THF (60 mL) and cooled to 0° C. Butyl lithium 1.6 M in Hexanes (9.81 mL, 16 mmol) was added drop-wise and the system stirred at 0° C. for 1 h. The reaction mixture was cooled to −78° C. and a solution of 1-tert-butyl 4-methyl piperidine-1,4-dicarboxylate (3.18 g, 13 mmol) in THF (30 mL) was added drop-wise over 30 minutes. The reaction mixture was stirred at −78° C. for 2 h and then Iodomethane (1.31 mL, 21 mmol) in THF (10 mL) was added in one portion and the reaction mixture stirred for 2 h. The system was allowed to warm to r.t overnight. The reaction mixture was quenched with saturated NH₄Cl (100 mL) and extracted into EtOAc (100 mL). The combined organics were washed with brine (70 mL) and dried (MgSO₄) and concentrated under reduced pressure to afford the crude 1-tert-butyl 4-methyl 4-methylpiperidine-1,4-dicarboxylate as a solid, which was used without further purification

### (c) Methyl 4-methylpiperidine-4-carboxylate

1-tert-Butyl 4-methyl 4-methylpiperidine-1,4-dicarboxylate (3.37 g, 13.1 mmol) was suspended in TLC (15 mL) and 4 M HCl in 1,4-dioxane (65.4 mL, 262 mmol) was added and the reaction mixture stirred at r.t until complete consumption of the starting material was observed by TLC analysis. The reaction mixture was concentrated under reduced pressure to afford the crude material. The solids were partitioned between saturated NaHCO₃ and DCM. The organics were dried (MgSO₄) and concentrated under reduced pressure to afford the crude product. Purification by flash chromatography, (eluant 0.5% TEA, 2% MeOH/DCM 1% TEA, 5% MeOH/DCM) gave methyl 4-methylpiperidine-4-carboxylate as an oil. Yield: 0.910 g (44%).

¹H NMR (400 MHz, CDCl₃): δ 1.23 (3H, s), 1.44-1.55 (2H, m), 2.09-2.20 (2H, m), 2.69-2.80 (2H, m), 2.98-3.08 (2H, m), 3.72 (3H, s), 3.99 (1H, br s).

MS ^{m}/_{z}: 158 (M+1).

### (d) 4-Methylpiperidine-4-carboxylic acid hydrochloride

Methyl 4-methylpiperidine-4-carboxylate (0.300 g, 1.9-mmol) was suspended in THF (30 mL) and potassium trimethylsilanolate (2.4 g, 19 mmol) was added. The system was heated at reflux overnight and then cooled to r.t. 4 M HCl in 1,4-dioxane (12 mL, 48 mmol) was added and the system concentrated under reduced pressure to afford crude 4-methylpiperidine-4-carboxylic acid hydrochloride as a solid, which was used without further purification.

### (e) 1-[3-Cyano-5-ethoxycarbonyl)-6-methylpyridin-2-yl]-4-methylpiperidine-4-carboxylic acid

Ethyl 6-chloro-5-cyano-2-methylnicotinate (0.28 g, 1.3 mmol) and 4-methylpiperidine-4-carboxylic acid hydrochloride (0.34 g, 1.9 mmol) were suspended in DMF (20 mL) and DIPEA (1.1 mL, 6.3 mmol) was added. The reaction mixture was stirred at r.t until complete consumption of the starting material was observed by HPLC analysis. The reaction mixture was diluted with EtOAc (100 mL) and washed with saturated NH₄Cl (70 mL), water (2×70 mL) and brine (50 mL). The organics were dried (MgSO₄) and concentrated under reduced pressure to afford the crude material. Flash column chromatography (1:3 EtOAc/hexanes, 0.5% AcOH to 1:2 EtOAc/hexanes, 0.5% AcOH) gave 1-[3-cyano-5-(ethoxycarbonyl)-6-methylpyridin-2-yl]-4-methylpiperidine-4-carboxylic acid as a solid. Yield: 0.179 g (43%).

¹H NMR (400 MHz, DMSO-d₆): δ 1.20 (3H, s), 1.30 (3H, t, J=7.1 Hz), 1.44-1.54 (2H, m), 2.02-2.11 (2H, m), 2.63 (3H, s), 3.39-3.48 (2H, m), 4.15-4.29 (4H, m), 8.32 (1H, s), 12.52 (1H, br s).

MS ^{m}/_{z}: 332 (M+1).

### (f) 6-(4-{[(Benzylsulfonyl)amino]carbonyl}-4-methylpiperidin-1-yl)-5-cyano-2-methylnicotinic acid ethyl ester

1-[3-cyano-5-(ethoxycarbonyl)-6-methylpyridin-2-yl]-4-methylpiperidine-4-carboxylic acid (0.074 g, 0.22 mmol), EDCI (0.056 g, 0.29 mmol) and HOBt (0.039 g, 0.29 mmol) were dissolved in DCM (10 mL) at r.t The reaction mixture was stirred at r.t for 30 minutes and then 1-phenylmethane sulfonamide (0.054 g, 0.31 mmol) and DIPEA (0.23 mL, 1.3 mmol) were added. The reaction mixture was stirred at r.t until complete consumption of starting material was observed by HPLC analysis. The reaction mixture was diluted with DCM (20 mL) and washed with saturated NH₄Cl (20 mL). The organics were dried (MgSO₄) and concentrated under reduced pressure to afford the crude product. Flash chromatography (3:7 EtOAc/hexanes, 0.5% AcOH to 1:1 EtOAc/hexanes, 0.5% AcOH) followed by preparative HPLC gave 6-(4-{[(benzylsulfonyl)amino]carbonyl}-4-methylpiperidin-1-yl)-5-cyano-2-methylnicotinic acid ethyl ester as a solid. Yield: 0.038 g (35%).

¹H NMR (400 MHz, CDCl₃): δ 1.19 (3H, s), 1.38 (3H, t, J=7.0 Hz), 1.54-1.62 (2H, m), 2.00-2.09 (2H, m), 2.73 (3H, s), 3.52-3.62 (2H, m), 4.07-4.17 (2H, m), 4.33 (2H, q, J=7.0 Hz), 4.72 (2H, s), 7.26-7.27 (2H m), 7.37-7.39 (3H, m), 7.47 (1H, s), 8.35 (1H, s).

MS ^{m}/_{z}: 485 (M+1).

### Example 31

### N-(Benzylsulfonyl)-1-[3-chloro-5-(5-ethyl-1,3-oxazol-2-yl)pyridin-2-yl]piperidine-4-carboxamide

### (a) tert-Butyl 4-{[(benzylsulfonyl)amino]carbonyl}piperidine-1-carboxylate

Triethylamine (591 g, 5840 mmol) was added to a stirred suspension of 1-(tertbutoxycarbonyl)piperidine-4-carboxylic acid (448 g, 1954 mmol), LiCl (23.1 g, 545 mmol) and TBTU (657 g, 2046 mmol) in THF (3000 mL) under an atmosphere of nitrogen at r.t. A solution of 1-phenylmethanesulfonamide (352 g in 1300 mL THF, 2056 mmol) was added after 1.5 hours and the stirring was continued over night. The solvent was removed in vacuo to give a thick grey-beige slurry (volume about 2500 mL). EtOAc (3500 mL) was added followed by an aqueous solution of HCl (1960 mL 3.6 M HCl and 1960 mL water). The water phase was removed and the organic phase was washed with 2×1500 mL 1 M HCl. The organic phase was cooled to 0° C. which gave a precipitate of HOBt that was filtered off. Most of the solvent was removed in vacuo to give a thick grey-white slurry. EtOH (50%, 4000 mL) was added and the slurry was stirred for 1.5 hours. The precipitated product was filtered off, washed with 50% EtOH (500 mL+2×1500 mL) and dried in a vacuum oven at 25° C. to give tert-butyl 4-[(benzylsulfonyl)carbamoyl]piperidine-1-carboxylate as a white solid. Yield 584 g (78%).

¹H NMR (400 MHz, CDCl₃): δ 1.46 (9H, s), 1.54-1.61 (2H, m), 1.70-1.74 (2H, m), 2.19-2.27 (1H, m), 2.68-2.75 (2H, m), 4.07-4.12 (2H, m), 4.66 (2H, s), 7.32-7.41 (5H, m), 7.54 (1H, br s).

### (b) N-(benzylsulfonyl)piperidine 4-carboxamide

tert-Butyl 4-[(benzylsulfonyl)carbamoyl]piperidine 1-carboxylate (583 g, 1524 mmol) was suspended in formic acid (3000 mL) under a nitrogen atmosphere and the reaction was stirred for 20 minutes. The reaction was foaming due to the gas evolution and formic acid (500 mL) was used to wash down the foam from the reaction vessel walls. After 2 hours the foaming had stopped and the reaction was clear with a few solids left. The reaction was stirred over night and 2500 ml of formic acid was removed in vacuo. Water (1000 mL) was added and the reaction was filtered. The clear solution was evaporated and water (3000 mL) was added. A saturated ammonium hydroxide solution in water was used (totally 390 mL) was added and the pH was going from 3.10 to 6.10) to neutralize the acidic solution and at the endpoint (pH=6.10) a heavy precipitate of the product was formed. The mixture was stirred over night and the precipitate was filtered off and washed with water (1000 mL). Drying in a vacuum oven at 25° C. gave N-(benzylsulfonyl)piperidine-4-carboxamide as a white powder. Yield 372.4 g (87%).

¹H NMR (400 MHz, DMSO-d₆): δ 1.60-1.72 (2H, m), 1.75-1.84 (2H, m), 2.10-2.19 (1H, m), 2.77-2.87 (2H, m), 3.10-3.18 (2H, m), 4.23 (2H, s), 7.18-7.28 (5K m), 8.17 (1H, br s).

### (c) N-(Benzylsulfonyl)-1-[3-chloro-5-(5-ethyl-1,3-oxazol-2-yl)pyridin-2-yl]piperidine-4-carboxamide

A suspension of 2,3-dichloro-5-(5-ethyl-1,3-oxazol-2-yl)pyridine (0.300 g, 1.23 mmol), N-(benzylsulfonyl)piperidine-4-carboxamide (0.367 g, 1.30 mmol) and DIPEA (0.645 mL, 3.70 mmol) in DMA (10 mL) was stirred at 80° C. for 24 h The reaction mixture was cooled to r.t and poured into EtOAc (60 mL) and saturated. NH₄Cl (30 mL). The organics were washed with water (3×50 mL), brine (1×50 mL), dried (MgSO₄) and concentrated under reduced pressure to afford the crude material. Flash chromatography (3:7 EtOAc/hexanes with 0.5% AcOH) furnished N-(Benzylsulfonyl)-1-[3-chloro-5-(5-ethyl-1,3-oxazol-2-yl)pyridin-2-yl]piperidine-4-carboxamide as a solid. Yield: 0.297 g (49%).

¹H NMR (400 MHz, CDCl₃): δ 1.31 (3H, t, J=7.0 Hz), 1.86-1.91 (4H, m), 2.29-2.38 (1H, m), 2.75 (2H, q, J=7.0 Hz), 2.84-2.91 (2H, m), 3.97-4.02 (2H, m), 4.69 (2H, s), 6.82 (1H, s), 7.35-7.41 (5H, m), 7.47 (1H, br s), 8.15 (1H, d, J=2.0 Hz), 8.74 (1H, d, J=2.0 Hz).

MS ^{m}/_{z}: 489 (M+1).

### Example 32

### 6-(3-{[Benzylsulfonyl)amino]carbonyl}azetidin-1-yl)-S-cyano-2-methylnicotinic add cyclopentyl ester

### (a) Sodium cylopentanolate

Cyclopentyl alcohol (5 mL) was cooled to 0° C. Sodium hydride (95%, 0.018 g, 0.713 mmol) was slowly added. The solution was used crude assuming a 100% conversion.

### (b) 6-(3-{[(Benzylsulfonyl)amino]carbonyl}azetidin-4-yl)-5-cyano-2-methylnicotinic acid cyclopentyl ester

5-Cyano-2-methyl-6 (3-phenylmethanesulfonylaminocarbonyl-azetidin-1-yl)-nicotinic acid ethyl ester (0.070 g, 0.158 mmol, see Example 46) and molecular sieves (4 Angstrom, 0.070 g) were suspended in cyclopentanol (5 mL) and DMSO (2 mL) and stirred at r.t for 10 minutes. Sodium cylopentanolate (0.286 g, 3.48 mmol) in cyclopentyl alcohol (5 mL) was added and the solution was stirred for 10 minutes. HCl (conc.) was added dropwise to the mixture until the pH was lowered to pH 2. The reaction mixture was filtered and then concentrated under reduced pressure. Water (10 mL) was added to the solution and the combined aqueous was washed with EtOAc (3×40 mL), dried (MgSO₄) and concentrated under reduced pressure to afford the crude product as a solid. Flash chromatography (30% EtOAc with 0.5% AcOH) gave 6-(3-{[(benzylsulfonyl)amino]carbonyl}azetidin-1-yl)-5-cyano-2-methylnicotinic acid cyclopentyl ester as a solid. Yield: 0.031 g (41%).

¹H NMR (400 MHz, DMSO-d₆): δ 1.56-1.64 (2H, m), 1.69-1.80 (4H, m), 1.84-1.95 (2H, m), 2.63 (3H, m) 3.52-3.60 (1H, m), 4.26-4.35 (2H, m), 4.37-4.45 (2H, m), 4.76 (2H, s) 5.22-5.30 (1H, m), 7.31-7.43 (5H, m) 8.29 (1H, s), 11.8 (1H, s).

MS ^{m}/_{z}: 483 (M+1).

### Example 33

### 6-(4-{[(Benzylsulfonyl)amino]carbonyl}piperidin-1-yl)-5-cyano-2-methylnicotinic acid propyl ester

To a solution of 6-(4-{[(benzylsulfonyl)amino]carbonyl}piperidin-1-yl)-5-cyano-2-methylnicotinic acid ethyl ester (0.078 g, 0.17 mmol, See Example 42) in THF (15 mL) and n-Propanol (15 mL) were added 4 Angstrom molecular sieves (0.5 g). The reaction mixture was stirred for 1 h and then cooled to 0° C. After the addition of NaH (60% dispersion in mineral oil, 0.013 g, 0.33 mmol), the mixture was warmed to r.t and stirred for 2 h. Addition of acetic acid (5.0 mL), filtration through celite, concentration followed by azeotroping with toluene (2×50 mL) provided the crude material. Trituration with MeOH (1×25 mL) and then EtOAc (1×25 mL) furnished 6-(4-{[(benzylsulfonyl)amino]carbonyl}piperidin-1-yl)-5-cyano-2-methylnicotinic acid propyl ester as a solid. Yield: 0.027 g (34%).

¹H NMR (400 MHz, DMSO-d₆): δ 0.96 (3H, t, J=7.5 Hz), 1.59-1.66 (2H, m), 1.67-1.76 (2H, m), 1.82-1.86 (2H, m), 2.55-2.62 (1H, m), 2.65 (3H, s), 3.11-3.17 (2H, m), 4.17 (2H, t, J=6.7 Hz), 4.56-4.52 (2H, m), 4.70 (2H, s), 7.28-7.31 (2H, m), 7.38-7.43 (3H, m), 8.34 (1H, s), 11.61 (1H, br s).

MS ^{m}/_{z}: 485 (M+1).

### Example 34

### 6-(4-{[(Benzylsulfonyl)amino]carbonyl}piperidin-1-yl)-5-cyano-2-isopropylnicotinic acid ethyl ester

### (a) Ethyl 2-((dimethylamino)methylene)-4-methyl-3-oxopentanoate

1,1-Dimethoxy-N,N-dimethylmethanamine (4.96 mL, 37.2 mmol) was added drop-wise to ethyl 4-methyl-3-oxopentanoate (5.00 mL, 31.0 mmol) while stirring at r.t. The reaction mixture was allowed to stir at r.t for 18 h and was then concentrated under reduced pressure and azeotroped with toluene (2×20 mL) producing ethyl 2-((dimethylamino)methylene)-4-methyl-3-oxopentanoate as an oil which was used without purification. Yield: 6.61 g (100%).

¹H NMR (400 MHz, CDCl₃): δ 1.09 (6H, d, J=6.9 Hz), 1.31 (3H, t, J=7.3 Hz), 3.00 (6H, br s), 3.26 (1H, br s), 4.21 (2H, q, J=7.3 Hz), 7.60 (1H, s).

### (b) Ethyl 5-cyano-2-isopropyl-6-oxo-1,6-dihydropyridine-3-carboxylate

To a suspension of 2-cyanoacetamide (2.74 g, 32.6 mmol) in THF (100 mL) was added NaH (60% dispersion in mineral oil, 1.36 g, 34.1 mmol) added. The system was stirred at r.t until gas evolution ceased, at which point ethyl 2-((dimethylamino)methylene)-4-methyl-3-oxopentanoate (6.61 g, 31.0 mmol) was added in one portion. The reaction mixture was stirred at r.t for 18 h and concentrated under reduced pressure to afford crude intermediate. The solids were dissolved in a minimum amount of warm water and then acidified to pH 1 with 5 N HCl. Filtration followed by drying under vacuum produced ethyl 5-cyano-2-isopropyl-6-oxo-1,6-dihydropyridine-3-carboxylate. Yield: 6.46 g (89%).

¹H NMR (400 MHz, DMSO-d₆): δ 1.25 (6H, d, J=7.1 Hz), 1.29 (3H, t, J=7.3 Hz), 4.01-4.12 (1H, m), 4.23 (2H, q, J=7.3 Hz), 8.43 (1H, s), 12.56 (1H, br s).

MS ^{m}/_{z}: 235 (M+1).

### (c) Ethyl 6-chloro-5-cyano-2-isopropylnicotinate

A suspension of ethyl 5-cyano-2-isopropyl-6-oxo-1,6-dihydropyridine-3-carboxylate (6.46 g, 27.6 mmol) in POCl₃ (10.1 mL, 110 mmol) was heated at 100° C. for 6 h. The reaction mixture was poured onto ice and then basified with solid K₂CO₃. The aqueous phase was extracted with DCM (3×100 mL) and the organics was dried (MgSO₄) and concentrated under reduced pressure to afford ethyl 6-chloro-5-cyano-2-ispropylnicotinate, which was used without further purification Yield: 6.54 g (93%).

¹H NMR (400 MHz, CDCl₃): δ 1.29 (6H, d, J=6.8 Hz), 1.42 (3H, t, J=7.2 Hz), 3.88-3.98 (1H, m), 4.41 (2H, q, J=7.2 Hz), 8.37 (1H, s).

MS ^{m}/_{z}: 254 (M+1).

### (d) N-(Benylsulfonyl)piperidine 4-carboxamide hydrochloride

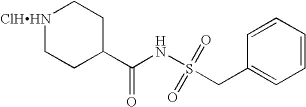

To a suspension of tert-butyl 4-{[(benzylsulfonyl)amino]carbonyl}piperidine-1-carboxylate (4.18 g, 10.9 mmol) in THF (100 mL) was added 4 M HCl in dioxane (54.6 mL, 218 mmol) and the reaction mixture was stirred at r.t for 18 h. The solids were collected by filtration and washed with EtOAc (100 mL) and then placed under vacuum to yield N-(benzylsulfonyl)piperidine-4-carboxamide hydrochloride as a solid. Yield: 2.50 g (72%).

¹H NMR (400 MHz, DMSO-d₆): δ 1.70-1.78 (2H, m), 1.83-1.88 (2H, m), 2.47-2.53 (1H, m), 2.80-2.89 (2H, m), 3.26-3.31 (2H, m), 4.71 (2H, s), 7.27-7.30 (2H, m), 7.39-7.41 (3H, m), 8.53 (1H, brs), 8.79 (1H, brs), 11.70 (1H, brs).

### (e) 6-(4-{[(Benzylsulfonyl)amino]carbonyl}piperidin-1-yl)-5-cyano-2-isopropylnicotinic acid ethyl ester

A mixture of ethyl 6-chloro-5-cyano-2-isopropylnicotinate (0.184 g, 0.728 mmol), N-(benzylsulfonyl)piperidine-4-carboxamide hydrochloride (0.232 g, 0.728 mmol) and DIPEA (0.380 mL, 2.18 mmol) in DMF (3.0 mL) was heated to 60° C. for 5 h. The reaction mixture was diluted with EtOAc (30 mL), washed with saturated NH₄Cl (2×15 mL) and brine (15 mL). The organics were dried (MgSO₄) and concentrated under reduced pressure to afford crude product. Flash chromatography (DCM followed by 1% MeOH, 1% HOAc in DCM) produced a solid, which was triturated with 1:1 Et₂₀/hexanes (25 mL) to afford 6-(4-{[(benzylsulfonyl)amino]carbonyl}piperidin-1-yl)-5-cyano-2-isopropylnicotinic acid ethyl ester as a solid. Yield: 0.300 g (81%).

¹H NMR (400 MHz, CDCl₃): δ 1.22 (6H, d, J=6.5 Hz), 1.38 (3H, t, J=7.3 Hz), 1.74-1.90 (4H, m), 2.41-2.48 (1H, m), 3.11-3.18 (2H, m), 3.95-4.05 (1H, m), 4.32 (2H, q, J=7.3 Hz), 4.64-4.69 (4H, m), 7.31-7.33 (2H, m), 7.37-7.43 (3H, m), 8:10 (1H, br s), 8.31 (1H, s).

MS ^{m}/_{z}: 499 (+1).

### Example 35

### 6-(4-{[(Benzylsulfonyl)amino]carbonyl}piperidin-1-yl)-5-cyano-2-ethylnicotinic acid ethyl ester

### (a) Ethyl 2-((dimethylamino)methylene)-3-oxopentanoate

1,1-Dimethoxy-N,N-dimethylmethanamine (5.09 mL, 42.0 mmol) was added dropwise to ethyl 3-oxopentanoate (5.0 mL, 35.0 mmol) while stirring at r.t. The reaction mixture was stirred at r.t for 18 h and then was concentrated under reduced pressure and azeotroped with toluene (2×20 mL) producing ethyl 2-((dimethylamino)methylene)-3-oxopentanoate as an oil which was used without purification. Yield: 6.98 g (100%).

¹H NMR (400 MHz, CDCl₃): δ 1.10 (3H, t, J=7.7 Hz), 1.32 (3H, t, J=7.7 Hz), 2.67-2.69 (2H, m), 3.01 (611, br s), 4.22 (2H, q, J=7.2 Hz), 7.64 (1H, s).

### (b) Ethyl 5-cyano-2-ethyl-6-oxo-1,6-dihydropyridine-3-carboxylate

To a suspension of 2-cyanoacetamide (3.09 g, 36.8 mmol) in THF (100 mL) was added NaH (60% dispersion in mineral oil, 1.54 g, 38.5 mmol) added. The mixture was stirred at r.t until gas evolution ceased, at which point ethyl 2-((dimethylamino)methylene)-3-oxopentanoate (6.98 g, 35.0 mmol) was added in one portion. The reaction mixture was stirred at r.t for 18 h and concentrated under reduced pressure to afford crude intermediate. The solids were dissolved in a minimum amount of warm water and then acidified to pH 1 with 5 M HCl. Filtration followed by drying under vacuum produced ethyl 5-cyano-2-ethyl-6-oxo-1,6-dihydropyridine-3-carboxylate as a solid. Yield: 6.28 g (81%).

¹H NMR (400 MHz, DMSO-d₆): δ 1.18 (3H, t, J=7.3 Hz), 1.29 (3H, t, J=7.0 Hz), 2.95 (2H, q, J=7.3 Hz), 4.24 (2H, q, J=7.0 Hz), 8.45 (1H, s), 12.79 (1H, br s).

MS ^{m}/_{z}: 221 (M+1).

### (c) Ethyl 6-chloro-5-cyano-2-ethylnicotinate

A suspension of ethyl S-cyano-2-ethyl-6-oxo-1,6-dihydropyridine-3-carboxylate (6.28 g, 28.5 mmol) in POCl₃ (10.4 mL, 114 mmol) was heated to 100° C. for 6 h. The reaction mixture was poured onto ice and then basified with solid K₂CO₃. The aqueous phase was extracted with DCM (3×100 mL) and the organics dried (MgSO₄) and concentrated under reduced pressure to afford ethyl 6-chloro-5-cyano-2-ethylnicotinate as a solid, which was used without further purification. Yield: 6.17 g (91%).

¹H NMR (400 MHz, CDCl₃): δ 1.32 (3H, t, J=7.4 Hz), 1.42 (3H, t, J=7.4 Hz), 3.23 (2H, q, J=7.4 Hz), 4.42 (2H, q, J=7.4 Hz), 8.45 (1H, s).

MS ^{m}/_{z}: 239 (M+1).

### (d) 6-(4-{[(Benzylsulfonyl)amino]carbonyl}piperidin-1-yl)-5-cyano-2-ethylnicotinic acid ethyl ester

A solution of ethyl 6-chloro-5-cyano-2-ethylnicotinate (0.143 g, 0.599 mmol), N-(benzylsulfonyl)piperidine-4-carboxamide hydrochloride (0.191 g, 0.599 mmol, See example 34(d)) and DIPEA (0.131 mL, 1.80 mmol) in DMF (3.0 mL) was heated to 60° C. for 5 h. The reaction mixture was diluted with EtOAc (30 mL), washed with saturated NH₄Cl (2×15 mL) and brine (15 mL). The organics were dried (MgSO₄) and concentrated under reduced pressure to afford crude product. Flash chromatography (DCM followed by 1% MeOH, 1% HOAc in DCM) produced a solid, which was triturated with 1:1 Et₂O/hexanes (25 mL) to afford 6-(4-{[(benzylsulfonyl)amino]carbonyl}piperidin-1-yl)-5-cyano-2-ethylnicotinic acid ethyl ester as a solid. Yield: 0.250 g (84%).

¹H NMR (400 MHz, CDCl₃): δ 1.25 (3H, t, J=7.4 Hz), 1.38 (3H, t, J=7.0 Hz), 1.74-1.82 (2H, m), 1.84-1.90 (2H, m), 2.39-2.47 (1H, m), 3.10-3.18 (4H, m), 4.32 (2K, q, J=7.0 Hz), 4.66-4.70 (4H, m), 732-7.35 (2H, m), 7.38-7.42 (3H, m), 7.70 (1H, br s), 8.35 (1H, s).

MS ^{m}/_{z}: 485 (M+1).

### Example 36

### 6-(3-([(Benzylsulfonyl)amino]carbonyl azetidin-1-yl)-5-cyano-2-methylnicotinic acid 2,2-dimethylpropyl ester

### (a) 1-{3-cyano-5-[(2,2-dimethylpropoxy)carbonyl]-6-methylpyridin-2-yl}azetidine-3-carboxylic acid

1-[3-cyano-5-(ethoxycarbonyl)-6-methylpyridine-2-yl]azetidine-3-carboxylic acid (0.218 g, 0.92 mmol) was suspended in THF (20 mL) and DMSO (4 mL) and sodium 2,2-dimethylpropan-1-olate (3.684 mL, 3.684 mmol) added. The reaction mixture was stirred at r.t until complete conversion of the starting material to product was observed by HPLC analysis. The reaction was stopped with 1 N HCl (10 mL) and the reaction mixture diluted with water (50 mL) and extracted into EtOAc (2×40 mL). The organics were dried (MgSO₄) and concentrated under reduced pressure to afford the crude material. Flash chromatography (3:7 EtOAc/hexanes, 0.5% AcOH to 1:1 EtOAc/hexanes, 0.5% AcOH) gave 1-{3-cyano-5-[(2,2-dimethylpropoxy)carbonyl]-6-methylpyridin-2-yl}azetidine-3-carboxylic acid as a solid. Yield: 0.167 g (55%).

### (b) 6-(3-{[(Benzylsulfonyl)amino]carbonyl}azetidin-1-yl)-5-cyano-2-methylnicotinic acid 2,2-dimethylpropyl ester

1-{3-Cyano-5-[(2,2-dimethylpropoxy)carbonyl]-6-methylpyridin-2-yl}azetidine-3-carboxylic acid (0.080 g, 0.24 mmol), EDCI (0.060 g, 0.31 mmol) and HOBt (0.042 g, 0.31 mmol) were dissolved in DCM (5 mL) at r.t. The reaction mixture was stirred at r.t for 30 minutes and then phenylmethanesulfonamide (0.058 g, 0.34 mmol) and DIPEA (0.25 mL, 1.45 mmol) were added. The reaction mixture was stirred at r.t until complete consumption of starting material was observed by HPLC analysis. The reaction mixture was diluted with DCM (20 mL) and washed with saturated NH₄CL (20 mL). The combined organics were dried (MgSO₄) and concentrated under reduced pressure to afford the crude product. Flash chromatography (3:7 EtOAc/hexanes, 0.5% AcOH to 1:1 EtOAc/hexanes, 0.5% AcOH) followed by preparative HPLC gave 6-(3-{[(benzylsulfonyl)amino]carbonyl}azetidin-1-yl)-5-cyano-2-methylnicotinic acid 2,2-dimethylpropyl ester as a solid. Yield: 0.016 g (14%).

¹H NMR (400 MHz, CDCl₃): δ 1.02 (9H, s), 2.74 (3H, s), 3.26-3.56 (1H, m), 3.97 (2H, s), 4.43-4.50 (4H, m), 4.69 (2H, s), 7.36-7.56 (5H, m), 8.27 (1H, s).

MS ^{m}/_{z}: 485 (M+1).

### Example 37

### N-(Benzylsulfonyl)-1-[3-cyano-5-(5-ethyl-1,3-oxazol-2-yl)-methylpyridin-2-yl]piperidine-4-carboxamide

### (a) Ethyl 5-cyano-2-methyl-6-oxo-1-{[2-(trimethylsilyl)ethoxy]methyl}-1,6-dihydropyridine-3-carboxylate

The sodium salt of ethyl 5-cyano-2-methyl-6-oxo-1,6-dihydropyridine-3-carboxylate (8.81 g, 38.6 mmol) was distributed equally into 8 Smith process vials. To each vial was added DCM (3 mL), [2-(chloromethoxy)ethyl](trimethyl)silane (1.78 g, 10.7 mmol), and then DIPEA (2.07 g, 16.0 mmol). Each vial was heated in a microwave oven, single node heating, at 120° C. for 10 minutes. Extra [2-(chloromethoxy)ethyl] (trimethyl)silane (0.445 g, 2.68 mmol) was added to each vial and the single node heating was continued at 120° C. for 10 minutes. The reaction mixtures were combined and vacuum filtered. Purification by flash chromatography on SiO₂ with heptane/EtOAc 4:1 or 3:1 afforded the pure product. Yield: 8.376 g (58%).

¹H NMR (400 MHz, CDCl₃): δ 8.16 (s, 1H), 5.46 (s, 2H), 4.13 (q, J=7.2 Hz, 2H), 3.52 (t, J=8.0 Hz, 2H), 2.78 (s, 3H), 1.19 (t, J=7.2 Hz, 3H), 0.75 (t, J=8.0 Hz, 2H), −0.18 (s, 9H).

MS ^{m}/_{z}: 335 (M−1).

### (b) 5-Cyano-2-methyl-6-oxo-1-{[2-(trimethylsilyl)ethoxy]methyl}-1,6-dihydropyridine-3-carboxylic acid

Ethyl 5-cyano-2-methyl-6-oxo-1-{[2-(trimethylsilyl)ethoxy]methyl}-1,6-dihydropyridine-3-carboxylate (8.371 g, 24.9 mmol) was dissolved in THF (50 mL) and 1M LiOH (100 mL) was added. The reaction mixture was stirred at rt for 3 h. The conversion was complete according to LC/MS. 4M HCl was added to pH 2-3. The WATER phase was extracted with EtOAc (3×100 mL). The organic phases were combined and dried with sodium sulphate and evaporated. To give a crude material. Yield: 8.35 g (109%). The isomeric ethyl 5-cyano-2-methyl-6-{[2-(trimethylsilyl)ethoxy]methoxy}nicotinate was formed as the main product according to LC/MS, which showed a product/byproduct ratio of 25:75. No attempt was made to separate the isomers.

MS ^{m}/_{z}: 307 (M−1).

### (c) 5-Cyano-N-(2-hydroxybutyl)-2-methyl-6-oxo-1-{[2-(trimethylsilyl)ethoxy]methyl}-1,6-dihydropyridine-3-carboxamide

A mixture (7.67 g, 24.9 mmol) of 5-cyano-2-methyl-6-oxo-1-{[2-(trimethylsilyl)ethoxy]methyl}-1,6-dihydropyridine-3-carboxylic acid and the isomer ethyl 5-cyano-2-methyl-6-{[2-(trimethylsilyl)ethoxy]methoxy}nicotinate, in a ratio of 25:75 according to LC/MS, was dissolved in DCM (125 mL). EDCI (6.2 g, 27.4 mmol) and HOBt (5.04 g, 37.3 mmol) were added and the reaction mixture was stirred at rt for 40 minutes. 1-aminopropan-2-ol (2.44 g, 27.7 mmol) in DIPEA (16.1 g, 124.4 mmol) was added and stirring at rt was continued for 1.5 h. According to LC/MS only the minor isomer had been converted at this point. Stirring at rt was continued for 16 h freer without any change in LC/MS. The organic phase was extracted with 10% potassium carbonate (2×125 mL), brine (2×125 mL), dried with sodium sulphate and evaporated. This gave 12.21 g crude product. Purification by flash chromatography on Si gel with heptane/EtOAc fractions, first 1:2, then 1:4, eluted afforded 5-cyano-N-(2-hydroxybutyl)-2-methyl-6-oxo-1-{[2-(trimethylsilyl)ethoxy]methyl}-1,6-dihydropyridine-3-carboxamide. Yield: 3.28 g (35%). When all product had been eluted, elution was done with heptane/EtOAc 1:4+1% formic acid. In this way, 2.46 g of ethyl 5-cyano-2-methyl-6-{[2-(trimethylsilyl)ethoxy]methoxy}nicotinate was recovered.

¹H NMR (400 MHz, CDCl₃): δ−0.13 (s, 9H), 0.87-0.77 (m, 5H), 1.44-1.31 (m, 2H), 2.58 (s, 3H), 3.15-3.06 (m, 1H), 3.46-3.38 (m, 1H), 3.60-3.50 (m, 4H), 5.41 (s, 2H), 7.26-7.21 (m, 1H), 7.77 (s, 1H)

MS ^{m}/_{z}: 378 (M−1).

### (d) 5-Cyano-2-methyl-6-oxo-N-(2-oxobutyl)-{[2-(trimethylsilyl)ethoxy]methyl}-1,6-dihydropyridine-3-carboxamide

Oxalyl chloride (0.39 g, 3.05 mmol) was dissolved in DCM (2 mL) under an atmosphere of nitrogen and the solution was cooled to −78° C. DMSO (0.37 g, 4.69 mmol) in DCM (1 mL) was added dropwise and the mixture was stirred at −78° C. for less than 5 minutes. 5-cyano-N-(2-hydroxybutyl)-2-methyl-6-oxo-1-{[2-(trimethylsilyl)ethoxy]methyl}-1,6-dihydropyridine-3-carboxamide (0.89 g, 2.35 mmol) in DCM (2 mL) was added during 2 minutes and stirring at −78° C. was continued for 1 h. TEA (1.19 g 11.7 mmol) was added. After string for 15 minutes the cooling bath removed and the reaction mixture was stirred at ambient temperature for 15 minutes. Water (10 mL) was added and the water phase was extracted with DCM (3×15 mL). The organic phases were combined and dried with sodium sulphate and evaporated to give the crude product which was used without further purification. Yield: 0.780 g (88%).

¹H NMR (500 MHz, CDCl₃): δ−0.12 (s, 9H), 0.81 (t, J=8.2 Hz, 2H), 0.97 (t, J=7.4 Hz, 12H), 2.40 (q, J=7.4 Hz, 2H), 2.63 (s, 3H), 3.55 (t, J=8.2 Hz, 21), 4.09 (d, J=5.3 Hz, 2H), 5.45 (s, 2H), 7.50 (t, J=5.3 Hz, 1H), 7.86 (s, 1H)

MS ^{m}/_{z}: 376 (M−1).

### (e) 5-(5-Ethyl-1,3-oxazol-2-yl)-6-methyl-2-oxo-1-{[2-trimethylsilyl)ethoxy]methyl}-1,2-dihydropyridine-3-carbonitrile

5-cyano-2-methyl-6-oxo-N-(2-oxobutyl)-1-{[2-(trimethylsilyl)ethoxy]methyl}-1,6-dihydropyridine-3-carboxamide (2.761 g, 7.31 mmol) was dissolved in THF (9.6 mL) and the solution was transferred equally into 3 Smith process vials. To each vial was added (Methoxycarbonylsulfamoyl)triethylammonium hydroxide, inner salt (1.162 g, 4.88 mmol). The vials were sealed and heated in a microwave oven, single node heating, at 80° C. for 2 minutes. LC/MS on each vial showed complete conversion. The reaction mixtures were combined and evaporated to give 6.431 g of a crude material. Filtration through a Si-plug (10 g) with Heptane/EtOAc 1:1 (100 mL) afforded 5-(5-ethyl-1,3-oxazol-2-yl)-6-methyl-2-oxo-1-{[2-(trimethylsilyl)ethoxy]methyl}-1,2-dihydropyridine-3-carbonitrile. Yield: 1.766 g (67%).

¹H NMR (500 MHz, CDCl₃): δ−0.20 (s, 9H), 0.74 (t, J=8.0 Hz, 2H), 1.09 (t, J=7.5 Hz, 3H), 2.55 (q, J=7.5 Hz, 2H), 2.82 (s, 3H), 3.52 (t, J=8.0 Hz, 2H), 5.46 (s, 2H), 6.62 (s, 1H), 8.09 (s, 1H) MS ^{m}/_{z}: 358 (M−1).

### (f) 5-(5-Ethyl-1,3-oxazol-2-yl)-6-methyl-2-oxo-1,2-dihydropyridine-3-carbonitrile

A TFA/DCM mixture (1:1, 10 mL) was added to (5-(5-ethyl-1,3-oxazol-2-yl)-6-methyl-2-oxo-1-{[2-(trimethylsilyl)ethoxy]methyl}-1,2-dihydropyridine-3-carbonitrile (1.682 g, 4.68 mmol) and the reaction mixture was stirred at rt for 4 h. According to LC/MS the reaction was complete. The reaction mixture was evaporated. DCM (10 mL) was added and the mixture was dried with sodium sulphate and evaporated. This gave 0.263 g crude material. Purification by flash chromatography on Si-gel with DCM/MeOH (69:1, then 39:1) afforded the title compound. Yield. 0.263 g (82%).

¹H NMR (300 MHz, DMSO-d₆): δ 1.24 (br t, J=7.5 Hz, 3H), 2.68 (s, 3H), 2.73 (br q, J=7.5 Hz, 2H), 7.00 (br s, 1H), 8.51 (s, 1H), 12.97 (s, 1H)

MS ^{m}/_{z}: 230 (M+1).

### (g) 2-Chloro-5-(5-ethyl-1,3-oxazol-2-yl)-6-methylnicotinonitrile

5-(5-ethyl-1,3-oxazol-2-yl)-6-methyl-2-oxo-1,2-dihydropyridine-3-carbonitrile (0.069 g, 0.30 mmol) was dissolved in DCM (0-8 mL) in a Smith process vial and oxalyl chloride (0.573 g, 4.51 mmol) and then DMF (0.022 g, 0.3 mmol) were added at 0° C. The reaction mixture was heated in the sealed vial on an oil bath at 50° C. for 2.5 h. LC/MS showed 33% wanted product and 45% starting material. Stirring at the same temperature was continued. After 1.5 h further, extra DMF (0.022 g, 0.30 mmol) was added. Stirring at the same temperature was performed for 7.5 h further. LC/MS showed 64% wanted product and 8% starting material. The reaction mixture was evaporated and mixed with a batch that was prepared in the following manner.

5-(5-ethyl-1,3-oxazol-2-yl)-6-methyl-2-oxo-1,2-dihydropyridine-3-carbonitrile (0.179 g, 0.78 mmol) was dissolved in DCM (2.4 ml) in a Smith process vial and oxalyl chloride (1.486 g, 11.70 mmol) and then DMF (0.057 g, 0.78 mmol) were added at 0° C. The reaction mixture was heated in the sealed vial on an oil bath at 50° C. for 4 h. LC/MS showed 40% wanted product and 22% starting material. Extra DMF (0.057 g, 0.78 mmol) was added. Stirring at the same temperature was performed for 16 h further. LC/MS showed 35% wanted product and no starting material. The material was evaporated. Purification of the combined batches was done by flash chromatography on Si gel with DCM/MeOH 199:1 as eluent to afford 2-chloro-S-(5-ethyl-1,3-oxazol-2-yl)-6-methylnicotinonitrile. Yield: 0.027 g (10%).

¹H NMR (400 MHz, CDCl₃): δ 1.34 (t, J=7.5 Hz, 3H), 2.80 (q, J=7.5 Hz, 22H), 3.00 (s, 3H), 6.97 (s, 1H), 8.52 (s, 1H)

### (h) 1-[3-Cyano-5-(5-ethyl-1,3-oxazol-2-yl)-6-methylpyridin-2-yl]piperidine 4-carboxylic acid

2-chloro-5-(5-ethyl-1,3-oxazol-2-yl)-6-methylnicotinonitrile (0.056 g, 0.23 mmol) was dissolved in dry EtOH (4 mL) in a Smith process vial. Piperidine-3-carboxylic acid (0.051 g, 0.40 mmol) was added and the sealed vial was heated in a microwave oven, single node heating, at 120° C. for 20 minutes. LC/MS showed the reaction to be incomplete. TEA (0.233 g, 2.30 mmol) and extra Piperidine-3-carboxylic acid (0.015 g, 0.11 mmol) were added and the sealed vial was then heated in a microwave oven, single node heating, at 100° C. for 20 minutes. LC/MS showed the reaction to be complete. The reaction mixture was evaporated. 1M HCl (3 mL) was added and the mixture was extracted with DCM (3×3 mL) by using a phase separator. This gave the crude product which was used without further purification. Yield: 0.086 g (110%)

¹H NMR (300 MHz, CDCl₃): δ 1.17 (t, J=7.5 Hz, 3H), 1.78-1.62 (m, 2H), 1.99-1.86 (m, 2H), 2.51-2.39 (m, 1H), 2.67-2.57 (m, 5H), 3.16-3.04 (m, 2H), 4.43-4.32 (m 2H), 6.70 (br s, 1H), 8.11 (s, 1H)

MS ^{m}/_{z}: 341 (M+1).

### (i) N-(Benzylsulfonyl)-1-[3-cyano-5-(5-ethyl-1,3-oxazol-2-yl)-6-methylpyridin-2-yl]piperidine 4-carboxamide

1-[3-cyano-5-(5-ethyl 1,3-oxazol-2-yl)-6-methylpyridin-2-yl]piperidine-4-carboxylic acid (0.026 g, 0.0075 mmol) was dissolved in DMF (1 mL). HATU (0.034 g, 0.090 mmol) and DIPEA (0.048 g, 0.38 mmol) were added and the reaction mixture was stirred at rt for 15 minutes before 1-phenylmethanesulfonamide (0.013 g, 0.075 mmol) was added. Stirring at rt was continued for 4 h. According to LC/MS around half of thwarting material had been converted at this point. Extra 1-phenylmethanesulfonamide (0.013 g, 0.075 mmol) was added and stirring at rt was continued over week-end (64 h). LC/MS shoed that still only around half of the starting material had been converted. Extra HATU (0.028 g, 0.075 mmol) was added and stirring at rt was continued for 5.5 h feather. According to LC/MS the reaction was complete at this point. Purification by preparative HPLC gave the pure product. Yield: 0.024 g (64%).

¹H NMR (400 MHz, DMSO-d₆): δ 1.26 (t, J=7.6 Hz, 3H), 1.74-1.61 (m, 2H), 1.90-1.81 (m, 2H), 2.58-2.54 (m, 1H), 2.79-2.72 (m, 5H), 3.17-3.08 (m, 2H), 4.51-4.44 (m, 2H), 4.70 (s, 2H), 7.04 (s, 1H), 7.34-7.29 (m, 2H), 7.45-7.39 (m, 3H), 8.38 (s, 1H), 11.62 (s, 1H)

MS ^{m}/_{z}: 494 (M+1).

### Example 38

### 6-(3{[(Benzylsulfonyl)amino]carbonyl}azetidin-1-yl)-5-cyano-2-methylnicotinic acid isopropyl ester

### (a) Sodium propan-2-olate

Isopropyl alcohol (5 mL) was cooled to 0° C. Sodium hydride (95%, 0.088 g, 3.48 mmol) was slowly added. The solution was used crude assuming a 100% conversion.

### (b) 1-[3-Cyano-5-(isopropoxycarbonyl)-6-methylpyridin-2-yl]azetidine-3-carboxylic acid

1-[3-cyano-5-(ethoxycarbonyl)-6-methylpyridin-2-yl]azetidine-3-carboxylic acid (0.400 g, 1.20 mmol) was dissolved in isopropyl alcohol (5 mL) and stirred at r.t for 10 minutes. Sodium propan-2-olate (0.286 g, 3.48 mmol) in isopropyl alcohol (5 mL) was added and the solution was stirred for 10 minutes. HCl (conc.) was added drop-wise to the mixture until the pH was lowered to pH 2. The reaction mixture was concentrated under reduced pressure. The reaction mixture was concentrated under reduced pressure. The aqueous was washed with EtOAc (3×40 mL), dried (MgSO₄) and concentrated under reduced pressure to afford the crude product as a solid. Flash chromatography (100% EtOAc to 100% EtOAc with 0.5% AcOH) yielded 1-[3-cyano-5-(isopropoxycarbonyl)-6-methylpyridin-2-yl]azetidine-3-carboxylic acid as a solid. Yield: 0.133 g (51.0%).

¹H NMR (400 MHz, CDCl₃): δ 1.34 (6H, d, J=6.2 Hz), 2.71 (3H, s), 3.59-3.67 (1H, m), 4.57-4.64 (4H, m), 5.15-5.24 (1H, m), 8.26 (1H, s).

MS ^{m}/_{z}: 304 (M+1).

### (c) 6-(3-{[(Benzylsulfonyl)amino]carbonyl}azetidin-1-yl)-5-cyano-2-methylnicotic acid isopropyl ester

1-[3-cyano-5-(isopropoxycarbonyl)-6-methylpyridin-2-yl]azetidine-3-carboxylic acid (0.047 g, 0.153 mmol), EDCI (0.035 g, 0.184 mmol) and HOBt (0.025 g, 0.184 mmol) were dissolved in DCM (1 mL) at r.t. The reaction mixture was stirred at r.t for 30 minutes and then 1-phenylmethnesulfonamide (0.032 g, 0.184 mmol) and DIPEA (0.134 mL, 0.767 mmol) were added The reaction mixture was stirred at r.t for 18 h. The reaction mixture was diluted with EtOAc (40 mL). The combined organics were washed with saturated NH₄Cl (2×40 mL), dried (MgSO₄) and concentrated under reduced pressure to afford the crude product. Trituration (4:1 Hexanes/CH₂C₁₂) gave 6-(3-{[(benzylsulfonyl)amino]carbonyl}azetidin-1-yl)-5-cyano-2-methylnicotinic acid isopropyl ester as a solid. Yield. 0.031 g (44.3%).

¹H NMR (400 MHz, DMSO-d₆): 1.30 (6H, d, J=6.2 Hz), 2.64 (3H, s), 3.52-3.59 (1H, m), 4.29-4.44 (4H, m), 4.75 (2H, m), 5.04-5.10 (1H, m), 7.32-7.40 (5H, m), 8.29 (1H, s), 11.8 (1H, s).

MS ^{m}/_{z}: 457 (M+1).

### Example 39

### 6-(4-{[(Benzylsulfonyl)amino]carbonyl}piperidin-1-yl)-cyano-2-methylnicotinic acid Isopropyl ester

### (a) Isopropyl 2-((dimethylamino)methylene)-3-oxobutanoate

Isopropyl 3-oxobutanoate (200 mL, 1365 mmol) was stirred at r.t and dimethoxy-N,N-dimethylmethanamine (242 mL, 1706 mmol) was added drop-wise. The reaction mixture was allowed to stir at r.t overnight. The reaction mixture was concentrated under vacuum and then azeotroped with toluene (3′ 300 mL) and placed under high vacuum to afford isopropyl 2-((dimethylamino)methylene)-3-oxobutanoate as an oil, which was used without further purification. Yield: 272 g (100%).

¹H NMR (400 MHz, CDCl₃): δ 1.30 (6H, d, J=6.2 Hz), 2.32 (3H, s), 5.07-5.17 (1H, m); 7.64 (1H, s).

### (b) Isopropyl 5-cyano-2-methyl-6-oxo-1,6-dihydropyridine-3-carboxylate

NaH (33.359 g, 834.07 mmol) was suspended in THF (700 mL) and 2-cyanoacetamide (58.905 g, 700.62 mmol) added portionwise at r.t. When gas evolution had stopped a solution of isopropyl 2-((dimethylamino)methylene)-3-oxobutanoate (147.72 g, 667.25 mmol) in THF (300 mL) was added and the system stirred at r.t overnight. The reaction mixture was concentrated under reduced pressure and the solids dissolved in the minimum amount of to hot water. 1N HCl was added to the solution until pH 1 and the solids isolated by filtration. The solids were dried under high vacuum to afford isopropyl 5-cyano-2-methyl-6-oxo-1,6-dihydropyridine-3-carboxylate as a solid, which was used without further purification. Yield: 123 g (84%).

¹H NMR (400 mL, CDCl₃): δ 1.37 (6H, d, J=6.2 Hz), 2.84 (3H, s), 5.18-5.28 (1H, m), 8.50 (1H, s), 13.04 (1H, s).

MS ^{m}/_{z}: 221 (M+1).

### (c) Isopropyl 6-chloro-5-cyano-2-methylnicotinate

Isopropyl 5-cyano-2-methyl-6-oxo-1,6-dihydropyridine-3-carboxylate (123.04 g, 558.70 mmol) was suspended in POCl₃ (204.58 mL, 2234.8 mmol) and heated at 100° C. for 5 h. The reaction mixture was cooled to r.t and concentrated under reduced pressure. The residue was diluted with DCM and poured onto ice. The biphasic mixture was stirred at r.t and slowly quenched with solid K2CO3 until all the POCl₃ had hydrolysed. The aqueous was extracted into DCM and the organics, dried (MgSO4) and passed through a silica plug. The organics were concentrated under reduced pressure to afford isopropyl 6-chloro-5-cyano-2-methylnicotinate as a solid, which was used without further purification. Yield: 106 g (79%).

¹H NMR (400 MEA, CDCl₃): δ 1.40 (6H, d, J=6.2 Hz), 2.90 (3H, s), 5.23-5.30 (1H, m), 7.26 (1H, s), 8.46 (1H, s).

MS ^{m}/_{z}: 239 (M+1).

### (d) 1-(3-Cyano-5-(isopropoxycarbonyl)-6-methylpyridin-2-yl)piperidine-4-carboxylic acid

Isopropyl 6-chloro-5-cyano-2-methylnicotinate (25.000 g, 104.75 mmol), piperidine-4-carboxylic acid (14.205 g, 109.98 mmol) and DIPEA (d 0.742) (54.735 mL, 314.24 mmol) were suspended in EtOH (200 mL) and heated at reflux for 1 h. The reaction mixture was cooled to r t and added drop-wise to KHSO₄ (71.316 g, 523.74 mmol) in water (2000 mL). The solids were collected by filtration and dried under vacuum to afford 1-(3-cyano-5-(isopropoxycarbonyl)-6-methylpyridin-2-yl)piperidine-4-carboxylic acid as a solid, which was used without further purification. Yield: 35 g (100%).

¹H NMR (400 MHz, CDCl₃): δ 1.35 (6H, d, J=6.2 Hz), 1.81-1.93 (2H, m), 2.04-2.12 (2H, m), 2.67-2.74 (4H, m), 3.26-3.36 (2H, m), 4.53-4.62 (2H, m), 5.15-5.23 (1H, m), 8.32 (1H, s).

MS ^{m}/_{z}: 332 (M+1).

### (e) 6-(4-{[(Benzylsulfonyl)amino]carbonyl}piperidin-1-yl)-5-cyano-2-methylnicotinic acid Isopropyl ester

1-[3-cyano-5-(isopropoxycarbonyl)-6-methylpyridin-2-yl]piperidine-4-carboxylic acid (30.00 g, 90.534 mmol), EDCI (26.03 g, 135.80 mmol), 1-phenylmethanesulfonamide (20.15 g, 117.69 mmol), HOBt (13.46 g, 99.59 mmol) and DIPEA (47.308 mL, 271.60 mmol) were suspended in DCM (400 mL) and stirred for 5 minutes until homogenous. Then the reaction mixture was refluxed for 4 h. The reaction mixture was cooled to r.t. and concentrated under reduced pressure. The crude reaction mixture was dissolved in EtOH (300 mL) and added drop-wise to a rapidly stirred solution of KHSO₄ (61.64 g, 452.67 mmol) in water (3000 mL). The product was collected by filtration, washed with water (3×400 mL) and dried under vacuum (44.00 g of dry product). The dry product was slurried in isopropyl alcohol (2000 mL) and stirred and heated at 50° C. for 2 h. The compound was isolated by filtration and dried under high vacuum to afford 6-(4-{[(benzylsulfonyl)amino]carbonyl}piperidin-1-yl)-5-cyano-2-methylnicotinic acid Isopropyl ester as a solid. Yield: 37.41 g (85%).

¹H NMR (400 MHz, CDCl₃): δ 1.35 (6H, d, J=6.2 Hz), 1.74-1.90 (4H, m), 2.37-2.45 (1H, m), 2.73 (3H, s), 3.10-3.17 (2H, m), 4.63-4.67 (4H, m), 5.17-5.23 (1H, m), 7.33-7A2 (5H, m), 7.48 (1H, br s), 8.33 (1H, s).

MS ^{m}/_{z}: 485 (M+1).

The crystalline form obtained was characterised by the presence, in X-ray powder diffraction (XRPD) measurements, of peaks at about the 2-Theta and relative intensity values detailed in Table 2 below.

TABLE 2
XRPD Peaks for Form I of
6-(4-{[(benzylsulfonyl)amino]carbonyl}piperidin-1-
yl)-5-cyano-2-methylnicotinic acid ethyl ester

| Peak label | Angle (2-Theta, °) | Relative Intensity (%) |
|---|---|---|
| a | 4.945 | 100.0 |
| b | 7.142 | 3.4 |
| c | 9.337 | 26.8 |
| d | 9.863 | 15.9 |
| e | 10.324 | 18.1 |
| f | 12.960 | 2.9 |
| g | 13.398 | 17.6 |
| h | 14.327 | 60.9 |
| i | 14.800 | 4.1 |
| j | 17.096 | 4.6 |
| k | 18.344 | 6.5 |
| l | 18.668 | 5.0 |
| m | 19.094 | 9.6 |
| n | 19.800 | 5.2 |
| o | 20.130 | 15.1 |
| p | 20.674 | 20.2 |
| q | 21.073 | 7.3 |
| r | 21.865 | 25.6 |
| s | 22.563 | 7.8 |
| t | 23.033 | 18.4 |
| u | 23.849 | 13.7 |
| v | 24.209 | 6.4 |
| x | 24.789 | 17.9 |
| y | 25.458 | 6.1 |
| z | 26.055 | 5.0 |
| aa | 26.390 | 4.3 |
| ab | 27.072 | 6.3 |
| ac | 27.718 | 4.8 |
| ad | 28.721 | 9.8 |
| af | 29.783 | 10.5 |
| ag | 30.260 | 4.8 |
| ah | 30.840 | 4.8 |
| ai | 31.250 | 4.3 |
| ak | 34.511 | 4.3 |
| al | 36.014 | 4.4 |
| am | 37.174 | 4.1 |
| an | 37.855 | 3.5 |
| ao | 41.783 | 3.2 |
| ap | 43.320 | 4.9 |
| aq | 44.472 | 3.8 |
| ar | 45.346 | 3.1 |

### Example 40

### 5-Cyano-6-[4-({[(4-cyanobenzyl)sulfonyl]amino}carbonyl)piperidin-1-yl]-2-methylnicotinic acid ethyl ester and Sodium [(4-cyanobenzyl)sulfonyl]({1-[3-cyano-5-(ethoxycarbonyl)-6-methylpyridin-2-yl]piperidin-4-yl}carbonyl)azanide

### (a) 1-[3-Cyano-5-(ethoxycarbonyl)methylpyridin-2-yl]piperidine 4-carboxylic acid

Ethyl 6-chloro-5-cyano-2-methylnicotinate (3.00 g, 13.35 mmol), Piperidine-4-carboxylic acid (1.897 g, 14.69 mmol), and TEA (2.703 g, 26.71 mmol) were mixed and the mixture was refluxed for 10 minutes. LC/MS showed full conversion. The reaction mixture was evaporated, water/EtOAc 1:1 (100 mL) was added and the water phase was acidified to pH3. The EtOAc phase was separated and the water phase was extracted with an additional EtOAc (40 mL). The combined organic phases were dried (Na₂SO₄), filtered and evaporated to give 3.8 g of a crude material.

Purification with preparative HPLC at pH=7 (0.1 M NH₄OAc/CH₃CN) with subsequent switch to pH=3 gave the pure product. Yield: 1.9 g (45%).

¹H NMR (400 MHz, CDCl₃): δ 1.38 (t, J=7.1 Hz, 3H), 1.94-1.82 (m, 2H), 2.13-2.05 (m, 2H), 2.75-2.66 (m, 5H), 3.37-3.27 (m, 2H), 4.33 (q, J=7.1 Hz, 2H), 4.63-4.55 (m, 2H), 8.36 (s, 1H

MS ^{m}/_{z}: 318 (M+1).

### (b) 5-Cyano-6-[4-({[(4-cyanobenzyl)sulfonyl]amino}carbonyl)piperidin-1-yl]-2-methylnicotinic acid ethyl ester and Sodium [(4-cyanobenzyl)sulfonyl]({1-[3-cyano-5-(ethoxycarbonyl)-6-methylpyridin-2-yl]piperidin-4-yl}carbonyl)azanide

1-[3-cyano-5-(ethoxycarbonyl)-6-methylpyridin-2-yl]piperidine-4-carboxylic acid (0.253 g, 0.8 mmol), 1-(4-cyanophenyl)methanesulfonamide (0.188 g, 0.96 mmol) and HATU (0.425 g, 1.12 mmol) were dissolved in DMF and TEA (0.161 g, 1.6 mmol) was added. After an additional 30 minutes DBU (0.243 g, 1.6 mmol) was added and the reaction was stirred at r.t over night. The reaction mixture was evaporated to dryness and the residue was partioned between EtOAc (40 ml) and water (40 mL). The organic phase was separated, dried (Na₂SO₄), filtered and evaporated to give a yellow oil. Purification by preparative-HPLC (Kromasil C8 10 uM, 50×300 mm, 100 mL/min, pH=7) afforded 0.091 g of pure 5-cyano-6-[4-({[(4-cyanobenzyl)sulfonyl]amino}carbonyl)piperidin-1-yl]-2-methylnicotinic acid ethyl ester as a white solid. This solid was diluted in CH₃CN (6 mL) and 0.1 M NaOH (1.9 mL) and freeze dried to afford Sodium [(4-cyanobenzyl)sulfonyl]({1-[3-cyano-5-(ethoxycarbonyl)-6-methylpyridin-2-yl]piperidin-yl}carbonyl)azanide as a white solid. Yield: 0.101 g (24°/o).

¹H NMR (400 MHz, DMSO-d₆): δ 1.32 (t, J=7.2 Hz, 3H), 1.64-1.52 (m, 2H), 1.84-1.76 (m, 2H), 2.30-2.20 (m, 1H), 2.65 (s, 3H), 3.25-3.16 (m, 2H), 4.30-4.22 (q, J=7.2 Hz, 2H), 4.36 (s, 2H), 4.48-4.40 (m, 2H), 7.43 (d, J=8.2 Hz, 2H), 7.74 (d, J=8.2 Hz, 2H), 8.32 (s, 1H)

MS ^{m}/_{z}: 496 (M+1)

### Example 41

### 6-[4-({[(4-Chlorobenzyl)sulfonyl]amino}carbonyl)piperidin-1-yl]-5-cyano-2-methylnicotinic acid ethyl ester and Sodium [(4-chlorobenzyl)sulfonyl]({1-[3-cyano-5-(ethoxycarbonyl)-6-methylpyridin-2-yl]piperidin-4-yl}carbonyl)azanide

1-[3-cyano-5-(ethoxycarbonyl)-6-methylpyridin-2-yl]piperidine-4-carboxylic acid (0.250 g, 0.79 mmol), 1-(4-chlorophenyl)methanesulfonamide (0.0.194 g, 0.94 mmol) and HATU (0.419 g, 1.10 mmol) were dissolved in DMF (5 mL) and TEA (0.161 g, 1.60 mmol) was added. After an additional 30 minutes BEMP (0.432 g, 1.58 mmol) was added and the reaction was stirred at r.t over night. The reaction mixture was evaporated to dryness and the residue was partioned between EtOAc (30 mL) and water (40 mL). The phases were separated and the water phase was extracted with EtOAc (30 mL). The combined organic phase was separated, dried (Na₂SO₄), filtered and evaporated to give an oil. Purification on preparative-HPLC (Kromasil C8 10 uM, 50×300 mm, 100 mL/min, pH=7) afforded 0.101 g of pure 6-[4 ({[(4-chlorobenzyl)sulfonyl]amino}carbonyl)piperidin-1-yl]-5-cyano-2-methylnicotinic acid ethyl ester as a white solid. This solid was dissolved in CH₃CN (6 mL) and 0.1 M NaOH (2.5 mL) and freeze dried to afford sodium [(4-chlorobenzyl)sulfonyl]({1-[3-cyano-5-(ethoxycarbonyl)-6-methylpyridin-2-yl]piperidin-4-yl}carbonyl)azanide 101 mg as a white solid. Yield: 0.118 g (28%).

¹H NMR (400 MHz, DMSO-d₆): δ 1.32 (t, J=7.2 Hz, 3H), 1.66-1.53 (m, 2H), 1.85-1.76 (m, 2H), 2.32-2.22 (m, 1H), 2.65 (s, 3H), 3.25-3.16 (m, 2H), 4.30-4.22 (m, 4H), 4.49-4.41 (m, 2H), 7.26 (d, J=8.4 Hz, 2H), 7.33 (d, J=8.4 Hz, 2M), 8.32 (s, 1H) MS ^{m}/_{z}: 505 (M+1).

### Example 42

### 6-(4-{[(Benzylsulfonyl)amino]carbonyl}piperidin-1-yl)-5-cyano-2-methylnicotinic acid ethyl ester

A solution of ethyl 6-chloro-5-cyano-2-methylnicotinate (47.5 g, 211 mmol) and triethyl amine (58.36 g, 577 mmol) in EtOH (314 ml) was added to a stirred mixture of N-(benzylsulfonyl)piperidine-4-carboxamide (53.55 g, 189.7 mmol, See Example 31(b)) and EtOH (100 ml) at r.t. and the mixture was heated to 100° C. (bath temperature, 20-100° C. during 40 minutes, 100° C. 15 minutes then cool to r.t.) for 15 minutes. A solution of KHSO₄ (142.93 g in 900 mL water) was added to make the product precipitate out. The precipitate was filtered off and washed with water (2×250 mL) to give 87 g of a crude product (84% pure). The crude product was slurried in 50% EtOH (1200 mL) and heated to 50° C. (bath temperature) for 2 hours and 45 minutes followed by stirring over night at r.t. Filtration gave a crude product which was farther washed by stirring with 25% EtOH (1600 mL) at 50° C. for 2 hours followed by 20% EtOH (1000 mL) at 50° C. for 2 hours. (An attempt to purify the material by using a 50% EtOH/water solution was not successful because it dissolved to much of the product). The solid obtained after the washings above (89% pure) was dissolved in 700 mL EtOAc at 70° C. and the solution was left to crystallise at r.t. over right. The crystals was filtered off and washed with EtOAc (200 mL) to give pure 6-(4{[(benzylsulfonyl)amino]carbonyl}piperidin-1-yl)-5-cyano-2-methylnicotinic acid ethyl ester as an orange solid (fine needles) after drying. Yield: 54.94 g of Recrystallization of the solids from the mother liquor using EtOAc gave another 10.50 g. Yield 65.44 g (73%). The product can also be crystallized from CHCl₃.

¹H NMR (400 MHz, CDCl₃): δ 1.38 (3H, t, J=7.0 Hz), 1.77-1.91 (4H, m), 2.37-2.44 (1H, m), 2.73 (3H, s), 3.10-3.17 (2H, m), 4.33 (2H, q, J=7.0 Hz), 4.64-4.68 (4H, m), 7.36-7.41 (5H, m), 8.36 (1H, s).

MS ^{m}/_{z}: 471 (M+1).

The product obtained from crystallization in EtOAc (Form I) was characterised by the presence, in X-ray powder diffraction (XRPD) measurements, of peaks at about the 2-Theta and relative intensity values detailed in Table 3 below and the product obtained from crystallization in CHCl₃ (Form II) in Table 4 below.

TABLE 3
XRPD Peaks for Form I of
6-(4-{[(benzylsulfonyl)amino]carbonyl}piperidin-1-yl)-
5-cyano-2-methylnicotinic acid ethyl ester

| Peak label | Angle (2-Theta, °) | Relative Intensity (%) |
|---|---|---|
| a | 6.763 | 100.0 |
| b | 9.500 | 3.4 |
| c | 9.661 | 3.6 |
| d | 10.019 | 6.1 |
| e | 13.377 | 4.7 |
| f | 13.540 | 3.7 |
| g | 14.381 | 7.4 |
| h | 14.764 | 5.3 |
| i | 19.070 | 6.6 |
| j | 19.437 | 6.5 |
| k | 20.380 | 37.1 |
| l | 21.604 | 4.6 |
| m | 22.902 | 4.5 |
| n | 23.252 | 3.5 |
| o | 24.878 | 5.0 |
| p | 25.459 | 6.0 |
| q | 25.762 | 24.3 |
| r | 25.821 | 12.5 |
| s | 27.298 | 3.7 |
| t | 27.736 | 4.4 |
| u | 34.301 | 4.2 |
| v | 41.444 | 4.4 |

TABLE 4
XRPD Peaks for Form II of
6-(4-{[(benzylsulfonyl)amino]carbonyl}piperidin-1-yl)-
5-cyano-2-methylnicotinic acid ethyl ester

| Peak label | Angle (2-Theta, °) | Relative Intensity (%) |
|---|---|---|
| A | 3.530 | 100.0 |
| B | 7.097 | 12.7 |
| C | 13.363 | 5.6 |
| D | 10.782 | 2.4 |
| E | 14.038 | 3.0 |
| F | 14.762 | 3.6 |
| G | 17.849 | 2.8 |
| H | 18.332 | 8.9 |
| I | 18.596 | 6.0 |
| J | 19.601 | 4.2 |
| K | 20.733 | 3.4 |
| L | 21.500 | 5.6 |
| M | 21.674 | 7.0 |
| N | 23.178 | 3.7 |
| O | 25.057 | 3.1 |
| P | 26.256 | 3.6 |
| Q | 26.738 | 2.9 |
| R | 33.755 | 2.5 |
| S | 41.031 | 2.9 |

The crystalline forms may be further characterised by the presence of one or more of the additional properties listed below:
(i) for Form I
   (I) when characterised by thermogravimetric analysis, a weight loss of approx. 0.8% occurs in the range from 25° C. up to 205° C., and/or
   (II) when characterised by differential scanning calorimetry, at a heating rate of 10° C. per minute in a closed cup with a pinhole under flowing nitrogen, a melting temperature (Tm) having an onset at about 194° C. and/or an associated endotherm of melting of about 96 J/g; and/or
   (III) when stored at 80% RH (ambient) less than 0.2% moisture is adsorbed,
(i) for Form I
   (I) when characterised by thermogravimetric analysis, a weight loss of approx. 0.2% occurs in the range from 25° C. up to 205° C., and/or
   (II) when characterised by differential scanning calorimetry, at a heating rate of 10° C. per minute in a closed cup with a pinhole under flowing nitrogen, a melting temperature (Tm) having an onset at about 193° C. and/or an associated endotherm of melting of about 105 J/g.

### Example 43

### N-[(1,2-Benzisoxazol-3-ylmethyl)sulfonyl]-1-[3-cyano-5-(5-ethyl-1,3-oxazol-2-yl)-6-methylpyridin-2-yl]piperidine 4-carboxamide

1-[3-cyano-5-(5-ethyl-1,3-oxazol-2-yl)-6-methylpyridin-2-yl]piperidine-4-carboxylic acid (0.026 g, 0.076 mmol) was dissolved in DMF (1 mL). HATU (0.044 g, 0.057 mmol) and DIPEA (0.049 g, 0.11 mmol) were added and the reaction mixture was stirred at rt for 15 minutes before 1-(1,2-benzisoxazol-3-yl)methanesulfonamide (0.012 g, 0.057 mmol) was added. Stirring at rt was continued for 20 h. Purification was done by preparative HPLC afforded the title compound. Yield: 0.014 g (46%).

¹H NMR (400 MHz, DMSO-d₆): δ 1.24 (t, J=7.5 Hz, 31), 1.66-1.54 (m, 2H), 1.84-1.77 (m, 2H), 2.29-2.20 (m, 1H), 2.77-2.70 (m, 5H), 3.21-3.12 (m, 21), 4.40-4.32 (m, 2H), 4.72 (s, 2H), 7.01 (s, 1H), 7.34 (t, J=7.8 Hz, 1H), 7.60 (t, J=7.8 Hz, 1H), 7.68 (d, J=8.1 Hz, 1H), 7.97 (d, J=8.1 Hz, 1H), 8.31 (s, 1H)

MS ^{m}/_{z}: 535 (M+1).

### Example 44

### N-(Benzylsulfonyl)-1-[3-cyano-5-(5-ethyl-1,3-oxazol-2-yl)-6-methylpyridin-2-yl]azetidine-3-carboxamide

### (a) 1-[3-Cyano-5-(5-ethyl-1,3-oxazol-2-yl)-6-methylpyridin-2-yl]azetidine-3-carboxylic acid

2-chloro-5-(5-ethyl-1,3-oxazol-2-yl)-6-methylnicotinonitrile (0.028 g, 0.11 mmol) was dissolved in dry EtOH (2 mL) in a Smith process vial. Azetidine-3-carboxylic acid (0.023 g, 0.23 mmol) and TEA (0.114-g, 1.13 mmol) were added and the sealed vial was heated in a microwave oven, single node heating, at 120° C. for 20 minutes. LC/MS showed full conversion. The reaction mix was evaporated. 1M HCl (2 mL) was added. The mixture was extracted with DCM (3×2 mL) by using a phase separator. The organic phase were combined, dried with sodium sulphate and evaporated. This gave 0.033 g crude product. Purification by flash chromatography on Si-gel with DCM/MeOH 39:1+1% formic acid as eluent gave the pure product. Yield: 0.026 g (74%).

¹H NMR (300 MHz, CDCl₃): δ 1.31 (t, J=7.5 Hz, 3H), 2.80-2.70 (m, 5H), 3.70-3.57 (m, 1H), 4.68-4.57 (m, 4H), 6.88 (br s, 1H), 8.20 (s, 1H)

MS ^{m}/_{z}: 313 (M+1).

### (b) N-(Benzylsulfonyl)-1-[3-cyano-5-(5-ethyl-1,3-oxazol-2-yl)-6-methylpyridin-2-yl]azetidine-3-carboxamide

1-[3-cyano-5-(5-ethyl-1,3-oxazol-2-yl)-6-methylpyridin-2-yl]piperidine-4-carboxylic acid (0.012 g, 0.038 mmol) was dissolved in DMF (0.5 mL). HATU (0.018 g, 0.046 mmol) and DIPEA (0.025 g, 0.19 mmol) were added and the reaction mixture was stirred at rt for 30 minutes before 1-phenylmethanesulfonamide (0.008 g, 0.046 mmol) was added. Stirring at rt was continued for 18 h. According to LC/MS no product had been formed at this point. EDCI ((0.007 g, 0.038 mmol) and HOBt (0.008 g, 0.058 mmol) were added and stirring at rt was continued for 28 h further. Extra 1-phenylmethanesulfonamide (0.005 g, 0.029 mmol) was added and stirring at rt was continued over week-end (64 h). According to LC/MS the reaction was complete at this point. Purification by preparative HPLC gave the pure product. Yield: 0.0005 g (5%).

¹H NMR (400 MHz CD₃Cl): δ 1.30 (t, J=7.6 Hz, 3H), 2.74 (q, J=1.6 Hz, 2H), 2.79 (s, 3H), 3.38-3.28 (m, 1H), 4.45-4.40 (m, 4H), 4.66 (s, 2H), 6.82 (s, 1H), 7.42-7.34 (m, 5H), 8.21 (s, 1H)

MS ^{m}/_{z}: 466 (M+1).

### Example 45

### N-[(4-Chlorobenzyl)sulfonyl]-4-[3-cyano-5-(5-ethyl-1,3-oxazol-2-yl)-6-methylpyridin-2-yl]piperidine-4-carboxamide

1-[3-cyano-5-(5-ethyl-1,3-oxazol-2-yl)-6-methylpyridin-2-yl]piperidine-4-carboxylic acid (0.026 g, 0.075 mmol) was dissolved in DMF (1 mL). HATU (0.019 g, 0.090 mmol) and DIPEA (0.048 g, 0.38 mmol) were added and the reaction mixture was stirred at rt for 15 minutes before 1-(4-chlorophenyl)methanesulfonamide (0.019 g, 0.090 mmol) was added. Stirring at rt was continued for 27.5 h. According to LC/MS the reaction was incomplete at this point. Extra HATU (0.028 g, 0.075 mmol) and 1-(4-chlorophenyl)methanesulfonamide (0.014 g, 0.068 mmol) were added and stirring at rt was continued for 26 h further. LC/MS showed the reaction to be almost complete. Purification by preparative HPLC gave the pure product. Yield: 0.008 g (18%).

¹H NMR (400 MHz, DMSO-d₆): δ 1.30-1.22 (t, J=7.5 Hz, 3H), 1.74-1.61 (m, 2H), 1.91-1.82 (m, 2H), 2.64-2.55 (m, 1H), 2.80-2.71 (m, 5H), 3.18-3.08 (m, 2H), 4.52-4.44 (m, 2H), 4.74 (s, 2H), 7.04 (s, 1H), 7.34 (d, J=8.3 Hz, 2M), 7.52 (d, J=8.3 Hz, 2H), 8.35 (s, 1H), 11.65 (s, 1H),

MS ^{m}/_{z}: 528 (M+1).

### Example 46

### 5-Cyano-2-methyl-6-(3-phenylmethanesulfonylaminocarbonyl-azetidin-1-yl)-nicotinic acid ethyl ester

1-[3-Cyano-5-(ethoxycarbonyl)-6-methylpyridine-2-yl]azetidine-3-carboxylic acid (20.00 g, 69.14 mmol), EDCI (19.88 g, 103.7 mmol), 1-phenyl-methane sulfonamide (15.39 g, 89.88 mmol), HOBt (10.276 g, 76.049 mmol) and DIPEA (36.127 mL, 207.41 mmol) were suspended in DCM (500 mL) and stirred at r.t for 5 minutes until homogenous. Then the reaction mixture was refluxed for 3 h. The reaction mixture was cooled to r.t and concentrated under reduced pressure. The crude reaction mixture was dissolved in EtOH (400 mL) and added drop-wise to a rapidly stirred solution of KHSO₄ (47.07 g, 345.68 mmol) in water (4000 mL). The product was collected by filtration, washed with water (3×500 mL) and dried under vacuum (30.61 g of dry product). The dry product was slurried in EtOH (1500 mL) and stirred and heated at 50° C. for 1 h. The compound was isolated by filtration and dried under high vacuum to afford the desired material as crystals. Yield: 27.65 g (90%).

¹H NMR (400 MHz, DMSO-d₆) δ 1.23 (t, J=7.2 Hz, 31), 2.57 (s, 3H), 3.43 (m, 1H), 4.17 (q, J=7.1 Hz, 2H), 4.23 (t, J=7.1 Hz, 211), 4.34 (t, J=8.9 Hz, 2H), 4.68 (s, 2H), 7.29 (m, 5H), 8.33 (s, 1H), 11.75 (s, 1H)

MS ^{m}/_{z}: 443 (M+1)

The crystals were characterised by the presence, in X-ray powder diffraction (XRPD) measurements, of peaks at about the 2-Theta and relative intensity values detailed in Table 5 below.

TABLE 5
XRPD Peaks for Form I of 5-Cyano-2-methyl-6-(3-
phenylmethanesulfonylaminocarbonyl-azetidin-1-
yl)-nicotinic acid ethyl ester

| Peak label | Angle (2-Theta, °) | Relative Intensity (%) |
|---|---|---|
| a | 5.388 | 100.0 |
| b | 7.293 | 12.0 |
| c | 10.049 | 6.2 |
| d | 10.796 | 28.8 |
| e | 13.263 | 4.1 |
| f | 13.949 | 16.4 |
| g | 14.597 | 21.8 |
| h | 16.177 | 3.1 |
| i | 17.691 | 3.8 |
| j | 18.888 | 5.0 |
| k | 20.053 | 10.7 |
| l | 20.477 | 12.6 |
| m | 20.990 | 15.9 |
| n | 21.608 | 26.7 |
| o | 22.577 | 9.1 |
| p | 23.991 | 16.0 |
| q | 24.872 | 9.6 |
| r | 26.203 | 7.9 |
| s | 26.761 | 7.7 |
| t | 27.188 | 11.8 |
| u | 31.221 | 4.9 |
| v | 34.030 | 5.6 |

### Example 47

### Ethyl 5-cyano-6-{3-[({[3-(4-methoxyphenoxy)propyl]sulfonyl}amino)carbonyl]azetidin-1-yl}-2-methylnicotinate

Prepared according to method A using 3-(4-methoxyphenoxy)propane-1-sulfonamide (0.105 g crude, 0.37 mmol). Yield: 0.041 g (32%).

¹H NMR (400 MD, DMSO-d₆) d 1.23 (t, J=7.1 Hz, 3H), 2.03 (quintet, J=6.6 Hz, 2H), 2.54 (s, 3H), 3.59 (s, 3H), 3.6-3.4 (m, 3H overlapped by water), 3.94 (t, J=6.0 Hz, 2H), 4.16 (q, J=7.1 Hz, 2H), 4.27 (m, 2H), 4.38 (t, J=8.6 Hz, 2H), 6.75 (m, 4H), 8.21 (s, 1H)

MS ^{m}/_{z}: 517 (M+1)

### Example 48

### Ethyl 4-amino-6-(3-{[(benzylsulfonyl)amino]carbonyl}azetidin-1-yl)-5-chloronicotinate

### (a) Ethyl 4-amino-6-(3-(tert-butoxycarbonyl)azetidin-1-yl)-5-chloronicotinate

Ethyl 4-amino-5,6-dichloronicotinate (0.560 g, 2.38 mmol) was dissolved in DMA (5 mL) and tert-butyl azetidine-3-carboxylate (0.65 g, 4.1 mmol) and DIPEA (1.2 mL, 7.1 mmol) were added. The reaction was heated at 90° C. After 4 hr, additional tert-butyl azetidine-3-carboxylate (0.32 g, 2.0 mmol) and DIPEA (1.0 mL, 5.9 mmol) were added and heating was continued. After 2 hr additional tert-butyl azetidine-3-carboxylate (0.45 g, 2.9 mmol) and DIPEA (1.0 mL, 5.9 mmol) were added. The reaction was heated an additional 1.5 br and then the reaction was cooled and concentrated under reduced pressure. The residue was dissolved in EtOAc (150 mL) and washed with saturated NH₄Cl (2×75 mL), brine and dried (MgSO₄). The solution was then concentrated under reduced pressure and the crude product was purified with flash chromatography (DCM to 5% EtOAc/DCM) to provide ethyl 4-amino-6-(3-(tert-butoxycarbonyl)azetidin-1-yl)-5-chloronicotinate as a solid. Yield: 0.38 g (45%).

¹H NMR (400 MHz, CDCl₃): δ 1.35 (3H, t, J=7.1 Hz), 1.48 (9H, s), 3.34-3.42 (1H, m), 4.30 (2H, q, J=7.1 Hz), 4.40-4.48 (4H, m), 8.53 (1H, s).

MS ^{m}/_{z}: 356 (M+1).

### (b) 1-(4-Amino-3-chloro-5-(ethoxycarbonyl)pyridin-2-yl)azetidine-3-carboxylic acid hydrochloride

Ethyl 6-(3-(tert-butoxycarbonyl)azetidin-1-yl)-4-amino-5-chloronicotinate (0.37 g, 1.0 mmol) was dissolved in a solution of 4M HCl in dioxane (5 mL). After 14 hr at room temperature the reaction was concentrated under reduced pressure and azeotroped with DCM and EtOAc to provide 1-(4-amino-3-chloro-5-(ethoxycarbonyl)pyridin-2-yl)azetidine-3-carboxylic acid hydrochloride which was used without further purification. Yield-0.35 g, (100%).

### (c) Ethyl 4-amino-6-(3-{[(benzylsulfonyl)amino]carbonyl}azetidin-1-yl)-5-chloronicotinate

1-(4-Amino-3-chloro-5-(ethoxycarbonyl)pyridin-2-yl)azetidine-3-carboxylic acid hydrochloride (0.077 g, 0.23 mmol) was combined with EDCI (0.057 g, 0.30 mmol) and HOBt (0.040 g, 0.30 mmol) in DCM (4 mL). Phenylmethanesulfonamide (0.055 g, 0.32 mmol) was then added, followed by DIPEA (0.24 mL, 1.4 mmol). The reaction was allowed to stir 14 hr. The reaction was then partitioned between EtOAc (75 mL) and NH₄Cl solution (20 mL). The organic was washed with NH₄Cl (20 mL) and then brine (20 mL). The organic phase was dried (MgSO₄) and concentrated. The crude reaction mixture was purified by column chromatography (30 to 50% EtOAc/hexanes, then added 0.5% HOAc). Ethyl 4-amino-6-(3-{[(benzylsulfonyl)amino]carbonyl}azetidin-1-yl)-5-chloronicotinate was isolated as a solid. Yield: 0.070 g (67%).

¹H NMR (400 MHz, CDCl₃): δ 1.37 (3H, t, J=7.1 Hz), 3.19-3.26 (1H, m), 4.28-4.38 (6H, m), 4.70 (2H, s), 7.38-7.40 (5H, m), 8.53 (1H, s).

MS ^{m}/_{z}: 453 (M+1).

### Example 49

### Ethyl 5-cyano-2-methyl-6-[3-({[(3-methylbenzyl)sulfonyl]amino}carbonyl)azetidin-1-yl]nicotinate

1-[3-Cyano-5-(ethoxycarbonyl)-6-methylpyridine-2-yl]azetidine-3-carboxylic acid (0.058 g, 0.2 mmol) was dissolved in DMF (1 mL) and HATU (0.099 g, 0.26 mmol), DIPEA (0.170 mL, 1 mmol) and 1-(3-methylphenyl)methanesulfonamide (0.037 g, 0.2 mmol) was added at r.t. and the reaction was stirred for 30 h. The solvent was removed in vacuo and the residue was purified by preparative HPLC (Kromasil C8 10 μM, 20×. 100 mm column, flow: 30 mL/minute using a gradient of 0.1 M NH₄OAc and CH₃CN) to give the pure product. Yield: 0.019 g (15%).

¹H NMR (400 M, DMSO-d₆): δ 1.32 (t, J=7.1 Hz, 3H), 2.33 (s, 3H), 2.65 (s, 3H), 3.53-3.62 (m, 1H), 4.21-4.34 (m, 4H), 4.39-4.47 (m, 2H), 4.72 (s, 2H), 7.13-7.32 (m, 4H), 8.33 (s, 1H), 11.81 (br s, 1H).

MS ^{m}/_{z}: 457 (M+1).

### Example 50

### 2,2-Dimethylpropyl 6-(4-{[(benzylsulfonyl)amino]carbonyl}piperidin-1-yl)-5-cyano-2-methylnicotinate

### (a) Ethyl 6-(4-(tert-butoxycarbonyl)piperidin-1-yl)-5-cyano-2-methylnicotinate

A solution of ethyl 6-chloro-5-cyano-2-methylnicotinate (6.00 g, 26.7 mmol), tert-butyl piperidine-4-carboxylate hydrochloride (6.51, 29.4 mmol) and DIPEA (23.3 mL, 134 mmol) in DMA (50 mL) were heated to 80° C. for 2 h. After cooling to r.t, the reaction mixture was diluted with EtOAc (300 mL), washed with saturated NH₄Cl (4×50 mL), brine (50 mL), dried (MgSO₄), passed through silica gel and concentrated. Flash chromatography produced ethyl 6-(4-(tert-butoxycarbonyl)piperidin-1-yl)-5-cyano-2-methylnicotinate as a solid. Yield: 8.85 g (89%).

¹H NMR (400 MHz, CDCl₃): δ ¹H NMR (400 MHz, CDCl₃): δ 1.37 (3H, t, J=7.1 Hz), 1.45 (9H, s), 1.75-1.84 (2H, m), 1.99-2.03 (2H, m), 2.49-2.57 (1H, m), 2.72 (3H, s), 3.24-3.31 (2H, m), 4.31 (2H, q, J=7.1 Hz), 4.55-4.60 (2H, m), 8.34 (1H, s).

MS ^{m}/_{z}: 374 (M+1).

### (b) 6-(4-(tert-Butoxycarbonyl)piperidin-1-yl)-5-cyano-2-methylnicotinic acid

To a solution of ethyl 6-(4-(tert-butoxycarbonyl)piperidin-1-yl)-5-cyano-2-methylnicotinate (6.65 g, 17.8 mmol) in THF 50 mL was added aqueous LiOH (1.0 M, 107 mL, 107 mmol) and the mixture was heated to reflux for 5 h. After cooling to r.t, the reaction was acidified to pH 3.5 with 2 M HCl and extracted into EtOAc (4×50 mL). The organic extracts were washed with brine, dried (MgSO₄), passed through silica gel and concentrated. Flash chromatography (20% EtOAc/hexanes with 1% HOAc) furnished 6-(4-(tertbutoxycarbonyl)piperidin-1-yl)-5-cyano-2-methylnicotinic acid as a solid. Yield: 1.8 g (29%)

¹H NMR (400 MHz, DMSO-d₆): δ 1.41 (9H, s), 1.53-1.63 (2H, m), 1.90-1.94 (2H, m.), 2.55-2.60 (1H, m), 2.64 (3H, s), 3.21-3.28 (2H, m), 4.40-4.44 (2H, m), 8.30 (1H, s), 12.91 (1H, br s).

MS ^{m}/_{z}: 350 (M+1).

### (c) 2,2-Dimethylpropyl 6-[4-(tert-butoxycarbonyl)piperidin-1-yl]-S-cyano-2-methylnicotinate

A solution of 6-(4-(tert-butoxycarbonyl)piperidin 1-yl)-5-cyano-2-methylnicotinic acid (0.845 g, 2.45 mmol), neopentyl alcohol (1.30 g, 14.7 mmol), EDCI (2.11 g, 11.0 mmol), HOBt (0.496 g, 3.67 mmol) and DIPEA (0.852 mL, 4.89 mmol) were heated to 80° C. for 2 days. The reaction mixture was diluted with EtOAc (50 mL), washed with saturated NH₄Cl (3×30 mL), brine, dried (MgSO₄), passed through silica gel and concentrated. Flash chromatography (3% EtOAc/hexanes) yielded 2,2-dimethylpropyl 6-[4-(tertbutoxycarbonyl)piperidin-1-yl]-5-cyano-2-methylnicotinate as a solid. Yield: 1.02 g (41%).

¹H NMR (400 MHz, CDCl₃): δ 1.02 (9H, s), 1.46 (9H, s), 1.76-1.85 (2H, m), 2.00-2.03 (2H, m), 2.49-2.57 (1H, m), 2.73 (3H, M, 3.25-3.31 (2H, m), 3.96 (2H, s), 4.56-4.60 (2H, m), 8.32 (1H, s).

MS ^{m}/_{z}: 416 (M+1).

### (d) 1-{3-Cyano-5-[(2,2-dimethylpropoxy)carbonyl]-6-methylpyridin-2-yl}piperidine-4-carboxylic acid

To a solution of 2,2-dimethylpropyl 6-[4-(tert-butoxycarbonyl)piperidin-1-yl]-5-cyano-2-methylnicotinate (0.415 g, 0.999 mmol) in DCM (10 mL) at 0° C. was added TFA (10 mL) and the reaction mixture was stirred for 2 h. Concentration produced 1-{3-cyano-5-[(2,2-dimethylpropoxy)carbonyl]-6-methylpyridin-2-yl}piperidine-4-carboxylic acid which was used crude assuming 100% conversion.

MS ^{m}/_{z}: 513 (M+1).

### (e) 2,2-Dimethylpropyl 6-(4-{[(benzylsulfonyl)amino]carbonyl}piperidin-1-yl)-5-cyano-2-methylnicotinate

A solution of 1-{3-cyano-5-[(2,2-dimethylpropoxy)carbonyl]-6-methylpyridin-2-yl}piperidine-4-carboxylic acid (0.120 g, 0.334 mmol), EDCI (0.0832 g, 0.434 mmol) and DIPEA (0.291 mL, 1.67 mmol) in DCM (3 mL) were stirred at r.t for 30 minutes. Phenylmethanesulfonamide (0.0686 g, 0.401 mmol) was added and stifling was continued for 18 h. Additional EDCI (0.0832 g, 0.434 mmol) and phenylmethanesulfonamide (0.0686 g, 0.401 mmol) were added and the reaction mixture was stirred for 3 days, diluted with EtOAc (50 mL), washed with saturated NH₄Cl (3×30 mL), brine, dried (MgSO₄) and concentrated. Flash chromatography (1:4 EtOAc/hexanes with 1% HOAc) followed by reverse phase HPLC purification afforded 2,2-dimethylpropyl 6-(4 {[(benzylsulfonyl)amino]carbonyl}piperidin-1-yl)-5-cyano-2-methylnicotinate as a solid. Yield: 0.0175 g (10%).

¹H NMR (400 MHz, CDCl₃): δ 1.03 (9H, s), 1.78-1.91 (4H, m), 2.83-2.46 (1H, m), 2.74 (3H, s), 3.11-3.18 (2H, s), 3.97 (2H, s), 4.65-4.70 (4H, m), 7.34-7.35 (2K m), 7.39-7.41 (3H, m), 8.34 (1H, s).

MS ^{m}/_{z}: 513 (M+1).

### Example 51

### Ethyl 5-cyano-2-methyl-6-[3-({[(4-methylbenzyl)sulfonyl]amino}carbonyl)azetidin-1-yl]nicotinate

Thionyl chloride (0.119 g, 1 mmol) was added to a solution of 1-[3-Cyano-5-(ethoxycarbonyl)-6-methylpyridine-2-yl]azetidine-3-carboxylic acid (0.058 g, 0.2 mmol) in DCM (1 mL) at 0° C. and the reaction mixture was allowed to reach r.t and stirred for 30 minutes. The DCM and excess thionyl chloride was evaporated (the residue was re-dissolved in DCM (1 mL) and evaporated, this was repeated once) and the residue was dissolved in pyridine at 0° C. followed by addition of 1-(4-methylphenyl)methanesulfonamide (0.044 g, 0.24 mmol). After stirring for 2 h at rt. DMAP (a few crystals) was added and the stirring was continued for 19 h. BENT (0.055 g, 0.2 mmol) was added and the stirring was continued for 22 h at r.t. LC/MS showed that only the starting materials were present. HATU (0.152 g, 0.4 mmol) and DIPEA (0.259 g, 2 mmol) was added to the mixture at r.t. and the stirring was continued for 20 h at r.t. The solvent was removed in vacuo and the reside was purified by preparative HPLC (Kromasil C8 10 μM, 20×100 mm column, flow: 30 mL/minute using a gradient of 0.1 M NH₄OAc and CH₃CN) to give the pure product. Yield: 0.019 g (15%).

¹H NMR (400 MHz, DMSO-d₆): δ 1.32 (t, J=7.0 Hz, 3H), 2.32 (s, 3H), 2.66 (s, 3H), 3.51-3.60 (m, 1H), 4.20-4.37 (m, 4H), 4.38-4.47 (m, 2H), 4.70 (s, 2H), 7.17-7.28 (m, 4H), 8.34 (s, 1H), 11.77 (br s, 1H).

MS ^{m}/_{z}: 457 (M+1).

### Example 52

### Ethyl 5-cyano-6-[4-({([(4-fluorobenzyl)sulfonyl]amino}carbonyl)piperidin-1-yl]-2-methylnicotinate and Sodium ({1-[3-cyano-5-(ethoxycarbonyl)-6-methylpyridin-2-yl]piperidin-4-yl}carbonyl)[(4-fluorobenzyl)sulfonyl]azanide

1-[3-cyano-5-(ethoxycarbonyl)-6-methylpyridin-2-yl]piperidine-4-carboxylic acid (0.350 g, 1.10 mmol), EDCI (0.274 g, 1.43 mmol)), 1-(4-fluorophenyl)methanesulfonamide (0.271 g, 1.43 mmol) and HOBt (0.194 g, 1.43 mmol) were suspended in DCM (8 mL) and DIPEA (0.713 g, 5.51 mmol) was added to the slurry. The reaction became homogenous after 30 minutes and the siring was continued over night. The solvent was removed in vacuo and the residue was dissolved in EtOAc (20 mL). The organic phase was washed with 0.5 M KHSO₄ (5 mL), water (5 mL) and evaporated to give a crude product. Purification by preparative HPLC (Kromasil C8) afforded 0.429 g of ethyl 5-cyano-6-[4-({[(4-fluorobenzyl)sulfonyl]amino}carbonyl)piperidin-1-yl]-2-methylnicotinate as a white solid.

¹H NMR (400 MHz, DMSO-d₆) δ 1.30 (t, J=7.2 Hz, 3), 1.56-1.69 (m, 2H), 1.80-1.88 (m, 2H), 2.57 (m, 1H), 2.64 (s, 3H), 3.13 (m, 2H), 4.24 (q, J=7.2 Hz, 2H), 4.53 (m, 2H), 4.68 (s, 2H), 7.20-7.27 (m, 2H), 7.30-7.35 (m, 2H), 8.33 (s, 1H), 11.60 (br s, 1H).

This solid was dissolved in CH₃CN (3 mL), 0.1 M NaOH (8.5 mL) and finally freeze dried to give sodium ({1-[3-cyano-5-(ethoxycarbonyl)-6-methylpyridin-2-yl]piperidin-4-yl}carbonyl)[(4-fluorobenzyl)sulfonyl]azanide as a white solid. Yield: 0.444 g (76%).

### Example 53

### Ethyl 6-[4-({[(3-bromobenzyl)sulfonyl]amino}carbonyl)piperidin-1-yl]-5-cyano-2-methylnicotinate

HATU (0.205 g, 0.54 mmol) and DIPEA (0.194 g, 1.5 mmol) was added to a stirred solution of 1-[3-cyano-5-(ethoxycarbonyl)-6-methylpyridin-2-yl]piperidine-4-carboxylic acid (0.095 g, 0.30 mmol) in DMF (1.5 mL) at r.t. followed by 1-(3-bromophenyl)methanesulfonamide (0.090 g, 0.36 mmol) and the reaction was stirred for 16 h. The solvent was removed and the crude product was purified by preparative HPLC (Kromasil C8, 250 mm×50 mm i.d. flow 50 minute, using a linear gradient of 0.1 M NH₄OAc/CH₃CN 95/5 to 0/100 over 40 minutes. Yield: 0.035 g (21%).

¹H NMR (300 M, DMSO-d₆): δ 1.32 (t, J=7.0 Hz, 3H), 1.58-1.74 (m, 2H), 1.80-1.90 (m, 2H), 2.00-2.15 (m, 1H), 2.66 (s, 3H), 3.10-3.22 (m, 2H), 4.27 (q, J=7.0 Hz, 2H), 4.51-4.61 (m, 2H), 4.75 (s, 2H), 7.29-7.35 (m, 1H), 7.37-7.44 (m, 1H), 7.47-7.51 (m, 1H), 7.60-7.66 (m, 11, 8.36 (s, 1H), 11.68 (br s, 1H).

MS ^{m}/_{z}: 550 (M+1).

### Example 54

Cyclopropyl 64-{[(benzylsulfonyl)amino]carbonyl}piperidin-1-yl)-5-cyano-2-methylnicotinate

### (a) Benzyl 2-[(dimethylamino)methylene]-3-oxobutanoate

Benzyl 3-oxobutanoate (82 mL, 475 mmol) was stirred at r.t and 1,1-dimethoxy-N,N-dimethylmethanamine (76 mL, 570 mmol) was added drop-wise. The reaction mixture was allowed to stir at r.t overnight. The reaction mixture was concentrated under vacuum and then azeotroped with toluene (3×200 mL) and placed under high vacuum to afford Benzyl 2-[(dimethylamino)methylene]-3-oxobutanoateas an oil, which was used without further purification Yield: 117 g (100%).

¹H NMR (400 MHz, CDCl₃): δ 2.32 (3H, s), 3.02 (6H, br s), 5.22 (2H, s), 7.29-7.43 (5H, m), 7.70 (1H, s).

### (b) Benzyl 5-cyano-2-methyl-6-oxo-1,6-dihydropyridine-3-carboxylate

NaH (19.9 g, 498 mmol) was added to a stirred r.t suspension of 2-cyanoacetamide (39.9 g, 475 mmol) in THF (1 L). The reaction mixture was stirred at r.t until gas evolution stopped. Benzyl 2-[(dimethylamino)methylene]-3-oxobutanoateas (117.4 g, 474.7 mmol) was added portion-wise and the reaction mixture stirred at r.t overnight. 1N HCl was added and the system stirred at r.t for 1 h and then the reaction mixture was diluted with EtOAc and extracted. The organics were dried (MgSO₄) and concentrated under reduced pressure to afford benzyl 5-cyano-2-methyl-6-oxo-1,6-dihydropyridine-3-carboxylate as a solid, which was used without further purification. Yield: 111 g (88%).

¹H NMR (400 MHz, DMSO-d₆): δ 2.63 (3H, s), 5.29 (2H, s), 7.34-7.47 (5H, m), 8.72 (1H, s), 12.82 (1H, s).

MS ^{m}/_{z}: 267 (M−1).

### (c) 6-Chloro-5-cyano-2-methylnicotinic acid

Benzyl 5-cyano-2-methyl-6-oxo-1,6-dihydropyridine-3-carboxylate was suspended in POCl₃ (43.44 mL, 474.5 mmol) and heated at 100° C. overnight. The reaction mixture was cooled to r.t and poured onto ice. The aqueous was neutralized with solid NaHCO₃ and extracted into DCM. The organics were dried (MgSO₄) and concentrated under reduced pressure to afford the material. Flash chromatography (gradient elution 30-50% EtOAc/Hexanes, 0.5% AcOH) gave crude 6-Chloro-5-cyano-2-methylnicotinic acid as a solid. Yield: 24.2 g (26%/0).

¹H NMR (400 MHz, CDCl₃): δ 3.00 (3H, s), 8.50 (1H, s).

MS ^{m}/_{z}: 195 (M−1).

### (d) 6-Chloro-5-cyano-2-methylnicotinoyl chloride

6-chloro-5-cyano-2-methylnicotinic acid (4.00 g, 20.4 mmol) and oxalyl chloride (2.66 mL, 30.5 mmol) were suspended in DCM (75 mL) and heated at 80° C. for 1 h. The reaction mixture was concentrated under reduced pressure and azeotroped with Hexanes and Toluene. The reaction mixture was then concentrated under reduced pressure to afford the crude 6-chloro-5-cyano-2-methylnicotinoyl chloride, which was used without further purification.

### (e) Cyclopropanol

Cyclopropanol prepared according to the literature: J Org. Chem. 41(7), 123740, 1976 and J. Org. Chem. 45(21), 4129-35, 1980.

### (e) Cyclopropyl 6-chloro-5-cyano-2-methylnicotinate

6-Chloro-5-cyano-2-methylnicotinoyl chloride (2.00 g, 9.30 mmol), cyclopropanol (0.54 mL, 9.30 mmol) and DIPEA (1.62 mL, 9.30 mmol), were suspended in DCM (40 mL) and stirred at r.t for 16 h. Water (40 mL) was added to the solution and stirred for 5 minutes. The layers were separated and the organics were washed with water (2×40 mL), dried (MgSO₄) and concentrated under reduced pressure to afford the crude product as a solid. Flash chromatography (40% DCM in Hexanes) afforded cyclopropyl 6-chloro-5-cyano-2-methylnicotinate as a solid. Yield: 0.500 g (23%).

¹H NMR (400 MHz, CDCl₃): δ 0.85-0.92 (4H, m), 2.90 (3H, s), 4.38-4.45 (1H, m), 8.41 (1H, s).

### (f) 1-(3-Cyano-5-(cyclopropoxycarbonyl)-6-methylpyridin-2-yl)azetidine-3-carboxylic acid

Cyclopropyl 6-chloro-5-cyano-2-methylnicotinate (0.084 g, 0.354 mmol), 3-azetidinecarboxylic acid (0.090 g, 0.887 mmol), and DIPEA (0.442 mL, 2.54 mmol), were suspended in EtOH (4 mL) and then refluxed for 1 h. The reaction mixture was cooled to r.t and concentrated under reduced pressure. EtOAc (50 mL) was added and the reaction mixture was washed with saturated NH₄Cl (2×50 mL), dried (MgSO₄) and concentrated under reduced pressure to afford the crude product as a solid. Flash chromatography (20% EtOAc in Hexanes then 20% EtOAc in Hexanes with 0.1% AcOH) gave 1-(3-cyano-5-(cyclopropoxycarbonyl)-6-methylpyridin-2-yl)azetidine-3-carboxylic acid as a solid. Yield: 0.180 g (71%/3).

¹H NMR (400 MHz, DMSO-d₆): δ 0.73-0.83 (4H, m), 2.61 (3H, s), 3.50-3.60 (1H, m), 4.21-4.27 (1H, m), 4.31-4.39 (2H, m), 4.45-4.54 (2H, m), 8.26 (1H, s), 12.8 (1H, s).

### (g) Cyclopropyl 6-(3-(benzylsulfonylcarbamoyl)azetidin-1-yl)-5-cyano-2-methylnicotinate

1-(3-cyano-5-(cyclopropoxycarbonyl)-6-methylpyridin-2-yl)azetidine-3-carboxylic acid (0.165 g, 0.548 mmol), EDCI (0.157 g, 0.821 mmol), phenylmethanesulfonamide (0.113 g, 0.657 mmol), HOBt (0.081 g, 0.602 mmol) and DIPEA (0.286 mL, 1.64 mmol) were suspended in DCM (10 mL) and then refluxed for 3 h. The reaction mixture was cooled to r.t and concentrated under reduced pressure. The crude reaction mixture was dissolved in IPA (10 mL) and added drop-wise to a rapidly stirring solution of KHSO₄ (0.373 g, 2.74 mmol) in water (100 mL). The product was collected by filtration, washed with water (3×20 mL) and dried under vacuum The dry product was slurried in IPA (100 mL), stirred and heated at 50° C. for 1 h. The solution was then cooled at 0° C. for 3 h. The material was isolated by filtration and dried under high vacuum to give cyclopropyl 6-(3-(benzylsulfonylcarbamoyl)azetidin-1-yl)-5-cyano-2-methylnicotinate as a solid, which was used without further purification. Yield: 0.146 g (53%).

¹H NMR (400 MHz, DMSO-d₆): δ 0.73-0.83 (4H, m), 2.63 (3H, s), 3.52-3.59 (1H, m), 4.22-4.47 (5H, m), 4.75 (2H, s), 7.31-7.43 (5H, m), 8.28 (1H, s), 11.8 (1H, s).

MS ^{m}/_{z}: 455 (M+1).

### Example 55

### 2,2,2-Trifluoroethyl 6-(4-{[(benzylsulfonyl)amino]carbonyl}piperidin-1-yl)-5-cyano-2-methylnicotinate

### (a) 2,2,2-Trifluoroethyl 6-chloro-5-cyano-2-methylnicotinate

Crude 6-chloro-5-cyano-2-methylnicotinoyl chloride (0.500 g, 2.32 mmol), 2,2,2-trifluoroethanol (1.69 mL, 23.2 mmol) and DIPEA (2.02 μL, 11.63 mmol) were suspended in DCM (10 mL) and stirred at r.t until complete consumption of starting material was observed by HPLC analysis. The reaction mixture was diluted with DCM and washed with saturated NaHCO₃. The organics were dried (MgSO₄) and concentrated under reduced pressure to afford the crude material. Flash chromatography (100% DCM) gave 2,2,2-trifluoroethyl 6-chloro-5-cyano-2-methylnicotinate as a solid. Yield: 0.155 g (24%).

### (b) 2,2,2-Trifluoroethyl 6-(4-{[(benzylsulfonyl)amino]carbonyl}piperidin-1-yl)-5-cyano-2-methylnicotinate

2,2,2-Trifluoroethyl 6-chloro-5-cyano-2-methylnicotinate (0.057 g, 0.204 mmol), N-(benzylsulfonyl)piperidine-4-carboxamide hydrochloride (0.085 g, 0.266 mmol, See Example 34(d)) and DIPEA (0.18 mL, 1.023 mmol) were suspended in DMF (5 mL) and stirred at r.t for 4 h. The reaction mixture was added drop-wise to a stirred solution of KHSO₄ (0.195 g, 1.43 mmol) in water (50 mL). The product was collected by filtration and dried under high vacuum to give 2,2,2-trifluoroethyl 6-(4 {[(benzylsulfonyl)amino]carbonyl}piperidin 1-yl)-5-cyano-2-methylnicotinate as a solid Yield 0.095 g (88%).

¹H NMR (400 MHz, DMSO-d₆): δ 1.58-1.72 (2H, m), 1.81-1.91 (2H, m), 2.55-2.69 (4H, m), 3.13-3.24 (2H; m), 4.54-4.64 (2H, m), 4.70 (2H, s), 4.90-4.98 (2H, m), 7.26-7.33 (2H, m), 7.36-7.45 (3H, m), 8.34 (1H, s), 11.61 (1H, s).

MS ^{m}/_{z}: 525 (M+1).

### Example 56

### 2,2,2-Trifluoroethyl 6-(3-{[(benzylsulfonyl)amino]carbonyl}azetidin-1-yl)-5-cyano-2-methylnicotinate

### (a) 1-(3-Cyano-6-methyl-5-((2,2,2-trifluoroethoxy)carbonyl)pyridin-2-yl)azetidine-3-carboxylic acid

2,2,2-Trifluoroethyl 6-chloro-5-cyano-2-methylnicotinate (0.155 g, 0.556 mmol), azetidine-3-carboxylic acid (0.068 g, 0.67 mmol) and DIPEA (0.485 mL, 2.78 mmol) were suspended in EtOH (10 mL) and heated at Reflux for 1 h. The reaction mixture was cooled to r.t and added drop-wise to KHSO₄ (0.53 g, 3.89 mmol) in water (100 mL). The solids were collected by filtration and dried under vacuum to afford the crude desired material. Flash chromatography (eluant 30-50% EtOAc/Hexanes 0.5% AcOH) gave 1-(3-Cyano-6-methyl-5-((2,2,2-trifluoroethoxy)carbonyl)pyridin-2-yl)azetidine-3-carboxylic acid as a solid. Yield: 0.136 g (71%).

¹H NMR (400 MHz, CDCl₃): δ 2.72 (3H, s), 3.60-3.69 (1H, m), 4.58-4.70 (6H, m), 8.29 (1H, s).

MS ^{m}/_{z}: 344 (M+1).

### (b) 2,2,2-Trifluoroethyl 6-(3-{[(benzylsulfonyl)amino]carbonyl}azetidin-1-yl)-5-cyano-2-methylnicotinate

1-(3-Cyano-6-methyl-5-((2,2,2-trifluoroethoxy)carbonyl)pyridin-2-yl)azetidine-3-carboxylic acid (0.068 g, 0.198 mmol), phenylmethanesulfonamide (0.044 g, 0.258 mmol), EDCI (0.057 g, 0.297 mmol), HOBt (0.029 g, 0.218 mmol) and DIPEA (0.10 mL, 0.59 mmol) were suspended in DCM (5 mL) and heated at reflux for 4 h. The reaction mixture was diluted with DCM and washed with saturated NH₄Cl and the organics dried (MgSO₄) and concentrated under reduced pressure to afford the crude material. Flash chromatography (gradient elution 30-50% EtOAc/Hexanes, 0.5% AcOH) gave 2,2,2-trifluoroethyl 6-(3-{[(benzylsulfonyl)amino]carbonyl}azetidin-1-yl)-5-cyano-2-methylnicotinate as a solid. Yield: 0.061 g (62%).

¹H NMR (400 MHz, DMSO-d): δ 2.65 (3H, s), 3.52-3.62 (1H, m), 4.28-4.38 (2H, m), 4.39-4.51 (2H, m), 4.76 (2H, s), 4.86-4.97 (2H, m), 7.30-7.43 (5H, m), 8.13 (1H, s), 11.83 (1H, s).

MS ^{m}/_{z}: 497 (M+1).

### Example 57

### 2,2,2-Trifluoroethyl 6-[3-({[(4-chlorobenzyl)sulfonyl]amino}carbonyl)azetidin-1-yl]-5-cyano-2-methylnicotinate

1-(3-Cyano-6-methyl-5-((2,2,2-trifluoroethoxy)carbonyl)pyridin-2-yl)azetidine-3-carboxylic acid (0.068 g, 0.198 mmol), (4-chlorophenyl)methanesulfonamide (0.053 g, 0.258 mmol), EDCI (0.057 g, 0.297 mmol), HOBt (0.0294 g, 0.2.18 mmol) and DIPEA (0.104 mL, 0.594 mmol) were suspended in DCM (5 mL) and heated at reflux for 4 h. The reaction mixture was diluted with DCM and washed with saturated NH₄Cl and the organics dried, MgSO₄ and concentrated under reduced pressure to afford the crude material. Flash chromatography (gradient elution 30-50% EtOAc/Hexanes, 0.5% AcOH) gave 2,2,2-trifluoroethyl 6-[3-({[(4-chlorobenzyl)sulfonyl]amino}carbonyl)azetidin-1-yl]-5-cyano-2-methylnicotinate as a solid. Yield: 0.067 g (64%).

¹H NMR (400 M, DMSO-d₆): δ 2.65 (3H, s), 3.54-3.64 (1H, m), 4.27-4.38 (2H, m), 4.41-4.52 (2H, m), 4.78 (2H, s), 4.85-4.97 (2H, m), 7.33-7.41 (2H, m), 7.42-7.50 (2H, m), 8.31 (1H, s), 11.85 (1H, s).

MS ^{m}/_{z}: 531 (M+1).

### Example 58

### Cyclopropyl 6-(4-(benzylsulfonylcarbamoyl)piperidin-1-yl)-5-cyano-2-methylnicotinate

Cyclopropyl 6-chloro-5-cyano-2-methylnicotinate (0.084 g, 0.354 mmol), N-(benzylsulfonyl)piperidine-4-carboxamide hydrochloride (0.100 g, 0.354 mmol, See Example 34(d)), and DIPEA (0.185 mL, 1.06 mmol), were suspended in EtOH (3 mL) and then refluxed for 1 h. The reaction mixture was cooled to r.t and concentrated under reduced pressure. EtOAc (50 mL) was added and the reaction mixture was washed with saturated NH₄Cl (2×50 mL), dried (MgSO₄) and concentrated under reduced pressure to afford the crude product as a solid Flash chromatography (20% EtOAc in Hexanes then 20% EtOAc in Hexanes with 0.1% AcOH) gave cyclopropyl 6-(4-(benzylsulfonylcarbamoyl)piperidin-1-yl)-5-cyano-2-methylnicotinate as a solid Yield: 0.098 g (57.2%).

¹H NMR (400 MHz, DMSO-d₆): δ 0.73-0.83 (4H, m), 1.59-1.71 (2H, m), 1.79-1.88 (2H, m), 2.54-2.61 (1H, m), 2.64 (3H, s), 3.08-3.20 (2H, m), 4.21-4.30 (1H, m), 4.49-4.59 (2H, m), 4.70 (2H, s), 7.26-7.33 (2H, m), 7.73-7.44 (3H, m), 8.31 (1H, s), 11.6 (1H, s).

MS ^{m}/_{z}: 483 (M+1).

### Example 59

### Cyclobutyl 6-(3-{[(benzylsulfonyl)amino]carbonyl}azetidin-1-yl)-5-cyano-2-methylnicotinate

5-Cyano-2-methyl-6-(3-phenylmethanesulfonylaminocarbonyl-azetidin-1-yl)-nicotinic acid ethyl ester (0.080 g, 6.181 mmol) and molecular sieves (4 Å, 0.100 g) were dissolved in cyclobutanol (1 mL) and DMSO (2 mL) and stirred at room temperature for 1 h. Sodium hydride (95%, 0.014 g, 0.542 mmol) was added to the reaction mixture and stirred for 2 h at room temperature. EtOAc (30 mL) was added and the reaction mixture was filtered through celite. HCl (conc.) was added drop-wise to the mixture until the pH was lowered to pH 2. The reaction mixture was concentrated under reduced pressure. WATER (30 mL) was added and the aqueous was washed with EtOAc (3×50 mL), dried (MgSO₄) and concentrated under reduced pressure to afford the crude product as a solid. Flash chromatography (20% EtOAc in Hexanes with 0.1% AcOH) and Trituration (1/1—Et₂O/Hexanes) gave cyclobutyl 6-(3-{[(benzylsulfonyl)amino]carbonyl}azetidin 1-yl)-5-cyano-2-methylnicotinate as a solid. Yield: 0.020 g (23%).

¹H NMR (400 MHz, DMSO-d₆): δ 1.61-1.71 (1H, m), 1.77-1.85 (1H, m), 2.10-2.22 (2H, m), 2.29-2.40 (2H, m), 2.64 (3H, s), 3.53-3.60 (1H, m), 4.27-4.35 (2H, m), 4.38-4.46 (2H, m), 4.75 (2H, s), 5.04-5.13 (1H, m), 7.31-7.43 (5H, m), 8.35 (1H, s), 11.8 (1H, s).

MS ^{m}/_{z}: 469 (M+1).

### Example 60

### 2-Hydroxyethyl 6-(3-{[(benzylsulfonyl)amino]carbonyl}azetidin-1-yl)-5-cyano-2-methylnicotinate

5-Cyano-2-methyl-6-(3-phenylmethanesulfonylaminocarbonyl-azetidin-1-yl)-nicotinic acid ethyl ester and molecular sieves (4 Å, 0.100 g) were dissolved in ethylene glycol (2 mL) and DMSO (2 mL) and stirred at room temperature for 1 h. Sodium hydride (95%, 0.017 g, 0.678 mmol) was added to the reaction mixture and stirred for 2 h at room temperature. EtOAc (50 mL) was added and the reaction mixture was filtered through celite. HCl (conc.) was added drop-wise to the mixture until the pH was lowered to pH 2. The reaction mixture was concentrated under reduced pressure. Water (30 mL) was added and the aqueous was washed with EtOAc (3×50 mL), dried (MgSO₄) and concentrated under reduced pressure to afford the crude product as a solid. Heating (50% EtOAc in Hexanes at 50° C.) gave 2-hydroxyethyl 6-(3-{[benzylsulfonyl)amino]carbonyl}azetidin-1-yl)-5-cyano-2-methylnicotinate as a solid. Yield: 0.038 g (37.1%).

¹H NMR (400 MHz, DMSO-d₆): δ 2.65 (3H, s), 3.54-3.60 (1H, m), 3.65-3.71 (2H, m), 4.16-4.23 (2H, m), 4.28-4.33 (2H, m), 4.39-4.48 (2H, m), 4.76 (2H, s), 4.91-4.99 (1H, m), 7.31-7.43 (5H, m), 8.46 (1H, s), 11.8 (1H, s).

MS ^{m}/_{z}: 459 (M+1).

### Example 61

### Benzyl 6-(3-{[(benzylsulfonyl)amino]carbonyl}azetidin-1-yl)-5-cyano-2-methylnicotinate

5-Cyano-2-methyl-6-(3-phenylmethanesulfonylaminocarbonyl-azetidin-1-yl)-nicotinic acid ethyl ester (0.100 g, 0.226 mmol) and molecular sieves (4 Å, 0.100 g) were dissolved in benzyl alcohol (5 mL) and DMSO (1 mL) and stirred at room temperature for 1 h. Sodium hydride (95%, 0.017 g, 0.678 mmol) was added to the reaction mixture and stirred for 60 h at room temperature. EtOAc (50 mL) was added and the reaction mixture was filtered through celite. HCl (conc.) was added drop-wise to the mixture until the pH was lowered to pH 2. The reaction mixture was concentrated under reduced pressure. Water (30 mL) was added and the aqueous was washed with EtOAc (3×50 mL), dried (MgSO₄) and concentrated under reduced pressure to afford the crude product as a solid. Flash chromatography (Hexanes then 20% EtOAc in Hexanes, and 20% EtOAc in Hexanes with 0.1% AcOH) gave Benzyl 6-(3-{[(benzylsulfonyl)amino]carbonyl}azetidin-1-yl)-5-cyano-2-methylnicotinate as a solid. Yield: 0.013 g (12%).

¹H NMR (400 M, DMSO-d₆): δ 2.65 (3H, s), 3.51-3.59 (1H, m), 4.27-4.47 (4H, m), 4.75 (2H, s), 5.28 (2H, s), 7.29-7.52 (10H, m), 8.35 (1H, s), 11.8 (1H, s).

### Example 62

### Isopropyl 5-cyano-6-[4-({[(3,4-dichlorobenzyl)sulfonyl]amino}carbonyl)piperidin-1-yl]-2-methylnicotinate

### (a) 1-(3,4-Dichlorophenyl)methanesulfonamide

1-(3,4-dichlorophenyl)methanesulfonyl chloride (1.00 g, 3.85 mmol) and ammonia (1.0 M in THF, 38.5 mL, 38.5 mmol) were suspended in THF (2 mL) and stirred at room temperature for 2 h. The reaction mixture was concentrated under reduced pressure. EtOAc (50 mL) was added and the organics were washed with saturated NaHCO₃ (2×30 mL), dried (MgSO₄) and concentrated under reduced pressure to afford the crude product which was used without further purification. Yield. 0.900 g (97.3%).

¹H NMR (400 MHz, DMSO-d₆): δ 4.31 (2H, s), 6.90 (2H, s), 7.33-7.40 (1H, m), 7.59-7.70 (2H, m).

### (b) Isopropyl 5-cyano-6-[4-({[(3,4-dichlorobenzyl)sulfonyl]amino}carbonyl)piperidin-1-yl]-2-methylnicotinate

1-(3-cyano-5-(isopropoxycarbonyl)-6-methylpyridin-2-yl)piperidine-4-carboxylic acid (0.200 g, 0.604 mmol), EDCI (0.174 g, 0.905 mmol), HOBt (0.090 g, 0.664 mmol), (3,4-dichlorophenyl)methanesulfonamide (0.174 g, 0.724 mmol) and DIPEA (0.315 mL, 1.81 mmol) were suspended in DCM (10 mL) at room temperature and stirred for 16 h. The reaction mixture was diluted with EtOAc (40 mL). The combined organics were washed with saturated NH₄Cl (2×40 mL), dried (MgSO₄) and concentrated under reduced pressure to afford the crude product. Flash chromatography (20% EtOAc in Hexanes then 20% EtOAc in Hexanes with 0.1% AcOH) gave Isopropyl 5-cyano-6-[4-({[(3,4-dichlorobenzyl)sulfonyl]amino}carbonyl)piperidin-1-yl]-2-methylnicotinate as a solid. Yield: 0.292 g (87%).

¹H NMR (400 MHz, DMSO-d₆): δ 1.30 (6H, d, J=6.2 Hz), 1.56-1.70 (2H, m), 1.78-1.89 (2H, m), 2.54-2.62 (1H, m), 2.65 (3H, s), 3.08-3.20 (2H, m), 4.46-4.59 (2H, m), 4.77 (2H, s), 5.02-5.14 (1H, m), 7.25-7.32 (1H, m), 7.54 (1H, s), 7.67-7.76 (1H, m), 8.33 (1H, s), 11.7 (1H, s).

MS ^{m}/_{z}: 553 (M+1).

### Example 63

### Ethyl 5-cyano-6-[3-({[(3,4-dichlorobenzyl)sulfonyl]amino}carbonyl)azetidin-1-yl]-2-methylnicotinate

1-(3-cyano-5-(ethoxycarbonyl)-6-methylpyridin-2-yl)azetidine-3-carboxylic acid (0.200 g, 0.691 mmol), EDCI (0.199 g, 1.04 mmol), HOBt (0.103 g, 0.760 mmol), 1-(3,4-dichlorophenyl)methanesulfonamide (0.199 g, 0.830 mmol) and DIPEA (0.361 mL, 2.07 mmol) were suspended in DCM (10 mL) at room temperature and stirred for 16 h. The reaction mixture was diluted with EtOAc (40 mL). The combined organics were washed with saturated NH₄Cl (2×40 mL), dried (MgSO₄) and concentrated under reduced pressure to afford the crude product. Flash chromatography (20% EtOAc in Hexanes then 20% EtOAc in Hexanes with 0.1% AcOH) gave ethyl 5-cyano-6-[3-({[(3,4-dichlorobenzyl)sulfonyl]amino}carbonyl)azetidin-1-yl]-2-methylnicotinate as a solid. Yield: 0.248 g (70%).

¹H NMR (400 MHz, DMSO-d₆): δ 1.30 (3H, t, J=7.1 Hz), 2.64 (3H, s), 3.53-3.64 (1H, m), 4.19-4.35 (4H, m), 4.38-4.48 (2H, m), 4.82 (2H, s), 7.31-7.39 (1H, m), 7.59-7.70 (2H, s), 8.31 (1H, s), 11.9 (1H, s).

MS ^{m}/_{z}: 511 (M+1).

### Example 64

### Ethyl 5-cyano-6-[4-({[(3,4-dichlorobenzyl)sulfonyl]amino}carbonyl)piperidin-1-yl]-2-methylnicotinate

1-(3-cyano-5-(ethoxycarbonyl)-6-methylpyridin-2-yl)piperidine-4-carboxylic acid (0.200 g, 0.630 mmol), EDCI (0.181 g, 0.945 mmol), HOBt (0.094 g, 0.693 mmol), (3,4-dichlorophenyl)methanesulfonamide (0.182 g, 0.756 mmol) and DIPEA (0.329 mL, 1.89 mmol) were suspended in DCM (10 mL) at room temperature and stirred for 16 h. The reaction mixture was diluted with EtOAc (40 mL). The combined organics were washed with saturated NH₄Cl (2×40 mL), dried (MgSO₄) and concentrated under reduced pressure to afford the crude product Flash chromatography (20% EtOAc in Hexanes then 20% EtOAc in Hexanes with 0.1% AcOH) gave ethyl 5-cyano-6-[4-({[(3,4-dichlorobenzyl)sulfonyl]amino}carbonyl)piperidin-1-yl]-2-methylnicotinate as a solid. Yield: 0.273 g (80.3%).

¹H NMR (400 MHz, DMSO-d₆): δ 1.31 (3H, t, J=7.1 Hz), 1.57-1.70 (2H, m), 1.80-1.88 (2H, m), 2.65 (3H, s), 3.10-3.18 (2H, m), 4.31 (1H, s), 4.25 (2H, q, J=7.1 Hz), 4.50-4.58 (2H, m), 4.76 (2H, s), 7.25-7.32 (1H, m), 7.54 (1H, s), 7.68-7.75 (1H, m), 8.35 (1H, s), 11.7 (1H, s).

MS ^{m}/_{z}: 539 (M+1).

### Example 65

### Isopropyl 5-cyano-6-[4-({[(4-cyanobenzyl)sulfonyl]amino}carbonyl)piperidin-1-yl]-2-methylnicotinate

### (a) Sodium (4-cyanophenyl)methanesulfonate

4-(chloromethyl)benzonitrile (5.00 g, 33.0 mmol) and sodium sulfite (4.42 g, 42.9 mmol) were suspended in a 30% solution of acetone in water (100 mL) and stirred at reflux for 4 h The solution was concentrated under reduced pressure. 95% EtOH (300 mL) was added and the solution heated at 50° C. The solution was then cooled at 0° C. and filtered to afford the crude product as a solid, which was used without further purification. Yield: 7.43 g (103%).

¹H NMR (400 MHz, DMSO-d₆): δ 3.80 (2H, s), 7.45-7.53 (2H, m), 7.68-7.76 (2H, m).

### (b) (4-Cyanophenyl)methanesulfonyl chloride

Sodium (4-cyanophenyl)methanesulfonate (7.43 g, 33.9 mmol) was suspended in a DCM (250 mL) and stirred at 0° C. Phosphorous pentachloride (17.7 g, 84.7 mmol) was added then the solution was warmed and stirred at room temperature for 16 h. water (100 mL) was added and stirred for 5 minutes. The layers were separated and the organics were washed with brine (2×100 mL), dried (MgSO₄) filtered and concentrated to an oil which solidified on standing and used without further purification. Yield. 7.00 g (96%).

¹H NMR (400 MHz, DMSO-d₆): δ 3.86 (2H, s), 7.43-7.54 (2H, m), 7.70-7.79 (2H, m).

### (c) 1-(4-Cyanophenyl)methanesulfonamide

1-(4-cyanophenyl)methanesulfonyl chloride (1.00 g, 4.64 mmol) was suspended in a DCM (25 mL) and stirred at room temperature. Ammonium hydroxide (6.00 mL, 46.4 mmol) was added and the solution was stirred at room temperature for 3 h. The layers were separated and the aqueous was washed with DCM (2×40 mL). The combined organics were dried (MgSO₄), filtered and concentrated to a solid, which was used without further purification. Yield: 0.888 g (98%).

¹H NMR (400 MHz, DMSO-d₆): δ 4.40 (2H, s), 6.94 (2H, s), 7.51-7.61 (2H, m), 7.82-7.92 (2H, m).

### (d) Isopropyl 5-cyano-[4-({[(4-cyanobenzyl)sulfonyl]amino}carbonyl)piperidin-1-yl]-2-methylnicotinate

1-(3-cyano-5-(isopropoxycarbonyl)-6-methylpyridin-2-yl)piperidine-4-carboxylic acid (0.200 g, 0.604 mmol), EDCI (0.174 g, 0.905 mmol), HOBt (0.090 g, 0.664 mmol), (4-cyanophenyl)methanesulfonamide (0.118 g, 0.604 mmol) and DIPEA (0.315 mL, 1.81 mmol) were suspended in DCM (10 mL) at room temperature and stirred for 16 h. The reaction mixture was diluted with EtOAc (40 mL). The combined organics were washed with saturated NH₄Cl (2×40 mL), dried (MgSO₄) and concentrated under reduced pressure to afford the crude product. Flash chromatography (20% EtOAc in Hexanes then 20% EtOAc in Hexanes with 0.1% AcOH) gave isopropyl 5-cyano-6-[4-({[(4-cyanobenzyl)sulfonyl]amino}carbonyl)piperidin-1-yl]-2-methylnicotinate as a solid. Yield: 0.232 g (75 b/o).

¹H NMR (400 MHz, DMSO-d₆): δ 1.30 (6H, d, J=6.2 Hz), 1.56-1.70 (2H, m), 1.80-1.89 (2H, m), 2.54-2.63 (1H, m), 2.65 (3H, s), 3.08-3.19 (2H, m), 4.49-4.58 (2H, m), 4.84 (2H, s), 5.03-5.13 (1H, m), 7.47-7.54 (2H, m), 7.88-7.94 (2H, m), 8.33 (1H, s), 11.7 (1H, s).

MS ^{m}/_{z}: 510 (M+1).

### Example 66

### Ethyl 5-cyano-6-[3-{[(4-cyanobenzyl)sulfonyl]amino}carbonyl)azetidin-1-yl]-2-methylnicotinate

1-(3-cyano-5-(ethoxycarbonyl)-6-methylpyridin-2-yl)azetidine-3-carboxylic acid (0.200 g, 0.691 mmol), EDCI (0.199 g, 1.04 mmol), HOBt (0.103 g, 0.760 mmol), (4-cyanophenyl)methanesulfonamide (0.136 g, 0.691 mmol) and DIPEA (0.361 mL, 2.07 mmol) were suspended in DCM (10 mL) at room temperature and stirred for 16 h. The reaction mixture was diluted with EtOAc (40 mL). The combined organics were washed with saturated NH₄Cl (2×40 mL), dried (MgSO₄) and concentrated under reduced pressure to afford the crude product. Flash chromatography (20% EtOAc in Hexanes then 20% EtOAc in Hexanes with 0.1% AcOH) gave ethyl 5-cyano-6-[3-({[(4-cyanobenzyl)sulfonyl]amino}carbonyl)azetidin-1-yl]-2-methylnicotinate as a solid. Yield: 0.235 g (73%).

¹H NMR (400 MHz, DMSO-d₆): δ 1.30 (3H, t, J=7.1 Hz), 2.64 (3H, s), 3.51-3.64 (1H, m), 4.18-4.35 (4H, m), 4.38-4.49 (2H, m), 4.90 (2H, s), 7.53-7.61 (2H, m), 7.82-7.90 (2H, m), 8.32 (1H, s), 11.9 (1H, s).

MS ^{m}/_{z}: 468 (M+1).

### Example 67

### Isopropyl 5-cyano-6-[4-({[(4-fluorobenzyl)sulfonyl]amino}carbonyl)piperidin-1-yl]-2-methylnicotinate

### (a) 1-(4-Fluorophenyl)methanesulfonamide

Ammonia gas was bubbled through THF (50 mL) at 0° C. for 5 minutes. 1-(4-fluorophenyl)methanesulfonyl chloride (1.00 g, 4.80 mmol) was added to the reaction mixture and the system allowed to warm to r.t. Ammonia gas was bubbled through the system for 5 further minutes and the reaction mixture allowed to stir for a further 30 minutes. The reaction mixture was diluted with EtOAc (100 mL) and washed with saturated NH₄Cl (2×50 mL), brine, dried (MgSO₄) and concentrated under reduced pressure to afford 1-(4-fluorophenyl)methanesulfonamide as a solid, which was used without further purification. Yield. 0.91 g (100%).

¹H NMR (400 MHz, DMSO-d₆): δ 4.26 (2H, s), 6.82 (2H, s), 7.18-7.24 (2H, m), 7.38-7.42 (2H, m).

### (b) Isopropyl 5-cyano-6-[4-({[(4-fluorobenzyl)sulfonyl]amino}carbonyl)piperidin-1-yl]-2-methylnicotinate

1-(3-cyano-5-(isopropoxycarbonyl)-6-methylpyridin-2-yl)piperidine-4-carboxylic acid (0.190 g, 0.573 mmol), 1-(4-fluorophenyl)methanesulfonamide (0.141 g, 0.745 mmol), HOBt (0.101 g, 0.745 mmol) and EDCI (0.143 g, 0.745 mmol) were suspended in DCM (4 mL) and DIPEA (0.300 ml, 1.72 mmol) added. The reaction mixture was stirred overnight at r.t and then concentrated under reduced pressure. The crude reaction mixture was dissolved in MeOH (1.5 mL) and added dropwise to a rapidly stirring solution of KHSO₄ (0.380 g, 2.865 mmol) in water (20 mL). The product was collected by filtration, washed with water (3×10 mL) and dried under vacuum. The dry product was slurried in IPA (4 mL) and stirred and heated at 50° C. for 1 h. The compound was isolated by filtration and dried under high vacuum to give isopropyl 5-cyano-6-[4-({[(4-fluorobenzyl)sulfonyl]amino}carbonyl)piperidin-1-yl]-2-methylnicotinate as a solid. Yield: 0.150 g (54%).

¹H NMR (400 MHz, DMSO-d₆): δ 1.30 (6H, d, J=6.2 Hz), 1.57-1.70 (2H, m), 1.80-1.89 (2H, m), 2.50-2.65 (1H, m), 2.65 (3H, s), 3.09-3.20 (2H, m), 4.49-4.58 (2H, m), 4.70 (2H, s), 5.04-5.12 (1H, m), 7.21-7.29 (2H, m), 7.30-7.38 (2H, m), 8.32 (1H, s), 11.62 (1H, s).

MS ^{m}/_{z}: 503 (M+1).

### Example 68

### Isopropyl 6-[4-({[(4-chlorobenzyl)sulfonyl]amino}carbonyl)piperidin-1-yl]-5-cyano-2-methylnicotinate

### (a) 1-(4-Chlorophenyl)methanesulfonamide

Ammonia gas was bubbled through THF (50 mL) at 0° C. for 5 minutes. 1-(4-fluorophenyl)methanesulfonyl chloride (1.00 g, 4.80 mmol) was added to the reaction mixture and the system allowed to warm to r.t Ammonia gas was bubbled through the system for 5 further minutes and the reaction mixture allowed to stir for a flier 30 minutes. The reaction mixture was diluted with EtOAc (100 mL) and washed with saturated NH₄Cl (2×50 mL), brine, dried (MgSO₄) and concentrated under reduced pressure to afford 1-(4-chlorophenyl)methanesulfonamide as a solid, which was used without further purification. Yield: 0.91 g (100%).

### (b) Isopropyl 6-[4-({[(4-chlorobenzyl)sulfonyl]amino}carbonyl)piperidin-1-yl]-5-cyano-2-methylnicotinate

1-(3-cyano-5-(isopropoxycarbonyl)-6-methylpyridin-2-yl)piperidine-4-carboxylic acid (0.178 g, 0.537 mmol), (4-chlorophenyl)methanesulfonamide (0.144 g, 0.698 mmol), HOBt (0.0944 g, 0.698 mmol) and EDCI (0.134 g, 0.698 mmol) were suspended in DCM (4 mL) and DIPEA (0.476 mL, 2.69 mmol) added. The reaction mixture was stirred overnight at r.t and then concentrated under reduced pressure. The crude reaction mixture was dissolved in MeOH (1.5 mL) and added drop-wise to a rapidly stirring solution of KHSO₄ (0.380 g, 2.865 mmol) in water (20 mL). The product was collected by filtration, washed with water (3×10 mL) and dried under vacuum. The dry product was slurried in IPA (5 mL) and stirred and heated at 50° C. for 1 h. The compound was isolated by filtration and dried under high vacuum to give isopropyl 6-[4-({[(4-chlorobenzyl)sulfonyl]amino}carbonyl)piperidin-1-yl]-5-cyano-2-methylnicotinate as a solid. Yield: 0.150 g (52%).

¹H NMR (400 MHz, DMSO-d₆): δ 1.30 (6H, d, J=6.2 Hz), 1.60-1.70 (2H, m), 1.80-1.89 (2H, m), 2.50-2.65 (1H, m), 2.65 (3H, s), 3.09-3.19 (2H, m), 4.50-4.58 (2H, m), 4.72 (2H, s), 5.04-5.12 (1H, m), 7.31 (2H, d, J=8.4 Hz), 7.49 (2H, d, J=8.4 Hz), 8.32 (1H, s), 11.63 (1H, s).

MS ^{m}/_{z}: 519 (M+1).

### Example 69

### Ethyl 6-(3-{[(benzylsulfonyl)amino]carbonyl}azetidin-1-yl)-5-cyano-2-isopropylnicotinate

### (a) 1-[3-Cyano-5-(ethoxycarbonyl)-6-isopropylpyridin-2-yl]azetidine-3-carboxylic acid

To a solution of ethyl 6-chloro-5-cyano-2-isopropylnicotinate (0.286 g, 1.13 mmol) and DIPEA (0.591 mL, 3.40 mmol) in DMF (3.0 ml) was added azetidine-3-carboxylic acid (0.132 g, 1.31 mmol) and the resulting heterogenous mixture was heated to 90° C. for 3 h. The reaction mixture was diluted with EtOAc (75 mL), washed with saturated NHC. (3×50 mL), brine (50 mL), dried (MgSO₄) and filtered through silica gel. Concentration followed by flash chromatography (1% HOAc, 20% EtOAc, hexanes) gave 1-(3-cyano-5-(ethoxycarbonyl)-6-isopropylpyridin-2-yl)azetidine-3-carboxylic acid as a solid. Yield: 0.118 g (33%).

1H NMR (400 MHz, CDCl₃): δ 1.20 (6H, d, J=6.6 Hz), 1.37 (3H, t, J=7.5 Hz), 3.60-3.68 (1H, m), 3.95-4.02 (1H, m), 4.31 (2H, q, J=7.5 Hz), 4.57-4.65 (4H, m), 8.24 (1H, s).

MS ^{m}/_{z}: 318 (M+1).

### (b) Ethyl 6-(3-{[(benzylsulfonyl)amino]carbonyl}azetidin-1-yl)-5-cyano-2-isopropylnicotinate

A solution of 1-(3-cyano-5-(ethoxycarbonyl)-6-isopropylpyridin-2-yl)azetidine-3-carboxylic acid (0.115 g, 0.362 mmol), EDCI (0.0834 g, 0.435 mmol), HOBtxH₂O (0.0666 g, 0.435 mmol), 1-phenylmethanesulfonamide (0.0620 g, 0.362 mmol) and DIPEA (0.189 mL, 1.09 mmol) in DCM (3.0 mL) was stirred at r.t for 18 h. The reaction mixture was diluted with EtOAc (50 mL) and washed with saturated NH₄Cl (2×40 mL) and brine (40 mL). The organics were dried (MgSO₄) and concentrated under reduced pressure to afford crude product. Flash chromatography (1% MeOH, 1% HOAc, DCM) gave ethyl 6-(3-{[(benzylsulfonyl)amino]carbonyl}azetidin 1-yl)-5-cyano-2-isopropylnicotinate as a solid. Yield: 0.125 g (70%).

¹H NMR (400 MHz, CDCl₃): δ 1.20 (6H, d, J=6.6 Hz), 1.38 (3H, t, J=6.8), 3.29-3.36 (1H, m), 3.96-4.02 (1H, m), 4.32 (2H, q, J=6.8 Hz), 4.45-4.46 (4H, m), 4.69 (2H, s), 7.36-7.43 (51H, m), 7.56 (1H, br s), 8.24 (1H, s).

MS ^{m}/_{z}: 471 (M+1).

### Example 70

### Ethyl 6-(3-{[(benzylsulfonyl)amino]carbonyl}azetidin-1-yl)-5-cyano-2-ethylnicotinate

### (a) 1-(3-Cyano-5-(ethoxycarbonyl)-6-ethylpyridin-2-yl)azetidine-3-carboxylic acid

To a solution of ethyl 6-chloro-5-cyano-2-ethylnicotinate (0.290 g, 1.22 mmol) and DIPEA (0.635 mL, 3.65 mmol) in DMF (3.0 mL) was added azetidine-3-carboxylic acid (0.135 g, 134 mmol) and the resulting heterogenous mixture was heated to 90° C. for 3 h. The reaction mixture was diluted with EtOAc (75 mL), washed with saturated NH₄Cl (3×50 mL), brine (50 mL), dried (MgSO₄) and filtered through silica gel. Concentration followed by flash chromatography (1% HOAc, 20% EtOAc, hexanes) gave 1-(3-cyano-5-(ethoxycarbonyl)-6-ethylpyridin-2-yl)azetidine-3-carboxylic acid as a solid. Yield: 0.047 g 1%).

¹H NMR (400 MHz, CDCl₃): δ 1.22 (3H, t, J=7.4 Hz), 1.37 (3H, t, J=7.0 Hz), 3.10 (2H, q, J=7.4 Hz), 3.60-3.68 (1H, m), 4.31 (2H, q, J=7.4 Hz), 4.58-4.66 (4H, m), 8.27 (1H, s).

MS ^{m}/_{z}: 304 (M+1).

### (b) Ethyl 6-(3-{[(benzylsulfonyl)amino]carbonyl}azetidin-1-yl)-5-cyano-2-ethylnicotinate

A solution of 1-(3-cyano-5-(ethoxycarbonyl)-6-ethylpyridin-2-yl)azetidine-3-carboxylic acid (0.0450 g, 0.148 mmol), EDCI (0.0341 g, 0.178 mmol), HOBt×H₂O (0.0273 g, 0.178 mmol), 1-phenylmethanesulfonamide (0.0254 g, 0.148 mmol) and DIPEA (0.0775 mL, 0.445 mmol) in DCM (3.0 mL) was stirred at room temperature for 18 h. The reaction mixture was diluted with EtOAc (50 mL) and washed with saturated NH₄Cl (2×40 mL) and brine (40 mL). The organics were dried (MgSO₄) and concentrated under reduced pressure to afford crude product. Flash chromatography (1% MeOH, 1% HOAc, DCM) gave ethyl 6-(3{[(benzylsulfonyl)amino]carbonyl}azetidin-1-yl)-5-cyano-2-ethylnicotinate as a solid. Yield: 0.055 g (77%).

¹H NMR (400 z, CDCl₃): δ 1.23 (3H, t, J=7.6 Hz), 1.38 (3H, t, J=7.1 Hz), 3.11 (2H, q, J=7.6 Hz), 3.29-3.36 (1H, m), 4.32 (2H, q, J=7.1 Hz), 4.45-4.47 (4H, m), 4.69 (2H, s), 7.36-7.44 (5H, m), 7.57 (1H, br s), 8.28 (1H, s).

MS ^{m}/_{z}: 457 (M+1).

### Example 71

### Ethyl 5-cyano-2-methyl-6-[3-({[(1-phenylethyl)sulfonyl]amino}carbonyl)azetidin-1-yl]nicotinate

### (a) N-(tert-Butyl)-1-phenylmethanesulfonamide

To a solution of phenylmethanesulfonyl chloride (10.6 g, 55.7 mmol) and tert-butyl amine (23.6 mL, 223 mmol) in DCM (200 mL) at 0° C. was added DIPEA (29.1 mL, 167 mmol). The reaction mixture was warmed to r.t, stirred for 16 h and then concentrated. The mixture was diluted with EtOAc (1000 mL), washed with saturated NH₄Cl (2×250 mL), saturated NaHCO₃ (2×250 mL), brine (50 mL), dried (MgSO₄) and filtered through silica gel. The crude solid was sonicated in a mixture of Et₂O (100 mL) and hexanes (50 mL) producing crystals, which were collected and washed with 1:1 Et₂O/hexanes (50 mL) and pure hexanes (50 mL). Yield: 5.32 g (44%).

¹H NMR (400 MHz, CDCl₃): δ 1.35 (9H, s), 3.93 (1H, br s), 4.24 (2H, s), 7.35-7.41 (5H, m).

### (b) N-(tert-Butyl)-1-phenylethanesulfonamide

To a solution of N-(tert-butyl)-1-phenylmethanesulfonamide (0.918 g, 4.04 mmol) in THF (40 mL) cooled to −78° C. was added dropwise tert-butyl lithium (1.70 M in pentane, 4.75 mL, 8.08 mmol). The reaction was warmed to 0° C. for 1 h and then cooled to −78° C. Dropwise addition of iodomethane (0.252 mL, 4.04 mmol) produced a cloudy mixture which was stirred at −78° C. for 15 minutes then 0° C. for 1 h. The reaction mixture was quenched with saturated NH₄Cl (25 mL), diluted with EtOAc (75 mL), washed with saturated NH₄Cl (3×50 mL), brine (50 mL), dried (MgSO₄) and filtered through silica gel. Concentration followed by flash chromatography (DCM) gave N-(tert-butyl)-1-phenylethanesulfonamide as an oil. Yield: 0.900 g (92%).

¹H NMR (400 MHz, CDCl₃): δ 1.30 (9H, s), 1.78 (3K d, J=7.3 Hz), 3.69 (1H, br s), 4.18 (1H, q, J=7.3 Hz), 7.33-7.39 (3H, m), 7.42-7.45 (2H, m).

### (c) 1-Phenylethanesulfonamide

A solution of N-(tert-butyl)-1-phenylethanesulfonamide (0.900 g, 3.73 mmol) was stirred in TFA (50 mL) for 24 h. Concentration followed by azeotropping from toluene (2×50 mL) produced the crude product, which was dissolved in DCM (25 mL) and passed through silica gel producing 1-phenylethanesulfonamide as a solid. Yield: 0.210 g (30%).

¹H NMR (400 MHz, CDCl₃): δ 1.83 (31H d, J=7.2 Hz), 4.27-4.33 (3H, m), 7.38-7.42 (3H, m), 7.45-7.48 (2H, m).

### (d) Ethyl 5-cyano-2-methyl-6-[3-({[(1-phenylethyl)sulfonyl]amino}carbonyl)azetidin-1-yl]nicotinate

A solution of 1-(3-cyano-5-(ethoxycarbonyl)-6-methylpyridin-2-yl)azetidine-3-carboxylic acid (0.0700 g, 0.242 mmol), EDCI (0.0649 g, 0.339 mmol), HOBtxH₂O (0.0519 g, 0.339 mmol), 1-phenylethanesulfonamide (0.0628 g, 0.0.339 mmol) and DIPEA (0.126 mL, 0.726 mmol) in DCM (3.0 mL) was stirred at room temperature for 18 h. The reaction mixture Was diluted with EtOAc (50 mL) and washed with saturated NH₄Cl (2×40 mL) and brine (40 mL). The organics were dried (MgSO₄) and concentrated under reduced pressure to afford crude product. Flash chromatography (1% MeOH, 1% HOAc, DCM) gave Ethyl 5-cyano-2-methyl-6-[3-({[(1-phenylethyl)sulfonyl]amino}carbonyl)azetidin-1-yl]nicotinate as a solid. Yield: 0.105 g (95%).

¹H NMR (400 MHz, CDCl₃): δ 1.38 (3H, t, J=7.0 Hz), 1.88 (3H, d, J=7.2 Hz), 2.72 (3H, s), 3.17-3.24 (1H, m), 4.25-4.48 (6H, m), 4.86 (1H, q, J=7.2 Hz), 7.40-7.44 (5H, m), 8.28 (1H, s).

MS ^{m}/_{z}: 457 (M+1).

### Example 72

### Propyl 6-(3-{[(benzylsulfonyl)amino]carbonyl}azetidin-1-yl)-5-cyano-2-methylnicotinate

5-Cyano-2-methyl-6-(3-phenylmethanesulfonylaminocarbonyl-azetidin-1-yl-nicotinic acid ethyl ester (0.080 g, 0.181 mmol) and molecular sieves (4 Å, 0.100 g) were dissolved in n-propanol (2 mL) and DMSO (2 mL) and stirred at r.t for 1 h. Sodium hydride (95%, 0.014 g, 0.542 mmol) was added to the reaction mixture and stirred overnight at r.t. HCl (conc.) was added drop-wise to the mixture until the pH was lowered to pH 2. The solid was filtered and collected. The reaction mixture was concentrated under reduced pressure. WATER (30 mL) was added and the aqueous was washed with EtOAc (3×50 mL), dried (MgSO₄) and concentrated under reduced pressure to afford the crude product as a solid, which was combined with the filtered solid. Flash chromatography (10% EtOAc in hexanes with 0.1% AcOH) gave propyl 6-(3-{[(benzylsulfonyl)amino]carbonyl}azetidin-1-yl)-5-cyano-2-methylnicotinateas a solid. Yield: 0.020 g (24.2%).

¹H NMR (400 MHz, CDCl₃): δ 1.02 (3H, t, J=7.4 Hz), 1.74-1.82 (2H, m), 2.73 (3H, s), 3.29-3.36 (1H, m), 4.22 (2H, t, J=6.7 Hz), 4.44-4.50 (4H, m), 4.69 (2H, s), 7.35-7.45 (5H, m) 8.29 (1H, s).

MS ^{m}/_{z}: 457 (M+1).

### Example 73

### Isobutyl 6-(3-{[(benzylsulfonyl)amino]carbonyl}azetidin-1-yl)-5-cyano-2-methylnicotinate

5-Cyano-2-methyl-6-(3-phenylmethanesulfonylaminocarbonyl-azetidin-1-yl)-nicotinic acid ethyl ester (0.080 g, 0.181 mmol) and molecular sieves (4 Å, 0.100 g) were dissolved in isobutanol (2 mL) and DMSO (2 mL) and stirred at r.t for 1 h. Sodium hydride (95%, 0.014 g, 0.542 mmol) was added to the reaction mixture and stirred for 4 h at r.t. EtOAc (30 mL) was added and the reaction mixture was filtered through celite. HCl (conc.) was added drop-wise to the mixture until the pH was lowered to pH 2. The reaction mixture was concentrated under reduced pressure. Water (30 mL) was added and the aqueous was washed with EtOAc (3×50 mL), dried (MgSO₄) and concentrated under reduced pressure to afford the crude product as a solid. Flash chromatography (10% EtOAc in hexanes with 0.1% AcOH) gave Isobutyl 6-(3-{[(benzylsulfonyl)amino]carbonyl}azetidin-1-yl)-5-cyano-2-methylnicotinate as a solid. Yield: 0.040 g (47.0%).

¹H NMR (400 MHz, CDCl₃): δ 1.01 (6H, d, J=6.7 Hz), 2.02-2.10 (1H, m), 2.73 (3H, s), 3.30-3.37 (1H, m), 4.05 (2H, d, J=6.6 Hz), 4.42-4.50 (4H, m), 4.69 (2H, s), 7.36-7.44 (5H, m), 8.28 (1H, s).

MS ^{m}/_{z}: 471 (M+1).

### Example 74

### Isopropyl 5-cyano-2-methyl-6-{4-[({[4-(trifluoromethyl)benzyl]sulfonyl}amino)carbonyl]piperidin-1-yl}nicotinate

### (a) 1-[4-(Trifluoromethyl)phenyl]methanesulfonamide

1-(4-(Trifluoromethyl)phenyl)methanesulfonyl chloride (1.00 g, 3.87 mmol) and ammonia (1.0 M in THF, 38.7 mL, 38.7 mmol) were suspended in THF (2 mL) and stirred at room temperature for 16 h. The reaction mixture was concentrated under reduced pressure. EtOAc (50 mL) was added and the organics were washed with saturated NaHCO₃ (2×30 mL), dried (MgSO₄) and concentrated under reduced pressure to afford 1-(4-(trifluoromethyl)phenyl)methanesulfonamide which was used without further purification. Yield: 0.900 g (97.3%).

¹H NMR (400 MHz, DMSO-d₆): δ 4.40 (2H, s), 6.94 (2H, s), 7.60 (2H, d, J=8.1 Hz), 7.75 (2H, d, J=8.1 Hz).

### (b) Isopropyl 5-cyano-2-methyl-6-{4-[({[4-(trifluoromethyl)benzyl]sulfonyl}amino)carbonyl]piperidin-1-yl}nicotinate

1-(3-Cyano-5-(isopropoxycarbonyl)-6-methylpyridin-2-yl)piperidine-4-carboxylic acid (0.206 g, 0.604 mmol), EDCI (0.174 g, 0.905 mmol), 1-(4-(trifluoromethyl)phenyl)methanesulfonamide (0.173 g, 0.724 mmol), HOBt (0.090 g, 0.664 mmol) and DIPEA (0.315 mL, 1.81 mmol) were suspended in DCM (10 mL) and then refluxed for 3 h. The reaction mixture was cooled to r.t and concentrated under reduced pressure. The crude reaction mixture was dissolved in IPA (10 mL) and added drop-wise to a rapidly stirring solution of KHSO₄ (0.411 g, 3.02 mmol) in water (100 mL). The product was collected by filtration, washed with water (3×20 mL) and dried under vacuum. The dry product was purified by preparing a slurry in IPA (100 mL) and stirring and heating at 50° C. for 1 h. The product was isolated by filtration and dried under high vacuum to give isopropyl isopropyl 5-cyano-2-methyl-6-{4-[({[4-(trifluoromethyl)benzyl]sulfonyl}amino)carbonyl]piperidin-1-yl}nicotinate. Yield: 0.173 g (52.0%).

¹H NMR (400 MHz, CDCl₃): δ 1.35 (6H, d, J=6.2 Hz), 1.77-1.97 (4H, m), 2.41-2.51 (1H, m), 2.73 (3H, s), 3.09-3.20 (21H, m), 4.62-4.71 (2H, m), 4.75 (2H, s), 5.15-5.25 (1H, m), 7.50 (2H, d, J=8.0 Hz), 7.56 (1H, s), 7.67 (2H, d, J=8.0 Hz), 8.34 (1H, s). MS ^{m}/_{z}: 553 (M+1).

### Example 75

### Isopropyl 5-cyano-2-methyl-6-[4-({[(4-methylbenzyl)sulfonyl]amino}carbonyl)piperidin-1-yl]nicotinate

1-(4-methylphenyl)methanesulfonamide (0.099 g, 0.534 mmol) dissolved in DCM (2 m L) and DIPEA (0.155 g, 1.2 mmol) was added to a solution of 1-[3-cyano-5-(isopropoxycarbonyl)-6-methylpyridin-2-yl]piperidine-4-carboxylic acid (0.124 g, 0.374 mmol) and TBTU (0.213 g, 0.663 mmol) in DCM (2 mL) and the reaction was stirred at r.t for 15 h. NaHCO₃ (aq) was added and the mixture was extracted with DCM (3 times). The combined organic phase was dried (MgSO₄), filtered and evaporated to give a crude product. Purification by preparative HPLC (Kromasil C8, 21.5×100 mm column, flow 25 mL/minute using a gradient of 0.1 M NH₄OAc and CH₃CN) gave the pure product as a solid. Yield. 0.103 g (55%).

1H NMR (500 MHz, DMSO-d₆): 1.31 (6H, d), 1.64 (2H, m), 1.84 (2H, m), 2.31 (3H, s), 2.58 (1H, m), 2.65 (3H, s), 3.13 (2H, m), 4.54 (2H, m), 4.64 (2H, s), 5.08 (1H, m), 7.16-7.23 (4H, dd), 8.33 (1H, s), 11.56 (1H, s).

MS ^{m}/_{z}: 499 (M+1)

### Example 76

### Isopropyl 5-cyano-2-methyl-6-[4-({[(3-methylbenzyl)sulfonyl]amino}carbonyl)piperidin-1-yl]nicotinate

TBTU (0.100 g, 0.311 mmol) and DIPEA (0.074 g, 0.574 mmol) was added to a solution of 1-[3-cyano-5-(isopropoxycarbonyl)-6-methylpyridin-2-yl]piperidine-4-carboxylic acid (0.104 g, 0.314 mmol) in DCM (2 mL) and the reaction was stirred at r.t for 40 minutes. 1-(3-methylphenyl)methanesulfonamide (0.071 g, 0.383 mmol) was added together with DCM (2 mL) and the stirring was continued for 18 h. NaHCO₃ (aq) was added and the mixture was extracted with DCM (3 times). The combined organic phase was filtered through a phase separator and evaporated to give a crude product. Purification by preparative HPLC (Kromasil C8, 21.5×100 mm column, flow 25 mL/minute using a gradient of 0.1 M NH₄OAc and CH₃CN) gave the pure product as a solid. Yield: 0.119 g (76%).

1H NMR (500 MHz, DMSO-d₆): 1.31 (6H, d), 1.64 (2H, m), 1.82 (2H, m), 2.31 (3H, s), 2.58 (1H, m), 2.65 (3H, s), 3.14 (2H, m), 4.54 (2H, m), 4.65 (2H, s), 5.08 (1H, m), 7.08-7.31 (4H, m), 8.33 (1H, s), 11.58 (1H, s).

MS ^{m}/_{z}: 499 (M+1)

### Example 77

### Isopropyl 5-cyano-6-[4-({[(3-fluorobenzyl)sulfonyl]amino}carbonyl)piperidin-1-yl]-2-methylnicotinate

TBTU (0.097 g, 0.302 mmol) and DIPEA (0.074 g, 0.574 mmol) was added to a solution of 1-[3-cyano-5-(isopropoxycarbonyl)-6-methylpyridin-2-yl]piperidine-4-carboxylic acid (0.100 g, 0.302 mmol) in DCM (2 mL) and the mixture was stirred at r.t for 2.5 h. 1-(3-fluorophenyl)methanesulfonamide (0.074 g, 0.360 mmol) was added together with DCM (2 mL) and the stirring was continued for 18 h. NaHCO₃ (aq) was added and the mixture was extracted with DCM (3 times). The combined organic phase was filtered through a phase separator and evaporated to give a crude product. Purification by preparative HPLC (Kromasil C8, 21.5×100 mm column, flow 25 mL/minute using a gradient of 0.1 M NH₄OAc and CH₃CN) gave the pure product as a solid. Yield: 0.115 g (76%).

1H NMR (500 MHz, DMSO-d₆): 1.31 (6H, d), 1.63 (2H, m), 1.83 (21H, m), 2.59 (1H, m), 2.65 (3H, s), 3.14 (2H, m), 4.54 (2H, m), 4.75 (2H, s), 5.08 (1H, m), 7.12-7.47 (4H, m), 8.32 (1H, s), 11.68 (1H, s).

MS ^{m}/_{z}: 503 (M+1)

### Example 78

### Isopropyl 5-cyano-6-[4-({[(2-fluorobenzyl)sulfonyl]amino}carbonyl)piperidin-1-yl]-2-methylnicotinate

TBTU (0.097 g, 0.302 mmol) and DIPEA (0.074 g, 0.574 mmol) was added to a solution of 1-[3-cyano-5-(isopropoxycarbonyl)-6-methylpyridin-2-yl]piperidine-4-carboxylic acid (0.100 g, 0.302 mmol) in DCM (2 mL) and the mixture was stirred at r.t for 2.5 h. The mixture was added to 1-(2-fluorophenyl)methanesulfonamide (0.068 g, 0.360 mmol) in DCM (2 mL) and the stirring was continued for 18 h. NaHCO₃ (aq) was added and the mixture was extracted with DCM (3 times). The combined organic phase was filtered through a phase separator and evaporated to give a crude product. Purification by preparative HPLC (Kromasil C8, 21.5×100 mm column, flow 25 mL/minute using a gradient of 0.1 M NH₄OAc and CH₃CN) gave the pure product as a solid. Yield: 0.115 g (76%).

¹H NMR (500 MHz, DMSO-d₆): 1.29 (6H, d), 1.64 (2H, m), 1.87 (2H, m), 2.61 (1H, m), 2.64 (3H, s), 3.15 (2H, m), 4.53 (2H, m), 4.75 (2H, s), 5.07 (1H, m), 7.24-7.47 (4H, m), 8.31 (1H, s), 11.74 (1H, s).

MS ^{m}/_{z}: 503 (M+1).

### Example 79

### Isopropyl 6-[4-({([(3-chlorobenzyl)sulfonyl]amino}carbonyl)piperidin-1-yl]-5-cyano-2-methylnicotinate

TBTU (0.097 g, 0.302 mmol) and DIPEA (0.074 g, 0.574 mmol) was added to a solution of 1-[3-cyano-5-(isopropoxycarbonyl)-6-methylpyridin-2-yl]piperidine-4-carboxylic acid (0.100 g, 0.302 mmol) in DCM (2 mL) and the mixture was stirred at r.t for 2.5 h. The mixture was added to 1-(3-chlorophenyl)methanesulfonamide (0.074 g, 0.360 mmol) in DCM (2 mL) and the stirring was continued for 18 h. NaHCO₃ (aq) was added and the mixture was extracted with DCM (3 times). The combined organic phase was filtered through a phase separator and evaporated to give a crude product. Purification by preparative HPLC (Kromasil C8, 21.5×100 mm column, flow 25 mL/minute using a gradient of 0.1 M NH₄OAc and CH₃CN) gave the pure product as a solid. Yield: 0.118 g (75%).

¹H NMR (500 MHz, DMSO-d₆): 1.29 (6H, d), 1.62 (2H, m), 1.82 (2H, m), 2.58 (1H, m), 2.63 (3H, s), 3.13 (2H, m), 4.52 (2H, m), 4.74 (2H, s), 5.07 (1H, m), 7.24-7.47 (4H, m), 8.31 (1H, s), 11.66 (1H, s).

MS ^{m}/_{z}: 520 (M+1)

### Example 80

### Isopropyl 6-[4-({[(2-chlorobenzyl)sulfonyl]amino}carbonyl)piperidin-1-yl]-5-cyano-2-methylnicotinate

TBTU (0.097 g, 0.302 mmol) and DIPEA (0.074 g, 0.574 mmol) was added to a solution of 1-[3-cyano-5-(isopropoxycarbonyl)-6-methylpyridin-2-yl]piperidine-4-carboxylic acid (0.100 g, 0.302 mmol) in DCM (2 mL) and the mixture was stirred at r.t for 2.5 h. The mixture was added to 1-(2-chlorophenyl)methanesulfonamide (0.074 g, 0.360 mmol) in DCM (2 mL) and the stirring was continued for 18 h. NaHCO₃ (aq) was added and the mixture was extracted with DCM (3 times). The combined organic phase was filtered through a phase separator and evaporated to give a crude product. Purification by preparative HPLC (Kromasil C8, 21.5×100 mm column, flow 25 mL/minute using a gradient of 0.1 M NH₄OAc and CH₃CN) gave the pure product as a solid. Yield: 0.116 g (74%).

1H NMR (500, DMSO-d₆): 1.29 (6H, d), 1.65 (2H, m), 1.89 (2H, m), 2.63 (1H, m), 2.63 (3H, s), 3.15 (2H, m), 4.53 (2H, m), 4.85 (2H, s), 5.07 (1H, m), 7.38-7.54 (4H, m), 8.31 (1H, s), 11.80 (1H, s).

MS ^{m}/_{z}: 520 (M+1)

### Example 81

### Ethyl 5-cyano-2-methyl-6-[4-({[(4-methylbenzyl)sulfonyl]amino}carbonyl)piperidin-1-yl]nicotinate and sodium ({1-[3-cyano-5-(ethoxycarbonyl)-6-methylpyridin-2-yl]piperidin-4-yl}carbonyl)[(4-methylbenzyl)sulfonyl]azanide

TBTU (1.162 g, 3.62 mmol) and DIPEA (2.04 g, 15.76 mmol) was added to a solution of 1-[3-cyano-5-(ethoxycarbonyl)-6-methylpyridin-2-yl]piperidine-4-carboxylic acid (0.100 g, 0.302 mmol) in DCM (53 mL) and the mixture was stirred at r.t for 5 minutes. 1-(4-methylphenyl)methanesulfonamide (0.67 g, 3.62 mmol) was added and the stirring was continued over night. The reaction mixture was concentrated and partioned between EtOAc (200 mL), water (50 mL) and formic acid (5 mL). The organic phase was separated and evaporated to give a crude pink colored solid which was purified by preparative HPLC (Kromasil C8 a gradient of 0.1 M NH₄OAc and CH₃CN) to give ethyl 5-cyano-2-methyl-6-[4-({[(4-methylbenzyl)sulfonyl]amino}carbonyl)piperidin-yl]nicotinate as an off white solid. Yield: 0.687 g (45%).

¹H NMR (400 MHz, DMSO-d₆) δ 1.30 (3H, t, J=7.1 Hz), 1.56-1.68 (2H, m), 1.79-1.87 (2H, m), 2.29 (3H, s), 2.41-2.60 (1H, m, concealed under the DMSO-peak), 2.64 (3H, s), 3.09-3.18 (2H, m), 3.29 (1H, s), 4.24 (2H, q, J=7.1 Hz), 4.48-4.56 (2H, m), 4.59 (2H, s), 7.13-7.21 (4H, m), 8.33 (1H, s)

MS ^{m}/_{z}: 485 (M+1).

Ethyl 5-cyano-2-methyl-6-[4-({[(4-methylbenzyl)sulfonyl]amino}carbonyl)piperidin-1-yl]nicotinate (0.687 g, 1.38 mmol) was dissolved in CH₃CN/water and 1 M NaOH (1.38 mL) was added and the mixture was freeze dried to give Sodium ({1-[3-cyano-5-(ethoxycarbonyl)-6-methylpyridin-2-yl]piperidin-4-yl}carbonyl)[(4-methylbenzyl)sulfonyl]azanide as a white solid. Yield: 0.726 g (104%, containing some residual water).

1H NMR (400 MHz, DMSO-d₆) δ 1.30 (3H, t, J=7.2 Hz), 1.51-1.65 (2H, m), 1.74-1.82 (2H, m), 2.17-2.23 (1H, m), 2.25 (3H, s), 2.63 (3H, s), 3.14-3.25 (2H, m), 4.17 (2H, s), 4.24 (2H, q, J=7.1 Hz), 4.37-4.45 (2H, m), 7.03 (2H, d, J=8.1 Hz), 7.10 (2H, d, J=8.1 Hz), 8.30 (1H, s)

### Example 82

### Ethyl 5-cyano-6-{4-[({([2-methoxycarbonyl)benzyl]sulfonyl}amino)carbonyl]piperidin-1-yl}-2-methylnicotinate

Prepared according to method B starting from methyl 2-[(aminosulfonyl)methyl]benzoate. Yield=77 mg (69%).

¹H NMR (400 MHz, DMSO-d₆) δ 1.30 (3H, t, J=7.1 Hz), 1.56-1.71 (2H, m), 1.81-1.89 (2H, m), 2.48-2.53 (1H, m), 2.65 (3H, s), 3.08-3.20 (2H, m), 3.83 (3H, s), 4.25 (2H, q, J=7.1 Hz), 4.46-4.61 (2H, m), 5.14 (2H, s), 7.35 (1H, d, J=7.5 Hz), 7.46-7.68 (2H, m), 7.85 (1H, d, J=7.9 Hz), 8.34 (1H, s), 11.58-11.66 (1H, m)

MS ^{m}/_{z}: 529 (M+1).

### Example 83

### Ethyl 5-cyano-6-[4-{[(3-fluorobenzyl)sulfonyl]amino}carbonyl)piperidin-1-yl]-2-methylnicotinate

Prepared according to method B staring from 1-(3-fluorophenyl)methanesulfonamide. Yield=76 mg (74%).

1H NMR (400, DMSO-d₆) δ 1.30 (3H, t, J=7.2 Hz), 1.56-1.69 (2H, m), 1.78-1.87 (2H, m), 2.41-2.60 (1H, m, concealed-under DMSO-peak), 2.65 (3H, s), 3.10-3.20 (2H, m), 4.25 (2H, q, J=7.1 Hz), 4.52 (2H, d, J=13.3 Hz), 4.72 (2H, s), 7.08-7.16 (2H, m), 7.19-7.29 (1H, m), 7.45 (1H, q, J=7.4 Hz), 8.34 (1H, s), 11.67 (1H, s)

MS ^{m}/_{z}: 489 (M+1).

### Example 84

### Isopropyl 5-cyano-2-methyl-6-[4-({[2-(2-methylphenyl)ethyl]sulfonyl}carbamoyl)piperidin-1-yl]nicotinate

TBTU (0.097 g, 0.302 mmol) and DIPEA (0.074 g, 0.574 mmol) was added to a solution of 1-[3-cyano-5-(isopropoxycarbonyl)-6-methylpyridin-2-yl]piperidine-4-carboxylic acid (0.100 g, 0.302 mmol) in DCM (2 mL) and the mixture was stirred at r.t for 2.5 h. The mixture was added to 2-(2-methylphenyl)ethanesulfonamide (0.074 g, 0.360 mmol) in DCM (2 mL) and the stirring was continued for 18 h. NaHCO₃ (aq) was added and the mixture was extracted with DCM (3 times). The combined organic phase was filtered through a phase separator and evaporated to give a crude product. Purification by preparative HPLC (Kromasil C8, 21.5×100 mm column, flow 25 mL/minute using a gradient of 0.1 M NH₄OAc and CH₃CN) gave the pure product as a solid. Yield: 0.098 g (63%).

¹H NMR (500 MHz, DMSO-d₆): 1.29 (6H, d), 1.59 (2H, m), 1.89 (2H, m), 2.24 (3H, s), 2.63 (1H, m), 2.63 (3H, s), 2.95 (2H, m), 3.15 (2H, m), 3.59 (2H, m), 4.52 (2H, m), 5.07 (1H, m), 7.11-7.18 (4H, m), 8.30 (1H, s), 11.80 (1H, s).

MS ^{m}/_{z}: 513 (M+1), 511 (M−1).

### Example 85

### Ethyl 6-(4-{[(benzylsulfonyl)amino]carbonyl}piperidin-1-yl)-5-(4-methoxy-4-oxobutoxy)-2-methylnicotinate

### (a) Ethyl 5-acetyl-2-methyl-6-oxo-1,6-dihydropyridine-3-carboxylate

Ethyl 5-acetyl-2-methyl-6-oxo-1,6-dihydropyridine-3-carboxylate was prepared from 3-oxobutanamide employing the same methodology that produced ethyl 5-cyano-2-methyl-6-oxo-1,6-dihydropyridine-3-carboxylate.

### (b) Ethyl 5-hydroxy-2-methyl-6-oxo-1,6-dihydropyridine-3-carboxylate

To a solution of H₂O₂ (30% in water, 74.8 mL, 792 mmol) in EtOH (500 mL) cooled to 0° C. was added drop-wise TFA (89.5 mL, 1.16 mol). A suspension of ethyl 5-acetyl-2-methyl-6-oxo-1,6-dihydropyridine-3-carboxylate (11.8 g, 52.8 mmol) in EtOH (500 mL) was added slowly to the reaction mixture, which was then heated to reflux for 24 h and then allowed to cool to r.t. In a separate flask, TFA (89.5 mL, 1.16 mol) was added drop-wise to a solution of H₂O₂ (30% in water, 74.8 mL, 792 mmol) in EtOH (100 mL) cooled to 0° C. and stirred for 15 minutes. This solution was then added to the reaction mixture, which was heated to reflux for an additional 18 h After cooling to r.t, the mixture was concentrated under reduced pressure and azeotroped from toluene (8×100 mL). Addition of CH₃CN (100 mL) produced a solid which was collected and washed CH₃CN (100 mL). Yield: 2.50 g (24%).

¹H NMR (400 MHz, CDCl₃): δ 1.37 (3H, t, J=7.1 Hz), 2.66 (3H, s), 4.31 (2H, q, J=7.1 Hz), 6.35 (1H, br s), 7.50 (1H, s), 11.75 (1H, br s).

MS ^{m}/_{z}: 196 (M−1).

### (c) Ethyl 5-(4-methoxy-4-oxobutoxy)-2-methyl-6-oxo-1,6-dihydropyridine-3-carboxylate

To a solution of ethyl 5-hydroxy-2-methyl-6-oxo-1,6-dihydropyridine-3-carboxylate (0.824 g, 4.18 mmol) in DMF (25 mL) cooled to 0° C. was added LiH (0.0385 g, 4.60 mmol) and the resulting mixture was stirred for 1.5 h. Methyl 4-bromobutanoate (0.832 g 4.60 mmol) and TBAI (0.005 g, 0.0135 mmol) were added and the reaction was heated to 60° C. for 20 h. After cooling to r.t the mixture was diluted with EtOAc (200 mL), washed with saturated NH₄Cl (3×100 mL), brine, dried (MgSO₄) and passed through silica gel. Flash chromatography (1:1 EtOAc/hexanes) furnished ethyl 5-(4-methoxy-4-oxobutoxy)-2-methyl 6-oxo-1,6-dihydropyridine-3-carboxylate as a solid, Yield: 0.60 g (48%)

¹H NMR (400 MHz, CDCl₃): δ 1.41 (3H, t, J=7.1 Hz), 2.15-2.22 (2H, m), 2.60 (2H, t, J=7.1 Hz), 2.73 (3H, s), 3.71 (3H, s), 4.13 (2H, t, J=6.0 Hz), 4.39 (2H, q, J-7.1 Hz), 7.70 (1H, s).

MS ^{m}/_{z}: 298 (M+1).

### (d) Ethyl 6-chloro-5-(4-methoxy-4-oxobutoxy)-2-methylnicotinate

A suspension of ethyl 5-(4-methoxy-4-oxobutoxy)-2-methyl-6-oxo-1,6-dihydropyridine-3-carboxylate (0.600 g, 2.02 mmol) in POCl₃ (25 mL) was heated to 60° C. for 5 h. After cooling to r.t, the reaction mixture was concentrated under reduced pressure, diluted with EtOAc (200 mL), washed with saturated NaHCO₃ (2×100 mL), brine, dried (MgSO₄) and passed through silica gel. Concentration afforded ethyl 6-chloro-5-(4-methoxy-4-oxobutoxy)-2-methylnicotinate. Yield: 0.589 g (92%).

¹H NMR (400 MHz, CDCl₃): δ 1.41 (3H, t, J=7.1 Hz), 2.15-2.22 (2H, m), 2.60 (2H, t, J=7.1 Hz), 2.74 (3H, s), 3.71 (3H, s), 4.13 (2H, t, J=6.0 Hz), 4.39 (2H, q, J=7.1 Hz), 7.70 (1H, s).

MS ^{m}/_{z}: 316 (M+1).

### (e) Ethyl 6-(4-{[(benzylsulfonyl)amino]carbonyl}piperidin-1-yl)-S-(4-methoxy-4-oxobutoxy)-2-methylnicotinate

A suspension of ethyl 6-chloro-5-(4-methoxy-4-oxobutoxy)-2-methylnicotinate (0.300 g, 1.23 mmol), N-(benzylsulfonyl)piperidine-4-carboxamide hydrochloride (0.413 g, 1.30 mmol) and DIPEA (0.371 mL, 2.13 mmol) in NW (2 mL) was stirred at 80° C. for 24 h. The reaction mixture was cooled to r.t and poured into EtOAc (60 mL) and saturated NH₄Cl (30 mL). The organics were washed with water (3×50 mL), brine (1×50 mL), dried MgSO₄) and concentrated under reduced pressure to afford the crude material. Flash chromatography (1:4 EtOAc/hexanes with 1% AcOH) furnished ethyl 6-(4-{[(benzylsulfonyl)amino]carbonyl}piperidin-1-yl)-5-(4-methoxy-4-oxobutoxy)-2-methylnicotinate as a solid. Yield: 0.060 g (29%).

¹H NMR (400 MHz, CDCl₃): δ 1.38 (3H, t, J=7.1 Hz), 1.78-1.84 (4H, m), 2.11-2.17 (2H, m), 2.32-2.40 (1H, m), 2.52 (2H, t, J=7.2 Hz), 2.65 (3H, s), 2.80-2.87 (2H, m), 3.68 (3H, s), 4.01 (2H, t, J=6.2 Hz), 4.29-4.36 (4H, m), 4.68 (2H, s), 7.35-7.40 (5H, m), 7.54 (1H, s).

MS ^{m}/_{z}: 562 (M+1).

### Example 86

### 4-{[2-(4-{[(Benzylsulfonyl)amino]carbonyl}piperidin-1-yl)-5-(ethoxycarbonyl)-6-methylpyridin-3-yl]oxy}butanoic acid

A solution of ethyl 6-(4-{[(benzylsulfonyl)amino]carbonyl}piperidin-1-yl)-5-(4-methoxy-4-oxobutoxy)-2-methylnicotinate (0.050 g, 0.089 mmol) in THF (4 mL) was cooled to 0° C. and treated with NaOH (1.00 M, 0.18 mL, 0.18 mmol). The reaction was warmed to r.t and stirred for 18 h. After dilution with EtOAc (100 mL), the mixture was washed with saturated NH₄Cl (2×50 mL), brine (50 mL), dried (MgSO₄), and concentrated Flash chromatography (50% EtOAc/hexanes with 1% HOAc) furnished 4-(2-(4-(benzylsulfonylcarbamoyl)piperidin-1-yl)-5-(ethoxycarbonyl)-6-methylpyridin-3-yloxy)butanoic acid as a solid Yield: 0.036 g (67%).

¹H NMR (400 MHz, CDCl₃): δ 1.38 (3H, t, J=7.1 Hz), 1.82-1.86 (4H, m), 2.06-2.13 (2H, m), 2.39-2.45 (1H, m), 2.50 (2H, t, J=6.9 Hz), 2.65 (3H, s), 2.77-2.84 (2H, m), 4.02 (2H, t, J=6.6 Hz), 4.24-4.27 (2H, m), 4.32 (2H, q, J=7.1 Hz), 4.68 (2H, s), 7.36-7.40 (5H, m), 7.59 (1H, s).

MS ^{m}/_{z}: 548 (M+1).

### Example 87

### Ethyl 6-(3-{[(benzylsulfonyl)amino]carbonyl}azetidin-1-yl)-5-(4-methoxy-4-oxobutoxy)-2-methylnicotinate

### (a) 1-[5-(ethoxycarbonyl)-3-(4-methoxy-4-oxobutoxy)-6-methylpyridin-2-yl]azetidine-3-carboxylic acid

Azetidine-3-carboxylic acid (0.380 g, 3.76 mmol) and tetrabutylammonium hydroxide (2.3 g, 3.55 mmol, as a 40% solution in water) were combined in MeOH. The azetidine acid was dissolved and the solution was then concentrated in vacuo and azeotroped with toluene twice. The resulting oil was pumped under vacuum. NMP (9 mL) was added along with dried 4 A molecular sieves. The resulting solution was used as a 0.35 M solution of tetrabutylammonium azetidine-3-carboxylate. Ethyl 6-chloro-5-(4-methoxy-4-oxobutoxy)-2-methylnicotinate (0.060 g, 0.19 mmol) was dissolved in an NMP solution of tetrabutylammonium azetidine-3-carboxylate (2.0 mL, 0.70 mmol). The reaction was stirred at room temperature. After 2 hr the reaction was complete and was poured into EtOAc (75 mL), washed with NH₄Cl (2×40 mL) and dried (MgSO₄). The solution was concentrated in vacuo to provide the product, 1-[S-(ethoxycarbonyl)-3-(4-methoxy-4-oxobutoxy)-6-methylpyridin-2-yl]azetidine-3-carboxylic acid, which was used without further purification. Yield 0.070 g (97%).

¹H NMR (400 MHz, CDCl₃): 1.37 (3H, t, J=7.1 Hz), 2.08-2.15 (2H, m), 2.51 (2H, t, J=7.3 Hz), 2.63 (3H, s), 3.52-3.59 (1H, m), 3.70 (3H, s), 3.96 (2H, t, J=6.1 Hz), 4.31 (2H, q, J=7.1 Hz), 4.22-4.44 (4H, m), 7.40 (1H, s).

MS ^{m}/_{z}: 379 (M−1).

### (b) Ethyl 6-(3-{[(benzylsulfonyl)amino]carbonyl}azetidin-1-yl)-5-(4-methoxy-4-oxobutoxy)-2-methylnicotinate

1-(5-(Ethoxycarbonyl)-3-(4-methoxy-4-oxobutoxy)-6-methylpyridin-2-yl)azetidine-3-carboxylic acid (0.070 g, 0.18 mmol), HOBt (0.032 g, 0.24 mmol), 1-phenylmethanesulfonamide (0.044 g, 0.26 mmol) and EDCI (0.046 g, 0.24 mmol) were partially dissolved in dry DCM (2.5 mL) and then DIPEA (0.16 mL, 0.92 mmol) was added. The reaction was allowed to stir overnight at r.t. The reaction was then concentrated with vacuum and the residue was dissolved in MeOH (0.5 mL). The resulting solution was added slowly to a solution of KHSO₄ (0.125 g, 0.92 mmol) in water (7 mL). No clear precipitate formed so the mixture was partitioned between EtOAc (40 m) and water (10 mL). The organic phase was washed with saturated NH₄Cl then brine and dried (MgSO₄). The solution was concentrated in vacuo and purified by column chromatography (30% EtOAc/hexanes to 50% EtOAc/hexanes then 0.1% HOAc added) to provide ethyl 6-(3-{[(benzylsulfonyl)amino]carbonyl}azetidin-1-yl)-5-(4-methoxy-4-oxobutoxy)-2-methylnicotinate as an oil. Yield: 0.032 g (33%).

¹H NMR (400 MHz, CDCl₃): δ 1.37 (3H, t, J=7.1 Hz), 2.06-2.13 (2H, m), 2.48 (2H, t, J=7.3 Hz), 2.62 (3H, s), 3.29-3.36 (1H, m), 3.67 (3H, s), 3.96 (2H, t, J=6.1 Hz), 4.28-4.34 (6H, m), 4.67 (2H, s), 7.37-7.38 (5H, m), 7.41 (1H, s).

MS ^{m}/_{z}: 534 (M+1).

### Example 88

### Ethyl 6-(4-{[(anilinosulfonyl)amino]carbonyl}piperidin-1-yl)-5-cyano-2-methylnicotinate

1-(3-Cyano-5-(ethoxycarbonyl)-6-methylpyridin-2-yl)piperidine-4-carboxylic acid (0.155 g, 0.488 mmol) was partially dissolved in dry DCE (4 mL). CDI (0.103 g, 0.635 mmol) was added and the reaction mixture was heated at 50° C. for 3 hours. N-Phenylsulfamide [Bioorganic & Medicinal Chemistry Letters 2003, 18, 837] (0.101 g, 0.586 mmol) was added followed by DBU (0.0875 mL, 0.586 mmol) and the reaction was heated at 50° C. for an additional 16 hours. The reaction was cooled and concentrated. The residue was partitioned between EtOAc (75 mL) and aqueous NH₄Cl (50 mL) and the organic phase was further washed with NH₄Cl (40 mL) and brine (40 mL). The solution was then concentrated to provide a white solid which was triturated with MeOH to provide the desired product, ethyl 6-(4-{[(anilinosulfonyl)amino]carbonyl}piperidin-1-yl)-5-cyano-2-methylnicotinate, as a white solid. Yield 0.16 g (70%/O).

NMR (400 MHz, DMSO-d₆): δ 1.30 (3H, t, J=7.1 Hz), 1.36-1.46 (2H, m), 1.63-1.65 (2H, m), 2.45-2.50 (1H, obs), 2.62 (3H, s), 3.09 (2H, d, J=11.6 Hz), 4.24 (2H, q, J=7.1 Hz), 4.36-4.39 (2H, m), 7.10 (1H, t, J=7.4 Hz), 7.15 (2H, d, J=7.7 Hz), 7.31 (2H, t, J=7.9 Hz), 8.31 (1H, s), 10.38 (1H, s), 11.73 (1H, s).

MS ^{m}/_{z}: 472 (M+1).

### Example 89

### Ethyl 5-cyano-2-methyl-6-{4-[({[methyl(phenyl)amino]sulfonyl}amino)carbonyl]piperidin-1-yl}nicotinate

Ethyl 5-cyano-2-methyl-6-(4-(N-phenylsulfamoylcarbamoyl)piperidin-1-yl)nicotinate (0.047 g, 0.10 mmol) was dissolved in dry DMF (1 mL) and cooled to 0° C. Sodium hydride (0.010 g, 60% w/w, 0.25 mmol) was added and the reaction was warmed to room temperature and then at 35° C. for 5 minutes. The reaction was returned to 0° C. and then iodomethane (6.0 μL, 0.010 mmol) was added. After 30 minutes, the reaction was warmed to room temperature and stirred for 2 hours. The reaction was then acidified with HOAc (0.2 mL) and partitioned between EtOAc (75 mL) and aqueous NH₄Cl (50 mL). The organic phase was washed with NH₄Cl (30 mL), water (30 mL), dried (MgSO₄) and concentrated in vacuo. The crude product was purified by column chromatography (30 to 40% EtOAc/hexanes) to provide the desired product, ethyl 5-cyano-2-methyl-6-(4-(N-methyl-N-phenylsulfamoylcarbamoyl)piperidin-1-yl)nicotinate. Yield: 0.012 g (25%).

NMR spectroscopy using NOE confirmed the desired regioisomer.

¹H NMR (400 MHz, CDCl₃): δ 1.38 (3H, t, J=7.1 Hz), 1.71-1.86 (4H, m), 2.35-2.42 (1H, m), 2.72 (3H, s), 3.09-3.16 (2H, m), 3.52 (3H, s), 4.32 (2H, q, J=7.1 Hz), 4.63-4.67 (2H, m), 7.31-7.41 (5H, m), 8.35 (1H, s).

MS ^{m}/_{z}: 486 (M+1).

### Example 90

### Isopropyl 5-cyano-2-methyl-6-[3-({[(4-methylbenzyl)sulfonyl]amino}carbonyl)azetidin-1-yl]nicotinate

Prepared according to method C starting from 1-(4-methylphenyl)methanesulfonamide. Yield=4 mg. (4%)

MS ^{m}/_{z}: 471 (M+1)

### Example 91

### Isopropyl 5-cyano-6-[3-({[(3-fluorobenzyl)sulfonyl]amino}carbonyl)azetidin-1-yl]-2-methylnicotinate

Prepared according to method C starting from 1-(3-fluorophenyl)methanesulfonamide. Yield=6.4 mg. (4.5%)

¹H NMR (400 MHz, DMSO-d₆): δ 1.32 (d, J=6.3 Hz, 6H), 2.65 (s, 3H), 3.51-3.61 (m, 1H), 4.26-4.34 (m, 2H), 4.38-4.47 (m, 2H), 4.75-4.81 (br s, 2H), 5.04-5.12 (m, 1H), 7.16-7.28 (m, 3H), 7.39-7.48 (m, 1H), 8.30 (s, 1H), 11.88-11.94 (br s, 1H).

MS ^{m}/_{z}: 475 (M+1)

### Example 92

### Isopropyl 5-cyano-2-methyl-6-[3-({[(2-phenylethyl)sulfonyl]amino}carbonyl)azetidin-1-yl]nicotinate

Prepared according to method C starting from 2-phenylethanesulfonamide. Yield=31 mg. (22%)

¹H NMR (400 M&, DMSO-d₆): δ 1.31 (d, J=6.3 Hz, 6H), 2.63 (s, 3H), 3.00-3.07 (m, 2H), 3.52-3.61 (m, 1), 3.70-3.77 (m, 2H), 4.24-4.32 (m, 2H), 4.38-4.47 (m, 2H), 5.04-5.12 (m, 1H), 7.20-7.35 (m, 5H), 8.29 (s, 1H), 11.88-12.03 (br s, 1H).

MS ^{m}/_{z}: 471 (M+1)

### Example 93

### Isopropyl 5-cyano-6-[3-({[(cyclopentylmethyl)sulfonyl]amino}carbonyl)azetidin-1-yl]-2-methylnicotinate

Prepared according to method C starting from 1-cyclopentylmethanesulfonamide. Yield=28 mg. (21%)

¹H NMR (400 MHz, DMSO-d₆): δ 1.24-1.34 (m, 8H), 1.48-1.66 (m, 4H), 1.82-11.92 (m, 2H), 2.17-2.27 (m, 1H), 2.64 (s, 3H), 3.46 (d, J=6.8 Hz, 2H), 3.57-3.67 (m, 1H), 4.32-4.39 (m, 2H), 4.42-4.51 (m, 2H), 5.04-5.13 (m, 1M, 8.29 (s, 1H), 11.91 (br s, 1H).

MS ^{m}/_{z}: 449 (M+1)

### Example 94

### Isopropyl 5-cyano-6-{3-[({[2-(methoxycarbonyl)benzyl]sulfonyl}amino)carbonyl]azetidin-1-yl}-2-methylnicotinate

Prepared according to method C starting from methyl 2-[(aminosulfonyl)methyl]benzoate. Yield=46 mg. (30%)

¹H NMR (400 MHz, DMSO-d₆): δ 1.31 (d, J=6.3 Hz, 6H), 2.65 (s, 3H), 3.50-3.60 (m, 1H), 3.83 (s, 31), 4.29-4.36 (m, 2H), 4.38-4.47 (m, 2H), 5.03-5.14 (m, 1H), 5.22 (s, 2H), 7.45-7.48 (m, 1H), 7.52-7.58 (m, 1H), 7.59-7.65 (m, 1H), 7.83-7.87 (m, 1H), 8.31 (s, 1H), 11.83-11.87 (br s, 1H).

MS ^{m}/_{z}: (M+1)

### Example 95

### Isopropyl 5-cyano-6-[3-{[(2-fluorobenzyl)sulfonyl]amino}carbonyl)azetidin-1-yl]-2-methylnicotinate

Prepared according to method C starting from 1-(2-fluorophenyl)methanesulfonamide. Yield=53 mg. (37%)

¹H NMR (400 MHz, DMSO-d₆): δ 1.32 (d, J=6.3 Hz, 6H), 2.66 (s, 3H), 3.57-3.66 (m, 1H), 3.33-4.41 (m, 2H), 4.42-4.52 (m, 2H), 4.83 (s, 2H), 5.03-5.14 (m, 1H), 7.24-7.31 (m, 2H), 7.45-7.53 (m, 2H), 8.31 (s, 1H), 11.99 (br s, 1H).

MS ^{m}/_{z}: 515 (M+1)

### Example 96

### Isopropyl 6-[3-({[(4-chlorobenzyl)sulfonyl]amino}carbonyl)azetidin-1-yl]-5-cyano-2-methylnicotinate

Prepared according to method C starting from 1-(4-chlorophenyl)methanesulfonamide Yield=76 mg. (52%)

¹H NMR (400 MHz, DMSO-d₆): δ 1.32 (d, J=6.3 Hz, 6H), 2.66 (s, 3H), 3.51-3.61 (s, 1H), 4.28-4.36 (m, 2H), 4.38-4.47 (m, 2H), 4.75 (s, 2H), 5.04-5.13 (m, 1H), 7.35-7.40 (m, 2H), 7.43-7.48 (m, 2H), 8.31 (s, 1H), 11.87 (br s, 1M).

MS ^{m}/_{z}: 491 (M+1)

### Example 97

### Isopropyl 5-cyano-6-[3-({[(4-fluorobenzyl)sulfonyl]amino}carbonyl)azetidin-1-yl]-2-methylnicotinate

Prepared according to method C starting from 1-(4-fluorophenyl)methanesulfonamide Yield=19 mg. (13%)

¹H NMR (400 MHz, DMSO-d₆): δ 1.32 (d, J=6.3 Hz, 6H), 2.65 (s, 3H), 3.51-3.61 (m, 1H), 4.27-4.35 (m, 2H), 4.38-4.48 (m, 2H), 4.76 (br s, 2H), 5.04-5.13 (m, 1H), 7.14-7.26 (m, 2H), 7.37-7.48 (m, 2H, 8.31 (s, 1H), 11.81 (br s, 1H).

MS ^{m}/_{z}: 475 (M+1)

### Example 98

### Isopropyl 5-cyano-6-[3-({[(4-cyanobenzyl)sulfonyl]amino}carbonyl)azetidin-1-yl]-2-methylnicotinate

Prepared according to method C Starting from 1-(4-cyanophenyl)methanesulfonamide Yield=39 mg. (27%)

¹H NMR (400 MHz, DMSO-d₆/CD₃OD 9:1): δ 1.31 (6H, d, J=6.3H), 2.65 (3H, s), 3.54-3.63 (1H, m), 4.28-4.35 (2H, m), 4.39-4.48 (2H, m), 4.77 (2H, s), 5.03-5.14 (1H, m), 7.19-7.25 (2H, m), 7.38-7.44 (2H, m), 8.30 (1H, s).

MS ^{m}/_{z}: 482 (M+1)

### Example 99

### Methyl 6-(4-{[(benzylsulfonyl)amino]carbonyl}piperidin-1-yl)-5-cyano-2-methylnicotinate

### (a) 5-Cyano-2-methyl-6-oxo-1,6-dihydropyridine-3-carboxylic acid

KOH (1.43 g, 25.5 mmol) dissolved in EtOH (25 mL, 95%) was added to ethyl 5-cyano-2-methyl-6-oxo-1,6-dihydropyridine-3-carboxylate (1.69 g, 8.2 mmol) in EtOH (30 mL) to give a thick slurry which was heated to reflux (90 degrees in the oil bath) fir 12 hours. The mixture was concentrated and 2 M HCl was added. The precipitate formed was filtered, washed with water and dried to give 5-cyano-2-methyl-6-oxo-1,6-dihydropyridine-3-carboxylic acid as a white solid. Yield: 1.425 g (98%).

1H NMR (500 MHz, DMSO-d₆): keto-form: 2.61 (3H, a), 8.40 (1H, s), 12.91 (1H, br s). ˜86% and enol-form: 2.36 (3H, s), 8.09 (1H, s), 10.50 (1H, br s). ˜14%

MS ^{m}/_{z}: 179 (M+1), 177 (M−1).

### (b) 6-Chloro-5-cyano-2-methylnicotinoyl chloride

Oxalylchloride (3.38 mL, 40 mmol) was added dropwise to a cold (ice/water bath) suspension of 5-cyano-2-methyl-6-oxo-1,6-dihydropyridine-3-carboxylic acid (0.710 g, 3.99 mmol) in dry DCM (25 mL) followed by dry DMF (0.1 mL). The reaction was stirred for 20 minutes at 0 degrees and then at room temperature for 30 minutes followed by reflux for 16 hours. The mixture was evaporated and the remaining black residue was co-evaporated with dry DCM (two times). The crude product was used in the next step without further purification.

### (c) Methyl 6-chloro-5-cyano-2-methylnicotinate

DIPEA (0.35 mL, 2.0 mmol) was added to a solution of crude 6-chloro-5-cyano-2-methylnicotinoyl chloride (0.222 g, 1.03 mmol) in Methanol (4 mL). The reaction was stirred at r.t for 1 hour. The reaction mixture was used directly in the next step without isolation.

### (d) 1-[3-Cyano-5-(methoxycarbonyl)-6-methylpyridin-2-yl]piperidine 4-carboxylic acid

Piperidine-4-carboxylic acid (0.136 g, 1.05 mmol) was added to the solution from above (assuming 100% conversion, 0.210 g, 1 mmol) and the mixture was heated to 120 degrees in a single node microwave oven for 5 minutes. NH₄Cl (aq) was added and the reaction was extracted with DCM (3 times). The combined organic layer was separated dried and evaporated. Purification by preparative HPLC (Kromasil C8, 21.5×250 mm column, flow 25 mL/min using a gradient of 0.1 M NH₄OAc and CH₃CN) gave 0.181 g of 1-[3-cyano-5-(methoxycarbonyl)-6-methylpyridin-2-yl]piperidine-4-carboxylic acid. Yield (60% over three steps).

¹H NMR (50 MHz, DMSO-d₆): 1.54-1.63 (2H, m), 1.85-1.92 (2H, m), 2.39-2.47 (1H, m), 2.62 (3H, s), 3.22-3.29 (2H, m), 3.77 (3H, s), 4.38-4.44 (2H, m), 8.30 (1H, s).

### (e) Methyl 6-(4-{[(benzylsulfonyl)amino]carbonyl}piperidin-1-yl)-5-cyano-2-methylnicotinate

TBTU (0.106 g, 0.33 mmol) was added to a solution of 1-[3-cyano-5-(methoxycarbonyl)-6-methylpyridin-2-yl]piperidine-4-carboxylic acid (0.090 g, 0.297 mmol) and DIPEA. (0.2 mL, 1.15 mmol) in DCM (4 mL). Phenylmethanesulfonamide (0.060 g, 0.409 mmol) was added after 30 minutes and the reaction was stirred for 20 hours at r.t. NaHCO₃ (aq) was added and the mixture was extracted with DCM (3 times). The combined organic layer was dried and evaporated to give a crude product which was purified by reverse phase HPLC (Kromasil C8, 21.5×250 mm column, flow 25 mL/min using a gradient of 0.1 M NH₄OAc and CH₃CN) to give methyl 6-(4-{[(benzylsulfonyl)amino]carbonyl}piperidin-1-yl)-5-cyano-2-methylnicotinate as a powder. Yield: 0.040 g (29%).

1H NMR (500 MHz, DMSO-d₆): 1.58-1.68 (2H, m), 1.80-1.87 (2H, m) 2.54-2.61 (1H, m), 2.64 (3H, s), 3.13 (2H, apparent t), 3.78 (3H, s), 4.53 (2H, apparent d), 4.68 (2H, s), 7.26-7.31 (2H, m), 7.36-7.42 (3H, m), 8.32 (1H, s), 11.60 (1H, bs).

### Example 100

### Methyl 5-cyano-2-methyl-6-[4-({[(4-methylbenzyl)sulfonyl]amino}carbonyl)piperidin-1-yl]nicotinate

Prepared in the same way as described in Example 99 by replacing phenylmethanesulfonamide for 1-(4-methylphenyl)methanesulfonamide. Yield: 0.034 g (24%).

1H NMR (500 MHz, DMSO-d₆): 1.58-1.63 (2H, m), 1.80-1.87 (2H, m), 2.30 (3H, s), 2.53-2.62 (1H, m), 2.64 (3H, s), 3.13 (2H, apparent), 3.78 (3H, s), 4.53 (2H, apparent d), 4.63 (2H, s), 7.14-7.22 (4H, m), 8.33 (1H, s), 11.55 (1H, bs).

### Example 101

### S-Ethyl 6-(4-{[(benzylsulfonyl)amino]carbonyl}piperidin-1-yl)-5-cyano-2-methylpyridine-3-carbothioate

### (a) S-Ethyl 6-chloro-5-cyano-2-methylpyridine-3-carbothioate

A solution of EtSH (0.22 mL, 3.0 mmol) and DIPEA (1 mL, 5.74 mmol) in THF (5 mL was added dropwise to a cold (0 degrees ice/water bath) solution of 6-chloro-5-cyano-2-methylnicotinoyl chloride (0.60 g, 2.29 mmol, See Example 99 above). The reaction was stirred at 0 degrees for 10 minutes followed by r.t. for 50 minutes. The mixture was evaporated and the residue was co-evaporated with THF (3 times) to give S-ethyl 6-chloro-5-cyano-2-methylpyridine-3-carbothioate which was used in the next step without further purification. Yield: 0.671 g (100%).

### (b) 1-{3-Cyano-5-[(ethylthio)carbonyl]-6-methylpyridin-2-yl}piperidine-4-carboxylic acid

A mixture of piperidine-4-carboxylic acid (0.362 g, 2.80 mmol), S-ethyl 6-chloro-5-cyano-2-methylpyridine-3-carbothioate (0.674 g, 2.80 mmol) and DIPEA (0.5 mL, 2.87 mmol) in DMF (10 mL) was heated at 100 degrees for 5 minutes using a single node microwave oven. NH₄Cl (aq) was added and the mixture was extracted with DCM (3 times). The combined organic phase was dried and evaporated to give a crude product which was purified by reverse phase HPLC (Kromasil C8, 21.5×250 mm column, flow 25 mL/min using a gradient of 0.1 M NH₄OAc and CH₃CN) to give 1-{3-cyano-5-[(ethylthio)carbonyl]-6-methylpyridin-2-yl}piperidine-4-carboxylic acid. Yield: 0.453 g (48% over three steps).

¹H NMR (500 MHz, DMSO-d₆): 1.27 (3 h, t, J=7.3 Hz), 1.56-1.65 (2H, m), 1.92-1.98 (2H, m), 2.28 (3H, s), 2.57-2.64 (1H, m), 2.98 (2H, q, J=7.3 Hz), 3.24-3.28 (2H, m), 4.42-4.48 (2H, m), 8.28 (1H, s), 12.31 (1H, bs).

### (c) S-Ethyl 6-(4-{[(benzylsulfonyl)amino]carbonyl}piperidin-1-yl)-5-cyano-2-methylpyridine-3-carbothioate

TBTU (0.104 g, 0.324 mmol) and DIPEA (0.1 mL, 0.574 mmol) was added to a solution of 1-{3-cyano-5-[(ethylthio)carbonyl]-6-methylpyridin-2-yl}piperidine-4-carboxylic acid (0.090 g, 0.270 mmol) in DCM (4 mL) at r.t. and the reaction was stirred for 45 minutes before 1-phenylmethanesulfonamide (0.055 g, 0.321 mmol) was added and the stirring was continued for an additional 15 hours. NaHCO3 (aq) was added and the mixture was extracted with DCM (3 times). The combined organic phase was dried and concentrated. Evaporation followed by purification by reverse phase HPLC (Kromasil C8, 21.5×250 mm column, flow 25 mL/min using a gradient of 0.1 M NH₄OAc and CH₃CN) gave S-ethyl 6-(4-{[(benzylsulfonyl)amino]carbonyl}piperidin-1-yl)-5-cyano-2-methylpyridine-3-carbothioate. Yield: 0.053 g (40%).

1H NMR (500 MHz, DMSO-d₆): 1.26 (3H, t, J=7.5), 1.65 (2H, m), 1.85 (2H, m), 2.56 (3H, s), 2.59 (1H, m), 3.00 (21, q, J=7.5), 3.16 (2H, m), 4.55 (2H, m), 4.70 (2H, s), 7.30 (2H, m), 7.41 (3H, m), 8.31 (1H, s), 11.61 (1H, s).

MS ^{m}/_{z}: 487 (M+1), 485 (M−1).

### Example 102

S-Ethyl 5-cyano-2-methyl-6-[4-({[(4-methylbenzyl)sulfonyl]amino}carbonyl)piperidin-1-yl]pyridine-3-carbothioate

Prepared in the same way as described in Example 101 by replacing phenylmethanesulfonamide for 1-(4-methylphenyl)methanesulfonamide. Yield: 0.065 g (48

1H NMR (500 MHz, DMSO-d₆): 1.26 (3H, t, J=7.5), 1.65 (2H, m), 1.85 (2H, m), 2.31 (3H, s), 2.56 (3H, s), 2.59 (1H, m), 3.00 (2H, q, J=7.5), 3.16 (2H, m), 4.55 (2H, m), 4.64 (2H, s), 7.17 (2H, m), 7.22 (2H, m), 8.31 (1H, s), 11.56 (1H, s).

MS ^{m}/_{z}: 501 (M+1), 499 (M−1).

### Example 103

### S-Ethyl 6-[4-({[(4-chlorobenzyl)sulfonyl]-amino}carbonyl)piperidin-1-yl]-5-cyano-2-methylpyridine-3-carbothioate

Prepared in the same way as described in Example 101 by replacing phenylmethanesulfonamide for 1-(4-chlorophenyl)methanesulfonamide. Yield: 0.061 g (43%).

1H NMR (500 MHz, DMSO-d): 1.26 (311, t, J=7.5), 1.65 (21H, m), 1.86 (2H, m), 2.56 (3H, s), 2.59 (1H, m), 3.00 (2H, q, J=7.5), 3.16 (2H, m), 4.55 (2H, m), 4.72 (2H, s), 7.31 (2H, m), 7.49 (2H, m), 8.31 (1H, s), 11.63 (1H, s).

MS ^{m}/_{z}: 521 (M+1), 519 (M−1).

### Example 104

### S-Ethyl 6-[4-({[(4-fluorobenzyl)sulfonyl]amino}carbonyl)piperidin-1-yl]-5-cyano-2-methylpyridine-3-carbothioate

Prepared in the same way as described in Example 101 by replacing phenylmethanesulfonamide for 1-(4-fluorophenyl)methanesulfonamide. Yield: 0.058 g (43%).

1H NMR (500 MHz, DMSO-d₆): 1.26 (3H, t, J=7.4), 1.64 (2H, m), 1.86 (2H, m), 2.56 (3H, s), 2.59 (1H, m), 3.00 (2H, q, J-7.5), 3.16 (2H, m), 4.55 (2H, m), 4.70 (2H, s), 7.25 (2H, m), 7.34 (2H, m), 8.31 (1H, s), 11.62 (1H, s).

MS ^{m}/_{z}: 505 (M+1), 503 (M−1).

### Example 105

### Ethyl 6-(3-{[(benzylsulfonyl)amino]carbonyl}azetidin-1-yl)-5-methoxy-2-methylnicotinate

### (a) Ethyl 5-methoxy-2-methyl-6-oxo-1,6-dihydropyridine-3-carboxylate

LiH (0.0223 g, 2.80 mmol) was added to a cooled (0° C.) solution of ethyl 5-hydroxy-2-methyl-6-oxo-1,6-dihydropyridine-3-carboxylate (0.502 g, 2.55 mmol) in DMF (15 mL) and the resulting mixture was stirred for 1.5 h. Iodomethane (0.175 mL, 2.80 mmol) was added and the reaction was heated to 60° C. for 20 h. After cooling to r.t the mixture was diluted with EtOAc (200 mL), washed with saturated NH₄Cl (3×100 mL), brine, dried (MgSO₄) and passed through silica gel. Flash chromatography (1:1 EtOAc/hexanes) furnished ethyl 5-methoxy-2-methyl-6-oxo-1,6-dihydropyridine-3-carboxylate as a solid. Yield: 0.140 g (26%)

¹H NMR (400 MHz, CDCl₃): δ 1.38 (3H, t, J=7.1 Hz), 2.68 (3H, s), 3.88 (3H, s), 4.33 (2H, q, J=7.1 Hz), 7.31 (1H, s), 12.07 (111, br s).

MS ^{m}/_{z}: 212 (M+1).

### (b) Ethyl 6-chloro-5-methoxy-2-methylnicotinate

A suspension of ethyl 5-methoxy-2-methyl-6-oxo-1,6-dihydropyridine-3-carboxylate (0.065 g, 0.31 mmol) in POCl₃ (15 mL) was heated to 60° C. for 6 h. After cooling to r.t, the reaction mixture was concentrated under reduced pressure, diluted with EtOAc (100 mL), washed with saturated NaHCO₃ (2×50 mL), brine (50 mL), dried (MgSO₄) and passed through silica gel. Concentration afforded ethyl 6-chloro-5-methoxy-2-methylnicotinate. Yield: 0.049 g (69%).

¹H NMR (400 MHz, CDCl₃): δ 1.42 (3H, t, J=7.1 Hz), 2.74 (3H, s), 3.95 (3H, s), 4.40 (2H, q, J=7.1 Hz), 7.71 (1H, s).

MS ^{m}/_{z}: 230 (M+1).

### (c) 1-(5-(Ethoxycarbonyl)-3-methoxy-methylpyridin-2-yl)azetidine-3-carboxylic acid

A mixture of ethyl 6-chloro-5-methoxy-2-methylnicotinate (0.045 g, 0.20 mmol), azetidine-3-carboxylic acid (0.0258 g, 0.255 mmol) and DIPEA (0.205 mL, 1.18 mmol) in NMP (2.0 mL) was heated to 110° C. for 4 days. After cooling to r.t, the reaction mixture was diluted with EtOAc (100 mL), washed with saturated NH₄Cl (3×50 mL), brine (50 mL), dried (MgSO₄) and concentrated. Flash chromatography (35% EtOAc/hexanes with 1% HOAc) afforded 1-(5-(ethoxycarbonyl)-3-methoxy-6-methylpyridin-2-yl)azetidine-3-carboxylic acid.

MS ^{m}/_{z}: 295 (M+1).

### (d) Ethyl 6-(3-{[(benzylsulfonyl)amino]carbonyl}azetidin-1-yl)-5-methoxy-2-methylnicotinate

A solution of 1-(5-(ethoxycarbonyl)-3-methoxy-6-methylpyridin-2-yl)azetidine-3-carboxylic acid (0.0550 g, 0.187 mmol), phenylmethanesulfonamide (0.352 g, 0.206 mmol), EDCI (0.0394 g, 0.206 mmol), HOBt×H₂O (0.0315 g, 0.206 mmol) and DIPEA (0.0977 mL, 0.561 mmol) in DCM (2.0 mL) was stirred at r.t for 18 h. The reaction mixture was diluted with EtOAc (60 mL), washed with saturated NH₄Cl (30 mL), brine (50 mL), dried (MgSO₄) and concentrated. Flash chromatography (1:4 EtOAc/hexanes with 1% AcOH) furnished ethyl 6-(3-{[(benzylsulfonyl)amino]carbonyl}azetidin-1-yl)-5-methoxy-2-methylnicotinate as a solid. Yield 0.025 g (28%).

¹H NMR (400 MHz, CDCl₃): δ 1.38 (3H, t, J=7.1 Hz), 2.64 (3H, s), 3.24-3.31 (1H, m), 3.77 (3H, s), 4.21-4.35 (6H, m), 4.65 (2H, s), 7.36-7.41 (5H, m), 7.44 (1H, s).

MS ^{m}/_{z}: 448 (M+1).

### Example 106

### Ethyl 6-[4-({[(benzylsulfonyl)amino]carbonyl}amino)piperidin-1-yl]-5-cyano-2-methylnicotinate

### (a) Ethyl 6-(4-tert-butoxycarbonylamino)piperidin-1-yl)₅-cyano-2-methylnicotinate

Ethyl 6-chloro-5-cyanonicotinate (2.00 g, 8.90 mmol) and tert-butyl piperidin-4-ylcarbamate (1.78 g, 8.90 mmol) were dissolved in EtOH (50 mL) at room temperature. DIPEA (4.65 mL, 26.7 mmol) was added and the system heated at 94° C. for 4 h. The reaction mixture was cooled to room temperature and the solvent concentrated under reduced pressure. The material was partitioned between EtOAc (50 mL) and saturated aqueous NH₄Cl (2×30 mL). The organics were washed with brine (30 mL), dried (MgSO₄) and concentrated under reduced pressure to afford the crude product. No purification was done. Yield: 3.30 g (95.4%).

¹H NMR (400 MHz, CDCl₃): δ 1.37 (3H, t, J=7.1 Hz), 1.46 (1H, s), 2.05-2.14 (2H, m), 2.72 (3H, s), 3.15-3.26 (2H, m), 3.71-3.83 (1H, m), 4.32 (2H, q, J=7.1 Hz), 4.42-4.51 (1H, m), 4.58-4.67 (2H, m), 8.34 (1H, s).

MS ^{m}/_{z}: 389 (M+1).

### (b) Ethyl 6-(4-aminopiperidin-1-yl)-5-cyano-2-methylnicotinate dihydrochloride

Ethyl 6-(4-(tert-butoxycarbonylamino)piperidin-1-yl)-5-cyano-2-methylnicotinate (3.30 g. 8.50 mmol) was dissolved HCl (4 M in dioxane, 31.9 mL, 127 mmol). The reaction mixture was stirred at room temperature for 48 h and concentrated under reduced pressure to yield ethyl 6-(4-aminopiperidin-1-yl)-5-cyano-2-methylnicotinate dihydrochloride as a solid, which was used crude assuming 100% conversion.

¹H NMR (400 MHz, d₆-DMSO): δ 131 (3H, t, J=7.1 Hz), 1.53-1.68 (2H, m), 2.02-2.12 (2H, m), 2.65 (3H, s), 3.14-3.27 (2H, m), 3.30-3.43 (1H, m), 4.25 (2H, q, J=7.1 Hz), 4.50-4.60 (2H, m), 8.17-8.29 (2H, m), 8.37 (1H, s).

MS ^{m}/_{z}: 362 (M+1).

### (c) Ethyl 6-[4-({[(benzylsulfonyl)amino]carbonyl}amino)piperidin-1-yl]-5-cyano-2-methylnicotinate

CDI (0.152 g, 0.934 mmol) and 1-phenylmethanesulfonamide (0.200 g, 1.17 mmol) were dissolved in DCE (2 mL) and stirred for 16 h at r.t. Ethyl 6-(4-aminopiperidin-1-yl)-5-cyano-2-methylnicotinate dihydrochloride (0.200 g, 0.554 mmol) in DCE (2 mL) and DIPEA (0.482 mL, 2.77 mmol) were added to this solution and stirred at r.t for 5 h. The reaction mixture was concentrated under reduced pressure and diluted with EtOAc (40 mL). The combined organics were washed with 2 N HCl (2×30 mL), saturated NH₄Cl (2×30 mL), and water (2×30 mL), dried (MgSO₄) and concentrated under reduced pressure to afford the crude product Trituration (40% hexanes, 40% Et₂O, and 20% DCM) gave ethyl 6-[4-({[(benzylsulfonyl)amino]carbonyl}amino)piperidin-1-yl]-5-cyano-2-methylnicotinateas a solid. Yield: 0.185 g (68.8%)

¹H NMR (400 MHz, DMSO-d₆): δ 1.31 (3H, t, J=7.1 Hz), 1.39-1.56 (2H, m), 1.88-2.00 (2H, m), 2.64 (3H, s), 3.77-3.89 (1H, m), 4.25 (2H, q, J=7.1 Hz), 4.39-4.49 (2H, m), 4.69 (2H, s), 6.32-6.41 (1H, m), 7.29-7.45 (5H, m), 8.34 (1H, s), 9.90 (1H, s).

MS ^{m}/_{z}: 486 (M+1).

### Example 107

### Ethyl 6-(4-{[(benzylsulfonyl)amino]carbonyl}piperazin-1-yl)-5-cyano-2-methylnicotinate

### (a) tert-Butyl 4-[3-cyano-5-(ethoxycarbonyl)-6-methylpyridin-2-yl]piperazine-1-carboxylate

Ethyl 6-chloro-5-cyanonicotinate (0.500 g, 2.23 mmol) and tert-butyl piperazine-1-carboxylate (0.623 g, 3.35 mmol) were dissolved in EtOH (50 mL) at r.t. DIPEA (1.16 mL, 6.68 mmol) was added and the system heated at 55° C. for 6 h. The reaction mixture was cooled to r.t and the solvent concentrated under reduced pressure. The material was partitioned between EtOAc (50 mL) and saturated aqueous NH₄Cl (2×30 mL). The organics were washed with brine (30 mL), dried (MgSO₄) and concentrated under reduced pressure to afford the crude product. Flash Chromatography (20% EtOAc in Hexanes) gave tert-Butyl 1 (3-cyano-5-(ethoxycarbonyl)-6-methylpyridin-2-yl)piperazine-1-carboxylate. Yield: 0.743 g (89.2%).

¹H NMR (400 MHz, CDCl₃): δ 1.38 (3H, q, J=7.1 Hz), 1.49 (9H, s), 2.73 (3H, s), 3.53-3.61 (4H, m), 3.86-3.95 (4H, m), 4.32 (2H, q, J=7.1 Hz), 8.36 (1H, s).

MS ^{m}/_{z}: 375 (M+1).

### (b) Ethyl 5-cyano-2-methyl-6-piperazin-1-ylnicotinate dihydrochloride

tert-Butyl 4-(3-cyano-5-(ethoxycarbonyl)-6-methylpyridin-2-yl)piperazine-1-carboxylate (3.30 g. 8.50 mmol) was dissolved HCl (2 M in Et₂O, 2.98 mL, 5.95 mmol). The reaction mixture was stirred at r.t for 24 h and concentrated under reduced pressure to yield ethyl 5-cyano-2-methyl-6-61 piperazin-1-yl)nicotinate dihydrochloride as a solid, which was used crude assuming 100% conversion.

¹H NMR (400 CD₃OD): δ 1.31 (31, t, J=7.1 Hz), 2.67 (3H, s), 3.19-3.30 (4H, m), 3.99-4.09 (4H, m), 4.27 (2H, q, J=7.1 Hz), 8.43 (1H, s), 9.28 (1H, m).

MS ^{m}/_{z}: 275 (M+1).

### (c) Ethyl 6-(4-{[(benzylsulfonyl)amino]carbonyl}piperazin-1-yl)-5-cyano-2-methylnicotinate

CDI (0.152 g, 0.934 mmol) and 1-phenylmethanesulfonamide (0.200 g, 1.17 mmol) were dissolved in DCE (2 mL) and stirred for 16 h at r.t. Ethyl 5-cyano-2-methyl-6-(piperazin-1-yl)nicotinate dihydrochloride (0.200 g, 0.576 mmol) in DCE (2 mL) and DIPEA (0.502 mL, 2.88 mmol) were added to this solution and stirred at r.t for 5 h. The reaction mixture was concentrated under reduced pressure and diluted with EtOAc (40 mL). The combined organics were washed with 2 M HCl (2×30 mL), saturated NC (2×30 mL), and 00 (2×30 mL), dried (MgSO₄) and concentrated under reduced pressure to afford the crude product. Trituration (40% hexanes, 40% Et₂O, and 20% DCM then 10% MeOH in Et₂O) gave ethyl 6-(4-{[(benzylsulfonyl) amino]-carbonyl}piperazin-1-yl)-5-cyano-2-methylnicotinate as a solid. Yield: 0.156 g (57%)

¹H NMR (400 MHz, DMSO-d₆): δ 1.31 (31, t, J=7.1 Hz), 2.66 (3H, s), 3.52-3.62 (4H, m), 3.80-3.90 (4H, m), 4.26 (2H, q, J=7.1 Hz), 4.76 (2H, s), 7.33-7.42 (5H, m), 8.35 (1H, s), 10.5-10.6 (1H, m).

MS ^{m}/_{z}: 472 (M+1).

### Example 108

### 4-{[2-(3-{[Benzylsulfonyl)amino]carbonyl}azetidin-1-yl)-5-ethoxycarbonyl)-6-methylpyridin-3-yl]oxy}butanoic acid

A solution of ethyl 6-(3-{[(benzylsulfonyl)amino]carbonyl}azetidin-1-yl)-5-(4-methoxy-4-oxobutoxy)-2-methylnicotinate (0.050 g, 0.089 mmol) in THF (4 mL) was cooled to 0° C. and treated with NaOH (1.00 M, 0.18 mL, 0.18 mmol). The reaction was warmed to r.t. and stirred for 18 h. After dilution with EtOAc (100 mL), the mixture was washed with saturated NH₄Cl (2×50 mL), brine (50 mL), dried (MgSO₄), and concentrated. Flash chromatography (50% EtOAc/hexanes with 1% HOAC) furnished 4-{[2-(3-{[(benzylsulfonyl)amino]carbonyl}azetidin-1-yl)-5-(ethoxycarbonyl)-6-methylpyridin-3-yl]oxy}butanoic acid as a solid. Yield: 0.036 g (67%).

MS ^{m}/_{z}: 548 (M+1).

### Example 109

### Ethyl 5-cyano-2-methyl-6-{3-[({[(1-oxidopyridin-2-yl)methyl]sulfonyl}amino)carbonyl]azetidin-1-yl}nicotinate

### a) 1-Pyridin-2-ylmethanesulfonamide

To a solution of SMOPS (4.8 g, 0.028 mol) in DMSO (dry, 50 mL) was added 2-bromomethylpyridine HBr (5 g, 0.019 mol) at r.t. After 40 min pH of the solution was adjusted to 8 by the addition of aqueous bicarbonate solution. The reaction mixture was extracted with EtOAc (4×100 mL), the organic layers were combined, dried over anhydrous sodium sulphate, filtered and the solvents were removed in vacuo. The residue was redissolved in a mixture of solvents consisting of THF (200 mL) and methanol (10 mL) and treated with a solution of sodium methoxide (4 mL, 25%) over a period of 10 min. After stirring for 40 min, the reaction mixture was concentrated in vacuo and dissolved in water (20 mL). Followed by addition of a solution of hydroxylamine-O-sulfonic acid (12.66 g, 0.099 mol), sodium acetate (7 g) in water (60 mL) followed by stirring at r.t. After 48 h pH of the solution was adjusted to 9 by the addition of aqueous bicarbonate solution and the mixture subjected to freeze drying. The solid thus obtained was treated with methanol, methanolic layer separated and concentrated. The residue was purified by flash chromatography on silica using a gradient of EtOAc in pet ether followed by EtOAc and then with MeOH in EtOAc to give 1-Pyridin-2-ylmethanesulfonamide. Yield: 400 mg (12%).

¹H NMR (400 MHz, DMSO-d₆) δ 4.42-4.45 (2H, m), 6.90-6.95 (21H, m), 7.33-7.39 (1H, m), 7.45-7.50 (1H, m), 7.78-7.85 (1H, m), 8.53-8.59 (1H, m)

MS ^{m}/_{z}: 173 (M+1).

### b) 1-(1-oxidopyridin-2-yl)methanesulfonamide

1-Pyridin-2-ylmethanesulfonamide (100 mg, 0.55 mmol) was dissolved in DCM (2 mL) and cooled in an ice bath before m-CPBA (184 mg, 0.61 mmol) dissolved in DCM (1 ml) was added. Reaction was stirred at r.t. for two hours followed by removal of solvents in vacuo. The crude solid was dissolved in CH3CN/H2O (4 mL), and purified on preparative HPLC (C8, 10 um, 20×250 mm). 25 ml/min, 5% CH₃CN in 0.2% HOAc. 1-(1-Oxidopyridin-2-yl)methanesulfonamide was isolated as a light yellow solid. Yield 65 mg (60%).

MS ^{m}/_{z}: 189 (M+1);

### c) Ethyl 5-cyano-2-methyl-6-{3-[({[(1-oxidopyridin-2-yl)methyl]sulfonyl}amino)carbonyl]azetidin-1-yl}nicotinate

Prepared according to method B starting from 1-(1-oxidopyridin-2-yl)methanesulfonamide. Yield=13 mg (14%).

¹H NMR (400 MHz, DMSO-d₆) δ 1.30 (3H, t, J=7.1 Hz), 2.63 (3H, s), 3.40-3.53 (1H, m), 4.23 (2H, q, J=7.1 Hz), 4.31-4.51 (4H, m), 5.02 (2H, s), 7.27-7.49 (2H, m), 7.57-7.69 (1H, m), 8.20-8.25 (1H, m), 8.29 (1H, s)

MS ^{m}/_{z}: 460 (M+1).

### Example 110

### Ethyl 5-cyano-2-methyl-6-[3-({[(pyridin-3-ylmethyl)sulfonyl]amino}carbonyl)azetidin-1-yl]nicotinate

Prepared according to method B starting from 1-pyridin-3-ylmethanesulfonamide which was prepared from the corresponding bromide in a similar fashion to example 109 step a. Yield=6 mg (7%).

¹H NMR (400 MHz, DMSO-d6) δ 1.30 (3H, t, J=6.9 Hz), 2.63 (3H, s), 3.38-3.49 (1H, m), 4.23 (2H, q, J=7.0 Hz), 4.28-4.55 (4H, m), 4.64 (2H, s), 7.30-7.48 (1H, m), 7.66-7.83 (1H, m), 8.29 (1H, s), 8.47 (1H, s), 8.50-8.57 (1H, m)

MS ^{m}/_{z}: 444 (M+1).

### Example 111

### Ethyl 5-cyano-2-methyl-6-{4-[({[(1-oxidopyridin-2-yl)methyl]sulfonyl}amino)carbonyl]piperidin-1-yl}nicotinate

Prepared according to method B starting from 1-(1-oxidopyridin-2-yl)methanesulfonamide (see example 109 a and b). Yield=27 mg (28%).

¹H NMR (400 MHz, DMSO-d6) δ 1.30 (3H, t, J=7.2 Hz), 1.56-1.72 (2H, m), 1.86-1.99 (2H, m), 2.47-2.55 (1H, m), 2.64 (3H, s), 3.12-3.24 (2H, m), 4.25 (2H, q, J=7.1 Hz), 4.45-4.60 (2H, m), 5.02 (2H, s), 7.30-7.49 (2H, m), 7.55-7.65 (1H, m), 8.25-8.37 (2H, m), 11.62-11.92 (1H, m)

MS ^{m}/_{z}: 488 (M+1).

### Example 112

### Ethyl 5-cyano-2-methyl-6-[4-({[(pyridin-3-ylmethyl)sulfonyl]amino}carbonyl)piperidin-1-yl]nicotinate

Prepared according to method B starting from 1-pyridin-3-ylmethanesulfonamide which was prepared from the corresponding bromide in a similar fashion to example 109 step a. Yield=32 mg (34%).

¹H NMR (400 MHz, DMSO-d6) δ 1.30 (3H, t, J=7.1 Hz), 1.55-1.71 (2H, m), 1.79-1.89 (2H, m), 2.46-2.56 (1H, m), 2.65 (3H, s), 3.09-3.21 (2H, m), 4.25 (2H, q, J=7.1 Hz), 4.49-4.59 (2H, m), 4.73 (21H, s), 7.38-7.50 (1H, m), 7.66-7.78 (1H, m), 8.34 (1H, s), 8.47 (1H, s), 8.52-8.62 (1H, m), 11.58-11.85 (1H, m)

MS ^{m}/_{z}: 472 (M+1).

### Example 113

### Ethyl 6-(4-{[(benzylsulfonyl)amino]carbonyl}piperidin-1-yl)-5-cyano-2-(dimethylamino)nicotinate

### a) ethyl 5-cyano-6-hydroxy-2-oxo-1,2-dihydropyridine-3-carboxylate

Na (2.76 g, 120 mmol) was added piecewise to 22 mL ethanol and was heated at 80° C. for 45 minutes. This was added to a slurry of 2-cyanoacetamide (4.2 g, 50 mmol) in 6 mL warm ethanol. The mixture was stirred for 20 min. followed by addition of diethyl (ethoxymethylene)malonate (10.8 g, 50 mmol). The reaction mixture was refluxed for 16 h followed by cooling to room temperature. The crude product was filtered off and the solid material was triturated with 2×20 mL diethyl ether followed by 2×20 mL heptane. The solid was dried under vacuum to give ethyl S-cyano-6-hydroxy-2-oxo-1,2-dihydropyridine-3-carboxylate

¹H NMR (500 MHz, DMSO-d₆): δ 1.05 (3H, t, J=7 Hz), 3.40 (2H, d, J=7 Hz), 7.88 (1H, s)

### b) Ethyl 2,6-dichloro-5-cyanonicotinate

To ethyl 5-cyano-6-hydroxy-2-oxo-1,2-dihydropyridine-3-carboxylate (1.56 g, 7.50 mmol) in toluene 15 mL was added thionyl chloride (5.35 g, 45 mmol) followed by DMF (55 mg, 75 mmol). The reaction mixture was heated at 85° C. for 16 h. An additional amount of thionyl chloride (5.35 g, 45 mmol) followed by DMF (55 mg, 75 mmol) was added followed by heating at 100° C. for 4 h. The solvents were removed in vacuo for a part of the material which was used in the consecutive step.

### c) Ethyl 6-chloro-5-cyano-2-(dimethylamino)nicotinate

Ethyl 2,6-dichloro-5-cyanonicotinate (147 mg, 0.600 mmol) in 1.5 mL MeCN was cooled to 0° C. followed by addition of N-methylmethanamine (10.5 μL, 0.150 mmol) in 0.15 mL MeCN. stirring at 0° C. for 15 min. followed by addition of an additional amount of N-methylmethanamine (10.5 μL, 0.150 mmol) in 0.15 mL. The reaction mixture was stirred at r.t. for 16 h. The solvents were removed in vacuo and part of the material was used immediately in the next step.

### d) 1-[3-cyano-6-(dimethylamino)-5-(ethoxycarbonyl)pyridin-2-yl]piperidine-4-carboxylic acid

Ethyl 6-chloro-5-cyano-2-(dimethylamino)nicotinate (76 mg, 0.300 mmol) from the step above was dissolved in 1.5 mL ethanol/water 1:1 followed by addition of piperidine-4-carboxylic acid (116 mg, 0.90 mmol) was added, followed by TEA (91 mg, 0.90 mmol). The reaction mixture was heated in a single node microwave oven at 120° C. for 20 min. The solvents were removed in vacuo to give 201 mg crude material.

Purification was done by reverse phase HPLC. A: MeCN, B: 0.1 M ammonium acetate/MeCN 95:5, C: 5% MeCN in 50 mM HCOOH/50 mM ammonium formiate. Start: A/B/C 5:95:0. Injected at flow=10 mL/min. Increased to flow=50 mL/min. continuous over 3 minutes after the injection. Then changed to A/B/C 5:0:95 and flow increased to 100 mL/min. Increased to 100:0:0 over 17 minutes in steps of 5% points. Column: Kromasil C8, 250 mm×50.8 ID. Product was eluted when A/C was 70:30. This gave 1-[3-cyano-6-(dimethylamino)-5-(ethoxycarbonyl)pyridin-2-yl]piperidine-4-carboxylic acid. Yield=44 mg (42%).

¹H NMR (500 MHz, CDCl₃): δ 1.35 (3H, t, J=7.0 Hz), 1.78-1.89 (2H, m), 1.99-2.01 (2H, m), 2.61-2.67 (1H, m), 3.3-3.5 (6H, m), 3.17-3.24 (2H, m), 4.28 (2H, q, 7.0 Hz), 4.42-4.49 (2H, m), 8.13 (1H, s)

### e) Ethyl 6-(4-{[(benzylsulfonyl)amino]carbonyl}piperidin-1-yl)-5-cyano-2-(dimethylamino)nicotinate

1-[3-cyano-6-(dimethylamino)-5-(ethoxycarbonyl)pyridin-2-yl]piperidine-4-carboxylic acid (24 mg, 0.069 mmol) was dissolved in DCM (1 mL) followed by addition of TBTU (37 mg, 0.097 mmol) and DIPEA (0.047 mL, 0.28 mmol). After 2 minutes 1-phenylmethanesulfonamide (14 mg, 0.083 mmol) was added. The reaction mixture was stirred at room temperature for 6 h followed by addition of 0.094 mL DIPEA. Stirring at room temperature was continued for an additional 16 hr. 1-phenylmethanesulfoneamide (14 mg, 0.083 mmol) and TBTU (37 mg, 0.097 mmol) was added followed by stirring at rt for an additional 22 h.

Purification was done by reverse phase HPLC: A: MeCN, B: 0.1 M ammonium acetate/MeCN 95:5, C: 5% MeCN in 50 mM HCOOH/50 mM ammonium formiate. Start: A/B/C 5:95:0. Injected at flow=10 m/min Increased to flow=20 mL/min. just after injection. Then changed to A/B/C 5:0:95. Increased to 50:0:50 over 30 min. in 9 equal steps. Then to 100:0:0 over 10 min. in 5 steps. Flow: 20 mL/min. Column: Kromasil C8, 250 mm×20 ID. This gave Ethyl 6-(4-{[(benzylsulfonyl)amino]carbonyl}piperidin-1-yl)-5-cyano-2-(dimethylamino)nicotinate. Yield=8 mg (23%).

¹H NMR (500 MHz, CDCl₃): δ 1.35 (3H, t, J=7.2 Hz), 1.71-1.86 (4H, m), 2.33-2.42 (1H, m), 2.98-3.04 (2H, m), 3.05 (6H, s), 4.28 (2H, q, J=7.2 Hz), 4.48-4.54 (2H, m), 4.65 (2H, s), 7.31-7.35 (2H, m), 7.36-7.43 (3H, m), 8.12 (1H, s).

MS ^{m}/_{z}: 500 (M+1)

### Example 114

### Ethyl 5-cyano-2-methyl-6-[4-({[(pyridin-4-ylmethyl)sulfonyl]amino}carbonyl)piperidin-1-yl]nicotinate

Prepared according to method B stag from 1-pyridin-4-ylmethanesulfonamide which was prepared from the corresponding bromide in a similar fashion to example 109 step a. Yield=20 mg (21%).

¹H NMR (400 MHz, DMSO-d₆) δ 1.30 (31, t, J=7.1 Hz), 1.56-1.70 (2H, m), 1.79-1.89 (2H, m), 2.46-2.56 (1H, m), 2.65 (3H, s), 3.08-3.21 (2H, m), 4.25 (2H, q, J=7.1 Hz), 4.48-4.58 (2H, m), 4.73 (2H, s), 7.29-7.33 (2H, m), 8.34 (1H, s), 8.58-8.62 (2H, m), 11.65-11.93 (1H, m)

MS ^{m}/_{z}: 472 (M+1).

### Example 115

### Ethyl 5-cyano-2-methyl-6-[3-({[(pyridin-2-ylmethyl)sulfonyl]amino}carbonyl)azetidin-1-yl]nicotinate

Prepared according to method B starting from 1-pyridin-2-ylmethanesulfonamide which was prepared from the corresponding bromide in a similar fashion to example 109 step a. Yield=7 mg (8%).

¹H NMR (400 MHz, DMSO-d6) δ 1.30 (3H, t, J=7.1 Hz), 2.64 (3H, s), 3.43-3.57 (1H, m), 4.24 (2H, q, J=7.1 Hz), 4.36-4.56 (4H, m), 4.80 (2H, s), 7.30-7.57 (2H, m), 7.75-7.89 (1H, m), 8.31 (1H, s), 8.49-8.59 (1H, m), 11.52-11.99 (1H, m)

MS ^{m}/_{z}: 444 (M+1).

### Example 116

### Ethyl 5-cyano-6-[3-{[(3,5-dimethylbenzyl)sulfonyl]amino}carbonyl)azetidin-1-yl]-2-methylnicotinate

Prepared according to method B starting from 1-(3,5-dimethylphenyl)methanesulfonamide which was prepared from the corresponding bromide in a similar fashion to example 109. Yield=5 mg (5%).

MS ^{m}/_{z}: 471 (M+1).

### Example 117

### Isopropyl 5-cyano-6-[4-({[(cyclopentylmethyl)sulfonyl]amino}carbonyl)piperidin-1-yl]-2-methylnicotinate

To 1-[3-cyano-5-(isopropoxycarbonyl)-6-methylpyridin-2-yl]piperidine-4-carboxylic acid (100 mg, 0.301 mmol) were added TBTU (97 mg, 0.302 mmol), dry DCM (2 mL), DIPEA (0.1 mL, 0.574 mmol) and the mixture was stirred at room temperature for 2.5 h. The mixture was added to 1-cyclopentylmethanesulfonamide (58.8 mg, 0.360 mmol), dry DCM (2 ml) was added and the reaction mixture was stirred at room temperature for 18 h NaHCO₃ (aq) was added and the mixture was extracted three times with DCM. The combined organic layers was passed through a phase separator and the solvents were removed in vacuo. The crude product was purified using preparative HPLC on a (Kromasil C8 10 μm, 21.5×250 mm) using a gradient of 25-55% acetonitrile/aqueous NH4OAc buffer pH 7 to give isopropyl 5-cyano-6-[4-({[(cyclopentylmethyl)sulfonyl]amino}carbonyl)piperidin 1-yl]-2-methylnicotinate. Yield: 80 mg (56%).

¹H NMR (500 MHz, DMSO-d6) δ 1.22-1.93 (12H, m), 1.30 (6H, app d, J=6.2 Hz), 2.11-2.20 (1H, m), 2.64 (3H, s), 2.64-2.69 (1H, m), 3.14-3.21 (2H, m), 3.42 (2H, d, J=7.0 Hz), 4.51-4.57 (2H, m), 5.08 (1H, app q, J=6.2 Hz), 8.32 (1H, s), 11.71 (1H, s).

MS ^{m}/_{z}: 477.3 (M+1), 475.3 (M−1).

### Example 118

### Ethyl 5-cyano-6-[4-({[(2,5-dimethylbenzyl)sulfonyl]amino}carbonyl)piperidin-1-yl]-2-methylnicotinate

Prepared according to method B starting from 1-(2,5-dimethylphenyl)methanesulfonamide which was prepared from the corresponding bromide in a similar fashion to example 109. Yield=18 mg (18%).

¹H NMR (400 MHz, DMSO-d6) δ 1.30 (3H, t, J=7.1 Hz), 1.59-1.76 (2H, m), 1.83-1.95 (2H, m), 2.25 (3H, s), 2.33 (3H, s), 2.47-2.56 (1H, m), 2.65 (3H, s), 3.11-3.23 (2H, m), 4.25 (2H, q, J=7.1 Hz), 4.51-4.60 (2H, m), 4.64 (2H, s), 6.95 (1H, s), 7.04-7.15 (2H, m), 8.34 (1H, s), 11.54-11.87 (1H, m)

MS ^{m}/_{z}: 499 (M+1).

### Example 119

### Ethyl 5-cyano-6-[4-({[(4-isopropylbenzyl)sulfonyl]amino}carbon-yl)]-2-methylnicotinate

### a) Sodium (4-isopropylphenyl)methanesulfonate

1-(chloromethyl)-4-isopropylbenzene (2.53 g, 15.0 mmol) and disodium sulfite (2.46, 19.5 mmol) were added to a mixture of water (8 mL) and acetone (0.8 mL). The reaction mixture was heated in a microwave oven, single node heating, at 150 degr. for 5 min. The reaction mixture was transferred to a round-bottom flask with acetone (40 mL) and water. (4 mL) The mixture was refluxed for 5.5 h. The solvents were removed in vacuo and the remaining solids were slurried in 20 mL hot abs. ethanol and the crystals were filtered off. The filter cake was rinsed with 10 mL abs. ethanol followed by 2×15 mL heptane and finally dried under vacuum for 2 h. This gave Sodium (4-isopropylphenyl)methanesulfonate. Yield=3.3 g. (92%)

### b) 1-(4-isopropylphenyl)methanesulfonamide

Sodium (4-isopropylphenyl)methanesulfonate (1.9 g, 8.0 mmol) followed by dioxane (32 mL) and thionyl chloride (2.92 mL, 40 mmol) was distributed into 4 vials and heated at 100° C. for 20 minutes each using fixed hold time. The reaction mixtures were combined and the solvents were removed in vacuo. A solution of ammonia in THF (40 mL.) was added at r.t. and the reaction mixture was stirred for 16 h. Water (30 mL) was added and the organic phase was separated. The aq. phase was extracted with 2×30 mL ethyl acetate. The combined organic phases were dried over sodium sulphate, filtered and the solvents were removed in vacuo. The crude was purified by flash chromatography on Si gel with heptane/ethyl acetate 2:1 (Rf of product=0.22) as eluent to give 1-(4-isopropylphenyl)methanesulfonamide. Yield=376 mg, 1.76 mmol. (22%)

### c) Ethyl 5-cyano-6-[4-({[(4-isopropylbenzyl)sulfonyl]amino}carbonyl)piperidin-1-yl]-2-methylnicotinate

1-[3-cyano-5-(ethoxycarbonyl)-6-methylpyridin-2-yl]piperidine-carboxylic acid (159 mg, 0.500 mmol) was dissolved in DCM (4 mL) and HATU (265 mg, 0.700 mmol)) was added, followed by DIPEA (0.341 mL, 2 mmol). The reaction mixture was stirred at room temperature for 5 minutes before the addition of 1-(4-isopropylphenyl)methanesulfonamide (128 mg, 0.600 mmol). Stirring at rt was continued over 15 h. The reaction mixture was concentrated and then dissolved in DMSO (8 mL).

Purification was done by reverse phase HPLC. A: MeCN, B: 0.1 M ammonium acetate/MeCN 95:5, C: 5% MeCN in 50 mM HCOOH/50 mM ammonium formiate. Start: A/B/C 5:95:0. Injected at flow=10 mL/min. Increased to flow=50 mL/min. continuous over 3 minutes after the injection. Then changed to A/B/C 5:0:95 and flow increased to 100 mL/min. Increased to 100:0:0 over 17 minutes in equal steps, each of 5% points. The product was eluted when switching to pure acetonitrile. Column: Kromasil C8, 250 mm×50.8 ID. This gave Ethyl 5-cyano-6-[4-({[(4-isopropylbenzyl)sulfonyl]amino}carbonyl)piperidin-1-yl]-2-methylnicotinate. Yield 0.144 g (56%)

¹H-NMR (400 MHz, DMSO-d6) δ 1.18 (6H, d, J=6.8 Hz), 1.32 (3H, t, J=7.1 Hz), 1.54-1.67 (2H, m), 1.75-1.85 (2H, m), 2.20-2.30 (1H, m), 2.65 (3H, s), 2.80-2.89 (1H, m), 3.18-3.28 (2H, m), 4.19 (2H, s), 426 (2H, q, J=7.1 Hz), 4.38-4.47 (2H, m), 7.09-7.18 (4H, m), 8.32 (1H, s).

MS ^{m}/_{z}: 513 (M+1)

### Example 120

### Benzyl 6-(4-{[(benzylsulfonyl)amino]carbonyl}piperidin-1-yl)-5-cyano-2-methylnicotinate

### a) Benzyl 6-chloro-5-cyano-2-methylnicotinate

6-Chloro-5-cyano-2-methylnicotinoyl chloride (120 mg, 0.56 mmol) was dissolved in dry THF (4 ml), DIPEA (0.2 ml) and phenylmethanol (0.059 ml) were added. The reaction mixture was stirred at r.t. for 15 h. The solvents were removed in vacuo to give benzyl 6-chloro-5-cyano-2-methylnicotinate which was used in the next step without purification.

### b) Benzyl 6-(4-{[(benzylsulfonyl)amino]carbonyl}piperidin-1-yl)-5-cyano-2-methylnicotinate

Benzyl 6-chloro-5-cyano-2-methylnicotinate (129 mg, 0.45 mmol) was dissolved in THF (2 ml), MeOH (2 ml), DIPEA (0.11 mL, 0.574 mmol) and N-(benzylsulfonyl)piperidine-4-carboxamide (140 mg, 0.496 mmol) were added. The reaction mixture was heated to 12° C. for 5 min using microwave single node heating. NaHCO₃ (aq) was added and the mixture was extracted three times with DCM. The combined organic layer was passed through a phase separator and the solvents were removed in vacuo. The crude product was purified using preparative HPLC on a (Kromasil C8 10 μm, 21.5×250 mm) using a gradient of 30-55% acetonitrile/aqueous NH₄OAc buffer pH 7 to give Benzyl 6-(4-{[(benzylsulfonyl)amino]carbonyl}piperidin-1-yl)-5-cyano-2-methylnicotinate. Yield=38 mg (15%).

¹H NMR (500 MHz, DMSO-d6) δ 1.64 (2H, m), 1.84 (2H), 2.58 (1H, m), 2.66 (3H, s), 3.15 (2H, m), 4.54 (2H, m), 4.69 (2H, s), 5.30 (2H, s), 7.28-7.49 (10H, m), 8.38 (1H, s), 11.61 (H, s).

MS ^{m}/_{z}: 533.3 (M+1), 531.3 (M−1).

### Example 121

### Ethyl 5-cyano-2-methyl-6-{4-[({[(4-methylcyclohexyl)methyl]sulfonyl}amino)carbonyl]piperidin-1-yl}nicotinate

### a) 1-(4-methylphenyl)methanesulfonamide

To a stirred solution of SMOPS (5.646 g, 0.0324 mol) in DMSO (dry, 50 mL) was added alpha-bromo-p-xylene (5 g, 0.027 mol) at r.t. and continued stirring for 45 mL The reaction mixture was extracted with EtOAc (4×100 mL), the organic layers were combined, dried over anhydrous sodium sulphate and the solvents were removed in vacuo. The residue was redissolved in THF (100 mL) and methanol (25 mL) followed by addition of sodium methoxide (5.8 mL, 0.027 mol, 25%). After stirring for 15 min. the reaction mixture was concentrated and dissolved in water (10 mL). A solution of hydroxylamine-O-sulfonic acid (17.31 g, 6.1350 mol) and sodium acetate (7 g) in H₂O (40 mL) was added to the reaction mixture which was stirred at r.t. for 12 h. The pH of the solution was adjusted to 9 by addition of aqueous bicarbonate solution and the mixture was extracted with EtOAc (3×50 ml), washed with brine, dried over (Na₂SO₄), and the solvents were removed in vacuo. The residue thus obtained was treated with water (100 mL) and stirred for 10 min. Solid obtained was filtered and dried to afford 1-(4-methylphenyl)methanesulfonamide. Yield=3.3 g, (66%).

¹H NMR (300 MHz, DMSO-d₆) δ 2.55 (3H, s), 4.05 (2H, s), 6.8 (2H, s), 7.1-7.3 (4H, m)

### b) 1-(4-methylcyclohexyl)methanesulfonamide

PtO₂ (2 g) was added to stirred solution of 1-(4-methylphenyl)methanesulfonamide (2 g, 0.0180 mol) in acetic acid (50 mL) at r.t. in a parr shaker and continued stirring for ˜48 h under H₂ (pressure at 6 kg/cm²). After completion of the reaction, reaction mixture was filtered, washed with acetic acid (30 mL) and concentrated. The crude product was purified using flash column chromatography using 10% EtOAc in pet. ether to afford 1-(4-methylcyclohexyl)methanesulfonamide. Yield=520 mg (25.2%).

¹H NMR (300 MD, DMSO-d6) δ 0.83-2.2 (13H, m) 2.84-2.94 (2H m), 6.74 (2H, s)

MS ^{m}/_{z}: 191.8 (M+1).

### c) Ethyl 5-cyano-2-methyl-6-{4-[({[(4-methylcyclohexyl)methyl]sulfonyl}amino)carbonyl]piperidin-1-yl}nicotinate

Prepared according to method B starting from 1-(4-methylcyclohexyl)methanesulfonamide. Yield=23 mg (23%).

¹H NMR (400 MHz, DMSO-d6) ? 0.80-0.97 (4H, m), 0.99-1.21 (2H, m), 1.30 (3H, t, J=7.2 Hz), 1.39-1.57 (4H, m), 1.57-1.73 (2H, m), 1.78-1.98 (3H, m), 2.00-2.11 (1H, m), 2.47-2.6 (1H, m), 2.64 (3H, s), 3.12-3.23 (2H, m), 3.22-3.40 (3H, m), 4.25 (2H, q, J=7.1 Hz), 4.47-4.61 (2H, m), 8.33 (1H, s), 11.66-11.81 (1H, m)

MS ^{m}/_{z}: 491 (M+1).

### Example 122

### Ethyl 5-cyano-6-[3-({[(4-isopropylbenzyl)sulfonyl]amino}carbonyl)azetidin-1-yl]-2-methylnicotinate

1-[3-cyano-5-(ethoxycarbonyl)-6-methylpyridin-2-yl]azetidine-3-carboxylic acid (145 mg, 0.500 mmol) was dissolved in 4 mL DCM/DMF 1:1. HBTU (0.265, 0.700 mmol) and DIPEA (0.341 mL, 2 mmol) were added. Further, 2 mL DMF was added. 1-(4-isopropylphenyl)methanesulfonamide (128 mg, 0.600 mmol) was added and the reaction mixture was stirred for 18 h. Extra HBTU (0.095 g, 0.25 mmol) and DIPEA (0.17 mL, 1 mmol.) was added and stirring at rt was continued for 22 h. The solution was concentrate and then dissolved in 8 mL DMSO. The product was isolated using by reverse phase HPLC. A: MeCN, B: 0.1 M ammonium acetate/MeCN 95:5, C: 5% MeCN in 50 mM HCOOH/50 mM ammonium formiate. Start: A/B/C 5:95:0. Injected at flow=10 mL/mL Increased to flow=50 mL/min. continuous over 3 minutes after the injection. Then changed to A/B/C 5:0:95 and flow increased to 100 mL/min. Increased to 100:0:0 over 17 minutes in equal steps, each of 5% points. Column: Kromasil C8, 250 mm×50.8 ID.

This gave Ethyl 5-cyano-6-[3-({[(4-isopropylbenzyl)sulfonyl]amino}carbonyl)azetidin-1-yl]-2-methylnicotinate. Yield 0.198 g, (82%)

¹H-NMR (400 MHz, DMSO-d6): δ 1.18 (6H, d, J=6.9 Hz), 1.32 (3H, t, J=7.1 Hz), 2.63 (3H, s), 2.73-2.87 (1H, m), 3.17-3.26 (1H, m), 4.21-4.27 (4H, m), 4.27-4.48 (4H, m), 7.08 (2H, d, J=8.1 Hz), 7.17 (2H, d, J=8.1 Hz), 8.28 (1H, s).

MS ^{m}/_{z}: 485 (M+1)

### Example 123

### Ethyl 5-cyano-2-methyl-6-[4-({[(2-phenylethyl)sulfonyl]amino}carbonyl)piperidin-1-yl]nicotinate

Prepared according to method B starting from 2-phenylethanesulfonamide. Yield=22 mg (22%).

¹H NMR (400 MHz, DMSO-d₆) δ 1.30 (3H, t, J=7.1 Hz), 1.50-1.65 (2H, m), 1.82-1.94 (2H, m), 2.50-2.53 (1H, m), 2.64 (3H, s), 2.93-3.00 (2H, m), 3.10-3.21 (2H, m), 3.58-3.70 (2H, m), 4.25 (2H, q, J=7.1 Hz), 4.46-4.58 (2H, m), 7.16-7.37 (5H, m), 8.33 (1H, s), 11.69-11.85 (1H, m)

MS ^{m}/_{z}: 485 (M+1).

### Example 124

### Ethyl 5-cyano-2-methyl-6-[4-({[(pyridin-2-ylmethyl)sulfonyl]amino}carbonyl)piperidin-1-yl]nicotinate

Prepared according to method B starting from 1-pyridin-2-ylmethanesulfonamide which was prepared from the corresponding bromide in a similar fashion to example 109 step a. Yield=7 mg (7%).

¹H NMR (400 MHz, DMSO-d6) δ 1.31 (3H, t, J=7.1 Hz), 1.61-1.74 (2H, m), 1.83-1.92 (2H, m), 2.47-2.56 (1H, m), 2.65 (3H, s), 3.13-3.25 (2H, m), 4.25 (2H, q, J=7.1 Hz), 4.48-4.58 (2H, m), 4.77 (2H, s), 7.35-7.42 (1H, m), 7.46 (1H, d, J=7.9 Hz), 7.79-7.88 (1H, m), 8.34 (1H, s), 8.52-8.58 (1H, m), 11.41-11.70 (1H, m)

MS ^{m}/_{z}: 472 (M+1).

### Example 125

### Ethyl 5-cyano-6-[3-({[(2,5-dimethylbenzyl)sulfonyl]amino}carbonyl)azetidin-1-yl]-2-methylnicotinate

Prepared according to method B starting from 1-(2,5-dimethylphenyl)methanesulfonamide 1-(2,5-dimethylphenyl)methanesulfonamide which was prepared from the corresponding bromide in a similar fashion to example 109. Yield=6 mg (6%).

¹H NMR (400 MHz, DMSO-d6) δ 1.30 (3H, t, J=7.2 Hz), 2.24 (3H, s), 2.34 (3H, s), 2.63 (3H, s), 3.52-3.68 (1H, m), 4.24 (2H, q, J=7.1 Hz), 4.31-4.41 (2H, m), 4.41-4.51 (2H, m), 4.70 (2H, s), 7.01 (1H, s), 7.05-7.15 (2H, m), 8.31 (1H, s), 11.76-12.09 (1H, m)

MS ^{m}/_{z}: 471 (M+1).

### Example 126

### Ethyl 6-(3-{[(benzylsulfonyl)amino]carbonyl}azetidin-1-yl)-5-chloro-2-methylnicotinate

### a) Ethyl 5-chloro-2-methyl-6-oxo-1,6-dihydropyridine-3-carboxylate

Ethyl 2-methyl-6-oxo-1,6-dihydropyridine-3-carboxylate (2.00 g, 11.0 mmol), (Raileanu D., et. al. Tetrahedron, Vol 30 pp 623-32, 1974) was dissolved in DMF (35 mL) under a nitrogen atmosphere. NCS (1.53 g, 11.5 mmol) taken up in DMF (5.0 mL) at r.t. The reaction mixture was heated at 100° C. for 1 h. An additional amount of NCS (500 mg, 3.8 mmol) was added and the reaction mixture was stirred for 0.5 h. The reaction mixture was diluted with DCM were washed with water and brine once each. The aqueous phase was extracted with DCM twice and the combined organic phases were passed through a phase separator and the solvents were removed in vacuo. The crude product was purified by flash chromatography on silica (Biotage horizon) first EtOAc/heptane 1:1 followed by EtOAc to give ethyl 5-chloro-2-methyl-6-oxo-1,6-dihydropyridine-3-carboxylate as a yellow solid Yield=1.362 g (52%).

¹H NMR (400 MHz, DMSO-d₆) δ 1.37 (3H, t, J=7.3 Hz), 2.74 (3H, s), 4.32 (211, q, J=7.3) Hz, 8.19 (1H, s)

### b) ethyl 5,6-dichloro-2-methylnicotinate

Ethyl 5-chloro-2-methyl-6-oxo-1,6-dihydropyridine-3-carboxylate (1.2 g, 5.1 mmol) was dissolved in DCM (25 mL) followed by addition of oxalylchloride (2.2 mL, 26 mmol). Two drops of DMF was added and the mixture was heated at 42° C. After 3 h oxalylchloride (2 mL, 24 mmol) was added. After an hour further oxalylchloride (1.1 ml, 13 mmol) and DMF (0.03 mL) were added and the reaction mixture was stirred at 50° C. over night. The reaction mixture was diluted with DCM and poured onto an ice/water mix. The phases were separated and the organic phase was washed with sat NaHCO3×2 followed by brine. The combined aqueous phases were extracted with DCM and the combined organic phases were filtered through a phase separator and the solvents were removed in vacuo. The crude product was co-concentrated with DCM three times which gave ethyl 5,6-dichloro-2-methylnicotinate as a dark brown solid. Yield, 0.949 g (52%)

¹H NMR (400 MHz, DMSO-d6) δ 1.32 (3H, t, J=8.0 Hz), 2.67 (3H, s), 4.32 (2H, q, J=7.2 Hz), 8.37 (1H, s)

### c) Ethyl 6-(3-{[(benzylsulfonyl)amino]carbonyl}azetidin-1-yl)-5-chloro-2-methylnicotinate

Ethyl 5,6-dichloro-2-methylnicotinate (202 mg 0.846 mmol) and N-(benzylsulfonyl)azetidine-3-carboxamide (237 mg, 0.930 mmol) were dissolved in EtOH (5 mL), water (8 mL) and MeCN (3 mL). Heated in microwave oven, single node heating, for 20 minutes at 120° C. TEA (0.47 mL, 3.4 mmol) was added and the mixture was heated in microwave oven for 20 minutes at 120° C. The reaction mixture was diluted with DCM and the organics were washed with 2% KHSO4 twice. The combined aqueous phases were extracted with DCM. The combined organic phases were passed through a phase separator followed by removal of the solvents in vacuo. The crude product was purified using preparative HPLC on a (Kromasil C8. 50.8×300 mm), in order to avoid precipitation the compound was loaded onto the column using 5% acetonitrile/aqueous NH4OAc buffer pH 7. The product was eluted using a gradient of 5-90% acetonitrile/aqueous NH4OAc buffer pH 3 to give pure Ethyl 6-(3-{[(benzylsulfonyl)amino]carbonyl}azetidin-1-yl)-5-chloro-2-methylnicotinate. Yield: 204 mg (53%).

¹H NMR (400 MHz, DMSO-d6) δ 1.28 (3H, t, J=7.3 Hz), 2.58 (3H, s), 3.53-3.43 (1H, m), 4.15-4.42 (6H, m), 4.74 (2H, s), 7.25-7.43 (5H, m), 7.93 (1H, s), 11.77 (1H, s),

MS ^{m}/_{z}: 452 (M+1)

### Example 127

### Ethyl 6-(3-{2-[(benzylsulfonyl)amino]-2-oxoethyl}azetidin-1-yl)-5-cyano-2-methylnicotinate

### a) Azetidin-3-ylacetic acid

[1-(tert-Butoxycarbonyl)azetidin-3-yl]acetic acid (1.0 g, 4.65 mmol) was dissolved in DCM (8 mL) followed by addition of TFA (5 mL). The reaction mixture was stirred at r.t. for 2 h. The solvents were removed and the crude Azetidin-3-ylacetic acid (1.31 g, TFA left) was used in step b without purification.

¹H-NMR (500 MHz, DMSO-d6) δ 2.61-2.65 (2H, m), 2.98-3.09 (1H, m), 3.68-3.77 (2H, m), 3.95-4.03 (2H, m)

### b) {1-[3-cyano-5-(ethoxycarbonyl)-6-methylpyridin-2-yl]azetidin-3-yl}acetic acid

Azetidin-3-ylacetic acid (460.5 mg, 5.00 mmol) from previous step was dissolved in EtOH (8 mL) and ethyl 6-chloro-5-cyano-2-methylnicotinate (1.02 g), DIPEA (2 mL) were added. The reaction mixture was heated at 100° C. for 5 min using microwave single node heating. NH₄Cl (aq) was added and the mixture was extracted with DCM three times. The combined organic layers were run through a phase separator and the solvents were removed in vacuo. The crude product was purified by prep HPLC.

Column: Kromasil C8 10 μm, 50.8×300 nm, Mobilephase A: 100% AcN, Mobilephase B 5%, AcN, 95% NH₄AcO (aq) (pH7), Gradient: 20=>60% A over 60 min, Flow: 56 ml/min and UV: 280 nm.

This gave {1-[3-cyano-5-(ethoxycarbonyl)-6-methylpyridin-2-yl]azetidin-3-yl}acetic acid yield=526 mg. (43.3%)

¹H-NMR (500 MHz, DMSO-d₆) δ 1.29 (3H, t, j=7.1), 2.60 (3H, s), 2.63-2.66 (2H, m). 2.93-3.02 (1H, m), 3.95-4.05 (2H, m), 4.34 (2H, q, j=7.1), 4.37-4.47 (2H, m)

### c) Ethyl 6-(3-{2-[(benzylsulfonyl)amino]-2-oxoethyl}azetidin-1-yl)-cyano-2-methylnicotinate

{1-[3-cyano-5-(ethoxycarbonyl)-6-methylpyridin-2-yl]azetidin-3-yl}acetic acid (130 mg, 0.429 mmol), TBTU (190 mg, 0.592 mmol), DIPEA (0.2 mL, 1.15 mmol) were dissolved in dry DCM (4 ml) and the mixture was stirred at room temperature for 1 h 20 min. The mixture was added to 1-phenylmethanesulfonamide (100 mg, 0.584 mmol) and the reaction mixture was stirred at room temperature for 25 h. NaHCO₃ (aq) was added and the mixture was extracted three times with DCM. The combined organic layer was passed through a phase separator and the solvents were removed in vacuo. The crude product was purified using preparative HPLC on a (Kromasil C8 10 μm, 21.5×250 mm) using a gradient of 25-45% acetonitrile/aqueous NH₄OAc buffer pH 7 to give ethyl 6-(3-(2-[(benzylsulfonyl)amino]-2-oxoethyl)azetidin-1-yl)-5-cyano-2-methylnicotinate. Yield=119 mg (61%)

¹H-NMR (500 MHz, DMSO-d6) δ 1.30 (3H, t, J=7.2 Hz), 2.62 (3H, s), 2.69 (2H, m), 3.05 (1H, m), 4.02 (2H, m), 4.23 (2H, q, J=7.2 Hz), 4.47 (2H, m), 4.70 (2H, s), 7.31 (2H, m), 7.41 (3H, m), 8.28 (1H, s), 11.67 (1H, s).

MS ^{m}/_{z}: 457.1 (M+1), 455.0 (M−1).

### Example 128

### Ethyl 5-cyano-6-[4-({[(cyclopentylmethyl)sulfonyl]amino}carbonyl)piperidin-1-yl]-2-methylnicotinate

Prepared according to method B starting from 1-cyclopentylmethanesulfonamide. Yield=10 mg (10%).

MS ^{m}/_{z}: 471 (M+1).

¹H NMR (400 MHz, DMSO-d6) δ 1.18-1.28 (2H, m), 1.30 (3H, t, J=7.1 Hz), 1.43-1.53 (2H, m), 1.54-1.69 (4H, m), 1.80-1.95 (4H, m), 2.08-2.22 (1H, m), 2.22 (3H, s), 2.65-2.69 (1H, m), 3.11-3.23 (2H, m), 3.38 (2H, d, J=6.8 Hz), 4.25 (2H, q, J=7.1 Hz), 4.48-4.59 (2H, m), 8.33 (1H, s), 11.48-12.17 (1H, m)

MS ^{m}/_{z}: 463 (M+1).

### Example 129

### Ethyl 5-cyano-[3-(2-{[(4-fluorobenzyl)sulfonyl]amino}-2-oxoethyl)azetidin-1-yl]-2-methylnicotinate

{1-[3-cyano-5-(ethoxycarbonyl)-6-methylpyridin-2-yl]azetidin-3-yl}acetic acid (130 mg, 0.43 mmol), TBTU (190 mg, 0.59 mmol), DIPEA (0.2 mL, 1.2 mmol) were dissolved in dry DCM (4 mL) and the reaction mixture was stirred at room temperature for 1 h 20 min. The mixture was added to 1-(4-fluorophenyl)methanesulfonamide (189 mg, 0.53 mmol) and the reaction mixture was stirred at room temperature for 25 h. NaHCO₃ (aq) was added and the mixture was extracted three times with DCM. The combined organic layers was passed through a phase separator and the solvents were removed in vacuo. The crude product was purified using preparative HPLC on a (Kromasil C8 10 μm, 21.5×250 mm) using a gradient of 25-45% acetonitrile/aqueous NH4OAc buffer pH 7 to give ethyl 5-cyano-6-[3-(2-{[(4-fluorobenzyl)sulfonyl]amino}-2-oxoethyl)azetidin-1-yl]-2-methylnicotinate. Yield=134 mg (66%).

¹H-NMR (500 MHz, DMSO-d6): δ 1.30 (3H, t, J=7.2 Hz), 2.62 (3H, s), 2.70 (2H, m), 3.04 (1H, m), 4.02 (2H, m), 4.23 (2H, q, J=7.2 Hz), 4.46 (2H, m), 4.71 (2H, s), 7.26 (2H, m), 7.35 (2H, m), 8.28 (1H, s), 11.69 (1H, s).

MS (^{m}/_{z}): 475.1 (M+1), 473.0 (M−1).

### Example 130

### Ethyl 5-cyano-6-[4-({[(3-fluoro-4-methylbenzyl)sulfonyl]amino}carbonyl)piperidin-1-yl]-2-methylnicotinate

Prepared according to method B staring from 1-(3-fluoro-S methylphenyl)methanesulfonamide, which was prepared from the corresponding bromide in a similar fashion to example 109 step a. Yield=2 mg (2%).

MS ^{m}/_{z}: 503 (M+1).

### Example 131

### Ethyl 6-(4-{[(benzylsulfonyl)amino]carbonyl}piperidin-1-yl)-5-chloro-2-methylnicotinate

### a) 1-[3-chloro-5-(ethoxycarbonyl)-6-methylpyridin-2-yl]piperidine-4-carboxylic acid

Ethyl 5,6-dichloro-2-methylnicotinate (428 mg, 1.79 mmol) was dissolved in MeCN (6 mL) followed by addition of piperidine-4-carboxylic acid (255 mg, 1.74 mmol), Water (9 mL) and TEA (1.5 mL, 10.8 mmol) were added. The reaction mixture was heated for 15 min at 120° C. in a single node microwave oven. An additional amount of piperidine-4-carboxylic acid (128 mg, 0.34 mmol) and TEA (0.5 mL, 3.60 mmol) were added and the mixture was heated in a single node microwave oven for 15 min at 120° C. The reaction mixture was diluted with DCM and washed with 2% KHSO4. The aqueous phase was extracted twice with DCM and the combined organic phases were filtered through a phase separator and followed by removal of solvents in vacuo. The crude was co-concentrated with DCM to give 1-[3-chloro-5-(ethoxycarbonyl)-6-methylpyridin-2-yl]piperidine-4-carboxylic acid. Yield=391 mg (60%).

¹H-NMR (400 MHz, DMSO-d6) δ 1.29 (3H, t, J=7.1 Hz), 1.71-1.58 (2H, m), 1.96-1.85 (2H, m), 2.59 (3H, s), 3.03-2.91 (2H, m), 4.02-3.91 (2H, m), 4.24 (2K, q, J=7.1 Hz), 8.02 (1H, s), 12.43-12.07 (1H, bs).

### b) Ethyl 6-(4-{[(benzylsulfonyl)amino]carbonyl}piperidin-1-yl)-5-chloro-2-methylnicotinate

1-[3-chloro-5-(ethoxycarbonyl)-6-methylpyridin-2-yl]piperidine-4-carboxylic acid (391 mg, 1.08 mmol), DIPEA (0.9 mL, 5.4 mmol)) and TBTU (404 mg, 1.25 mmol) were dissolved in dry DCM (8 mL) and stirred for 15 min at room temperature followed by addition of 1-Phenylmethanesulfonamide (221 mg, 1.30 mmol). The reaction mixture was stirred over night after which an additional amount of TBTU (14 mg, 0.044 mmol) and 1-Phenymethanesulfoneamide (8 mg, 0.047 mmol) were added. The reaction mixture was then stirred for another 3 h before it was diluted with DCM and washed twice with 1% KHSO₄ (aq)×2. The aqueous phase was extracted with twice with DCM and the combined organic phases were passed through a phase separator and followed by removal of solvents in vacuo. The crude product was purified using preparative HPLC on a (Kromasil C8, 55×300 mm), in order to avoid precipitation the compound was loaded onto the column using 5% acetonitrile/aqueous NH₄OAc buffer pH 7. The product was eluted using a gradient of 30-100% acetonitrile/aqueous NH₄OAc buffer pH 3. The fractions containing the product was concentrated in vacuo, dissolved in ethyl acetate and washed with brine. The aqueous phase was extracted twice with ethylacetate and the combined organic phases were concentrated in vacuo to give Ethyl 6-(4-{[(benzylsulfonyl)amino]carbonyl}piperidin-1-yl)-5-chloro-2-methylnicotinate as a white/yellow solid. Yield=267 mg (51%).

¹H NMP (400 MHz, DMSO-d6) δ 1.29 (3H, t, J=7.1 Hz), 1.74-1.59 (2H, m), 1.84-1.74 (2H, m), 2.49 (1H, s), 2.60 (3H, s), 2.86 (2H, t, J=12.7 Hz), 4.10-4.00 (2H, m), 4.25 (2H, q, J=7.1 Hz), 4.67 (2H, s), 7.44-7.23 (5H, m), 8.03 (1H, s), 11.57 (1H, s)

MS ^{m}/_{z}: 480 (M+1).

### Example 132

### 4-fluorobenzyl 6-(4-{[(benzylsulfonyl)amino]carbonyl}piperidin-1-yl)-5-cyano-2-methylnicotinate

### a) 4-fluorobenzyl 6-chloro-5-cyano-2-methylnicotinate

Ethyl 5,6-dichloro-2-methylnicotinate (120, 0.56 mmol) was dissolved in dry THF (4 mL), DIPEA (0.2 mL, 1.15 mmol) and (4-fluorophenyl)methanol (0.062 mL, 0.57 mmol) were added. The reaction mixture was stirred at r.t. for 115 h. The solvents were removed in vacuo and the crude product was used in the next step without purification.

### b) 4-fluorobenzyl 6-(4-{[(benzylsulfonyl)amino]carbonyl}piperidin-1-yl)-5-cyano-2-methylnicotinate

The crude 4-fluorobenzyl 6-chloro-5-cyano-2-methylnicotinate (137 mg, 0.47 mmol) was dissolved in THF (2 mL), MeOH (2 mL), DIPEA (00.1 mL) and N-(benzylsulfonyl)piperidine-4-carboxamide (140 mg, 0.49 mmol) were added. The reaction mixture was heated to 120° C. for 5 min using a microwave single node heating. NaHCO₃ (aq) was added and the mixture was extracted three times with DCM. The combined organic layer was passed through a phase separator and the solvents were removed in vacuo. The crude product was purified using preparative HPLC on a (Kromasil C8 10 μm, 21.5×250 mm) using a gradient of 30-55% acetonitrile/aqueous NH4OAc buffer pH 7 to give 4-fluorobenzyl 6-(4-{[(benzylsulfonyl)amino]carbonyl}piperidin-1-yl)-5-cyano-2-methylnicotinate. Yield=39 mg (16%).

¹H NMR (500 MHz, DMSO-d6) δ 1.64 (2H, m), 1.84 (2H, m), 2.58 (1H, m), 2.65 (3H, s), 3.15 (2H, m), 4.54 (2H, m), 4.69 (2H, s), 5.27 (2H, s), 7.23 (2H, m), 7.29 (2H, m), 7.40 (3H, m), 7.54 (2H, m), 8.38 (1H, s), 11.61 (1H, s).

LCMS⁺/_{z}: 551.2 (M+1), 549.3 (M−1).

### Example 133

### Ethyl 5-cyano-6-[4-({[(4-ethylbenzyl)sulfonyl]amino}carbonyl)piperidin-1-yl]-2-methylnicotinate

1-[3-cyano-5-(ethoxycarbonyl)-6-methylpyridin-2-yl]piperidine-4-carboxylic acid (95 mg, 0.30 mmol) was dissolved in DCM (1 mL) and HATU (148 mg, 0.39 mmol) followed by DIPEA (155 mg, 1.20 mmol) were added. The mixture was stirred at rt for 5 min before the addition 1-(4-ethylphenyl)methanesulfonamide (66 mg, 0.33 mmol), made from the corresponding sulfonyl chloride in a similar manner to example 65b. The reaction mixture was stirred for 18 h followed buy addition of 1-(4-1-(4-ethylphenyl)methanesulfonamide (10 mg, 0.05 mmol) in 0.2 mL DCM, followed by HATU (20 mg, 0.053 mmol) and stirring at rt was continued for 22 h. The solvents were removed in vacuo and the crude material pas dissolved in DMSO (10 mL) and purified by reverse phase preparative HPLC. Solvents used: A: MeCN, B: 0.1 M ammonium acetate/MeCN 95:5, C: 5% MeCN in 50 mM HCOOH/50 mM ammonium formiate. Start: A/B/C 5:95:0. Injected at flow-20 mL/min. Increased to flow=100 mL/min. 3 min. after the injection. Then changed to A/B/C 5:0:95. Increased to 100:0:0 over 20 min. in 9 equal steps. Column: Kromasil C8, 250 mm×50.8 ID. The relevant fractions was concentrated and freeze-dried over night to give 0.071 g of the title compound. The Na-salt was made by slurrying the material in acetonitrile (0.4 mL) and adding 1.0 eq. 0.1 M NaOH (1.42 mL) and some water (ca. 10 mL). After stirring for 5 min. almost all material had gone into solution. The solids were removed by filtration and the solution phase was freeze-dried. This gave ethyl 5-cyano-6-[4-({[(4-ethylbenzyl)sulfonyl]amino}carbonyl)piperidin-1-yl]-2-methylnicotinate. Yield=0.076 g. (49%)

1H NMR (400 MHz, DMSO-d₆) δ 1.17 (3H, t, J=7.6 Hz), 1.32 (3H, t, J=7.1 Hz), 1.54-1.67 (2H, m), 1.76-1.85 (2H, m), 2.20-2.30 (1H, m), 2.56 (2H, q, J=7.6 Hz), 2.65 (3H, s), 3.17-3.27 (2H, m), 4.20 (2H, s), 4.26 (2H, q, J=7.1 Hz), 4.39-4.47 (2H, m), 7.09 (2H, br d, J=8.1 Hz), 7.15 (2H, br d, J=8.1 Hz), 8.32 (1H, s).

MS ^{m}/_{z}: 499 (M+1)

### Example 134

Prepared according to method B starting from 1-(3,4-difluorophenyl)methanesulfonamide, made from the corresponding sulfonyl chloride in a similar manner to example 65b. Yield=4 mg (4%).

MS ^{m}/_{z}: 479 (M+1).

### Example 135

### Ethyl 5-cyano-6-[4-({[(4-methoxybenzyl)sulfonyl]amino}carbonyl)piperidin-1-yl]-2-methylnicotinate

1-[3-cyano-5-(ethoxycarbonyl)-6-methylpyridin-2-yl]piperidine-4-carboxylic acid (40 mg. 0.126 mmol) was dissolved in 0.5 mL DCM and TBTU (57 mg, 0.18 mmol) and DIPEA (0.064 mL, 0:38 mmol) were added. The solution was stirred at rt for 5 min followed by addition to 1-(4-methoxyphenyl)methanesulfonamide (32 mg, 0.16 mmol), which was prepared from the corresponding chloride in a similar fashion to example 109, in DCM. The reaction mixture was stirred for 2.5 days followed by purification by reverse phase HPLC. Solvents used: A: MeCN, B: 0.1 M ammonium acetate/MeCN 95:5, C: 5% MeCN in 50 mM HCOOH/50 mM ammonium formiate. Start: A/B/C 5:95:0. Injected at flow=10 mL/min. Increased to flow=20 mL/min. just after injection. Then changed to A/B/C 5:0:95. Increased to 50:0:50 over 30 min. in 9 equal steps. Then to 100:0:0 over 10 min. in 5 steps. Flow: 20 mL/min. Column: Kromasil C8, 250 mm×20 ID. The material stuck on the column and did not elute until A/B/C was 95/0/5 to 100/0/0.

For the relevant fractions the organic solvents were removed in vacuo followed by freeze drying. 1 eq. 0.1 M NaOH was added. The material was freeze dried again to give ethyl 5-cyano-6-[4-({[(4-methoxybenzyl)sulfonyl]amino}carbonyl)piperidin-1-yl]-2-methylnicotinate. Yield=34 mg (45%).

¹H NMR (400 MHz, DMSO-d6) δ 1.32 (3H, d, J=7.1 Hz, 1.55-1.68 (2H, m), 1.77-1.86 (2H, m), 2.27-2.36 (1H, m), 2.66 (3H, s), 3.16-3.25 (2H, m), 3.74 (3H, s), 4.22-4.30 (4H, m), 4.42-4.51 (2H, m), 6.85 (2H, br d, J=8.5 Hz), 7.16 (2H, br d, J-8.5 Hz), 8.33 (1H, s).

MS ^{m}/_{z}: 523 (M+1)

### Example 136

### Ethyl 5-cyano-2-methyl-6-[4-({[(3-methylbenzyl)sulfonyl]amino}carbonyl)piperidin-1-yl]nicotinate

1-[3-cyano-5-(ethoxycarbonyl)-6-methylpyridin-2-yl]piperidine-4-carboxylic acid (143 mg, 0.45 mmol) was dissolved in dry DCM (4 mL), TBTU (168 mg, 0.52 mmol)) and DIPEA (0.16 ml, 0.92 mmol) were added. The mixture was stirred at room temperature for 30 min and 1-(3-methylphenyl)methanesulfonamide (103 mg, 0.56 mmol) was added. The reaction mixture was stirred at room-temperature for 22 h NaHCO₃ (aq) was added and the mixture was extracted three times with DCM. The combined organic layer was passed through a phase separator and the solvents were removed in vacuo. The crude product was purified using preparative HPLC on a (Kromasil C8 10 μm, 21.5×250 mm) using a gradient of 25-45% acetonitrile/aqueous NH₄OAc buffer pH 7 to give ethyl 5-cyano-2-methyl-6-[4-({[(3-methylbenzyl)sulfonyl]amino}carbonyl)piperidin-1-yl]nicotinate. Yield=160 mg (73%).

¹H NMR (500 MHz, DMSO-d6): δ 7.31 (3H, t, J=7.0), 1.64 (2H, m), 1.82 (2H, m), 2.31 (3H, s), 2.59 (1H, m), 2.65 (3H, s), 3.14 (2H, m), 4.26 (2H, q, J=7.0), 4.54 (2H, m), 4.65 (2H, s), 7.10 (2H, m), 7.21 (1H, m), 7.29 (1H, m), 8.35 (1H, s), 11.58 (1H, s).

MS^{m}/_{z}: 485.2 (M⁺¹), 483.2 (M⁻¹).

### Example 137

### Ethyl 5-cyano-6-[3-{[(4-ethylbenzyl)sulfonyl]amino}carbonyl)azetidin-1-yl]-2-methylnicotinate

1-[3-cyano-5-(ethoxycarbonyl)-6-methylpyridin-2-yl]azetidine-3-carboxylic acid (145 mg, 0.500 mmol) was dissolved in 2 mL DCM/DMF 1:1 and TBTU (265 mg, 0.700 mmol), DIPEA (0.34 mL, 2 mmol) and 1 mL DMF. The mixture was stirred at rt for 5 min before the addition of 1-(4-ethylphenyl)methanesulfonamide (120 mg, 0.600 mmol), made from the corresponding sulfonyl chloride in a similar manner to example 65b, in 1 mL DCM. The reaction mixture was stirred over week-end followed by addition of more (4-ethylphenyl)methanesulfonamide (10 mg, 0.05 mmol) in 0.2 mL DCM, followed by extra TBTU (20 mg, 0.05 mmol) and stirring at rt was continued for 22 h further. The solvents were removed in vacuo followed by addition of DMSO (10 mL).

Purification was performed by reverse phase HPLC. Solvents used: A: MeCN, B: 0.1 M ammonium acetate/MeCN 95:5, C: 5% MeCN in 50 mM HCOOH/50 mM ammonium formiate. Start: A/B/C 5:95:0. Injected at flow=20 mL/min Increased to flow=100 mL/min. 3 min. after the injection. Then changed to A/B/C 5:0:95. The flow had to be reduced to 50 mL/min. (flow=100 mL/min. gave automatic cut-off due to increase in internal pressure). Increased to 50:0:50 over 15 min. in 5 steps. The to 100:0:0 over 15 min. in 5 steps. Column: Kromasil C8, 250 mm×50.8 ID. The relevant fraction was concentrated in vacuo and freeze-dried over night. This gave 0.111 g product. The Na-salt was made by slurrying the material in acetonitrile (1 mL) and adding 1.0 eq. 0.1 M NaOH (2.36 mL) and some water (ca. 10 mL). After stirring for 5 min. almost all material had gone into solution. The solids were removed by filtration (syringe+filter) and the liquid was freeze-dried. This gave Ethyl 5-cyano-6-[3-({[(4-ethylbenzyl)sulfonyl]amino}carbonyl)azetidin-1-yl]-2-methylnicotinate. Yield=120 mg (49%).

1H NMR (400 MHz, DMSO-d6) δ 1.16 (3H, t, J=7.6 Hz), 1.32 (31, t, J=7.1 Hz), 2.55 (2H, q, J=7.6 Hz), 2.64 (3H, s), 3.15-3.25 (1H, m), 4.22-4.27 (4H, m), 4.27-4.40 (4H, m), 7.05 (2H, br d, J=8.0 Hz), 7.15 (2H, br d, J=8.0 Hz), 8.28 (1H, s).

MS ^{m}/_{z}: 471 (M+1)

### Example 138

### Ethyl 5-chloro-2-methyl-6-[3-({[(4-methylbenzyl)sulfonyl]amino}carbonyl)azetidin-1-yl]nicotinate

A solution of 1-[3-Chloro-5-(ethoxycarbonyl)-6-methylpyridin-2-yl]azetidine-3-carboxylic acid (235 mg, 0.788 mmol), DIPEA (686 μl, 3.94 mmol) and TBTU (303 mg, 0.945 mmol) in dry DCM (5 mL) was stirred for 10 min at rt followed by addition of a solution of 1-(4-methylphenyl)methanesulfonamide (175 mg, 0.945 mmol) in dry DCM (1 mL). The reaction mixture was stirred over night followed by addition 2% KHSO₄ (aq), the phases were separated and the organic phase was washed with 2% KHSO₄ (aq). The aqueous phase was extracted twice with DCM and the combined organic phases were filtered through a phase separator and the solvents were removed in vacuo. The crude product was purified using preparative HPLC on a (Kromasil C8, 50.8×300 mm), in order to avoid precipitation the compound was loaded onto the column using 5% acetonitrile/aqueous NH₄OAc buffer pH 7. The product was eluted using a gradient of 5-90% acetonitrile/aqueous NH₄OAc buffer pH 3 to give Ethyl 5-chloro-2-methyl-6-[3-({[(4-methylbenzyl)sulfonyl]amino}carbonyl)azetidin 1-yl]nicotinate as a white solid after freeze drying. Yield=289 mg (78%).

¹H NMR (400 MHz, DMSO-d₆) δ 1.28 (3H, t, J=7.4 Hz), 2.28 (3H, s), 2.59 (3H, s), 3.41-3.53 (1H, m), 4.16-4.29 (4H, m), 4.29-4.39 (2H, m), 4.67 (2H, s), 7.11-7.25 (4H, m), 7.93 (1H, s), 11.71 (1H, s).

MS ^{m}/_{z}: 466 (M+1).

### Example 139

### Ethyl 5-cyano-6-[4-({[(3,4-difluorobenzyl)sulfonyl]amino}carbonyl)piperidin-1-yl]-2-methylnicotinate

Prepared according to method B starting from 1-(3,4-difluorophenyl)methanesulfonamide, made from the corresponding sulfonyl chloride in a similar manner to example 65b. Yield=16 mg (16%).

¹H NMR (400 MHz, DMSO-d6) δ 1.30 (3H, t, J=7.1 Hz), 1.53-1.70 (2H, m), 1.76-1.90 (2H, m), 2.47-2.54 (1H, m), 2.64 (3H, s), 3.15 (2H, app. t, J=11.6 Hz), 4.25 (2H, q, J=7.1 Hz), 4.47-4.57 (2H, m), 4.67 (2H, s), 7.08-7.18 (1H, m), 7.30-7.40 (1H, m), 7.41-7.53 (1H, m), 8.34 (1H, s), 11.42-12.03 (1H, m)

MS ^{m}/_{z}: 507 (M+1).

### Example 140

### Ethyl 5-cyano-6-[3-({[(4-methoxybenzyl)sulfonyl]amino}carbonyl)azetidin-1-yl]-2-methylnicotinate

1-[3-cyano-5-(ethoxycarbonyl)-6-methylpyridin-2-yl]azetidine-3-carboxylic acid (43 mg, 0.150 mmol) was dissolved in 0.5 mL DCM followed by addition of TBTU (67 mg, 0.21 mmol) and DIPEA (0.076 mL, 0.45 mmol). The solution was stirred at rt for 5 min followed by addition of 1-(4-methoxyphenyl)methanesulfonamide (80 mg, 0.180 mmol), which was prepared from the corresponding chloride in a similar fashion to example 109, dissolved in DCM. The reaction mixture was stirred for 18 h followed by addition of TBTU (34 mg, 0.11 mmol) and DIPEA (0.152 mL, 0.89 mmol). The reaction mix was stirred for 5 min followed by addition of 1-(4-methoxyphenyl)methanesulfonamide (27 mg, 0.060 mmol, 45%) was dissolved in 0.2 mL DMF and added to the reaction mixture. Stirring at rt was continued for 16 h. The solvents were removed in vacuo and the crude material was dissolved in 5 mL ethyl acetate followed by extraction with 2×5 mL 1M NaHSO₄ and 1×5 mL brine. The organic layer was isolated, dried over sodium sulphate, filtered and the solvents were removed in vacuo to give 0.136 g of crude material.

Purification was performed by reverse phase HPLC. Solvents used: A: MeCN, B: 0.1 M ammonium acetate/MeCN 95:5, C: 5% MeCN in 50 mM HCOOH/50 mM ammonium formiate. Start: A/B/C 5:95:0. Injected at flow=10 mL/min. Increased to flow-20 mL/min. just after injection. Then changed to A/B/C 5:0:95. Increased to 50:0:50 over 30 min. in 9 equal steps. Then to 100:0:0 over 10 min. in 5 steps. Flow: 20 mL/min. Column: Kromasil C8, 250 mm×20 ID. The material stuck on the column and did not elute until A/B/C was 95/0/5 to 100/0/0.

The relevant fraction was evaporated and freeze dried, quantified (0.032 g) and 1 eq. 0.1 M NaOH was added. The material was freeze dried to give ethyl 5-cyano-6-[3-({[(4-methoxybenzyl)sulfonyl]amino}carbonyl)azetidin-1-yl]-2-methylnicotinate. Yield=34 mg (48%).

¹H NMR (400 MHz, DMSO-d6) δ 1.31 (3H, t, J=7.2 Hz), 2.63 (3H, s), 3.14-3.23 (1H, m), 3.72 (3H, s), 4.21 (2H, s), 4.25 (2H, q, J=7.2 Hz), 4.28-4.39 (4H, m), 6.79 (2H, br d, J=8.6 Hz), 7.16 (2H, br d, J=8.6 Hz), 8.28 (1H, s).

MS ^{m}/_{z}: 473 (M+1)

### Example 141

### Cyclopropyl 5-cyano-2-methyl-6-[4-({[(4-methylbenzyl)sulfonyl]amino}carbonyl)piperidin-1-yl]nicotinate

1-{3-cyano-5-[(cyclopropyloxy)carbonyl]-6-methylpyridin-2-yl}piperidine-4-carboxylic acid (40 mg, 0.12 mmol), was dissolved in DCM followed by addition of TBTU (46.8 mg, 0.15 mmol) and DIPEA (0.11 mL, 0.61 mmol) after 10 min. The reaction mixture was added to 1-(4-methylphenyl)methanesulfonamide (27 mg, 0.15 mmol) and was stirred over night. The solvents were removed in vacuo and the crude material was partitioned between EtOAc (5 ml)/1 MKHSO₄ (1 mL). The organic layer was washed with water (1 mL) and the solvents were removed in vacuo. The compound was purified by preparative HPLC to give cyclopropyl 5-cyano-2-methyl-6-[4-({[(4-methylbenzyl)sulfonyl]amino}carbonyl)piperidin-1-yl]nicotinate as a white solid. Yield=11 mg (97%)

¹H NMR (400 MHz, DMSO-d6) δ 0.71-0.85 (4H, m), 1.56-1.70 (2H, m), 1.78-1.90 (2H, m), 2.31 (3H, s), 2.55-2.62 (1H, m), 2.63 (3H, s), 3.07-3.20 (2H, m), 4.23-4.28 (1H, m), 4.48-4.60 (2H, m), 4.64 (2H, s), 7.17 (2H, d, J=8.1 Hz), 7.21 (2H, d, J=8.1 Hz), 8.30 (1H, s), 11.55 (1H, s)

MS ^{m}/_{z}: 497 (M+1).

### Example 142

### Ethyl 5-cyano-2-methyl-6-[3-({[(pyridin-4-ylmethyl)sulfonyl]amino}carbonyl)azetidin-1-yl]nicotinate

Prepared according to method B starting from 1-pyridin-4-ylmethanesulfonamide, which was prepared from the corresponding bromide in a similar fashion to example 109 step a. Yield=7 mg (8%).

¹H NMR (400 MHz, DMSO-d6) δ 1.30 (3H, t, J=6.9 Hz), 2.63 (3H, s), 3.39-3.49 (1H, m), 4.21-4.26 (2H, m), 4.28-4.55 (4H, m), 4.64 (2H, s), 7.28-7.42 (2H, m), 8.30 (1H, s), 8.48-8.62 (2H, m)

MS ^{m}/_{z}: 444 (M+1).

### Example 143

### Ethyl 6-(3-{[(benzylsulfonyl)amino]carbonyl}azetidin-1-yl)-5-cyano-2-(dimethylamino)nicotinate

### a) 1-[3-Cyano-6-(dimethylamino)-5-(ethoxycarbonyl)pyridin-2-yl]azetidine-3-carboxylic acid

Ethyl 6-chloro-5-cyano-2-(dimethylamino)nicotinate (507 mg, 1.60 mmol) was dissolved in ethanol/water 1:1 followed by addition of azetidine-3-carboxylic acid (242 mg, 2.39 mmol) and TEA (0.644 mL, 4.80 mmol). The reaction mixture was heated in a microwave oven, single node heating, at 120° C. for 20 min. The solvents were removed in vacuo and the residue was dissolved in 15 mL DMSO. Some undissolved material was removed by filtration prior to purification by reverse phase HPLC.

A: MeCN, B: 0.1 M ammonium acetate/MeCN 95:5, C: 5% MeCN in 50 mM HCOOH/50 mM ammonium formiate. Start: A/B/C 5:95:0. Injected at flow=10 mL/min. Increased to flow=50 mL/min. continuous over 3 minutes after the injection Then changed to A/B/C 5:0:95 and flow increased to 100 mL/min. Increased to 100:0:0 over 17 minutes in steps of 5% points. Column: Kromasil C8, 250 mm×50.8 ID.

This gave 1-[3-Cyano-6-(dimethylamino)-5-(ethoxycarbonyl)pyridin-2 yl]azetidine-3-carboxylic acid. Yield=0.064 g. (13%).

### b) Ethyl 6-(3-{[(benzylsulfonyl)amino]carbonyl}azetidin-1-yl)-5-cyano-2-(dimethylamino)nicotinate

1-[3-Cyano-6-(dimethylamino)-5-(ethoxycarbonyl)pyridin-2-yl]azetidine-3-carboxylic acid (64 mg, 0.200 mmol) was dissolved in DCM and TBTU (96 mg, 0.300 mmol) and DIPEA (0.136 mL, 0.800 mmol) were added. The mix was stirred for 5 minutes at r.t before the addition of 1-phenylmethanesulfonamide (48 mg, 0.28 mol). The reaction mixture was stirred at r.t. for 16 h. 1-Phenylmethanesulfonamide (48 mg, 0.28 mol), TBTU (96 mg, 0.300 mmol) and DIPEA (0.136 mL, 0.800 mmol) and stirring at r.t was continued for 20 h further. Bromo-tris-pyrrolidino-phosphonium hexafluorophosphate (93 mg, 0.20 mmol) was added and the mix was stirred at rt for 3.5 days (un-optimiz.). Thionyl chloride (0.044 mL, 0.600 mmol) was added and the reaction mixture was stirred for an additional 16 h.

Purification was done by reverse phase HPLC. A: MeCN, B: 0.1 M ammonium acetate/MeCN 95:5. Start: A/B 5:95. Injected at flow=10 mL/min. Increased to flow=20 mL/min. 3 min. after injection. Then changed to A/B/C 5:0:95. Increased to 100:0 over 20 min., increasing with same interval each single minute. Flow: 20 mL/min. Column: Kromasil C8, 250 mm×20 ID. This gave a product that was only 71% pure. Rest was guanidine by-product.

Re-purification was done by reverse phase HPLC. A: MeCN, B: 0.1 M ammonium acetate/MeCN 95:5, C: 5% MeCN in 50 mM HCOOH/50 mM ammonium formiate. Start: A/B/C 5:95:0. Injected at flow=10 mL/min. Increased to flow-20 mL/mL 3 min. after injection. Then changed to A/B/C 5:0:95. Increased to 100:0:0 over 20 min. in equal steps. Flow: 20 mL/min. Column: Kromasil C8, 250 mm×20 ID.

The above method gave ethyl 6-(3-{[(benzylsulfonyl)amino]carbonyl}azetidin-1-yl)-5-cyano-2-(dimethylamino)nicotinate. Yield 11 mg. (12%).

¹H NMR (400 MHz, CDCl₃) δ 1.30-1.40 (3H, m), 3.03 (6H, s), 3.00-3.06 (2H, m), 4.21-4.40 (5H, m), 4.67 (2H, s), 7.33-7.45 (5H, m), 8.10 (1H, s)

MS ^{m}/_{z}: 472 (M+1)

### Example 144

### Ethyl 6-(4-{[(benzylsulfonyl)amino]carbonyl}piperidin-1-yl)-5-cyano-2-methylnicotinate 1-oxide

### a) Ethyl 6-chloro-5-cyano-2-methylnicotinate I-oxide

Ethyl 6-chloro-5-cyano-2-methylnicotinate (1.00 g, 4.45 mmol) was dissolved in DCM (25 mL) and cooled on an ice-bath. Urea hydrogen peroxide (2.09 g, 22.3 mmol) was charged and trifluoroacetic acid anhydride (3.11 mL, 22.3 mmol) was added dropwise during 2-3 minutes. The cooling bath was removed after 15 min and the reaction mixture was stirred over night. Sodium Pyrosulphite 4.2 g in 15 mL water was added and the reaction mixture was stirred for 3 min, followed by addition of DCM (5 ml) and 1M KHSO₄ (2 ml) and stirring continued for 5 min. The aqueous layer was extracted three times with DCM and the combined organics were dried over sodium sulphate. Concentration yielded 900 mg of a light yellow sticky solid. The crude material was purified by preparative HPLC, 50×300 mm, C8, 10 um to give ethyl 6-chloro-5-cyano-2-methylnicotinate I-oxide. Yield=356 mg (33%)

¹H-NMR (400 MHz, DMSO-d6) δ 1.34 (3H, t, J-7.1 Hz), 2.68 (3H, s), 4.36 (2H, q, J=7.1H), 8.25 (1H, s)

### b) Ethyl 6-(4-{[(benzylsulfonyl)amino]carbonyl}piperidin-1-yl)-5-cyano-2-methylnicotinate 1-oxide

Ethyl 6-chloro-5-cyano-2-methylnicotinate 1-oxide (50 mg, 0.21 mmol) and N-(benzylsulfonyl)piperidine-4-carboxamide (59 mg, 0.21 mmol) were charged in a flask and dissolved in EtOH (2 mL) where upon DIPEA (0.072 mL, 0.42 mmol) was added. The reaction mixture was concentrated in vacuo after 10 min. The crude product was purified using preparative HPLC to give ethyl 6-(4-{[(benzylsulfonyl)amino]carbonyl}piperidin-1-yl)-5-cyano-2-methylnicotinate 1-oxide. Yield=65 mg: (64%)

¹H NMR (400 MHz, DMSO-d6) δ 1.32 (3H, t, J=7.1 Hz), 1.70-1.84 (4H, m), 2.04-2.25 (1H, m), 2.63 (3H, s), 3.16-3.25 (2H, m), 3.54 (1H, br s), 3.71-3.80 (2H, m), 4.26 (2H, s), 4.30 (21, q, J=7.1), 7.21-7.30 (5H, m), 7.95 (1H, s)

MS ^{m}/_{z}: 487 (M+1).

### Example 145

### Ethyl 5-acetyl-6-(4-{[(benzylsulfonyl)amino]carbonyl}piperidin-1-yl)-2-methylnicotinate

### a) Ethyl 5-acetyl-2-methyl-6-oxo-1,6-dihydropyridine-3-carboxylate

3-Oxobutanamide (54.5 g, 539 mmol) was suspended in 400 mL EtOH. NaOEt/EtOH (210 mL, 564 mmol, 21%) was added dropwise and the reaction mixture was stirred at r.t for 1 h. Ethyl (2E)-2-acetyl-3-(dimethylamino)acrylate (10 g, 513 mmol) dissolved in 400 mL EtOH was added dropwise and the reaction mixture was stirred over night. The reaction mixture was concentrated in vacuo and the residue was dissolved in water and acidified to pH 1 with concentrated HCl. The reaction was stirred for 2 h followed by pH adjustment to ˜8 using solid potassium carbonate and saturated sodium bicarbonate. The reaction mixture was extracted into EtOAc, and DCM, and each of the organics were washed with brine. The combined organics were dried over MgSO₄, passed through a silica plug. The solvents were removed in vacuo and the remaining solids were triturated using 400 mL ether/hexane (1:1). This gave Ethyl 5-acetyl-2-methyl-6-oxo-1,6-dihydropyridine-3-carboxylateas a solid.

### b) Ethyl 5-acetyl-chloro-2-methylnicotinate

Ethyl 5-acetyl-2-methyl-6-oxo-1,6-dihydropyridine-3-carboxylate (1.67 g, 7.48 mmol) was dissolved in POCl₃ (13 mL, 139 mmol) and the mixture was heated to 110° C. and refluxed over night. The temperature was lowered to r.t. followed by removal of POCl₃ under reduced pressure. The crude product was dissolved in DCM, washed with saturated NaHCO₃ twice followed by brine and water. The aqueous phase was extracted with DCM and the organic phases were combined and the solvents removed in vacuo. The crude material was co-concentrated from EtOH and DCM once each to give crude ethyl 5-acetyl-6-chloro-2-methylnicotinate material that was used immediately in the consecutive step.

### c) 1-[3-acetyl-5-(ethoxycarbonyl)-6-methylpyridin-2-yl]piperidine-4-carboxylic acid

Ethyl 5-acetyl-6-chloro-2-methylnicotinate (600 mg, 2.11 mmol)) and piperidine-4-carboxylic acid (299 mg, 2.32 mmol) were dissolved in water (4 ml) and MeCN (6 ml) followed by addition of TEA (1.18 mL, 8.44 mmol) was added. The reaction mixture was heated in a single node microwave oven for 20 min at 120° C. The reaction mixture was diluted with DCM. Washed with 1% KHSO₄ twice, the combined aqueous phases were extracted with DCM and the combined organic phases were filtered through a phase separator and the solvents were removed in vacuo to give 1-[3-acetyl-5-(ethoxycarbonyl)-6-methylpyridin-2-yl]piperidine-4-carboxylic acid as a crude product which was used immediately in the next step. Yield=2.42 g (114%).

¹H NMR (400 MHz, DMSO-d₆) δ 1.63-1.47 (2H, m), 1.92-1.78 (2H, m), 2.46 (3H, s), 2.61 (3H, s), 3.13-2.99 (2H, m), 3.87-3.74 (2H, m), 4.24 (2H, q, J=7.0 Hz), 8.21 (1H, s)

### d) Ethyl 5-acetyl-6-(4-{[(benzylsulfonyl)amino]carbonyl}piperidin-1-yl)-2-methylnicotinate

A solution of 1-[3-acetyl-5-(ethoxycarbonyl)-6-methylpyridin-2-yl]piperidine-4-carboxylic acid (872 mg, 2.61 mmol)), DIPEA (2.27 mL, 13.0 mmol) and TBTU (1.0 g, 3.11 mmol) dry DCM (15 mL) was stirred for 15 min at room temperature followed by addition of 1-phenylmethanesulfonamide (536 mg, 3.13 mmol). The reaction mixture was stirred over night followed by addition of TBTU (100 mg, 0.31 mmol) and phenylmethanesulfonamide (53 mg, 0.31 mmol). The reaction mixture was then stirred for 5 h followed by addition of DCM. The reaction mixture was washed twice with 1% KHSO4 (aq). The aqueous phase was extracted with DCM×2 and the combined organic phases were passed through a phase separator and the solvents were removed in vacuo. The crude product was purified using preparative HPLC on a (Kromasil C8, 50.8×300 mm), in order to avoid precipitation the compound was loaded out o the column using 5% acetonitrile/aqueous NH4OAc buffer pH 7. The product was eluted using a gradient of 40-10% acetonitrile/aqueous NH4OAc buffer pH 3. The fractions containing the product was concentrated in vacuo, dissolved in DCM and washed with water. The aqueous phase was extracted twice with DCM and the combined organic phases were concentrated in vacuo to give ethyl 5-acetyl-6-(4-{[(benzylsulfonyl)amino]carbonyl}piperidin-1-yl)-2-methylnicotinate as a white/yellow solid. Yield=607 mg (48%).

¹H NMR (400 MHz, CDCl₃) δ 1.36 (3H, t, J=7.2 Hz), 1.67-1.85 (4H, m), 2.29-2.40 (1H, m) 2.50 (3H, s), 2.70 (3H, s), 2.94-3.04 (2H, m), 3.91-4.01 (2H, m), 4.32 (2H, q, J=7.2 Hz), 4.64 (2H, s), 7.27-7.41 (4H, m), 7.51 (1H, s), 8.38 (1H, s),

MS ^{m}/_{z}: 488 (M+1).

### Example 146

### ethyl 6-{4-{[(benzylsulfonyl)amino]carbonyl}-4-[(tert-butoxycarbonyl)amino]piperidin-1-yl}-5-cyano-2-methylnicotinate

### a) benzyl 4-{[(benzylsulfonyl)amino]carbonyl}-4-[(tert-butoxycarbonyl)amino]piperidine-1-carboxylate

1-[(benzyloxy)carbonyl]-4-[(tert-butoxycarbonyl)amino]piperidine-4-carboxylic acid (468 mg, 1.24 mmol)), TBTU (440 mg, 1.36 mmol) and DIPEA (0.3 mL, 1.72 mmol) were dissolved in dry DCM (4 mL) and stirred at rt for 1 h. 1-Phenylmethanesulfonamide (217 mg, 1.27 mmol) was added and the reaction mixture was stirred at r.t. for 17 h. NaHCO₃ (aq) was added and the mixture was extracted with DCM three times. The combined organic layers were run through a phase separator and the solvents were removed in vacuo. The crude product was purified by prep HPLC, Column: Kromasil C8 10 μm, 21.5×250 mm, Mobilephase A: 100% can, Mobilephase B: 5% AcN, 95% NH₄AcO (aq) (pH7), Gradient: 20=>40% A over 35 min, Flow: 25 ml/min, UV: 220 nm to give benzyl 4-{[(benzylsulfonyl)amino]carbonyl}-4-[(tert-butoxycarbonyl)amino]piperidine-1-carboxylate. Yield=297 mg (45%).

LCMS: m/z: 530.4 (M−1).

### b) tert-Butyl (4-{[(benzylsulfonyl)amino]carbonyl}piperidin-4 yl)carbamate

Benzyl 4-{[(benzylsulfonyl)amino]carbonyl}-4-[(tert-butoxycarbonyl)amino]piperidine-1-carboxylate (297 mg, 0.56 mmol), Pd(OH)₂ (96 mg, 0.136 mmol, 20% wt) and ammonium formiate (544 mg, 8.63 mmol) were suspended in MeOH (10 mL) in a 20-mL microwave vial. The reaction mixture was heated to 120° C. for 5 min using microwave single node heating. Pd(OH)₂ (50 mg, 0.094 mmol, 20% wt) and ammonium formiate (300 mg, 4.76 mmol) were added and the reaction mixture was heated to 12° C. for 5 min. An additional amount of Pd(OH)₂ (50 mg, 0.094 mmol) and ammonium formiate (400 mg, 6.34 mmol) were added and the reaction mixture was heated to 12° C. for 10 min. The reaction mixture was filtered and evaporated. The crude product was used in the next step without further purification.

LCMS⁺/_{z}: 398.2 (M+1), 396.3 (M−1).

### c) ethyl 6-{4-{[(benzylsulfonyl)amino]carbonyl}-4-[(tert-butoxycarbonyl)amino]piperidin-1-yl}-5-cyano-2-methylnicotinate

tert-Butyl (4-{[(benzylsulfonyl)amino]carbonyl}piperidin-4-yl)carbamate (107 mg, 0.27 mmol) and ethyl 6-chloro-5-cyano-2-methylnicotinate (124 mg, 0.55 mmol) were dissolved in EtOH (7 mL) and H₂O (2 mL) and DIPEA (1.3 mL, 7.46 mmol) was added. The reaction mixture was heated to 12° C. for 5 min using microwave single node heating. NaHCO₃ (aq) was added and the mixture was extracted three times with DCM. The combined organic layer was run through a phase separator and the solvents were removed in vacuo. The crude product was purified by prep HPLC Column: Kromasil C8 10 μm, 21.5×250 mm, Mobilephase A: 100% AcN, Mobilephase B: 5% AcN, 95% NH₄AcO (aq) (pH7), Gradient: 25=>50% A over 35 min, Flow: 25 ml/min

UV: 296 nm to give ethyl 6-{4-{[(benzylsulfonyl)amino]carbonyl}-4-[(tert-butoxycarbonyl)amino]piperidin-1-yl}-5-cyano-2-methylnicotinate. Yield=9 mg (3%).

LCMS⁺/_{z}: 586.4 (M+1), 584.4 (M−1).

### Example 147

### ethyl 6-(4-amino-4-{[(benzylsulfonyl)amino]carbonyl}piperidin-1-yl)-cyano-2-methylnicotinate

### Ethyl 6-{4-{[(benzylsulfonyl)amino]carbonyl}-4-[(tert-butoxycarbonyl)amino]piperidin-1-yl}-5-cyano-2-methylnicotinate (7.6 mg, 0.013 mmol) was dissolved in DCM (5 mL) and TFA (2 mL) was added. The reaction mixture was stirred at r.t. for 1 h followed by removal of the solvents in vacuo. The product was redissolved in AcN/H₂O and freeze dried yielding ethyl 6-(4-amino-4-{[(benzylsulfonyl)amino]carbonyl}piperidin-1-yl)-5-cyano-2-methylnicotinate trifluoroacetate. Yield=8 mg (1000%).

LCMSm/_{z}: 486.3 (M+1), 484.3 (M−1).

### Further Examples

### General Experimental Procedure

Mass spectra was recorded on a Finnigan LCQ Duo ion trap mass spectrometer equipped with an electrospray interface (LC-ms) or LC-ms system consisting of a Waters ZQ using a LC-Agilent 1100 LC system. ¹H NMR measurements were performed on a Varian Mercury VX 400 spectrometer, operating at a 1H frequency of 400 and Varian UNITY plus 400, 500 and 600 spectrometers, operating at 1H frequencies of 400, 500 and 600 respectively. Chemical shifts are given in ppm with the solvent as internal standard. Protons on heteroatoms such as NH and OH protons are only reported when detected in NMR and can therefore be missing. Chromatography was performed using Biotage silica gel 40S, 40M, 12i or Merck silica gel 60 (0.063-0.200 mm). Flash chromatography was performed using either standard glass- or plastic-columns column or on a Biotage Horizon system. HPLC separations were performed on a Waters YMC-ODS AQS-3 120 Angstrom 3×500 mm or on a Waters Delta Prep Systems using Kromasil C8, 10 μm columns.

The purification system and LC-MS system used in Method A′ to E′ below was Waters Fraction Lynx II Purification System: Column: Sunfire Prep C18, 5 μm OBD, 19×100 mm column. Gradient 5-95% CH₃CN in 0.1 mM HCOOH (pH=3). MS triggered fraction collection was used. Mass spectra were recorded on either Micromass ZQ single quadropole or a Micromass quattro micro, both equipped with a pneumatically assisted electrospray interface.

Reactions performed in a microwave reactor were performed in a Personal Chemistry Smith Creator, Smith synthesizer or an Emrys Optimizer.

### LIST OF USED ABBREVIATIONS

| Abbreviation | Explanation |
|---|---|
| AcOH | Acetic acid |
| aq | Aqueous |
| br | Broad |
| Brine | A saturated solution of sodium chloride in water |
| BSA | Bovine Serum Albumine |
| (Boc)₂O | di-tert-butyl dicarbonate |
| BuLi | Butyl lithium |
| CDI | Carbonyldiimidazole |
| d | Doublet |
| DBU | 1,8-Diazabicyclo[5.4.0]undec-7-ene |
| DCM | Dichloromethane |
| DDQ | 2,3-Dichloro-5,6-dicyano-1,4-benzoquinone |
| DIPEA | N,N-Diisopropylethylamine |
| DMA | N,N-Dimethylacetamide |
| DMF | N,N-dimethylformamide |
| DMSO | Dimethylsulphoxide |
| EDCI | N-[3-(dimethylamino)propyl]-N′-ethylcarbodiimide |
| | hydrochloride |
| EtOAc | Ethyl acetate |
| EtOH | Ethanol |
| HEPES | [4-(2-hydroxyethyl)-1-piperazineethanesulfonic acid |
| HFA | Hydrofluoroalkanes |
| HOAc | Acetic acid |
| HOBT | 1-Hydroxybenzotriazole |
| HPLC | High-performance liquid chromatography |
| Hz | Hertz |
| J | Coupling constant |
| LDA | Litiumdiisopropyl amide |
| m | Multiplet |
| Me | methyl |
| MHz | Megahertz |
| mL | Millilitre |
| MS | Mass spectra |
| NCS | N-chlorosuccinimide |
| OAc | acetate |
| ⁱPrOAc | isopropyl acetate |
| q | Quartet |
| r.t | Room temperature |
| s | Singlet |
| t | triplet |
| TB | Tyrodes Buffer |
| TBME | tert-butylmethyl ether |
| TBTU | N-[(1H-1,2,3-benzotriazol-1- |
| | yloxy)(dimethylamino)methylene]-N- |
| | methylmethanaminium tetrafluoroborate |
| TEA | Triethylamine |
| Tf | trifluoromethylsulfonyl |
| TFA | Trifluoroacetic acid |
| THF | Tetrahydrofurane |
| TMEDA | N,N,N′,N′-tetramethylethylendiamine |
| Ts | p-toluenesulfonyl |

### Synthesis of Sulfone Amides

The synthesis of the sulfonamides used in the examples below was made with one of the three methods described below:

i) By reacting the corresponding sulfonyl chloride with ammonia in THF or MeOH or by treatment with ammonium hydroxide in methylene chloride. The sulfonamides obtained was used without further purification.

ii) By essentially following the procedure described by Seto, T. et. al in J. Organic Chemistry, Vol 68, No 10 (2003), pp. 4123-4125.
or

iii) By essentially following the procedure described by Wang, Z et. al. in Tetrahedron Letters, Vol 43 (2002), pp 8479-8483.

### Synthesis of Examples

The following general procedures (i.e. Method A′ to E′) were used to prepare some of the examples below and are referred to in each specific example.

### Method A′

### Exemplified be the Procedure from Example 10

DIPEA (64 mg, 0.5 mmol) was added to a solution of 1-[3-cyano-6-(difluoromethyl)-5-(ethoxycarbonyl)pyridin-2-yl]piperidine-4-carboxylic (35.3 mg, 0.1 mmol) and TBTU (38.5 mg, 0.12 mmol) in DCM (5 mL) and the mixture was stirred for 30 min at r.t before 1-(2-fluorophenyl)methanesulfonamide (23 mg, 0.12 mmol) dissolved in DCM (1 mL) was added. The reaction was allowed to stir over night LC-MS showed that starting material was left and more TBTU (19 mg, 0.06 mmol) and DIPEA (26 mg, 0.2 mmol) were added to the mixture and the stirring was continued for another 2 h. The reaction mixture was washed with 1/KHSO₄, the aqueous phase was extracted with DCM (1 ml) and the combined organic phases passed through a phase separator and evaporated in a vacuum centrifuge. The crude product obtained was purified by HPLC (See General experimental procedure) to give ethyl 5-cyano-2-(difluoromethyl)-6-[4-({[(2-fluorobenzyl)sulfonyl]amino}carbonyl)piperidin 1-yl]nicotinate. Yield: 41 mg (78%).

### Method B′

### Exemplified by the Procedure from Example 42

DIPEA (128 mg, 1.0 mmol) was added to a solution of {1-[3-cyano-5-(ethoxycarbonyl)-6-(trifluoromethyl)pyridin-2-yl]pyrrolidin-3-yl}acetic acid (74.2 mg, 0.2 mmol) and TBTU (77 mg, 0.24 mmol) in DCM (7 mL) and the mixture was sired for 30 min at r.t before 1-phenylmethanesulfonamide (41 mg, 0.24 mmol) dissolved in DCM (1 mL) was added and the reaction was left over night. The reaction mixture was washed with 1% KHSO₄, the aqueous phase was extracted with DCM and the combined organic phases passed through a phase separator and evaporated in vacuum centrifuge. The crude-product obtained was purified by HPLC (See General experimental procedure) to give ethyl 6-(3-{2-[(benzylsulfonyl)amino]-2-oxoethyl}pyrrolidin-1-yl)-5-cyano-2-(trifluoromethyl)nicotinate. Yield: 88 mg (84%).

### Method C′

### Exemplified by the Procedure from Example 55

DIPEA (43 mg, 0.3 mmol) and TBTU (64 mg, 0.20 mmol) was added to a solution of 1-[3-cyano-5-(ethoxycarbonyl)-6-(trifluoromethyl)pyridin-2-yl]piperidine-4-carboxylic acid (74.2 mg, 0.2 mmol) in DMF and the mixture was stirred for 2 hours at r.t before it was added to 1-(4-fluorophenyl)methanesulfonamide (38 mg, 0.22 mmol) dissolved in DMF. The reaction mixture was stirred over night and passed through SCX-2 ion exchange column. The crude product obtained was purified by HPLC (See General experimental procedure) to give ethyl 5-cyano-6-[4-({[(4-fluorobenzyl)sulfonyl]amino}carbonyl)piperidin-1-yl]-2-(trifluoromethyl)nicotinate. Yield: 4.3 mg (4%).

### Method D′

### Exemplified by the Procedure from Example 45

CDI (26 rag, 0.16 mmol) was added to a solution of 1-[3-cyano-5-(ethoxycarbonyl)-6-(trifluoromethyl)pyridin-2-yl]azetidine-3-carboxylic acid (51 mg, 0.15 mmol) (gas evolution) in CH₃CN and the mixture was heated to 50° C. for 2 hours. The above mixture was then added to a solution of 1-(4-fluorophenyl)methanesulfonamide (28 mg, 0.15 mmol) and DBU (23 mg, 0.15 mmol) in CH3CN and the reaction was stirred at r.t over night. Purification by HPLC (See experimental procedure) gave ethyl 5-cyano-6-[3-({[(4-fluorobenzyl)sulfonyl]amino})carbonyl)azetidin-1-yl]-2-(trifluoromethyl)nicotinate.

### Method E′

### Exemplified by the Procedure from Example 75

DIPEA (38 mg, 0.3 mmol) was added to a solution of 1-[3-cyano-6-(difluoromethyl)-5-(ethoxycarbonyl)pyridin-2-yl]piperidine-4-carboxylic (35.3 mg, 0.1 mmol) and TBTU (38.5 mg, 0.12 mmol) in DCM (2 mL) and the mixture was stirred for 10 min. at r.t before 1-(2-fluorophenyl)methanesulfonamide (19 mg, 0.10 mmol) was added. The reaction was allowed to stir over night. The reaction mixture was washed with 1M KHSO₄ and the organic phases passed through a phase separator and evaporated in a vacuum centrifuge. The crude product obtained was purified by HPLC (See General experimental procedure) to give ethyl 5-cyano-6-[4-({[(2-fluorobenzyl)sulfonyl]amino}carbonyl)piperidin-1-yl]-2-(fluoromethyl)nicotinate. Yield: 13 mg (25%).

### Example 148

### Ethyl 6-(4-{[(benzylsulfonyl)amino]carbonyl}piperidin-1-yl)-5-chloro-2-(difluoromethyl)nicotinate

### (a) ethyl 2-(difluoromethyl)-6-oxo-1,6-dihydropyridine-3-carboxylate

Ethyl 2-methyl-6-oxo-1,6-dihydropyridine-3-carboxylate (2.0 g, 11.04 mmol) (Sobczak, A et al, Synth. Commun, Vol. 35, No. 23, 2005, pp 2993-3001) was added to a solution of 2-methoxy-N-(2-methoxyethyl)-N-(trifluoro-λ⁴-sulfanyl)ethanamine (7.82 g, 22.08 mmol) in CH₃CN under an atmosphere of nitrogen. The reaction was refluxed over night after which further 2-methoxy-N-(2-methoxyethyl)-N-(trifluoro-λ⁴-sulfanyl)ethanamine (2.73 g, 7.7 mmol) was added and the stirring was continued until all starting material was consumed. The reaction was diluted with diethyl ester; filtered to remove black solids, washed with water and NaHCO₃ (aq, sat). Both phases were filtered again to remove more of black solids. The aqueous phase was extracted with diethyl ether (2 times) and the combined organic phase was dried (MgSO₄), filtered and concentrated and slurried in diethyl ether to remove yellow impurities. Drying of the remaining white solid gave ethyl 2-(difluoromethyl)-6-oxo-1,6-dihydropyridine-3-carboxylate. Yield: 370 mg (14%).

¹H NMR (400 MHz, CDCl₃) δ 1.38 (3H, t) J=7.2 Hz), 4.36 (2H, q, J=7.2 Hz), 6.69 (1H, d, I=10 Hz), 7.56 (1H, t, J=54 Hz), 7.99 (1H, d, J=10 Hz).

### (b) ethyl 5-chloro-2-difluoromethyl)-6-oxo-1,6-dihydropyridine-3-carboxylate

NCS (270 mg, 2.02 mmol) dissolved in DMF (2 mL) was added to a solution of ethyl 2-(difluoromethyl)-6-oxo-1,6-dihydropyridine-3-carboxylate (365 mg, 1.44 mmol) and the reaction was heated to 100° C. over night. Since staring material still remained further aliquots of NCS (135 mg, 1.01 mmol and 5 hours later 270 mg, 2.02 mmol) was added and the heating was continued until the starting material had disappeared. The reaction was diluted with DCM and washed with water and Brine. The water phase was extracted twice with DCM and the combined organic phase was passed through a phase separator and evaporated. Purification by flash chromatography (Horizon Flash 40+M, Eluent: a gradient of EtOAc/Heptane from 50 to 100% EtOAc was used)) gave ethyl 5-chloro-2-(difluoromethyl)-6-oxo-1,6-dihydropyridine-3-carboxylate as a yellow oil which was used in the next step without further analysis or purification. Yield: 88 mg (15%).

### (c) ethyl 5,6-dichloro-2-(difluoromethyl)nicotinate

Oxalylchloride (0.1 mL, 1.18 mmol) together with DMF (0.1 mL) was added to a solution of ethyl 5-chloro-2-(difluoromethyl)-6-oxo-1,6-dihydropyridine-3-carboxylate (85.5 mg, 0.217 mmol) in DCM and the mixture was heated to 42° C. for 3 hours. No product could be detected and therefore another 0.1 mL (1.18 mmol) oxalylchloride was added and the stirring was continued at 42° C. over night. He reaction was diluted with DCM and quenched by poring it on an ice/water mixture. The phases was separated and the organic phase was washed with NaHCO₃ (aq, sat) and Brine. The combined water phase was extracted with DCM and the combined organic phase was filtered through a phase separator and evaporated. The residue was co-concentrated twice with DCM to give ethyl 5,6-dichloro-2-(difluoromethyl)nicotinate as a yellow oil which was used in the next step without further purification. Yield: 113.5 mg (51%).

### (d) tert-butyl 4-[(benzylsulfonyl)carbamoyl]piperidine-1-carboxylate

Triethylamine (591 g, 5840 mmol) was added to a stirred suspension of 1-(tert-butoxycarbonyl)piperidine-4-carboxylic acid (448 g, 1954 mmol), LiCl (23.1 g, 545 mmol) and TBTU (657 g, 2046 mmol) in THF (3000 mL) under an atmosphere of nitrogen at r.t. A solution of 1-phenylmethanesulfonamide (352 g in 1300 mL THF, 2056 mmol) was added after 1.5 hours and the stirring was continued over night. The solvent was removed in vacuo to give a thick grey-beige slurry (volume about 2500 mL). EtOAc (3500 mL) was added followed by an aqueous solution of HCl (1960 mL 3.6 M HCl and 1960 mL water). The water phase was removed and the organic phase was washed with 2×1500 mL 1 M HCl. The organic phase was cooled to 0° C. which gave a precipitate of HOBT that was filtered off. Most of the solvent was removed in vacuo to give a thick grey-white slurry. EtOH (50%, 4000 mL) was added and the slurry was stirred for 1.5 hours. The precipitated product was filtered off, washed with 50% EtOH (500 mL+2×1500 mL) and dried in a vacuum oven at 25° C. to give tert-butyl 4-[(benzylsulfonyl)carbamoyl]piperidine-1-carboxylate as a white solid. Yield 584 g (78%).

### (e) N-(benzylsulfonyl)piperidine 4-carboxamide

tert-Butyl 4-[(benzylsulfonyl)carbamoyl]piperidine-1-carboxylate (583 g, 1524 mmol) was suspended in formic acid (3000 mL) under a nitrogen atmosphere and the reaction was stirred for 20 minutes. The reaction was foaming due to the gas evolution and formic acid (500 mL) was used to wash down the foam from the reaction vessel walls. After 2 hours the foaming had stopped and the reaction was clear with a few solids left. The reaction was stirred over night and 2500 mL of formic acid was removed in vacuo. Water (1000 mL) was added and the reaction was filtered. The clear solution was evaporated and water (3000 mL) was added. A saturated ammonium hydroxide solution in water was used (totally 390 mL was added and the pH was going from 3.10 to 6.10) to neutralize the acidic solution and at the endpoint (pH=6.10) a heavy precipitate of the product was formed. The mixture was stirred over night and the precipitate was filtered off and washed with water (1000 mL). Drying in a vacuum oven at 25° C. gave N-(benzylsulfonyl)piperidine-4-carboxamide as a white powder. Yield 372.4 g (87%).

### (f) Ethyl 6-(4-{[(benzylsulfonyl)amino]carbonyl}piperidin-1-yl)-5-chloro-2-(difluoromethyl)nicotinate

TEA (149 μL, 1.07 mmol) was added to a solution of ethyl 5,6-dichloro-2-(difluoromethyl)nicotinate (113 mg, 0.214 mmol)) and N-(benzylsulfonyl)piperidine-4-carboxamide (66 mg, 0.24 mmol) in CH₃CN (3 mL) and water (2 mL) The reaction was heated in a single node microwave at 120° C. over 20 minutes. The solvents were removed in vacuo and the crude mixture was diluted with DCM and washed twice with 1% KHSO₄ (aq). The combined aqueous phase was extracted with DCM and the combined organic phases were passed through a phase separator followed by removal of solvents in vacuo.

The crude product was purified using preparative HPLC on a (Kromasil C8, 50.8×300 mm), the compound was loaded onto the column using 5% acetonitrile/aqueous NH₄OAc buffer pH 7 and then eluted using a gradient of 30-100% acetonitrile/aqueous NH₄OAc buffer pH 3.

Product-fractions were combined and the solvent was removed in vacuo, and triturated with DCM followed by filtration. The solvents were removed in vacuo to give ethyl 6-(4-{[(benzylsulfonyl)amino]carbonyl}piperidin-1-yl)-5-chloro-2-(difluoromethyl)nicotinate as a white solid. Yield: 13 mg (11%).

¹H NMR (400 MHz, CDCl₃) δ 1.38 (3H, t, J=7.1 Hz), 1.73-1.91 (4H, m), 2.27-2.42 (1H, m), 2.87-3.05 (2H, m), 4.19-4.30 (2H, m), 4.30-4.41 (2H, m), 4.67 (2H, s), 7.29-7.43 (5H, m), 7.48-7.54 (1H, m), 8.16 (1H, s)

### Example 149

### Ethyl 6-(4-{[(benzylsulfonyl)amino]carbonyl}piperidin-1-yl)-5-cyano-2-(difluoromethyl)nicotinate

### (a) ethyl 5-cyano-2-(difluoromethyl)-6-oxo-4,6-dihydropyridine-3-carboxylate

1,1-Dimethoxy-N,N-dimethylmethanamine (4.8 mL, 36.1 mmol) was added to ethyl 4,4-difluoro-3-oxobutanoate (5.0 g, 30.1 mmol) (exothermic reaction). The orange solution was stirred at r.t over night, concentrated and co-evaporated with toluene. The residue was taken up in EtOH (99.5%, 10 mL) to give a red solution. Freshly prepared NaOEt (1M, 30 mL) was added to a solution of 2-cyanoacetamide (2.53 g, 30.1 mmol) in EtOH (99.5%, 30 mL) and the reaction was stirred at r.t for 1 hour and the above red solution was added dropwise. The red suspension formed was stirred over night and HOAc (6 mL) was added and the solution became clear. The solution was concentrated and slurried in water (50 mL) and stirred for 1 hour after which the precipitate was filtered off and dried in air to give ethyl 5-cyano-2-(difluoromethyl)-6-oxo-1,6-dihydropyridine-3-carboxylate as a brown solid. Yield-3.03 g (41%).

¹H-NMR (400 MHz, DMSO-d₆) δ 1.30 (3H, t, J=7.2 Hz), 4.28 (2H, q, J=7.2 Hz), 7.48 (H, t, J=52.5 Hz, F-coupling), 8.58 (1H, s).

### (b) ethyl 6-chloro-5-cyano-2-(difluoromethyl)nicotinate

Oxalylchloride (5.3 mL, 62.6 mmol) followed by DMF (0.097 mL) was added to a slurry of ethyl 5-cyano-2-(difluoromethyl)-6-oxo-1,6-dihydropyridine-3-carboxylate (3 g, 12.5 mmol) in DCM (45 mL) and the reaction was heated to 50° C. for a few hours, more oxalylchloride was added (1 mL, 11.8 mmol) and DMF (0.2 mL) was added twice with a few hours inbetween and the heating was continued at reflux over night. The reaction mixture was evaporated and the residue was taken up in DCM and washed with water and NaHCO₃ (aq, sat), The aqueous phase was extracted with DCM (twice) and the combined organic phase was concentrated and purified by flash chromatography (Horizon, Eluent a gradient of Heptane/EtOAc 7/1 to 100% EtOAc was used) to give ethyl 6-chloro-5-cyano-2-(difluoromethyl)nicotinate as a yellow oil. Yield: 2.0 g (60%).

¹H-NMR (400 MHz, DMSO-d₆) δ 1.34 (3H, t, J=7.0), 4.37 (2H, q, J=7.0 Hz), 7.46 (1H, t, J=53.2 Hz), 8.99 (1H, s).

### (c) ethyl 6-(4-{[(benzylsulfonyl)amino]carbonyl}piperidin-1-yl)-5-cyano-2-(difluoromethyl)nicotinate

TEA (0.4 mL, 2.89 mmol) was added to a solution of ethyl 6-chloro-5-cyano-2-(difluoromethyl)nicotinate (200 mg, 0.721 mmol) and N-(benzylsulfonyl)piperidine-4-carboxamide (224 mg, 0.793 mmol) in water (2.5 mL) and EtOH (2 mL). The mixture was heated in a single-node microwave oven at 120° C. for 20 minutes, The solvents were evaporated and the residue was taken up in DCM and washed with 1% KHSO₄ (twice). The combined aqueous phase was extracted with DCM (twice) and the combined organic phase was filtered through a phase separator and concentrated. Purification by HPLC (Kromasil C8, Eluent: A gradient of 40% CH₃CN to 100% CH₃CN/(50 mM HCOOH and 50 mM NH₄OOCH, pH=3) gave ethyl 6-(4-{[(benzylsulfonyl)amino]carbonyl}piperidin-1-yl)-5-cyano-2-(difluoromethyl)nicotinate as a white solid. Yield 250 mg (68%).

1H NMR (400 MHz, DMSO-d₆) δ 1.31 (3H, t, J=7.4 Hz), 1.73-1.59 (2H, m), 1.91-1.81 (2H, m), 2.61 (1H, m), 3.27-3.15 (2H, m), 4.28 (2H, q, I=7.4 Hz), 4.61-4.51 (2H, m), 4.69 (2H, s), 7.33-7.22 (2H, m), 7.44-7.34 (3H, m), 7.53 (1H, s), 8.50 (1H, s), 11.61 (1H, s)

### Example 150

### Ethyl 6-(4-{[(benzylsulfonyl)amino]carbonyl}piperidin-1-yl)-5-cyano-2-(trifluoromethyl)nicotinate

### (a) ethyl 6-chloro-5-cyano-2-(trifluoromethyl)nicotinate

Oxalylchloride (12.20 g, 96.1 mmol) and DMF (0.744 mL) were added to a solution of ethyl 5-cyano-6-oxo-2-(trifluoromethyl)-1,6-dihydropyridine-3-carboxylate (5 g, 19.22 mmol) (prepared essentially according to the Method Described in Mosti, L et al, Farmaco, Vol 47, No 4, 1992, pp 427-437) and the reaction was heated to 50° C. over night. The reaction was evaporated and the crude was dissolved in EtOAc and water. The phases was separated and the organic phase was washed with Brine and NaHCO₃ (aq, sat). The aqueous phase was extracted with EtOAc (3 times) and the combined organic phase was dried (Na₂CO₃), filtered 0.25 and concentrated to give ethyl 6-chloro-5-cyano-2-(trifluoromethyl)nicotinate as a brown solid which was used without further purification. Yield: 5.206 g (95%/0).

¹H NMR (400 MHz, DMSO-d₆) δ 1.31 (t, J=7.2 Hz, 3H), 4.38 (q, J=6.9 Hz, 2H), 9.07 (s, 1H)

### (b) Ethyl 6-(4-{[(benzylsulfonyl)amino]carbonyl}piperidin-1-yl)-5-cyano-2-(trifluoromethyl)nicotinate

TEA (142 mg, 1.41 mmol) was added to a solution of ethyl 6-chloro-5-cyano-2-(trifluoromethyl)nicotinate (140 mg, 0.352 mmol) and N-(benzylsulfonyl)piperidine-4-carboxamide (109 mg, 0.387 mmol) in water (2 mL) and EtOH (2.5 mL).). The mixture was heated in a single-node microwave oven at 120° C. for 20 minutes, The solvents were evaporated and the residue was taken up in DCM and washed with 1% KHSO₄ (twice). The combined aqueous phase was extracted with DCM (twice) and the combined organic phase was filtered through a phase separator and concentrated. Purification by HPLC (Kromasil C8, Eluent: A gradient of 30% CH₃CN to 100% CH₃CN/(50 mM HCOOH and 50 mM NH₄OOCH, pH=3) gave ethyl 6-(4-{[(benzylsulfonyl)amino]carbonyl}piperidin-1-yl)-5-cyano-2-(trifluoromethyl)nicotinate as a white solid. Yield: 107 mg (58%).

¹H NMR (400 MHz, DMSO-d₆) δ 1.29 (3K, t, J=7.5 Hz), 1.74-1.58 (2H, m), 1.91-1.79 (2H, m), 2.65-2.54 (1H, m), 3.27-3.15 (2H, m), 4.28 (2H, q, J=7.5 Hz), 4.55-4.46 (2H, m), 4.68 (2H, s), 7.33-7.23 (2H, m), 7.47-7.35 (3H, m), 8.54 (1H, s), 11.61 (1H, s).

### Example 151

### Ethyl 6-(3-{[(benzylsulfonyl)amino]carbonyl}azetidin-1-yl)-5-cyano-2-(difluoromethyl)nicotinate

### (a) 1-(tert-butoxycarbonyl)azetidine-3-carboxylic acid

(Boc)₂O (25.535 g, 117 mmol) dissolved in MeOH (70 mL) was added dropwise during 20 minutes to a stirred slurry of azetidine-3-carboxylic acid (10.11 g, 100 mmol) and Et₃N (27.8 mL, 200 mmol) in MeOH (105 mL) at r.t (mildly exothermic reaction) and the mixture was stirred over night (18 hours). The reaction was evaporated to dryness and THF (120 mL) was added and evaporated to give crude 1-(tert-butoxycarbonyl)azetidine-3-carboxylic acid which was used without further purification in the next step. Yield: 25.89 g (128%)

¹H NMR (400 MHz, CDCl₃) δ 1.43 (9H, s), 3.21-3.34 (1H, m), 4.00-4.13 (4H, m).

### (b) tert-butyl 3-[(benzylsulfonyl)carbamoyl]azetidine-1-carboxylate

TBTU (33.71 g, 105 mmol) and TEA (30.3 g, 300 mmol) was added to a solution of 1-(tert-butoxycarbonyl)azetidine-3-carboxylic acid from above (25.89 g, assumed to contain 100 mmol) and the reaction was stirred at r.t for 30 minutes. 1-phenylmethanesulfonamide (17.97 g, 105 mmol) and LiCl (1.844 g, 43.5 mmol) was added and the stirring was continued at r.t over night (23 hours). The reaction was concentrated to about ⅓ was left and EtOAc (500 mL) was added and the organic phase was washed with 2 M HCl (1×150 mL, 2×50 mL), water (2×50 mL). Drying (MgSO₄), filtration and evaporation of the solvent gave a brown powder (48.6 g). The powder was slurried in 150 mL TBME and stirred 3 hours. The solids was filtered off and washed with TBME (40 mL). This procedure was repeated twice with 100 mL TBME (washing with 25 □L) to give a brownish powder (33 g) still containing some HOBT. The powder was dissolved in about 100 mL warm EtOH and water (130 mL) was added to induce a crystallisation of the product. The crystals was filtered off and dried to give pure tert-butyl 3-[(benzylsulfonyl)carbamoyl]azetidine-1-carboxylate as an off white powder. Yield: 25.4 g (71%).

¹H NMR (400 MHz, DMSO-d₆) δ 1.39 (9H, s), 3.30 (1H, m, overlapping with the water signal in DMSO), 3.78-3.95 (4H, m), 4.73 (2H, s), 7.28-7.34 (2H, m), 7.36-7.41 (3H, m), 11.71 (1H, br s).

MS ^{m}/_{z}: 353 (M−1).

### (c) N-(benzylsulfonyl)azetidine-3-carboxamide

tert-butyl 3-[(benzylsulfonyl)carbamoyl]azetidine-1-carboxylate (25.4 g, 71.7 mmol) was added to HCOOH (300 mL) at r.t and the reaction was stirred over night (22 hours). The formic acid was removed in vacuo, water (40 mL) was added and removed in vacuo. Water (130 mL) was added to the residue followed by NH₄OH (aq) until pH reached 7.4 when a crystallization started. The crystals was filtered off and dried to give pure N-(benzylsulfonyl)azetidine-3-carboxamide as a white solid. Yield 15.73 g (86%).

¹H NMR (400 MHz, DMSO-d₆) δ 3.22 (1H, m), 3.87-3.96 (4H, m), 4.28 (2H, s), 7.20-7.32 (5H, m).

MS ^{m}/_{z}: 255 (M+1)

### (d) Ethyl 6-(3-{[(benzylsulfonyl)amino]carbonyl}azetidin-1-yl)-5-cyano-2-(difluoromethyl)nicotinate

TEA (291 mg, 2.88 mmol) was added to a solution of ethyl 6-chloro-5-cyano-2-(difluoromethyl)nicotinate (200 mg, 0.721 mmol) and N-(benzylsulfonyl)azetidine-3-carboxamide (201 mg, 0.793 mmol) in water (2 mL) and EtOH (2.5 mL). The mixture was heated in a single-node microwave oven at 120° C. for 20 minutes, The solvents were evaporated and the residue was taken up in DCM and washed with 1% KHSO₄ (twice). The combined aqueous phase was extracted with DCM (twice) and the combined organic phase was filtered through a phase separator and concentrated. Purification by HPLC (Kromasil C8, Eluent: A gradient of 40% CH₃CN to 100% CH₃CN/(50 mM HCOOH and 50 mM NH₄OOCH, pH=3) gave ethyl 6-(3-{[(benzylsulfonyl)amino]carbonyl}azetidin-1-yl)-5-cyano-2-(difluoromethyl)nicotinate as a white solid. Yield 264 mg (72%).

¹H NMR (400 MHz, DMSO-d₆) d 1.30 (3H, t, J=7.3 Hz), 3.64-3.53 (1H, m), 4.27 (2H, q, J=6.9 Hz), 4.53-4.31 (4H, m), 4.75 (2H, s), 7.40-7.30 (5H, m), 7.40 (1H, t, J=53.6 Hz), 8.47 (1H, s), 11.81 (1H, s)

MS ^{m}/_{z}: 478 (M+1)

### Example 152

### Ethyl 6-(3-{[(benzylsulfonyl)amino]carbonyl}azetidin-1-yl)-5-cyano-2-(trifluoromethyl)nicotinate

### (a) ethyl 6-chloro-5-cyano-2-(trifluoromethyl)nicotinate

Oxalylchloride (8.13 mL, 96.1 mmol) and DMF (0.744 mL, 9.61 mmol) were added to a solution of ethyl 5-cyano-6-oxo-2-(trifluoromethyl)-1,6-dihydropyridine-3-carboxylate (5.0 g, 19.22 mmol, prepared essentially according to the procedure described by Mosti L, et. al. Farmaco, Vol 47, No 4, 1992, pp. 427-437) and the reaction was heated to reflux over night. The solvent was evaporated and the residue was dissolved in EtOAc/water. The phases were separated and the organic phase was washed with Brine and NaHCO₃ (aq) (twice). The aqueous phase was extracted with EtOAc (three times) and the combined organic phases was dried (Na₂CO₃), filtered and concentrated to give ethyl 6-chloro-5-cyano-2-(trifluoromethyl)nicotinate which was used without further purification. Yield: 5.21 g (95%).

¹H NMR (400 MHz, DMSO-d₆) δ 1.31 (3H, t, J=7 Hz), 4.38 (21H, q, J=7 Hz), 9.07 (1H, s).

### (b) Ethyl 6-(3-{[(benzylsulfonyl)amino]carbonyl}azetidin-1-yl)-5-cyano-2-(trifluoromethyl)nicotinate

TEA (142 mg, 1.41 mmol) was added to a solution of ethyl 6-chloro-5-cyano-2-(trifluoromethyl)nicotinate (140 mg, 0.352 mmol) and N-(benzylsulfonyl)azetidine-3-carboxamide (98.4 mg, 0.387 mmol) in water (2 mL) and EtOH (2.5 mL). The mixture was heated in a single-node microwave oven at 120° C. for 20 minutes. The reaction was filtered to remove a precipitate and the solvents were evaporated. The residue was taken up in DCM and washed with 1% KHSO₄ (twice). The combined aqueous phase was extracted with DCM (twice) and the combined organic phase was filtered through a phase separator and concentrated. Purification by HPLC (Kromasil C8, Eluent: A gradient of 30% CH₃CN to 100% CH₃CN/(0.1% HCOOH (aq)) gave ethyl 6-(3-{[benzylsulfonyl)amino]carbonyl}azetidin-1-yl)-5-cyano-2-(difluoromethyl)nicotinate as a white solid. Yield 102 mg (58%).

¹H NMR (400 MHz, DMSO-d₆) δ 1.28 (31 t, J=7.3 Hz), 3.63-3.52 (1H, m), 4.27 (2H, q, J=7.3 Hz), 4.52-4.31 (4H, m), 4.74 (2H, s), 8.50 (1H, s), 11.80 (1H, s).

MS ^{m}/_{z}: 496 (M+1)

### Example 153

### Ethyl 6-(4-{[(benzylsulfonyl)amino]carbonyl}piperidin-1-yl)-5-cyano-2-(fluoromethyl)nicotinate

### (a) ethyl 5-cyano-2-(fluoromethyl)-4-oxo-1,6-dihydropyridine-3-carboxylate

1,1-dimethoxy-N,N-dimethylmethanamine (4.83 g, 40.5 mmol) was added to ethyl 4-fluoro-3-oxobutanoate (5.0 g, 33.75 mmol) at r.t (exothermic reaction) and the mixture was stirred over night, concentrated and co-evaporated with toluene. EtOH (99.5%, 10 mL) was added to give a red solution. Freshly prepared sodium ethoxide 1M solution (34.5 mL, 2.35 g, 34.5 mmol) was added to a solution of 2-cyanoacetamide (3.12 g, 37.13 mmol) in EtOH (99.5%, 30 mL) and after stirring at r.t for 35 minutes the red solution from above was added dropwise and the stirring continued over night. AcOH (6 mL) was carefully added (exothermic reaction) and the precipitate formed was filtered and washed with diethyl ester. Drying afforded ethyl 5-cyano-2-(fluoromethyl)-6-oxo-1,6-dihydropyridine-3-carboxylate as a beige solid. Yield 4.42 g (56%).

¹H NMR (400 MHz, DMSO-d₆) δ 1.24 (3H, t, J=7.2 Hz), 4.12 (2H, q, J=6.9 Hz), 5.42 (2H, d, J=47.5 Hz), 7.96 (1H, s).

MS ^{m}/_{z}: 225 (M+1).

### (b) ethyl 6-chloro-5-cyano-2-(fluoromethyl)nicotinate

Oxalylchloride (5.49 mL, 64.9 mmol) and DMF (0.5 mL, 6.5 mmol) were added to a solution of ethyl 5-cyano-2-(fluoromethyl)-6-oxo-1,6-dihydropyridine-3-carboxylate (3.0 g, 12.98 mmol) in DCM (120 mL) and the mixture was heated to reflux for 6 hours. The solvent was evaporated and the residue was dissolved in EtOAc/water. The phases were separated and the organic phase was washed with Brine and NaHCO₃ (aq). The aqueous phase was extracted with EtOAc (twice) and the combined organic phase was concentrated to give ethyl 6-chloro-5-cyano-2-(fluoromethyl)nicotinate as a beige solid which was used without further purification Yield: 2.92 g (90%).

¹H NMR (400 MHz, DMSO-d₆) δ 1.33 (t, J=7.1 Hz, 3H), 4.34 (q, J=7.1 Hz, 2H), 5.88 (s, 1H), 5.77 (s, 1H), 8.89 (s, 1H)

MS ^{m}/_{z}: 243 (M+1)

### (c) Ethyl 6-(4-{[(benzylsulfonyl)amino]carbonyl}piperidin-1-yl)-5-cyano-2-(fluoromethyl)nicotinate

TEA (326 mg, 3.23 mmol) was added to a solution of ethyl 6-chloro-5-cyano-2-(fluoromethyl)nicotinate (200 mg, 0.81 mmol) and N-(benzylsulfonyl)piperidine-4-carboxamide (251 mg, 0.89 mmol) in CH₃CN (1.5 mL) and 95% EtOH (2.5 mL). The mixture was heated in a single-node microwave oven at 120° C. for 20 minutes. The solvent was evaporated and the residue was taken up in DCM and washed with 1% KHSO₄ (twice). The combined aqueous phase was extracted with DCM and the combined organic phase was filtered through a phase separator and concentrated. Purification by HPLC (Kromasil C8, Eluent: A gradient of 40% CH₃CN to 100% CH₃CN/(0.1% HCOOH (aq)) gave ethyl 6-(4{[benzylsulfonyl)amino]carbonyl}piperidin-1-yl)-5-cyano-2-(fluoromethyl)nicotinaten as a beige solid. Yield: 257 mg (65%).

¹H NMR (400 MHz, DMSO-d₆) δ 1.30 (3H, t, J=7.2 Hz), 1.71-1.56 (2H, m), 1.89-1.79 (2H, m), 2.65-2.54 (1H, m), 3.24-3.12 (2H, m), 4.25 (2H, q, J=7.2 Hz), 4.64-4.53 (2H, m), 4.68 (2H, s), 5.63 (1H, s), 5.75 (1H, s), 7.33-7.23 (2H, m), 7.44-7.34 (3H, m), 8.40 (1H, s), 11.60 (1H, s).

MS ^{m}/_{z}: 489 (M+1)

### Example 154

### Ethyl 6-(3-{[(benzylsulfonyl)amino]carbonyl}azetidin-1-yl)-5-cyano-2-(fluoromethyl)nicotinate

TEA (326 mg, 3.23 mmol) was added to a solution of ethyl 6-chloro-5-cyano-2-(fluoromethyl)nicotinate (200 mg, 0.81 mmol) and N-(benzylsulfonyl)azetidine-3-carboxamide (225 mg, 0.89 mmol) in CH₃CN (1.5 mL) and 95% EtOH (2.5 mL). The mixture was heated in a single-node microwave oven at 120° C. for 20 minutes. The solvent was evaporated and the residue was taken up in DCM and washed with 1% KHSO4. The combined aqueous phase was extracted with DCM and the combined organic phase was filtered through a phase separator and concentrated. Purification by HPLC (Kromasil C8, Eluent: A gradient of 40% CH₃CN to 100% CH₃CN/(0.1% HCOOH (aq)) gave ethyl 6-(3-{[(benzylsulfonyl)amino]carbonyl}azetidin-1-yl)-5-cyano-2-(fluoromethyl)nicotinate as a beige solid. Yield 221 mg (59%)

¹H NMR (400 MHz, DMSO-d₆) δ 1.29 (3H, t, J=7.2 Hz), 3.62-3.51 (1H, m), 4.24 (2H, q, J=7.2 Hz), 4.39-4.29 (2H, m), 4.51-4.39 (2H, m), 4.74 (2H, s), 5.61 (1H, s), 5.73 (1H, s), 7.42-7.29 (5H, m), 8.38 (1H, s), 11.81 (1H, s).

MS ^{m}/_{z}: 461 (M+1).

### Example 155

### Ethyl 5-cyano-2-(difluoromethyl)-6-{4-[({[(4-methylcyclohexyl)methyl]sulfonyl}amino)carbonyl]piperidin-1-yl}nicotinate

### (a) 1-[3-cyano-6-(difluoromethyl)-5-(ethoxycarbonyl)pyridin-2-yl]piperidine-4-carboxylic acid

TEA (423 mg, 4.18 mmol) was added to a solution of ethyl 6-chloro-5-cyano-2-(difluoromethyl)nicotinate (290 mg, 1.05 mmol) and piperidine-4-carboxylic acid (148 mg, 1.15 mmol) in water/EtOH (4.5 mL). The mixture was heated in a single-node microwave oven at 120° C. for 10 minutes. The solvent was evaporated and the residue was taken up in DCM and washed with 1% KHSO₄. The combined aqueous phase was extracted with DCM (twice) and the combined organic phase was filtered through a phase separator and concentrated to give 1-[3-cyano-6-(difluoromethyl)-5-(ethoxycarbonyl)pyridin-2-yl]piperidine-4-carboxylic acid as a white solid which was used without further purification. Yield: 356 mg (94%).

1H-NMR (400 MHz, CDCl₃) δ 1.39 (3H, t, J=7.2 Hz), 1.84-1.97 (2H, m), 2.08-2.17 (2H, m), 2.69-2.79 (1H, m), 3.37-3.47 (2H, m), 4.37 (2H, q, J=7.2 Hz), 4.61-4.70 (2H, m), 7.39 (1H, t, CHF₂), 8.43 (1H, s).

MS ^{m}/_{z}: 354 (M+1)

### (b) Ethyl 5-cyano-2-difluoromethyl)-6-{4-[({[(4-methylcyclohexyl)methyl]sulfonyl}amino)carbonyl]piperidin-1-yl}nicotinate

DIPEA (64 mg, 0.5 mmol) was added to a solution of 1-[3-cyano-6-(difluoromethyl)-5-(ethoxycarbonyl)pyridin-2-yl]piperidine-4-carboxylic (35.3 mg, 0.1 mmol) and TBTU (38.5 mg, 0.12 mmol) in DCM (5 mL) and the mixture was stirred for 30 min at r.t before 1-(4-methylcyclohexyl)methanesulfonamide (23 mg, 0.12 mmol) dissolved in DCM (1 mL) was added. The reaction was allowed to stir over night. LC-MS showed that starting material was left so more TBTU (19 mg, 0.06 mmol) and DIPEA (26 mg, 0.2 mmol) were added to the mixture and the stirring was continued for another 2 h. The reaction mixture was washed with 1% KHSO₄, the aqueous phase was extracted with DCM (1 ml) and the combined organic phases passed through a phase separator and evaporated in vacuum centrifuge. The crude product obtained was purified by HPLC (Kromasil C₈, using a gradient of 20% to 100% CH₃CN/0.2% HOAc (aq)) to give ethyl 5-cyano-2-(difluoromethyl)-6-{4-[({[(4-methylcyclohexyl)methyl]sulfonyl}amino)carbonyl]piperidin-1-yl}nicotinate as a white solid. Yield: 22 mg (40%).

¹H NMR (400 MHz, CDCl₃-d₆) δ 8.61 (1H, s), 8.42 (1H, s), 7.36 (1H, t, J=54.3 Hz), 4.75 (2H, m), 4.35 (2H, q, J=7.3 Hz), 3.46 (1H, m), 3.38-3.22 (3H, m), 2.59 (1H, m), 2.30-2.18 (1H, m), 2.10-1.97 (2H, m), 1.96-1.79 (3H, m), 1.75-1.47 (6H, m), 1.37 (3H, t, J=7.2 Hz), 122-1.04 (2H, m), 0.92-0.83 (3H, m).

MS ^{m}/_{z}: 527 (M+1)

### Example 156

### Ethyl 5-cyano-2-(difluoromethyl)-6-[3-({[(2-fluorobenzyl)sulfonyl]amino}carbonyl)azetidin-1-yl]nicotinate

### (a) 1-[3-cyano-6-(difluoromethyl)-5-(ethoxycarbonyl)pyridin-2-yl]azetidine-3-carboxylic acid

TEA (423 mg, 4.18 mmol) was added to a solution of ethyl 6-chloro-5-cyano-2-(difluoromethyl)nicotinate (290 mg, 1.05 mmol) and azetidine-3-carboxylic acid (116 mg, 1.15 mmol) in 95% EtOH (4.5 mL). The mixture was heated in a single-node microwave oven at 120° C. for 10 minutes. The solvent was evaporated and the residue was taken up in DCM and washed with 1% KHSO₄. The combined aqueous phase was extracted with DCM (twice) and the combined organic phase was filtered through a phase separator and concentrated to give 1-[3-cyano-6-(difluoromethyl)-5-(ethoxycarbonyl)pyridin-2-yl]azetidine-3-carboxylic acid as a white solid which was used without further purification. Yield: 359 mg (101%).

¹H-NMR (400 MHz, CDCl₃) δ 1.39 (3H, t, J=7.1 Hz), 3.62-3.72 (1H, m), 4.36 (2H, q, J=7.1 Hz), 4.63-4.75 (4H, m), 7.34 (1H, t, J=54.2 Hz, CHF₂), 8.36 (1H, s).

MS ^{m}/_{z}: 326 (M+1)

### (b) Ethyl 5-cyano-2-(difluoromethyl)-6-[3-({[(2-fluorobenzyl)sulfonyl]amino}carbonyl)azetidin-1-yl]nicotinate

DIEA (64 mg, 0.5 mmol) was added to a solution of 1-[3-cyano-6-(difluoromethyl)-5-(ethoxycarbonyl)pyridin-2-yl]azetidine-3-carboxylic acid (32.5 mg, 0.1 mmol) and TBTU (38.5 mg, 0.12 mmol) in DCM (5 mL) and the mixture was stirred for 30 min at r.t before 1-(2-fluorophenyl)methanesulfonamide (23 mg, 0.12 mmol) dissolved in DCM (1 mL) was added. The reaction was allowed to stir over night. LC-MS showed that starting material was left so more TBTU (19 mg, 0.06 mmol) and DIPEA (26 mg, 0.2 mmol) were added to the mixture and the stirring was continued for another 2 h. The reaction mixture was washed with 1% KHSO4, the aqueous phase was extracted with DCM (1 ml) and the combined organic phases passed through a phase separator and evaporated in vacuum centrifuge. The crude product obtained was purified by HPLC (Kromasil C₈, using a gradient of 20% to 100% CH₃CN/0.2% HOAc (aq)) to give ethyl 5-cyano-2-(difluoromethyl)-6-[3-({[(2-fluorobenzyl)sulfonyl]amino}carbonyl)azetidin-1-yl]nicotinate as a white solid. Yield: 42 mg (83%).

¹H NMR (400 MHz, CDCl₃-d₆) δ 1.38 (3H, t, J=7.1 Hz), 3.50-3.40 (1H, m), 4.35 (2H, q, J=7.2 Hz), 4.67-4.51 (4H, m), 4.72 (2H, s), 7.22-7.08 (2H, m), 7.46-7.34 (2H, m), 7.44 (1H, t, CHF₂), 8.35 (1H, s).

MS ^{m}/_{z}: 497 (M+1)

### Example 157

### Ethyl 5-cyano-2-(difluoromethyl)-6-[4-({[(2-fluorobenzyl)sulfonyl]amino}carbonyl)piperidin-1-yl]nicotinate

Prepared according to Method A′ from 1-[3-cyano-6-(difluoromethyl)-5-(ethoxycarbonyl)pyridin-2-yl]piperidine-4-carboxylic acid and 1-(2-fluorophenyl)methanesulfonamide to give ethyl 5-cyano-2-(difluoromethyl)-6-[4-({[(2-fluorobenzyl)sulfonyl]amino}carbonyl)piperidin-1-yl]nicotinate. Yield: 41 mg (78%).

1H NMR (600 MHz, DMSO-d₆) δ 1.28 (3H, t, J=6.8 Hz), 1.60-1.68 (2H, m), 1.85-1.90 (2H, m), 2.57-2.64 (1H, m), 3.17-3.24 (2H, m), 4.25 (2H, q, J=7.0 Hz), 4.53-4.58 (2H, m), 4.72 (2H, s), 7.20-7.26 (2H, m), 7.35-745 (2H, m), 7.37 (1H, t, J=54.1 Hz), 8.47 (1H, s)

MS ^{m}/_{z}: 525 (M+1)

### Example 158

### Ethyl 5-cyano-2-(difluoromethyl)-6-[4-({[(3-fluorobenzyl)sulfonyl]amino}carbonyl)piperidin-1-yl]nicotinate

Prepared according to Method A′ from 1-[3-cyano-6-(difluoromethyl)-5-(ethoxycarbonyl)pyridin-2-yl]piperidine-4-carboxylic acid and 1-(3-fluorophenyl)methanesulfonamide to give Ethyl 5-cyano-2-(difluoromethyl)-6-[4-({[(3-fluorobenzyl)sulfonyl]amino}carbonyl)piperidin-1-yl]nicotinate. Yield: 21 mg (40%).

¹H NMR (600 MHz, DMSO-d₆) δ 8.45 (1H, s), 7.35 (1H, t, J=53.5 Hz), 738-7.43 (1H, m), 7.16-7.22 (1H, m), 7.05-7.11 (2H, m), 4.69 (2H, s), 4.48-4.55 (2H, m), 4.24 (2H, q, J=7.1 Hz), 3.14-321 (2H, m), 2.53-2.58 (1H, m), 1.78-1.84 (2H, m), 1.56-1.65 (2H, m), 1.27 (3H, t, J=7.1 Hz)

MS ^{m}/_{z}: 525 (M+1)

### Example 159

### Ethyl 5-cyano-2-(difluoromethyl)-6-[4-({[(4-fluorobenzyl)sulfonyl]amino}carbonyl)piperidin-1-yl]nicotinate

Prepared according to Method A′ from 1-[3-cyano-6-(difluoromethyl)-5-(ethoxycarbonyl)pyridin-2-yl]piperidine-4-carboxylic acid and 1-(4-fluorophenyl)methanesulfonamide to give Ethyl 5-cyano-2-(difluoromethyl)-6-[4-({[(4-fluorobenzyl)sulfonyl]amino}carbonyl)piperidin-1-yl]nicotinate. Yield: 19 mg (36%).

¹H NMR (600 MHz, DMSO-d₆) δ 1.28 (3H, t, J=7.2 Hz), 1.58-1.67 (2H, m), 1:81-1.87 (2H, m), 3.15-3.22 (2H, m), 4.26 (2H, q, J=7.1 Hz), 4.51-4.58 (2H, m), 4.66 (2H, s), 7.19-7.23 (2H, m), 7.28-7.32 (2H, m), 7.37 (1H, t, J=54.1 Hz), 8.47 (1H, s)

Note! One H is hidden in the DMSO signal

MS ^{m}/_{z}: 525 (M+1)

### Example 160

### Ethyl 6-[4-({[(2-chlorobenzyl)sulfonyl]amino}carbonyl)piperidin-1-yl]-5-cyano-2-(difluoromethyl)nicotinate

Prepared according to Method A′ from 1-[3-cyano-6-(difluoromethyl)-5-(ethoxycarbonyl)pyridin-2-yl]piperidine-4-carboxylic acid and 1-(2-chlorophenyl)methanesulfonamide to give Ethyl 6-[4-({[(2-chlorobenzyl)sulfonyl]amino}carbonyl)piperidin 1-yl]-5-cyano-2-(difluoromethyl)nicotinate.

Yield: 36 mg (67%).

¹H NMR (600 MHz, DMSO-d₆) δ 1.28 (31H, t, J=7.2 Hz), 1.60-1.69 (2H, m), 1.86-1.92 (2H, m), 3.18-3.24 (2H, m), 4.25 (2H, q, J=7.0 Hz), 4.51-4.59 (2H, m), 4.81 (2H, s), 7.26-7.53 (5H, m), 8.47 (1H, s). Note! One H is hidden in the DMSO signal

MS ^{m}/_{z}: 541 (M+1)

### Example 161

### Ethyl 6-[4-({[(3-chlorobenzyl)sulfonyl]amino}carbonyl)piperidin-1-yl]-5-cyano-2-(difluoromethyl)nicotinate

Prepared according to Method A′ from 1-[3-cyano-6-(difluoromethyl)-5-(ethoxycarbonyl)pyridin-2-yl]piperidine-4-carboxylic acid and 1-(3-chlorophenyl)methanesulfonamide to give Ethyl 6-[4-({[(3-chlorobenzyl)sulfonyl]amino}carbonyl)piperidin-1-yl]-5-cyano-2-(difluoromethyl)nicotinate. Yield: 42 mg (78%).

¹H NMR (600 MHz, DMSO-d₆) δ 1.27 (3H, t, J=6.8 Hz), 1.57-1.65 (2H, m), 1.78-1.84 (2H, m), 2.53-2.59 (1H, m), 3.14-3.21 (2H, m), 4.24 (2H, q, J=6.9 Hz), 4.49-4.56 (2H, m), 4.68 (2H, s), 7.18-7.46 (5H, m), 8.46 (1H, s)

MS ^{m}/_{z}: 541 (M+1)

### Example 162

### Ethyl 6-[4-({[(4-chlorobenzyl)sulfonyl]amino}carbonyl)piperidin-1-yl]-5-cyano-2-(difluoromethyl)nicotinate

Prepared according to Method A′ from 1-[3-cyano-6-(difluoromethyl)-5-(ethoxycarbonyl)pyridin-2-yl]piperidine-4-carboxylic acid and 1-(4-chlorophenyl)methanesulfonamide to give Ethyl 6-[4-({[(4-chlorobenzyl)sulfonyl]amino}carbonyl)piperidin-1-yl]-5-cyano-2-(difluoromethyl)nicotinate. Yield: 33 mg (61%).

¹H NMR (400 MHz, DMSO-d₆) δ 1.31 (3H, t, J=7.2 Hz), 1.58-1.72 (21H, m), 1.82-1.92 (2H, m), 2.56-2.68 (1H, m), 3.16-3.26 (2H, m), 4.28 (2H, q, J=7.2 Hz), 4.52-4.61 (2H, m), 4.70 (2H, s), 7.28-7.35 (2H, m), 7.39 (1H, t, J=54.1 Hz), 7.44-7.51 (2H, m), 8.50 (1H, s), 11.64 (1H, s)

MS ^{m}/_{z}: 541 (M+1)

### Example 163

### Ethyl 5-cyano-2-(difluoromethyl)-6-[4-({[(3-methylbenzyl)sulfonyl]amino}carbonyl)piperidin-1-yl]nicotinate

Prepared according to Method A′ from 1-[3-cyano-6-(difluoromethyl)-5-(ethoxycarbonyl)pyridin-2-yl]piperidine-4-carboxylic acid and 1-(3-methylphenyl)methanesulfonamide to give ethyl 5-cyano-2-(difluoromethyl)-6-[4-({[(3-methylbenzyl)sulfonyl]amino}carbonyl)piperidin-1-yl]nicotinate. Yield: 17 mg (32%).

¹H NMR (400 MHz, DMSO-d₆) δ 1.31 (3H, t, J=7.3 Hz), 1.59-1.73 (2H, m), 1.79-1.89 (2H, m), 2.29 (3H, s), 2.54-2.64 (1H, m), 3.16-3.26 (2H, m), 4.28 (2H, q, J=7.4 Hz), 4.53-4.61 (2H, m), 4.63 (2H, s), 7.04-7.10 (2H, m), 7.16-7.22 (1H, m), 7.24-7.31 (1H, m), 7.39 (1H, t, J=53.9 Hz), 8.49 (1H, s), 11.59 (1H, s)

MS ^{m}/ₛ: 521 (M+1)

### Example 164

### Ethyl 5-cyano-2-(difluoromethyl)-6-[4-({[(4-methylbenzyl)sulfonyl]amino}carbonyl)piperidin-1-yl]nicotinate

Prepared according to Method A′ from 1-[3-cyano-6-(difluoromethyl)-5-(ethoxycarbonyl)pyridin-2-yl]piperidine-4-carboxylic acid and 1-(4-methylphenyl)methanesulfonamide to give ethyl 5-cyano-2-(difluoromethyl)-6-[4-({[(4-methylbenzyl)sulfonyl]amino}carbonyl)piperidin-1-yl]nicotinate. Yield: 19 mg (36%).

¹H NMR (600 MHz, DMSO-d₆) δ 1.27 (3H, t, J=7.2 Hz), 1.57-1.65 (2H, m), 1.79-1.85 (2H, m), 2.26 (3H, s), 3.14-3.21 (2H, m), 4.24 (2H, q, J=7.3 Hz), 4.50-4.56 (2H, m), 4.58 (2H, s), 7.10-7.18 (4H, m), 7.36 (1H, t, J=53.4 Hz), 8.46 (1H, s). Note! One H is hidden in the DMSO signal.

MS ^{m}/_{z}: 521 (M+1)

### Example 165

### Ethyl 5-cyano-6-[4-({[(2,4-dichlorobenzyl)sulfonyl]amino}carbonyl)piperidin-1-yl]-2-(difluoromethyl)nicotinate

Prepared according to Method A′ from 1-[3-cyano-6-(difluoromethyl)-5-(ethoxycarbonyl)pyridin-2-yl]piperidine-4-carboxylic acid and 1-(2,4-dichlorophenyl)methanesulfonamide to give ethyl 5-cyano-6-[4-({[(2,4-dichlorobenzyl)sulfonyl]amino}carbonyl)piperidin-1-yl]-2-(difluoromethyl)nicotinate. Yield: 27 mg (47%).

¹H NMR (600 MHz, DMSO-d₆) δ 1.28 (3H, t, J=7.0 Hz), 1.59-1.68 (2H, m), 1.87-1.93 (2H, m), 2.54-2.60 (1H, m), 3.18-3.24 (2H, m), 4.26 (2H, q, J=6.8 Hz), 4.52-4.58 (2H, m), 4.81 (2H, s), 7.26-7.52 (3H, m), 7.69 (1H, s), 8.47 (1H, s)

MS^{m}/_{z}: 575 (M+1)

### Example 166

### Ethyl 5-cyano-2-(difluoromethyl)-6-[3-({[(3-fluorobenzyl)sulfonyl]amino}carbonyl)azetidin-1-yl]nicotinate

Prepared according to Method A′ from 1-[3-cyano-&(difluoromethyl)-5-(ethoxycarbonyl)pyridin-2-yl]azetidine-3-carboxylic acid and 1-(3-fluorophenyl)methanesulfonamide to give Ethyl 5-cyano-2-(difluoromethyl)-6-[3-({[(3-fluorobenzyl)sulfonyl]amino}carbonyl)azetidin-1-yl]nicotinate. Yield: 47 mg (95%).

¹H NMR (600 MHz, DMSO-d₆) δ 1.28 (3H, t, J=7.3 Hz), 3.51-3.59 (1H, m), 4.25 (2H, q, J=7.4 Hz), 4.26-4.51 (4H, m), 4.75 (2H, s), 7.12-7.22 (3H, m), 7.35-7.42 (1H, m), 7.37 (1H, t, J=53.2 Hz), 8.44 (1H, s)

MS ^{m}/_{z}: 497 (M+1)

### Example 167

### Ethyl 5-cyano-2-(difluoromethyl)-6-[3-({[(4-fluorobenzyl)sulfonyl]amino}carbonyl)azetidin-1-yl]nicotinate

Prepared according to Method A′ from 1-[3-cyano-6-(difluoromethyl)-5-(ethoxycarbonyl)pyridin-2-yl]azetidin-3-carboxylic acid and 1-(4 fluorophenyl)methanesulfonamide to give Ethyl 5-cyano-2-(difluoromethyl)-6-[3-({[(4-fluorobenzyl)sulfonyl]amino}carbonyl)azetidin-1-yl]nicotinate. Yield: 41 mg (83%).

¹H NMR (600 MHz, DMSO-d₆) δ 1.26 (3H, t, J=7.1 Hz), 3.49-3.57 (1H, m), 4.23 (2H, q, J=7.1 Hz), 4.26-4.50 (4H, m), 4.69 (2H, s), 7.12-7.19 (2H, m), 7.32-7.37 (2H, m), 7.36 (1H, t, J=54.2 Hz), 8.43 (1H, s)

MS ^{m}/_{z}: 497 (+1)

### Example 168

### Ethyl 6-[3-({[(2-chlorobenzyl)sulfonyl]amino}carbonyl)azetidin-1-yl]-5-cyano-2-(difluoromethyl)nicotinate

Prepared according to Method A′ from 1-[3-cyano-6-(difluoromethyl)-5-(ethoxycarbonyl)pyridin-2-yl]azetidine-3-carboxylic acid and 1-(2-chlorophenyl)methanesulfonamide to give ethyl 6-[3-({[(2-chlorobenzyl)sulfonyl]amino}carbonyl)azetidin-1-yl]-5-cyano-2-(difluoromethyl)nicotinate. Yield: 42 mg (82%).

¹H NMR (400 MHz, DMSO-d₆) δ 1.30 (3H, t, J=7.2 Hz), 3.58-3.68 (1H, m), 4.27 (2H, q, J=7.5 Hz), 4.36-4.57 (4H, m), 4.90 (2H, s), 7.35-7.46 (2H, m), 7.40 (1H, t, J=54.2 Hz), 7.47-7.56 (2H, m), 8.47 (1H, s), 12.03 (1H, s)

MS ^{m}/_{z}: 513 (M+1)

### Example 169

### Ethyl 6-[3-({[(3-chlorobenzyl)sulfonyl]amino}carbonyl)azetidin-1-yl]-5-cyano-2-(difluoromethyl)nicotinate

Prepared according to Method A′ from 1-[3-cyano-6-(difluoromethyl)-5-(ethoxycarbonyl)pyridin-2-yl]azetidine-3-carboxylic acid and 1-(3-chlorophenyl)methanesulfonamide to give ethyl 6-[3-({[(3-chlorobenzyl)sulfonyl]amino}carbonyl)azetidin-1-yl]-5-cyano-2-(difluoromethyl)nicotinate. Yield: 46 mg (90%).

¹H NMR (600 MHz, DMSO-d₆) δ 1.28 (3H, t, J=7.1 Hz), 3.51-3.59 (1H, m), 4.24 (2H, q, J=7.2 Hz), 4.25-4.54 (4H, m), 4.76 (2H, s), 7.26-7.30 (1H, m), 7.35-7.47 (4H, m), 8.44 (1H, s).

MS ^{m}/_{z}: 513 (M+1)

### Example 170

### Ethyl 6-[3-({[(4-chlorobenzyl)sulfonyl]amino}carbonyl)azetidin-1-yl]-5-cyano-2-(difluoromethyl)nicotinate

Prepared according to Method A′ from 1-[3-cyano-6-(difluoromethyl)-5-(ethoxycarbonyl)pyridin-2-yl]azetidine-3-carboxylic acid and 1-(4-chlorophenyl)methanesulfonamide to give ethyl 6-[3-({[(4-chlorobenzyl)sulfonyl]amino}carbonyl)azetidin-1-yl]-5-cyano-2-(difluoromethyl)nicotinate. Yield: 45 mg (88%).

¹H NMR (600 MHz, DMSO-d₆) δ 1.26 (3H, t, J=7.0 Hz), 3.50-3.57 (1H, m), 4.23 (2H, q, J=7.0 Hz), 4.27-4.50 (4H, m), 4.70 (2H, s), 7.30-7.34 (2H, m), 7.36 (1H, t, J=53.8 Hz), 7.38-7.43 (2H, m), 8.43 (1H, s).

MS ^{m}/_{z}: 513 (M+1)

### Example 171

### Ethyl 5-cyano-2-(difluoromethyl)-6-[3-({[(3-methylbenzyl)sulfonyl]amino}carbonyl)azetidin-1-yl]nicotinate

Prepared according to Method A′ from 1-[3-cyano-6-(difluoromethyl)-5-(ethoxycarbonyl)pyridin-2-yl]azetidine-3-carboxylic acid and 1-(3-methylphenyl)methanesulfonamide to give ethyl 5-cyano-2-(difluoromethyl)-6-[3-({[(3-methylbenzyl)sulfonyl]amino}carbonyl)azetidin-1-yl]nicotinate. Yield: 36 mg (73%).

¹H NMR (600 MHz, DMSO-d₆) δ 1.26 (3H, t, J=7.6 Hz), 2.22 (3H, s), 3.48-3.56 (1H, m), 4.23 (2H, q, J=7.0 Hz), 4.24-4.49 (4H, m), 4.64 (2H, s), 7.06-7.10 (21H, m), 7.12-7.16 (1H, m), 7.19-7.23 (1H, m), 7.36 (1H, t, J=54.9 Hz), 8.43 (1H, s)

MS ^{m}/_{z}: 493 (M+1)

### Example 172

### Ethyl 5-cyano-2-(difluoromethyl)-6-[3-({[(4-methylbenzyl)sulfonyl]amino}carbonyl)azetidin-1-yl]nicotinate

Prepared according to Method A′ from 1-[3-cyano-6-(difluoromethyl)-5-(ethoxycarbonyl)pyridin-2-yl]azetidine-3-carboxylic acid and 1-(4-methylphenyl)methanesulfonamide to give ethyl 5-cyano-2-(difluoromethyl)-6-[3-({[(4-methylbenzyl)sulfonyl]amino}carbonyl)azetidin-1-yl]nicotinate. Yield-31 mg (63%).

¹H NMR (600 MHz, DMSO-d₆) δ 1.26 (3H, t, J=6.9 Hz), 2.24 (3H, s), 3.47-3.55 (1H, m), 4.23 (2H, q, J=6.9 Hz), 4.26-4.49 (4H, m), 4.63 (2H, s), 7.11-7.19 (4H, m), 7.36 (1H, t, J=53.8 Hz), 8.43 (1H, s)

MS ^{m}/_{z}: 493 (M+1)

### Example 173

### Ethyl 5-cyano-6-[3-{[(2,4-dichlorobenzyl)sulfonyl]amino}carbonyl)azetidin-1-yl]-2-(difluoromethyl)nicotinate

Prepared according to Method A′ from 1-[3-cyano-6-(difluoromethyl)-5-(ethoxycarbonyl)pyridin-2-yl]azetidine-3-carboxylic acid and 1-(2,4-dichlorophenyl)methanesulfonamide to give ethyl 5-cyano-6-[3-({[(2,4-dichlorobenzyl)sulfonyl]amino}carbonyl)azetidin-1-yl]-2-(difluoromethyl)nicotinate. Yield: 7 mg (12%).

¹H NMR (600 MHz, DMSO-d₆) δ 1.26 (3H, t, J=7.3 Hz), 3.44-3.55 (1H, m), 4.23 (2H, q, J=7.3 Hz), 4.29-4.52 (4H, m), 4.67-4.83 (2H, m), 7.35 (1H, t, J=54.3 Hz), 7.38-7.50 (2H, m), 7.57-7.64 (1H, m), 8.42 (1H, s)

MS ^{m}/_{z}: 547 (M+1)

### Example 174

### Ethyl 5-cyano-2-(difluoromethyl)-6-{3-[({[(4-methylcyclohexyl)methyl]sulfonyl)amino}carbonyl]azetidin-1-yl}nicotinate

Prepared according to Method A′ from 1-[3-cyano-6-(difluoromethyl)-5-(ethoxycarbonyl)pyridin-2-yl]azetidine-3-carboxylic acid and 1-(4-methylcyclohexyl)methanesulfonamide to give ethyl 5-cyano-2-(difluoromethyl)-6-{3-[({[(4-methylcyclohexyl)methyl]sulfonyl}amino)carbonyl]azetidin-1-yl}nicotinate. Yield: 27 mg (55%).

¹H NMR (400 MHz, DMSO-d₆) δ 0.80-0.95 (3H, m), 1.01-1.20 (2H, m), 1.30 (3H, t, J=7.0 Hz), 1.40-1.58 (5H, m), 1.60-1.88 (2H, m), 2.04-2.15 (1H, m), 3.40-3.45 (2H, m), 3.59-3.69 (1H, m), 4.26 (2H, q, J=7.4 Hz), 4.33-4.58 (4H, m), 7.38 (1H, t, J=54.3 Hz), 8.46 (1H, s), 11.93 (1H, s)

MS ^{m}/_{z}: 499 (M+1)

### Example 175

### Ethyl 5-cyano-6-[3-({[(3-cyanophenyl)sulfonyl]amino}carbonyl)azetidin-1-yl]-2-(difluoromethyl)nicotinate

Prepared according to Method A′ from 1-[3-cyano-6-(difluoromethyl)-5-(ethoxycarbonyl)pyridin-2-yl]azetidine-3-carboxylic acid and 3-cyanobenzenesulfonamide to give ethyl 5-cyano-6-[3-({[(3-cyanophenyl)sulfonyl]amino}carbonyl)azetidin-1-yl]-2-(difluoromethyl)nicotinate. Yield: 47 mg (64%).

MS ^{m}/_{z}: 490 (M+1)

### Example 176

### Ethyl 5-cyano-6-[3-({[(4-cyanophenyl)sulfonyl]amino}carbonyl)azetidin-1-yl]-2-(difluoromethyl)nicotinate

Prepared according to Method A′ from 1-[3-cyano-6-(difluoromethyl)-5-(ethoxycarbonyl)pyridin-2-yl]azetidine-3-carboxylic acid and 4-cyanobenzenesulfonamide to give ethyl 5-cyano-6-[3-({[(4-cyanophenyl)sulfonyl]amino}carbonyl)azetidin 1-yl]-2-(difluoromethyl)nicotinate. Yield: 42 mg (57%).

MS ^{m}/_{z}: 490 (M+1)

### Example 177

### Ethyl 5-cyano-2-(difluoromethyl)-6-{3-[({[4-(trifluoromethoxy)phenyl]sulfonyl}amino)carbonyl]azetidin-1-yl}nicotinate

Prepared according to Method A′ from 1-[3-cyano-6-(difluoromethyl)-5-(ethoxycarbonyl)pyridin-2-yl]azetidine-3-carboxylic acid and 4-(trifluoromethoxy)benzenesulfonamide to give ethyl 5-cyano-2-(difluoromethyl)-6-{3-[({[4-(trifluoromethoxy)phenyl]sulfonyl}amino)carbonyl]azetidin-1-yl}nicotinate. Yield: 37 mg (45%).

MS ^{m}/_{z}: 549 (M+1)

### Example 178

### Ethyl 5-cyano-2-(difluoromethyl)-6-{3-[({[2-(trifluoromethoxy)phenyl]sulfonyl}amino)carbonyl]azetidin-1-yl}nicotinate

Prepared according to Method A′ from 1-[3-cyano-6-(difluoromethyl)-5-(ethoxycarbonyl)pyridin-2-yl]azetidine-3-carboxylic acid and 2-(trifluoromethoxy)benzenesulfonamide to give ethyl 5-cyano-2-(difluoromethyl)-6-{3-[({[2-(trifluoromethoxy)phenyl]sulfonyl}amino)carbonyl]azetidin-1-yl}nicotinate. Yield: 44 mg (53%).

MS ^{m}/_{z}: 549 (M+1)

### Example 179

### Ethyl 5-cyano-6-[3-({[(2-cyanobenzyl)sulfonyl]amino}carbonyl)azetidin-1-yl]-2-(difluoromethyl)nicotinate

Prepared according to Method A′ from 1-[3-cyano-6-(difluoromethyl)-5-(ethoxycarbonyl)pyridin-2-yl]azetidine-3-carboxylic acid and 1-(2-cyanophenyl)methanesulfonamide to give ethyl 5-cyano-6-[3-({[(2-cyanobenzyl)sulfonyl]amino}carbonyl)azetidin-1-yl]-2-(difluoromethyl)nicotinate. Yield: 52 mg (69%).

MS ^{m}/_{z}: 504 (M+1)

### Example 180

### Ethyl 5-cyano-2-(difluoromethyl)-6-(3-{[(2-naphthylsulfonyl)amino]carbonyl}azetidin-1-yl)nicotinate

Prepared according to Method A′ from 1-[3-cyano-6-(difluoromethyl)-5-(ethoxycarbonyl)pyridin-2-yl]azetidine-3-carboxylic acid and naphthalene-2-sulfonamide to give ethyl 5-cyano-2-(difluoromethyl)-6-(3-{[(2-naphthylsulfonyl)amino]carbonyl}azetidin-1-yl)nicotinate. Yield: 48 mg (62%).

MS ^{m}/_{z}: 515 (M+1)

### Example 181

### Ethyl 6-(3-{[(butylsulfonyl)amino]carbonyl}azetidin-1-yl)-5-cyano-2-(difluoromethyl)nicotinate

Prepared according to Method A′ from 1-[3-cyano-6-(difluoromethyl)-5-(ethoxycarbonyl)pyridin-2-yl]azetidine-3-carboxylic acid and butane-1-sulfonamide to give ethyl 6-(3-{[(butylsulfonyl)amino]carbonyl}azetidin-1-yl)-5-cyano-2-(difluoromethyl)nicotinate. Yield: 44 mg (65%).

MS ^{m}/_{z}: 445 (M+1)

### Example 182

### Ethyl 5-cyano-6-[4-({[(3-cyanophenyl)sulfonyl]amino}carbonyl)piperidin-1-yl]-2-(difluoromethyl)nicotinate

Prepared according to Method A′ from 1-[3-cyano-6-(difluoromethyl)-5-(ethoxycarbonyl)pyridin-2-yl]piperidine-4-carboxylic acid and 3-cyanobenzenesulfonamide to give ethyl 5-cyano-6-[4-({[(3-cyanophenyl)sulfonyl]amino}carbonyl)piperidin-1-yl]-2-(difluoromethyl)nicotinate. Yield: 9 mg (12%).

MS ^{m}/_{z}: 518 (M+1)

### Example 183

### Ethyl 5-cyano-6-[4-({[(4-cyanophenyl)sulfonyl]amino}carbonyl)piperidin-1-yl]-2-(difluoromethyl)nicotinate

Prepared according to Method A′ from 1-[3-cyano-6-(difluoromethyl)-5-(ethoxycarbonyl)pyridin-2-yl]piperidine-4-carboxylic acid and 4-cyanobenzenesulfonamide to give ethyl 5-cyano-6-[4-({[(4-cyanophenyl)sulfonyl]amino}carbonyl)piperidin-1-yl]-2-(difluoromethyl)nicotinate. Yield: 9 mg (12%).

MS ^{m}/_{z}: 518 (M+1)

### Example 184

### Ethyl 5-cyano-2-(difluoromethyl)-6-{4-[({[4-(trifluoromethoxy)phenyl]sulfonyl}amino)carbonyl]piperidin-1-yl}nicotinate

Prepared according to Method A′ from 1-[3-cyano-6-(difluoromethyl)-5-(ethoxycarbonyl)pyridin-2-yl]piperidine-4-carboxylic acid and 4-(trifluoromethoxy)benzenesulfonamide to give ethyl 5-cyano-2-(difluoromethyl)-6-{4-[({[4-(trifluoromethoxy)phenyl]sulfonyl}amino)carbonyl]piperidin-1-yl}nicotinate. Yield: 17 mg (19%).

MS ^{m}/_{z}: 577 (M+1)

### Example 185

### Ethyl 5-cyano-2-(difluoromethyl)-6-{4-[({[2-(trifluoromethoxy)phenyl]sulfonyl}amino)carbonyl]piperidin-1-yl}nicotinate

Prepared according to Method A′ from 1-[3-cyano-6-(difluoromethyl)-5-(ethoxycarbonyl)pyridin-2-yl]piperidine-4-carboxylic acid and 2-(trifluoromethoxy)benzenesulfonamide to give ethyl 5-cyano-2-(difluoromethyl)-6-{4-[({[2-(trifluoromethoxy)phenyl]sulfonyl}amino)carbonyl]piperidin-1-yl}nicotinate. Yield: 50 mg (58%).

MS ^{m}/_{z}: 577 (M+1)

### Example 186

### Ethyl 5-cyano-6-[4-({[(2-cyanobenzyl)sulfonyl]amino}carbonyl)piperidin-1-yl]-2-(difluoromethyl)nicotinate

Prepared according to Method A′ from 1-[3-cyano-6-(difluoromethyl)-5-(ethoxycarbonyl)pyridin-2-yl]piperidine-4-carboxylic acid and 2-(trifluoromethoxy)benzenesulfonamide to give ethyl 5-cyano-2-(difluoromethyl)-6-{4-[({[2-(trifluoromethoxy)phenyl]sulfonyl}amino)carbonyl]piperidin-1-yl}nicotinate. Yield: 14 mg (17%).

MS ^{m}/z=532 (M+1)

### Example 187

### Ethyl 5-cyano-2-(difluoromethyl)-6-(4-{[(2-naphthylsulfonyl)amino]carbonyl}piperidin-1-yl)nicotinate

Prepared according to Method A′ from 1-[3-cyano-6-(difluoromethyl)-5-(ethoxycarbonyl)pyridin-2-yl]piperidine-4-carboxylic acid and naphthalene-2-sulfonamide to give ethyl 5-cyano-2-(difluoromethyl)-6-(4-{[(2-naphthylsulfonyl)amino]carbonyl}piperidin-1-yl)nicotinate. Yield: 31 mg (38%).

MS^{m}/_{z}: 543 (M+1)

### Example 188

### Ethyl 6-(4-{[(butylsulfonyl)amino]carbonyl}piperidin-1-yl)-5-cyano-2-(difluoromethyl)nicotinate

Prepared according to Method A′ from 1-[3-cyano-6-(difluoromethyl)-5-(ethoxycarbonyl)pyridin-2-yl]piperidine-4-carboxylic acid and butane-1-sulfonamide to give ethyl 6-(4-{[(butylsulfonyl)amino]carbonyl}piperidin-1-yl)-5-cyano-2-(difluoromethyl)nicotinate. Yield: 36 mg (51%).

MS ^{m}/_{z}: 473 (M+1)

### Example 189

### Ethyl 6-(3-{2-[(benzylsulfonyl)amino]-2-oxoethyl}pyrrolidin-1-yl)-5-cyano-2-(trifluoromethyl)nicotinate

### (a) {1-[3-cyano-5-(ethoxycarbonyl)-6-(trifluoromethyl)pyridin-2-yl]pyrrolidin-3-yl}acetic acid

TEA (606 mg, 5.99 mmol) was added to a solution of ethyl 6-chloro-5-cyano-2-(trifluoromethyl)nicotinate (341 mg, 1.2 mmol) and pyrrolidin-3-ylacetic acid (209 mg, 1.62 mmol) in water/EtOH (4.5 mL). The mixture was heated in a single-node microwave oven at 120° C. for 20 minutes. The solvent was evaporated and the residue was taken up in DCM and washed with 1% KHSO₄. The combined aqueous phase was extracted with DCM and the combined organic phase was filtered through a phase separator and concentrated. Purification by HPLC (Kromasil C8, Eluent: A gradient of 5% CH₃CN to 100% CH₃CN/(0.2% HOAc (aq)) gave {1-[3-cyano-5-(ethoxycarbonyl)-6-(trifluoromethyl)pyridin-2-yl]pyrrolidin-3-yl}acetic acid as a white solid. Yield: 219 mg (49%).

¹H NMR (400 MHz, CDCl₃) δ 1.35 (3H, t, J=7.2 Hz), 1.85-1.68 (1H, m), 2.38-2.23 (1H, m), 2.64-2.47 (2H, m), 2.81-2.66 (1H, m), 3.57-3.40 (1H, m), 3.91-3.77 (1H, m), 4.08-3.97 (1H, m), 4.21-4.10 (1H, m), 4.33 (2H, q, J=7.3 Hz), 8.31 (1H, s).

MS ^{m}/_{z}: 371 (M+1)

### (b) ethyl 6-(3-{2-[(benzylsulfonyl)amino]-2-oxoethyl}pyrrolidin-1-yl)-5-cyano-2-(trifluoromethyl)nicotinate

Prepared according to Method B′ from {1-[3-cyano-5-(ethoxycarbonyl)-6-(trifluoromethyl)pyridin-2-yl]pyrrolidin-3-yl}acetic acid and 1-phenylmethanesulfonamide to give ethyl 6-(3-{2-[(benzylsulfonyl)amino]-2-oxoethyl}pyrrolidin-1-yl-5-cyano-2-(trifluoromethyl)nicotinate. Yield: 88 mg (84°/O).

¹H N (600 MHz, DMSO) d 1.26 (3H, t, J=7.3 Hz), 1.59-1.68 (1H, m), 2.09-2.17 (1H, m), 2.40-2.44 (2H, m), 3.64-3.77 (1H, m), 3.81-3.91 (1H, m), 3.94-4.06 (1H, m), 4.24 (2H, q, J=7.0 Hz), 4.68 (2H, s), 7.24-7.39 (5H, m), 8.45 (1H, s). Note! One H hidden in the DMSO peak and one H hidden in the HO peak

MS ^{m}/_{z}: 525 (M+1)

### Example 190

### Ethyl 5-cyano-[3-(2-oxo-2-{[(2-phenylethyl)sulfonyl]amino}ethyl)pyrrolidin-1-yl]-2-(trifluoromethyl)nicotinate

Prepared according to Method B′ from {1-[3-cyano-5-(ethoxycarbonyl)-6-(trifluoromethyl)pyridin-2-yl]pyrrolidin-3-yl}acetic acid and 2-phenylethanesulfonamide to give ethyl 5-cyano-6-[3-(2-oxo-2-{[(2-phenylethyl)sulfonyl]amino}ethyl)pyrrolidin-1-yl]-2-(trifluoromethyl)nicotinate. Yield: 73 mg (68%).

¹H NMR (600 MHz, DMSO) d 1.25 (3H, t, J=7.0 Hz), 1.58-1.66 (1H, m), 2.05-2.13 (1H, m), 2.37-2.40 (2H, m), 2.92-2.98 (2H, m), 3.62-3.67 (2H, m), 3.67-3.75 (1H, m), 3.80-3.99 (2H, m), 4.23 (2H, q, J=7.3 Hz), 7.15-7.31 (5H, m), 8.43 (1H, s). Note! One H hidden in the DMSO peak and one H hidden in the H₂O peak

MS ^{m}/_{z}: 537 (M−1)

### Example 191

### Ethyl 6-[3-(2-{[(5-chloro-2-thienyl)sulfonyl]amino}-2-oxoethyl)pyrrolidin-1-yl]-5-cyano-2-(trifluoromethyl)nicotinate

Prepared according to Method B′ from {1-[3-cyano-5-(ethoxycarbonyl)-6-(trifluoromethyl)pyridin-2-yl]pyrrolidin-3-yl}acetic acid and 5-chlorothiophene-2-sulfonamide to give ethyl 6-[3-(2-{[(5-chloro-2-thienyl)sulfonyl]amino}-2-oxoethyl)pyrrolidin-1-yl]-S-cyano-2-(trifluoromethyl)nicotinate. Yield: 86 mg (78%).

¹H NMR (500 MHz, DMSO) d 1.29 (3K, t, J=6.9 Hz), 1.60-1.69 (1H, m), 2.06-2.14 (1H, m), 2.44-2.48 (1H, m), 2.55-2.60 (1H, m), 3.33-3.39 (1H, m), 3.68-3.76 (1H, m), 3.84-3.96 (2H, m), 4.28 (2H, q, J=7.2 Hz), 7.22 (1H, d, J=4.2 Hz), 7.63 (1H, d, J=4.2 Hz), 8.41 (1H, s).

MS ^{m}/_{z}: 549 (M−1)

### Example 192

### Ethyl 5-cyano-6-[3-({[(4-fluorobenzyl)sulfonyl]amino}carbonyl)azetidin-1-yl]-2-(trifluoromethyl)nicotinate

### (a) 1-[3-cyano-5-(ethoxycarbonyl)-6-(trifluoromethyl)pyridin-2-yl]azetidine-3-carboxylic acid

TEA (0.908 g, 8.97 mmol) was added to a suspension of ethyl 6-chloro-5-cyano-2-(trifluoromethyl)nicotinate (1.0 g, 3.59 mmol) and azetidine-3-carboxylic acid (0.399 g, 3.95 mmol) in EtOH (10 mL) and the mixture was heated in a single-node microwave oven for 20 minutes. The solvent was evaporated and the residue was partioned between iPrOAc (10 mL)/water and Na₂CO₃. The aqueous phase was separated and made acidic by addition of concentrated HCl. The acidic water phase was extracted with iPrOAc (2×10 mL). The combined extracts was dried MgSO4) and evaporated to give 1-[3-cyano-5-(ethoxycarbonyl)-6-(trifluoromethyl)pyridin-2-yl]azetidine-3-carboxylic acid as a brown solid which was used without further purification. Yield 1.04 g (84%).

1H-NMR (500 MHz, DMSO-d₆) δ 1.27 (3H, t, J=7.1 Hz), 3.55-3.62 (1H, m), 4.28 (2H, q, J=57.1 Hz), 4.38-4.58 (4H, m), 8.46 (1H, s).

### (b) Ethyl 5-cyano-6-[3-({[(4-fluorobenzyl)sulfonyl]amino}carbonyl)azetidin-1-yl]-2-(trifluoromethyl)nicotinate

Prepared according to Method D′ from 1-[3-cyano-5-(ethoxycarbonyl)-6-(trifluoromethyl)pyridin-2-yl]azetidine-3-carboxylic acid and I-(t; fluorophenyl)methanesulfonamide to give ethyl 5-cyano-6-[3-({[(4 fluorobenzyl)sulfonyl]amino}carbonyl)azetidin-1-yl]-2-(trifluoromethyl)nicotinate.

MS ^{m}/z: 515 (M+1)

### Example 193

### Ethyl 5-cyano-6-[3-({[(3-fluorobenzyl)sulfonyl]amino}carbonyl)azetidin-1-yl]-2-(trifluoromethyl)nicotinate

Prepared according to Method D′ from 1-[3-cyano-5-(ethoxycarbonyl)-6-(trifluoromethyl)pyridin-2-yl]azetidine-3-carboxylic acid and 1-(3-fluorophenyl)methanesulfonamide to give ethyl 5-cyano-6-[3-({[(3-fluorobenzyl)sulfonyl]amino}carbonyl)azetidin-1-yl]-2-(trifluoromethyl)nicotinate.

MS ^{m}/_{z}: 515 (M+1)

### Example 194

### Ethyl 5-cyano-6-[3-({[(2-fluorobenzyl)sulfonyl]amino}carbonyl)azetidin-1-yl]-2-(trifluoromethyl)nicotinate

Prepared according to Method D′ from 1-[3-cyano-5-(ethoxycarbonyl)-6-(trifluoromethyl)pyridin-2-yl]azetidine-3-carboxylic acid and 1-(2-fluorophenyl)methanesulfonamide to give ethyl 5-cyano-6-[3-({[(2-fluorobenzyl)sulfonyl]amino}carbonyl)azetidin 1-yl]-2-(trifluoromethyl)nicotinate

MS ^{m}/_{z}: 515 (M+1)

### Example 195

### Ethyl 5-cyano-6-[3-({[(4-methylbenzyl)sulfonyl]amino}carbonyl)azetidin-1-yl]-2-(trifluoromethyl)nicotinate

Prepared according to Method D′ from 1-[3-cyano-5-(ethoxycarbonyl)-6-(trifluoromethyl)pyridin-2-yl]azetidine-3-carboxylic acid and 1-(4-methylphenyl)methanesulfonamide to give ethyl 5-cyano-6-[3-({[(4-methylbenzyl)sulfonyl]amino}carbonyl)azetidin-1-yl]-2-(trifluoromethyl)nicotinate.

MS ^{m}/_{z}: 511 (M+1)

### Example 196

### Ethyl 5-cyano-6-[3-({[(3-methylbenzyl)sulfonyl]amino}carbonyl)azetidin-1-yl]-2-(trifluoromethyl)nicotinate

Prepared according to Method D′ from 1-[3-cyano-5-(ethoxycarbonyl)-6-(trifluoromethyl)pyridin-2-yl]azetidine-3-carboxylic acid and 1-(3-methylphenyl)methanesulfonamide to give ethyl 5-cyano-6-[3-({[(3-methylbenzyl)sulfonyl]amino}carbonyl)azetidin-1-yl]-2-(trifluoromethyl)nicotinate.

MS ^{m}/_{z}: 511 (M+1)

### Example 197

### Ethyl 6-[3-({[(4-chlorobenzyl)sulfonyl]amino}carbonyl)azetidin-1-yl]-5-cyano-2-(trifluoromethyl)nicotinate

Prepared according to Method D′ from 1-[3-cyano-5-(ethoxycarbonyl)-6-(trifluoromethyl)pyridin-2-yl]azetidine-3-carboxylic acid and 1-(4-chlorophenyl)methanesulfonamide to give ethyl 6-[3-({[(4-chlorobenzyl)sulfonyl]amino}carbonyl)azetidin-1-yl]-5-cyano-2-(trifluoromethyl)nicotinate.

MS ^{m}/_{z}: 531 (M+1)

### Example 198

### Ethyl 6-[3-({[(2-chlorobenzyl)sulfonyl]amino}carbonyl)azetidin-1-yl]-S-cyano-2-(trifluoromethyl)nicotinate

Prepared according to Method D′ from 1-[3-cyano-5-(ethoxycarbonyl)-6-(trifluoromethyl)pyridin-2-yl]azetidine-3-carboxylic acid and 1-(2-chlorophenyl)methanesulfonamide to give ethyl 6-[3-({[(2-chlorobenzyl)sulfonyl]amino}carbonyl)azetidin-1-yl]-5-cyano-2-(trifluoromethyl)nicotinate.

MS ^{m}/_{z}: 531 (M+1)

### Example 199

### Ethyl 6-[3-({[(3-chlorobenzyl)sulfonyl]amino}carbonyl)azetidin-1-yl]-5-cyano-2-(trifluoromethyl)nicotinate

Prepared according to Method D′ from 1-[3-cyano-5-(e oxycarbonyl)-6-(trifluoromethyl)pyridin-2-yl]azetidine-3-carboxylic acid and 1-(3-chlorophenyl)methanesulfonamide to give ethyl 6-[3-({[(3-chlorobenzyl)sulfonyl]amino}carbonyl)azetidin-1-yl]-5-cyano-2-(trifluoromethyl)nicotinate.

MS ^{m}/_{z}: 531 (M+1)

### Example 200

### Ethyl 5-cyano-6-[3-({[(2,4-dichlorobenzyl)sulfonyl]amino}carbonyl)azetidin-1-yl]-2-(trifluoromethyl)nicotinate

Prepared according to Method D′ from 1-[3-cyano-5-(ethoxycarbonyl)-6-(trifluoromethyl)pyridin-2-yl]azetidine-3-carboxylic acid and 1-(2,4-dichlorophenyl)methanesulfonamide to give ethyl 5-cyano-6-[3-({[(2,4-dichlorobenzyl)sulfonyl]amino}carbonyl)azetidin-1-yl]-2-(trifluoromethyl)nicotinate.

MS ^{m}/_{z}: 565 (M+1)

### Example 201

### Ethyl 6-(3-{[(5-chloro-2-thienyl)sulfonyl]carbamoyl}azetidin-1-yl)-5-cyano-2-(trifluoromethyl)nicotinate

Prepared according to Method D′ from 1-[3-cyano-5-(ethoxycarbonyl)-6-(trifluoromethyl)pyridin-2-yl]azetidine-3-carboxylic acid and 5-chlorothiophene-2-sulfonamide to give ethyl 6-(3-{[(5-chloro-2-thienyl)sulfonyl]carbamoyl}azetidin-1-yl)-5-cyano-2-(trifluoromethyl)nicotinate.

MS ^{m}/_{z}: 523 (M+1)

### Example 202

### Ethyl 5-cyano-6-[4-({[(4-fluorobenzyl)sulfonyl]amino}carbonyl)piperidin-1-yl]-2-(trifluoromethyl)nicotinate

### (a) 1-[3-cyano-5-(ethoxycarbonyl)-6-(trifluoromethyl)pyridin-2-yl]piperidine-4-carboxylic acid

TEA (0.908 g, 8.97 mmol) was added to a suspension of ethyl 6-chloro-5-cyano-2-(trifluoromethyl)nicotinate (1.0 g, 3.59 mmol) and piperidine-4-carboxylic acid (0.510 g, 3.95 mmol) in EtOH (10 mL) and the mixture was heated in a single-node microwave oven for 15 minutes. The solvent was evaporated and the residue was partioned between iPrOAc (10 mL)/water and 20% Na₂CO₃ (1 mL). The aqueous phase was separated, 1 mL EtOH was added and the water phase was made acidic by addition of concentrated HCl. The acidic water phase was extracted with iPrOAc (2×10 mL). The organic phase was dried (MgSO₄), filtered and concentrated to give 1-[3-cyano-5-(ethoxycarbonyl)-6-(trifluoromethyl)pyridin-2-yl]piperidine-4-carboxylic acid as a brown solid which was used without further purification. Yield: 1.06 g (79%).

¹H NMR (500 MHz, DMSO-d₆) δ 1.28 (3H, t, J=7.1 Hz), 1.61-1.71 (2H, m), 1.95-2.02 (2H, m), 2.60-2.68 (1H, m), 3.31-3.38 (2H, m), 4.28 (2H, q, J=7.1 Hz), 4.41-4.48 (2H, m), 8.51 (1H, s).

### (b) Ethyl 5-cyano-6-[4-({[(4-fluorobenzyl)sulfonyl]amino}carbonyl)piperidin-1-yl]-2-(trifluoromethyl)nicotinate

Prepared according to Method C′ from 1-[3-cyano-5-(ethoxycarbonyl)-6-(trifluoromethyl)pyridin-2-yl]piperidine-4-carboxylic acid and 1-(4-fluorophenyl)methanesulfonamide to give ethyl 5-cyano-6-[4-({[(4-fluorobenzyl)sulfonyl]amino}carbonyl)piperidin-1-yl]-2-(trifluoromethyl)nicotinate. Yield: 4.3 mg (4%).

¹H NMR (600 MHz, CDCl₃) δ 1.36 (3H, t, J=7 Hz), 1.78-1.94 (4H, m), 2.49-2.55 (1H, m), 3.23 (2H, t, J=12.5 Hz), 4.35 (2H, q, J=7 Hz), 4.60 (2H, s), 4.67 (2H, br d, J=12.5 Hz), 7.06 (2H, t, J=8.5 Hz), 7.31 (2H, dd, J=5, 8.5 Hz), 8.34 (1H, s), 9.50 (1H, s).

MS ^{m}/_{z}: 543 (M+1)

### Example 203

### Ethyl 5-cyano-6-[4-({[(3-fluorobenzyl)sulfonyl]amino}carbonyl)piperidin-1-yl]-2-(trifluoromethyl)nicotinate

Prepared according to Method C′ from 1-[3-cyano-5-(ethoxycarbonyl)-6-(trifluoromethyl)pyridin-2-yl]piperidine-4-carboxylic acid and 1-(3-fluorophenyl)methanesulfonamide to give ethyl 5-cyano-6-[4-({[(3-fluorobenzyl)sulfonyl]amino}carbonyl)piperidin-1-yl]-2-(trifluoromethyl)nicotinate. Yield: 5.7 mg (5%).

¹H NMR (500 MHz, CDCl₃) δ 1.40 (3H, t, J=7.5 Hz), 1.81-1.97 (4H, m), 2.53-2.61 (1H, m), 3.28 (2H, t, J=12.5 Hz), 4.39 (2H, q, J=7.5 Hz), 4.67 (2H, s), 4.71 (2H, br d, J=12.5 Hz), 7.12-7.15 (3H, m), 7.36-7.41 (1H, m), 8.38 (1H, s), 9.68 (1H, s).

MS ^{m}/_{z}: 543 I+1)

### Example 204

### Ethyl 5-cyano-6-[4-({[(2-fluorobenzyl)sulfonyl]amino}carbonyl)piperidin-1-yl]-2-(trifluoromethyl)nicotinate

Prepared according to Method C′ from 1-[3-cyano-5-(ethoxycarbonyl)-6-(trifluoromethyl)pyridin-2-yl]piperidine-4-carboxylic acid and 1-(2-fluorophenyl)methanesulfonamide to give ethyl 5-cyano-6-[4-({[(2-fluorobenzyl)sulfonyl]amino}carbonyl)piperidin-1-yl]-2-(trifluoromethyl)nicotinate. Yield: 5.1 mg (5%).

¹H NMR (400 MHz, CDCl₃) δ 1.35 (3H, t, J=6.5 Hz), 1.80-1.99 (4H, m), 2.53-2.61 (1H, m), 3.27 (2H, t, J=13 Hz), 4.34 (2H, q, J=6.5 Hz), 4.67 (2H, br d, J=13 Hz), 4.69 (2H, s), 7.11 (1H, t, J=9 Hz), 7.17 (1H, t, J=7.5 Hz), 7.34-7.39 (2H, m), 8.33 (1H, s), 9.63 (1H, s).

MS ^{m}/_{z}: 543 (M+1)

### Example 205

### Ethyl 5-cyano-6-[4-({[(4-methylbenzyl)sulfonyl]amino}carbonyl)piperidin-1-yl]-2-(trifluoromethyl)nicotinate

Prepared according to Method C′ from 1-[3-cyano-5-(ethoxycarbonyl)-6-(trifluoromethyl)pyridin-2-yl]piperidine-4-carboxylic acid and 1-(4-methylphenyl)methanesulfonamide to give ethyl 5-cyano-6-[4-({[(4-methylbenzyl)sulfonyl]amino}carbonyl)piperidin-1-yl]-2-(trifluoromethyl)nicotinate. Yield: 3.4 mg (3%).

¹H NMR (400 MHz, CDCl₃) δ 1.36 (3H, t, J=7.5 Hz), 1.75-1.93 (4H, m), 2.34 (3H, s), 2.44-2.52 (1H, m), 3.23 (2H, t, J=12.5 Hz), 4.35 (2H, q, J=7.5 Hz), 4.58 (2H, s), 4.66 (2H, br d, J=12.5 Hz), 7.15-7.21 (4H, m), 8.33 (1H, s), 8.88 (1H, s).

MS ^{m}/_{z}: 539 (M+1)

### Example 206

### Ethyl 5-cyano-6-[4-{[(3-methylbenzyl)sulfonyl]amino}carbonyl)piperidin-1-yl]-2-(trifluoromethyl)nicotinate

Prepared according to Method C′ from 1-[3-cyano-5-(ethoxycarbonyl)-6-(trifluoromethyl)pyridin-2-yl]piperidine-4-carboxylic acid and 1-(3-methylphenyl)methanesulfonamide to give ethyl 5-cyano-6-[4-({[(3-methylbenzyl)sulfonyl]amino}carbonyl)piperidin-1-yl]-2-(trifluoromethyl)nicotinate. Yield: 2.8 mg (3%).

¹H NMR (400 MHz, CDCl₃) δ 1.31 (3H, t, J=7.5 Hz), 1.71-1.88 (4H, m), 2.28 (3H, s), 2.39-2.47 (1H, m), 3.18 (2H, t, J=13 Hz), 4.30 (2H, q, J=7.5 Hz), 4.54 (2H, s), 4.61 (2H, br d, J=13 Hz), 7.05-7.23 (4H, m), 8.29 (1H, s), 8.72 (1H, s).

MS ^{m}/_{z}: 539 (M+1)

### Example 207

### Ethyl 6-[4-({[(4-chlorobenzyl)sulfonyl]amino}carbonyl)piperidin-1-yl]-5-cyano-2-(trifluoromethyl)nicotinate

Prepared according to Method C′ from 1-[3-cyano-5-(ethoxycarbonyl)-6-(trifluoromethyl)pyridin-2-yl]piperidine-4-carboxylic acid and 1-(4-chlorophenyl)methanesulfonamide to give ethyl 6-[4-({[(4-chlorobenzyl)sulfonyl]amino}carbonyl)piperidin-1-yl]-5-cyano-2-(trifluoromethyl)nicotinate. Yield: 6.6 mg (6%).

¹H NMR (600 MHz, CDCl₃) δ 1.20 (3H, t, J=7.5 Hz), 1.63-1.70 (2H, m), 1.74-1.79 (2H, m), 2.39-2.41 (1H, m), 3.09 (2H, t, J=12.5 Hz), 4.18 (2H, q, J=7.5 Hz), 4.42 (2H, s), 4.52 (2H, br d, J=12.5 Hz), 7.12 (2H, d, J=8.5 Hz), 7.19 (2H, d, J=8.5 Hz), 8.18 (1H, s), 11.32 (1H, s).

MS ^{m}/_{z}=559 (M+1)

### Example 208

### Ethyl 6-[4-({[(2-chlorobenzyl)sulfonyl]amino}carbonyl)piperidin-1-yl]-5-cyano-2-(trifluoromethyl)nicotinate

Prepared according to Method C′ from 1-[3-cyano-5-(ethoxycarbonyl)-6-(trifluoromethyl)pyridin-2-yl]piperidine-4-carboxylic acid and 1-(2-chlorophenyl)methanesulfonamide to give ethyl 6-[4-({[(2-chlorobenzyl)sulfonyl]amino}carbonyl)piperidin-1-yl]-5-cyano-2-(trifluoromethyl)nicotinate. Yield: 7.8 mg (7%).

¹H NMR (600, CDCl₃) δ 1.35 (3H, t, J=7 Hz), 1.81-1.90 (2H, m), 1.96-2.00 (2H, m), 2.56-2.64 (1H, m), 3.26 (2H, t, J=12 Hz), 4.34 (2H, q, J=7 Hz), 4.68 (2H, br d, J=12 Hz), 4.84 (2H, s), 7.27-7.34 (2H, m), 7.42 (2H, t, J=7 Hz), 8.34 (1H, s), 10.03 (1H, s).

MS ^{m}/_{z}: 559 (M+1)

### Example 209

### Ethyl 6-[4-({[(3-chlorobenzyl)sulfonyl]amino}carbonyl)piperidin-1-yl]-5-cyano-2-(trifluoromethyl)nicotinate

Prepared according to Method C′ from 1-[3-cyano-5-(ethoxycarbonyl)-6-(trifluoromethyl)pyridin-2-yl]piperidine-4-carboxylic acid and 1-(3-chlorophenyl)methanesulfonamide to give ethyl 6-[4-({[(3-chlorobenzyl)sulfonyl]amino}carbonyl)piperidin-1-yl]-5-cyano-2-(trifluoromethyl)nicotinate. Yield: 7.3 mg (6%).

¹H NMR (500 MHz, CDCl₃) δ 1.40 (3H, t, J=7.5 Hz), 1.81-1.90 (2H, m), 1.91-1.97 (2H, m), 2.54-2.62 (1H, m), 3.28 (2H, t, J=12.5 Hz), 4.39 (2H, q, J=7.5 Hz), 4.64 (2H, s), 4.72 (2H, br d, J=12.5 Hz), 7.25 (1H, d, J=7.5 Hz), 7.34-7.42 (3H, m), 8.38 (1H, s), 10.02 (1H, s).

MS ^{m}/_{z}: 559 (M+1)

### Example 210

### Ethyl 5-cyano-6-[4-({[(2,4-dichlorobenzyl)sulfonyl]amino}carbonyl)piperidin-1-yl]-2-(trifluoromethyl)nicotinate

Prepared according to Method C′ from 1-[3-cyano-5-(ethoxycarbonyl)-6-(trifluoromethyl)pyridin-2-yl]piperidine-4-carboxylic acid and 1-(2,4-dichlorophenyl)methanesulfonamide to give ethyl 5-cyano-6-[4-({[(2,4-dichlorobenzyl)sulfonyl]amino}carbonyl)piperidin 1-yl]-2-(trifluoromethyl)nicotinate. Yield: 5.5 mg (5%).

¹H NMR (600 MHz, CDCl₃) δ 1.35 (311, t, J=7.5 Hz), 1.83-1.90 (2H, m), 1.97-2.01 (2H, m), 2.56-2.64 (1H, m), 3.29 (2H, t, J=12.5 Hz), 4.34 (2H, q, J=7.5 Hz), 4.68 (2H, br d, J=12.5 Hz), 4.80 (2H, s), 7.28 (1H, dd, J=2, 8.5 Hz), 7.37 (1H, d, J=8.5 Hz), 7.45 (1H, d, J=2 Hz), 8.33 (1H, s), 10.04 (1H, s).

MS ^{m}/_{z}: 593 (M+1).

### Example 211

### Ethyl 6-[4-({[(5-chloro-2-thienyl)sulfonyl]amino}carbonyl)piperidin-1-yl]-5-cyano-2-(trifluoromethyl)nicotinate

Prepared according to Method C′ from 1-[3-cyano-5-(ethoxycarbonyl)-6-(trifluoromethyl)pyridin-2-yl]piperidine-4-carboxylic acid and 5-chlorothiophene-2-sulfonamide to give ethyl 6-[4-({[(5-chloro-2-thienyl)sulfonyl]amino}carbonyl)piperidin-1-yl]-5-cyano-2-(trifluoromethyl)nicotinate. Yield: 19.1 mg (17%).

¹H NMR (400 MHz, CDCl₃) δ 1.34 (3H, t, J=7 Hz), 1.72-1.84 (2H, m), 1.91-1.97 (2H, m), 2.55-2.65 (1H, m), 3.27 (2H, t, J=12.5 Hz), 4.33 (2H, s, J=7.5 Hz), 4.61 (2H, br d, J=12.5 Hz), 6.91 (1H, d, J=4 Hz), 7.62 (1H, d, J=4 Hz), 8.30 (1H, s), 10.99 (1H, s).

MS ^{m}/_{z}: 551 (M+1)

### Example 212

### Ethyl 5-cyano-6-[3-({[(2-fluorobenzyl)sulfonyl]amino}carbonyl)azetidin-1-yl]-2-(fluoromethyl)nicotinate

### (a) 1-[3-cyano-5-(ethoxycarbonyl)-6-(fluoromethyl)pyridin-2-yl]azetidine-3-carboxylic acid

TEA (653 mg, 6.46 mmol) was added to a solution of ethyl 6-chloro-5-cyano-2-(fluoromethyl)nicotinate(400 mg, 1.61 mmol) and azetidine-3-carboxylic acid (179 mg, 1.78 mmol) in water/EtOH (4.5 mL). The mixture was heated in a single-node microwave oven at 120° C. for 20 minutes. The solvent was evaporated and the residue was taken up in DCM and washed with 1% KHSO4. The aqueous phase was extracted with DCM and the combined organic phase was filtered through a phase separator and concentrated. Purification by HPLC (Kromasil C8, Eluent: A gradient of 5% CH₃CN to 100% CH₃CN/(0.2% HOAc (aq)) gave 1-[3-cyano-5-(ethoxycarbonyl)-6-(fluoromethyl)pyridin-2-yl]azetidine-3-carboxylic acid as a white solid. Yield 302 mg (60%).

¹H NMR (400 MHz, CDCl₃) δ 1.31 (3H, t, J=7.3 Hz), 3.59-3.69 (1H, m), 4.31 (2H, q, J=7.3 Hz), 4.60-4.70 (4H, m), 5.69 (2H, d, J=47.3 Hz), 8.30 (1H, br s).

### (b) Ethyl 5-cyano-6-[3-({[(2-fluorobenzyl)sulfonyl]amino}carbonyl)azetidin-1-yl]-2-(fluoromethyl)nicotinate

Prepared according to Method E′ from 1-[3-cyano-5-(ethoxycarbonyl)-6-(fluoromethyl)pyridin-2-yl]azetidine-3-carboxylic acid and 1-(2-fluorophenyl)methanesulfonamide to give ethyl 5-cyano-6-[3-({[(2-fluorobenzyl)sulfonyl]amino}carbonyl)azetidin-1-yl]-2-(fluoromethyl)nicotinate. Yield: 21 mg (44%).

¹H NMR (400 MHz, DMSO-d₆) δ 1.30 (3H, t, J=7.2 Hz), 3.55-3.66 (1H, m), 4.25 (2H, q, J=7.2 Hz), 4.34-4.44 (2H, m), 4.43-4.56 (2H, m), 4.80 (2H, s), 5.68 (2H, d, J=47.1 Hz), 7.18-7.32 (2H, m), 7.37-7.52 (2H, m), 8.39 (1H, s), 11.80-12.19 (1H, m)

MS ^{m}/_{z}: 479 (M+1).

### Example 213

### Ethyl 5-cyano-6-[3-{[(3-fluorobenzyl)sulfonyl]amino}carbonyl)azetidin-1-yl]-2-(fluoromethyl)nicotinate

Prepared according to Method E′ from 1-[3-cyano-5-(ethoxycarbonyl)-6-(fluoromethyl)pyridin-2-yl]azetidine-3-carboxylic acid and 1-(3-fluorophenyl)methanesulfonamide to give ethyl 5-cyano-6-[3-({[(3-fluorobenzyl)sulfonyl]amino}carbonyl)azetidin-1-yl]-2-(fluoromethyl)nicotinate. Yield: 25 mg (53%).

¹H NMR (400 MHz, DMSO-d₆) δ 1.29 (3H, t, J=7.1 Hz), 3.54-3.64 (1H, m), 4.24 (2H, q, J=7.1 Hz), 4.28-4.36 (2H, m), 4.39-4.53 (2H, m), 4.79 (2H, s), 5.67 (2H, d, J=47.1 Hz), 7.13-7.27 (3H, m), 7.37-7.47 (1H, m), 8.38 (1H, s), 11.55-12.36 (1H, m)

MS ^{m}/_{z}: 479 (+1).

### Example 214

### Ethyl 5-cyano-6-[3-({[(4-fluorobenzyl)sulfonyl]amino}carbonyl)azetidin-1-yl]-2-(fluoromethyl)nicotinate

Prepared according to Method E′ from 1-[3-cyano-5-(ethoxycarbonyl)-6-(fluoromethyl)pyridin-2-yl]azetidine-3-carboxylic acid and 1-(4-fluorophenyl)methanesulfonamide to give ethyl 5-cyano-6-[3-({[(4-fluorobenzyl)sulfonyl]amino}carbonyl)azetidin-1-yl]-2-(fluoromethyl)nicotinate. Yield: 27 mg (56%).

¹H NMR (400 MHz, DMSO-d₆) δ 1.29 (3H, t, J=7.2 Hz), 3.55-3.77 (1H, m), 4.24 (2H, q, J=7.1 Hz), 4.29-4.37 (2H, m), 4.41-4.51 (2H, m), 4.73 (2H, s), 5.66 (2H, d, J=47.1 Hz), 7.15-7.23 (2H, m), 7.34-7.42 (2H, m), 8.37 (1H, s).

MS ^{m}/_{z}: 479 (M+1).

### Example 215

### Ethyl 6-[3-({[(2-chlorobenzyl)sulfonyl]amino}carbonyl)azetidin-1-yl]-5-cyano-2-(fluoromethyl)nicotinate

Prepared according to Method E′ from 1-[3-cyano-5-(ethoxycarbonyl)-6-(fluoromethyl)pyridin-2-yl]azetidine-3-carboxylic acid and 1-(2-chlorophenyl)methanesulfonamide to give ethyl 6-[3-({[(2-chlorobenzyl)sulfonyl]amino}carbonyl)azetidin-1-yl]-5-cyano-2-(fluoromethyl)nicotinate. Yield: 13 mg (27%).

¹H NMR (400 MHz, DMSO-d₆) δ 1.30 (3H, t, J=7.2 Hz), 3.59-3.69 (1H, m), 4.25 (2H; q, J=7.2 Hz), 4.36-4.56 (4H, m), 4.90 (2H, s), 5.67 (2H, d, J=47.3 Hz), 7.34-7.56 (4H, m), 8.38 (1H, s), 11.73-12.28 (1H, m)

MS^{m}/z: 495 (M+1).

### Example 216

### Ethyl 6-[3-({[(3-chlorobenzyl)sulfonyl]amino}carbonyl)azetidin-1-yl]-5-cyano-2-(fluoromethyl)nicotinate

Prepared according to Method E′ from 1-[3-cyano-5-(ethoxycarbonyl)-6-(fluoromethyl)pyridin-2-yl]azetidine-3-carboxylic acid and 1-(3-chlorophenyl)methanesulfonamide to give ethyl 6-[3-({[(3-chlorobenzyl)sulfonyl]amino}carbonyl)azetidin-1-yl]-5-cyano-2-(fluoromethyl)nicotinate. Yield: 28 mg (58%).

¹H NMR (400 MHz, DMSO-d₆) δ 1.30 (3H, t, J=7.2 Hz), 3.51-3.65 (1H, m), 4.25 (2H, q, J=7.2 Hz), 4.27-4.37 (2H, m), 4.40-4.53 (2H, m), 4.79 (2H, s), 5.67 (2H, d, J=47.1 Hz), 7.27-7.50 (4H, m), 8.36-8.40 (1H, m), 11.71-12.13 (1H, m)

MS ^{m}/_{z}: 495 (M+1).

### Example 217

### Ethyl 6-[3-({[(4-chlorobenzyl)sulfonyl]amino}carbonyl)azetidin-1-yl]-5-cyano-2-(fluoromethyl)nicotinate

Prepared according to Method E′ from 1-[3-cyano-5-(ethoxycarbonyl)-6-(fluoromethyl)pyridin-2-yl]azetidine-3-carboxylic acid and 1-(4-chlorophenyl)methanesulfonamide to give ethyl 6-[3-({[(4-chlorobenzyl)sulfonyl]amino}carbonyl)azetidin 1-yl]-5-cyano-2-(fluoromethyl)nicotinate. Yield: 33 mg (68%).

¹H NMR (400 MHz, DMSO-d₆) δ 1.29 (3H, t, J=7.2 Hz), 3.45-3.58 (1H, m), 4.24 (2H, q, J=7.2 Hz), 4.29-4.38 (2H, m), 4.38-4.50 (2H, m), 4.60 (2H, s), 5.66 (2H, d, J=47.1 Hz), 7.29-7.41 (4H, m), 8.36 (1H, s).

MS ^{m}/_{z}: 495 (M+1).

### Example 218

### Ethyl 5-cyano-2-(fluoromethyl)-6-[3-({[(3-methylbenzyl)sulfonyl]amino}carbonyl)azetidin-1-yl]nicotinate

Prepared according to Method E′ from 1-[3-cyano-5-(ethoxycarbonyl)-6-(fluoromethyl)pyridin-2-yl]azetidine-3-carboxylic acid and 1-(3-methylphenyl)methanesulfonamide to give ethyl 5-cyano-2-(fluoromethyl)-6-[3-({[(3-methylbenzyl)sulfonyl]amino}carbonyl)azetidin-1-yl]nicotinate. Yield: 41 mg (86%).

¹H NMR (400 MHz, DMSO-d₆) δ 1.30 (3H, t, J=7.2 Hz), 2.27 (3H, s), 3.51-3.60 (1H, m), 4.25 (2H, q, J=7.2 Hz), 4.29-4.37 (2H, m), 4.39-4.51 (2H, m), 4.69 (2H, s), 5.67 (2H, d, J=50.0 Hz), 7.07-7.32 (4H, m), 8.38 (1H, s), 11.59-12.03 (1H, m)

MS ^{m}/_{z}: 475 (M+1).

### Example 219

### Ethyl 5-cyano-2-(fluoromethyl)-6-[3-({[(4-methylbenzyl)sulfonyl]amino}carbonyl)azetidin-1-yl]nicotinate

Prepared according to Method E′ from 1-[3-cyano-5-(ethoxycarbonyl)-6-(fluoromethyl)pyridin-2-yl]azetidine-3-carboxylic acid and 1-(4-methylphenyl)methanesulfonamide to give ethyl 5-cyano-2-(fluoromethyl)-6-[3-({[(4-methylbenzyl)sulfonyl]amino}carbonyl)azetidin-1-yl]nicotinate. Yield: 12 mg (25%).

¹H NMR (400, DMSO-d₆) δ 1.29 (3H, t, J=7.2 Hz), 2.28 (3H, s), 3.53-3.60 (1H, m), 4.24 (2H, q, J=7.2 Hz), 4.29-4.36 (2H, m), 4.39-4.50 (2H, m), 4.67 (2H, s), 5.67 (2H, d, J=47.1 Hz), 7.15-7.23 (4H, m), 8.37-8.40 (1H, m), 11.48-12.04 (1H, m)

MS ^{m}/_{z}: 475 (M+1).

### Example 220

### Ethyl 5-cyano-6-[3-({[(2,4-dichlorobenzyl)sulfonyl]amino}carbonyl)azetidin-1-yl]-2-(fluoromethyl)nicotinate

Prepared according to Method E′ from 1-[3-cyano-5-(ethoxycarbonyl)-6-(fluoromethyl)pyridin-2-yl]azetidine-3-carboxylic acid and 1-(2,4-dichlorophenyl)methanesulfonamide to give ethyl 5-cyano-6-[3-({[(2,4-dichlorobenzyl)sulfonyl]amino}carbonyl)azetidin-yl]-2-(fluoromethyl)nicotinate. Yield: 27 mg (51%).

¹H NMR 400 MHz, DMSO-d₆) δ 1.29 (3H, t, J=7.2 Hz), 3.56-3.65 (1H, m), 4.24 (2H, q, J=7.2 Hz), 4.35-4.58 (4H, m), 4.86 (2H, s), 5.67 (2H, d, J=47.1 Hz), 7.41-7.70 (3H, m), 8.36-8.39 (1H, m).

MS ^{m}/_{z}: 529 (M+1).

### Example 221

### Ethyl 5-cyano-2-(fluoromethyl)-6-{3-[({[(4-methylcyclohexyl)methyl]sulfonyl}amino)carbonyl]azetidin-1-yl}nicotinate Prepared according to Method E′ from 1-[3-cyano-5-(ethoxycarbonyl)-6-(fluoromethyl)pyridin-2-yl]azetidine-3-carboxylic acid and 1-(4-methylcyclohexyl)methanesulfonamide to give ethyl 5-cyano-6-[3-({[(2,4-dichlorobenzyl)sulfonyl]amino}carbonyl)azetidin 1-yl]-2-(fluoromethyl)nicotinate. Yield: 28 mg (57%).

¹H NMR (400 MHz, DMSO-d₆) δ 0.75-0.92 (4H, m), 0.95-1.17 (3H, m), 1.25 (3H, t, J=7.1 Hz), 1.35-1.54 (4H, m), 1.55-1.64 (1H, m), 1.74-1.84 (1H, m), 2.00-2.10 (1H, m), 3.22-3.28 (1H, m), 3.51-3.63 (1H, m), 4.20 (2H, q, J=7.1 Hz), 4.29-4.39 (2H, m), 4.40-4.51 (2H, m), 5.61 (2H, d, J=47.3 Hz), 8.32 (1H, s).

MS ^{m}/_{z}: 481 (M+1).

### Example 222

### Ethyl 5-cyano-6-[4-({[(2-fluorobenzyl)sulfonyl]amino}carbonyl)piperidin-1-yl]-2-(fluoromethyl)nicotinate

### (a) 1-[3-cyano-5-(ethoxycarbonyl)-6-(fluoromethyl)pyridin-2-yl]piperidine-4-carboxylic acid

TEA (653 mg, 6.46 mmol) was added to a solution of ethyl 6-chloro-5-cyano-2-(fluoromethyl)nicotinate (400 mg, 1.61 mmol) and piperidine-4-carboxylic acid (229 mg, 1.78 mmol) in water/EtOH (4.5 mL). The mixture was heated in a single-node microwave oven at 120° C. for 20 minutes. The solvent was evaporated and the residue was taken up in DCM and washed with 1% KHSO4. The aqueous phase was extracted with DCM and the combined organic phase was filtered through a phase separator and concentrated. Purification by HPLC (Kromasil C8, Eluent: A gradient of 5% CH₃CN to 100% CH₃CN/(0.2% HOAc (aq)) gave 1-[3-cyano-5-(ethoxycarbonyl)-6-(fluoromethyl)pyridin-2-yl]azetidine-3-carboxylic acid as a white solid. Yield 76 mg (14%).

¹H NMR (400 MHz, CDCl₃) δ 1.36 (3H, t, J=7.2 Hz), 1.82-1.94 (2H, m), 2.05-2.14 (2H, m), 2.66-2.76 (1H, m), 3.32-3.42 (2H, m), 4.31 (2H, t, I=7.2 Hz), 4.61-4.69 (2H, m), 5.70 (2H, d, J=47.3 Hz), 8.36 (1H, br s).

### (b) Ethyl 5-cyano-6-[4-({[(2-fluorobenzyl)sulfonyl]amino}carbonyl)piperidin-1-yl]-2-(fluoromethyl)nicotinate

Prepared according to Method E′ from 1-[3-cyano-5-(ethoxycarbonyl)-6-(fluoromethyl)pyridin-2-yl]piperidine-4-carboxylic acid and 1-(2-fluorophenyl)methanesulfonamide to give ethyl 5-cyano-6-[4-({[(2-fluorobenzyl)sulfonyl]amino}carbonyl)piperidin-1-yl]-2-(fluoromethyl)nicotinate. Yield: 13 mg (25%).

¹H NMR (400 MHz, DMSO-d₆) δ 1.29 (3H, t, J=7.1 Hz), 1.56-1.75 (2H, r), 1.82-1.93 (2H, m), 2.56-2.64 (1H, m), 3.14-3.26 (2H, m), 4.25 (2H, q, J=7.1 Hz), 4.55-4.64 (2H, m), 4.68 (2H, s), 5.68 (2H, d, J=47.1 Hz), 7.18-7.30 (2H, m), 7.32-7.48 (2H, m), 8.39 (1H,

MS ^{m}/_{z}: 507 (M+1).

### Example 223

### Ethyl 5-cyano-[4-({[(3-fluorobenzyl)sulfonyl]amino}carbonyl)piperidin-1-yl]-2-(fluoromethyl)nicotinate

Prepared according to Method E′ from 1-[3-cyano-5-(ethoxycarbonyl)-6-(fluoromethyl)pyridin-2-yl]piperidine-4-carboxylic acid and 1-(3-fluorophenyl)methanesulfonamide to give ethyl 5-cyano-6-[4-({[(3-fluorobenzyl)sulfonyl]amino}carbonyl)piperidin-1-yl]-2-(fluoromethyl)nicotinate. Yield: 16 mg (31%).

¹H NMR (400 MHz, DMSO-d₆) δ 1.30 (3H, t, J=7.1 Hz), 1.56-1.71 (2H, m), 1.79-1.89 (2H, m), 2:55-2.61 (1H, m), 3.15-3.26 (2H, m); 4.25 (2H, q, J=7.1 Hz), 4.53-4.64 (2H, m), 4.70 (2H, s), 5.69 (2H, d, J=47.1 Hz), 7.07-7.17 (2H, m), 7:20-7.28 (1H, m), 7.39-7.49 (1H, m), 8.39-8.42 (1H, m), 11.47-12.06 (1H, m)

MS ^{m}/_{z}: 507 (M+1).

### Example 224

### Ethyl 5-cyano-6-[4-({[(4-fluorobenzyl)sulfonyl]amino}carbonyl)piperidin-1-yl]-2-(fluoromethyl)nicotinate

Prepared according to Method E′ from 1-[3-cyano-5-(ethoxycarbonyl)-6-(fluoromethyl)pyridin-2-yl]piperidine-4-carboxylic acid and 1-(4-fluorophenyl)methanesulfonamide to give ethyl 5-cyano-6-[4-({[(4 fluorobenzyl)sulfonyl]amino}carbonyl)piperidin-1-yl]-2-(fluoromethyl)nicotinate. Yield: 23 mg (45%).

¹H NMR (400 MHz, DMSO-d₆) δ 1.29 (3H, t, J=7.1 Hz), 1.56-1.70 (2H, m), 1.78-1.89 (2H, m), 2.52-2.56 (1H, m), 3.14-3.24 (2H, m), 4.25 (2H, q, J=7.1 Hz), 4.51-4.63 (4H, m), 5.68 (2H, d, J=47.1 Hz), 7.16-7.24 (2H, m), 7.27-7.34 (2H, m), 8.39 (1H, s).

MS ^{m}/_{z}: 507 (M+1).

### Example 225

### Ethyl 6-[4-({[(2-chlorobenzyl)sulfonyl]amino}carbonyl)piperidin-1-yl]-5-cyano-2-(fluoromethyl)nicotinate

Prepared according to Method E′ from 1-[3-cyano-5-(ethoxycarbonyl)-6-(fluoromethyl)pyridin-2-yl]piperidine-4-carboxylic acid and 1-(2-chlorophenyl)methanesulfonamide to give ethyl 6-[4-({[(2-chlorobenzyl)sulfonyl]amino}carbonyl)piperidin-1-yl]-5-cyano-2-(fluoromethyl)nicotinate. Yield: 24 mg (45%).

¹H NMR (400 MHz, DMSO-d₆) δ 1.29 (3H, t, J=7.2 Hz), 1.56-1.74 (2H, m), 1.84-1.95 (2H, m), 2.56-2.66 (1H, m), 3.16-3.27 (2H, m), 4.25 (2H, q, J=7.2 Hz), 4.54-4.65 (2H, m), 4.80 (2H, s), 5.68 (2H, d, J=47.3 Hz), 7.35-7.46 (3H, m), 7.48-7.55 (1H, m), 8.39 (1H, s).

MS ^{m}/_{z}: 523 (M+1).

### Example 226

### Ethyl 6-[4-({[(3-chlorobenzyl)sulfonyl]amino}carbonyl)piperidin-1-yl]-5-cyano-2-(fluoromethyl)nicotinate

Prepared according to Method E′ from 1-[3-cyano-5-(ethoxycarbonyl)-6-(fluoromethyl)pyridin-2-yl]piperidine-4-carboxylic acid and 1-(3-chlorophenyl)methanesulfonamide to give ethyl 6-[4-({[(3-chlorobenzyl)sulfonyl]amino}carbonyl)piperidin-1-yl]-5-cyano-2-(fluoromethyl)nicotinate. Yield: 24 mg (46%).

¹H NMR (400 MHz, DMSO-d₆) δ 1.30 (3H, t, J=7.1 Hz), 1.57-1.70 (2H, m), 1.76-1.88 (2H, m), 2.53-2.61 (1H, m), 3.15-3.27 (2H, m), 4.25 (2H, q, J=7.1 Hz), 4.55-4.63 (2H, m), 4.68 (2H, s), 5.68 (2H, d, J=47.3 Hz), 7.18-7.52 (4H, m), 8.40 (1H, s).

MS ^{m}/_{z}: 523 (M+1).

### Example 227

### Ethyl 6-[4-({[(4-chlorobenzyl)sulfonyl]amino}carbonyl)piperidin-1-yl]-5-cyano-2-(fluoromethyl)nicotinate

Prepared according to Method E′ from 1-[3-cyano-5-(ethoxycarbonyl)-6-(fluoromethyl)pyridin-2-yl]piperidine-4-carboxylic acid and 1-(4-chlorophenyl)methanesulfonamide to give ethyl 6-[4-({[(4-chlorobenzyl)sulfonyl]amino}carbonyl)piperidin-1-yl]-5-cyano-2-(fluoromethyl)nicotinate. Yield: 24 mg (46%).

¹H NMR (400 MHz, DMSO-d₆) δ 1.30 (3H, t, J=7.2 Hz), 1.56-1.71 (2H, m), 1.80-1.90 (2H, m), 2.54-2.60 (1H, m), 3.13-3.26 (2H, m), 4.25 (2H, q, J=7.1 Hz), 4.55-4.63 (2H, m), 4.66 (2H, s), 5.68 (2H, d, J=47.1 Hz), 7.30 (2H, d, J=8.5 Hz), 7.46 (2H, d, J=8.5 Hz), 8.38-8.41 (1H, m).

MS ^{m}/_{z}: 523 (M+1).

### Example 228

### Ethyl 5-cyano-2-(fluoromethyl)-6-[4-({[(3-methylbenzyl)sulfonyl]amino}carbonyl)piperidin-1-yl]nicotinate

Prepared according to Method E′ from 1-[3-cyano-5-(ethoxycarbonyl)-6-(fluoromethyl)pyridin-2-yl]piperidine-4-carboxylic acid and 1-(3-methylphenyl)methanesulfonamide to give ethyl 5-cyano-2-(fluoromethyl)-6-[4-({[(3-methylbenzyl)sulfonyl]amino}carbonyl)piperidin-1-yl]nicotinate. Yield: 6 mg (12%).

¹H NMR (400 MHz, DMSO-d₆) δ 1.30 (3H, t, J=7.1 Hz), 1.58-1.71 (2H, m), 1.79-1.88 (2H, m), 2.28 (3H, s), 2.52-2.58 (1H, m), 3.17-3.23 (2H, m), 4.25 (2H, q, J=7.1 Hz), 4.48-4.68 (4H, m), 5.68 (2H, d, J=47.1 Hz), 7.00-7.32 (4H, m), 8.40 (1H, s), 11.27-11.80 (1H, m).

MS ^{m}/_{z}: 503 (M+1).

### Example 229

### Ethyl 5-cyano-2-(fluoromethyl)-6-[4-({[(4-methylbenzyl)sulfonyl]amino}carbonyl)piperidin-1-yl]nicotinate

Prepared according to Method E′ from 1-[3-cyano-S-(ethoxycarbonyl)-6-(fluoromethyl)pyridin-2-yl]piperidine-4-carboxylic acid and 1-(4-methylphenyl)methanesulfonamide to give ethyl 5-cyano-2-(fluoromethyl)-6-[4-({[(4-methylbenzyl)sulfonyl]amino}carbonyl)piperidin-1-yl]nicotinate. Yield: 20 mg (40%).

¹H NMR (400 MHz, DMSO-d₆) δ 1.30 (3H, t, J=7.2 Hz), 1.57-1.72 (2H, m), 1.80-1.92 (2H, m), 2.30 (3H, s), 2.54-2.64 (1H, m), 3.11-3.25 (2H, m), 4.26 (2H, q, J=7.2 Hz), 4.52-4.68 (4H, m), 5.69 (2H, d, J=47.3 Hz), 7.11-7.28 (4H, m), 8.41 (1H, s), 11.33-11.86 (1H, m).

MS ^{m}/_{z}: 503 (M+1).

### Example 230

### Ethyl 5-cyano-6-[4-({[(2,4-dichlorobenzyl)sulfonyl]amino}carbonyl)piperidin-1-yl]-2-(fluoromethyl)nicotinate

Prepared according to Method E′ from 1-[3-cyano-5-(ethoxycarbonyl)-6-(fluoromethyl)pyridin-2-yl]piperidine-4-carboxylic acid and 1-(2,4-dichlorophenyl)methanesulfonamide to give ethyl 5-cyano-6-[4-({[(2,4-dichlorobenzyl)sulfonyl]amino}carbonyl)piperidin-1-yl]-2-(fluoromethyl)nicotinate. Yield: 21 mg (38%).

¹H NMR (400 MHz, DMSO-d₆) δ 1.30 (3H, t, J=7.2 Hz), 1.56-1.72 (2H, m), 1.83-1.94 (2H, m), 2.54-2.59 (1H, m), 3.15-3.27 (2H, m), 4.25 (2H, q, J=7.2 Hz), 4.53-4.63 (2H, m), 4.73 (2H, s), 5.68 (2H, d, J=47.3 Hz), 7.39-7.53 (2H, m), 7.62-7.70 (1H, m), 8.35-8.43 (1H, m).

MS ^{m}/_{z}: 557 (M+1).

### Example 231

### Ethyl 5-cyano-2-(fluoromethyl)-6-{4-[({[(4-methylcyclohexyl)methyl]sulfonyl}amino)carbonyl]piperidin-1-yl}nicotinate

Prepared according to Method E′ from 1-[3-cyano-5-(ethoxycarbonyl)-6-(fluoromethyl)pyridin-2-yl]piperidine-4-carboxylic acid and 1-(4 methylcyclohexyl)methanesulfonamide to give ethyl 5-cyano-2-(fluoromethyl)-6-{4-[({[(4-methylcyclohexyl)methyl]sulfonyl}amino)carbonyl]piperidin-1-yl}nicotinate. Yield: 18 mg (36%).

¹H NMR (400 MHz, DMSO-d₆) δ 0.80-0.90 (4H, m), 0.96-1.20 (3H, m), 1.29 (3H, t, J=7.2 Hz), 1.38-1.69 (7H, m), 1.77-1.97 (3H, m), 1.99-2.09 (1H, m), 2.59-2.71 (2H, m), 3.16-3.29 (2H, m), 4.25 (2H, q, J=7.2 Hz), 4.51-4.66 (2H, m), 5.67 (2H, d, J=47.3 Hz), 8.39 (1H, s).

MS ^{m}/_{z}: 509 (M+1).

### Example 232

### Ethyl 6-(3-{2-[(benzylsulfonyl)amino]-2-oxoethyl}azetidin-1-yl)-5-cyano-2-(difluoromethyl)nicotinate

### (a) tert-butyl 3-{2-[(benzylsulfonyl)amino]-2-oxoethyl}azetidine-1-carboxylate

DIPEA (0.3 mL, 1.72 mmol) was added to a mixture of [1-(tert-butoxycarbonyl)azetidin-3-yl]acetic acid (193 mg, 0.90 mmol) and TBTU (326 mg, 1.02 mmol) in dry DCM (4 mL). The reaction mixture was stirred at rt for 1 h and 1-phenylmethanesulfonamide (169 mg, 0.99 mmol) was added and the stirring was continued at r.t for 19 h. NaHCO₃ (aq) was added and the mixture was extracted with EtOAc (3 times). The combined organic layer was dried over anhydrous MgSO₄, filtered and evaporated to give tert-butyl 3-{2-[(benzylsulfonyl)amino]-2-oxoethyl}azetidine-1-carboxylate which was used in the next step without further purification. Yield: 383 mg (116%).

MS ^{m}/_{z}: 367 (M−1).

### (b) 2-azetidin-3-yl-N-(benzylsulfonyl)acetamide

The crude tert-butyl 3-{2-[(benzylsulfonyl)amino]-2-oxoethyl}azetidine-1-carboxylate from the previous step (383 mg, 0.90 mmol) was dissolved in DCM (5 mL) and TFA (4 mL) was added. The reaction mixture was stirred at r.t for 1.5 hours. The solvent was evaporated to give 2-azetidin-3-yl-N-(benzylsulfonyl)acetamide which was used in the next step without further purification. Yield: 240 mg (100%)

MS ^{m}/_{z}: 269 (M+1), 267 (M−1).

### (c) Ethyl 6-(3-{2-[(benzylsulfonyl)amino]-2-oxoethyl}azetidin-1-yl)-5-cyano-2-(difluoromethyl)nicotinate

DIPEA (1 mL) was added to a solution of the crude 2-azetidin-3-yl-N-(benzylsulfonyl)acetamide from the previous step and ethyl 6-chloro-5-cyano-2-(difluoromethyl)nicotinate (180 mg, 0.69 mmol) in EtOH (9 mL). The reaction mixture was heated to 120° C. for 5 min using microwave single node heating. NaHCO₃ (aq) was added and the mixture was extracted with DCM (3 times). The combined organic layer was run through a phase separator and evaporated. The crude product was purified by HPLC (Kromasil C8 10 μm, 21.5×250 mm using a gradient of CH₃CN/0.1 M NH₄OAc 20% to 50%, flow 25 mL/min) to give ethyl 6-(3-{2-[(benzylsulfonyl)amino]-2-oxoethyl}azetidin-1-yl)-5-cyano-2-(difluoromethyl)nicotinate. Yield: 156 mg (46% over 3 steps).

¹H NMR (500 MHz, DMSO-d₆): δ 1.31 (3H, t, J=7.1 Hz), 2.71 (2H, d, J=7.6 Hz), 3.04-3.11 (1H, m), 4.08 (2H, apparent br s), 4.28 (2H, q, J=7.1 Hz), 4.52 (2H, apparent br s), 4.70 (2H, s), 7.29-7.32 (2H, m), 7.37-7.44 (3H, m), 7.40 (1H, t, J=53 Hz, —CHF2), 8.44 (1H, s), 11.68 (1H, s).

MS ^{m}/_{z}: 493 (M+1), 491 (M−1)

1. A compound of formula I or a pharmaceutically acceptable salt thereof: 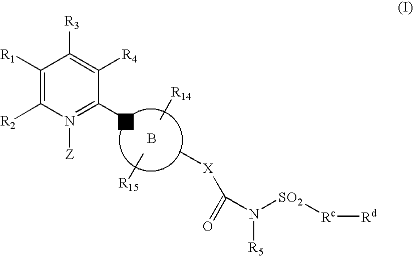
wherein
R₁ represents R₆OC(O), R₇C(O), R₁₆SC(O), R₁₇S, R₁₈C(S) or a group gII 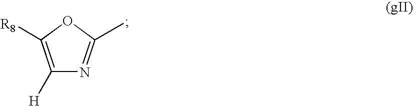
R₂ represents H, CN, halogen, NO₂, (C₁-C₁₂)alkyl (optionally interrupted by oxygen and/or optionally substituted by OH, aryl, cycloalkyl, heterocyclyl or one or more halogen atoms), (C₁-C₁₂)alkoxy (optionally substituted by one or more halogen atoms), (C₃-C₆)cycloalkyl, hydroxy(C₁-C₁₂)alkyl, (C₁-C₁₂)alkylC(O), (C₁-C₁₂)alkylthioC(O), (C₁-C₁₂)alkylC(S), (C₁-C₁₂)alkoxyC(O), (C₃-C₆)cycloalkoxy, aryl, arylC(O), aryl(C₁-C₁₂)alkylC(O), heterocyclyl, heterocyclylC(O), heterocyclyl(C₁-C₁₂)alkylC(O), (C₁-C₁₂)alkylsulfinyl, (C₁-C₁₂)alkylsulfonyl, (C₁-C₁₂)alkylthio, (C₃-C₆)cycloalkylthio, arylsulfinyl, arylsulfonyl, arylthio, aryl(C₁-C₁₂)alkylthio, aryl(C₁-C₁₂)alkylsulfinyl, aryl(C₁-C₁₂)alkylsulfonyl, heterocyclyl(C₁-C₁₂)alkylthio, heterocyclyl(C₁-C₁₂)alkylsulfinyl, heterocyclyl(C₁-C₁₂)alkylsulfonyl, (C₃-C₆)cycloalkyl(C₁-C₁₂)alkylthio, (C₃-C₆)cycloalkyl(C₁-C₁₂)alkylsulfinyl, (C₃-C₆)cycloalkyl(C₁-C₁₂)alkylsulfonyl or a group of formula NR^{a(2)}R^{b(2)} in which R^{a(2)} and R^{b(2)} independently represent H, (C₁-C₁₂)alkyl, (C₁-C₁₂)alkylC(O) or R^{a(2)} and R^{b(2)} together with the nitrogen atom represent piperidine, pyrrolidine, azetidine or aziridine; or
R₁ and R₂ together (with two carbon atoms of the pyridine ring) form a 5-membered or 6-membered cyclic lactone;
R₃ represents H, CN, NO₂, halogen, (C₁-C₁₂)alkyl (optionally interrupted by oxygen and/or optionally substituted by OH, aryl, cycloalkyl, heterocyclyl or one or more halogen atoms), (C₁-C₁₂)alkoxy (optionally substituted by one or more halogen atoms), (C₃-C₆)cycloalkyl, hydroxy(C₁-C₁₂)alkyl, (C₁-C₁₂)alkylC(O), (C₁-C₁₂)alkylthioC(O), (C₁-C₁₂)alkylC(S), (C₁-C₁₂)alkoxyC(O), (C₃-C₆)cycloalkoxy, aryl, arylC(O), aryl(C₁-C₁₂)alkylC(O), heterocyclyl, heterocyclylC(O), heterocyclyl(C₁-C₁₂)alkylC(O), (C₁-C₁₂)alkylsulfinyl, (C₁-C₁₂)alkylsulfonyl, (C₁-C₁₂)alkylthio, (C₃-C₆)cycloalkylthio, arylsulfinyl, arylsulfonyl, arylthio, aryl(C₁-C₁₂)alkylthio, aryl(C₁-C₁₂)alkylsulfinyl, aryl(C₁-C₁₂)alkylsulfonyl, heterocyclyl(C₁-C₁₂)alkylthio, heterocyclyl(C₁-C₁₂)alkylsulfinyl, heterocyclyl(C₁-C₁₂)alkylsulfonyl, (C₃-C₆)cycloalkyl(C₁-C₁₂)alkylthio, (C₃-C₆)cycloalkyl(C₁-C₁₂)alkylsulfinyl, (C₃-C₆)cycloalkyl(C₁-C₁₂)alkylsulfonyl or a group of formula NR^{a(3)}R^{b(3)} in which R^{a(3)} and R^{b(3)} independently represent H, (C₁-C₁₂)alkyl, (C₁-C₁₂)alkylC(O) or R^{a(3)} and R^{b(3)} together with the nitrogen atom represent piperidine, pyrrolidine, azetidine or aziridine;
R₄ represents H, CN, NO₂, halogen, (C₁-C₁₂)alkyl (optionally interrupted by oxygen and/or optionally substituted by OH, COOH, (C₁-C₆)alkoxycarbonyl, aryl, cycloalkyl, heterocyclyl or one or more halogen atoms), (C₃-C₆)cycloalkyl, hydroxy(C₁-C₁₂)alkyl, (C₁-C₁₂)alkylC(O), (C₁-C₁₂)alkylcycloalkyl, (C₁-C₁₂)alkoxy (wherein the alkoxy group may optionally be substituted by one or more halogen atoms, OH and/or COOH and/or (C₁-C₆)alkoxycarbonyl) (C₁-C₁₂)alkylthioC(O), (C₁-C₁₂)alkylC(S), (C₁-C₁₂)alkoxyC(O), (C₃-C₆)cycloalkoxy, aryl, arylC(O), aryl(C₁-C₁₂)alkylC(O), heterocyclyl, heterocyclylC(O), heterocyclyl(C₁-C₁₂)alkylC(O), (C₁-C₁₂)alkylsulfinyl, (C₁-C₁₂)alkylsulfonyl, (C₁-C₁₂)alkylthio, (C₃-C₆)cycloalkylthio, arylsulfinyl, arylsulfonyl, arylthio, aryl(C₁-C₁₂)alkylthio, aryl(C₁-C₁₂)alkylsulfinyl, aryl(C₁-C₁₂)alkylsulfonyl, heterocyclyl(C₁-C₁₂)alkylthio, heterocyclyl(C₁-C₁₂)alkylsulfinyl, heterocyclyl(C₁-C₁₂)alkylsulfonyl, (C₃-C₆)cycloalkyl(C₁-C₁₂)alkylthio, (C₃-C₆)cycloalkyl(C₁-C₁₂)alkylsulfinyl, (C₃-C₆)cycloalkyl(C₁-C₁₂)alkylsulfonyl or a group of formula NR^{a(4)}R^{b(4)} in which R^{a(4)} and R^{b(4)} independently represent H, (C₁-C₁₂)alkyl, (C₁-C₁₂)alkylC(O) or R^{a(4)} and R^{b(4)} together with the nitrogen atom represent piperidine, pyrrolidine, azetidine or aziridine;
Z represents O or is absent;
R₅ represents H or (C₁-C₁₂)alkyl;
R₆ represents (C₁-C₁₂)alkyl (optionally interrupted by oxygen, (with the proviso that any such oxygen must be at least 2 carbon atoms away from the ester-oxygen connecting the R₆ group) and/or optionally substituted by OH, aryl, cycloalkyl, heterocyclyl or one or more halogen atoms) (C₃-C₆)cycloalkyl, hydroxy(C₂-C₁₂)alkyl, aryl or heterocyclyl;
R₇ represents (C₁-C₁₂)alkyl (optionally interrupted by oxygen, and/or optionally substituted by OH, aryl, cycloalkyl, heterocyclyl or one or more halogen atoms) (C₃-C₆)cycloalkyl, hydroxy(C₁-C₁₂)alkyl, aryl or heterocyclyl;
R₈ represents H, (C₁-C₁₂)alkyl (optionally interrupted by oxygen, and/or optionally substituted by aryl, cycloalkyl, heterocyclyl or one or more halogen atoms), (C₃-C₆)cycloalkyl, hydroxy(C₁-C₁₂)alkyl, (C₁-C₁₂)alkoxy, (C₃-C₆)cycloalkoxy, aryl, heterocyclyl, (C₁-C₁₂)alkylsulfinyl, (C₁-C₁₂)alkylsulfonyl, (C₁-C₁₂)alkylthio, (C₃-C₆)cycloalkylthio, arylsulfinyl, arylsulfonyl, arylthio, aryl(C₁-C₁₂)alkylthio, aryl(C₁-C₁₂)alkylsulfinyl, aryl(C₁-C₁₂)alkylsulfonyl, heterocyclyl(C₁-C₁₂)alkylthio, heterocyclyl(C₁-C₁₂)alkylsulfinyl, heterocyclyl(C₁-C₁₂)alkylsulfonyl, (C₃-C₆)cycloalkyl(C₁-C₁₂)alkylthio, (C₃-C₆)cycloalkyl(C₁-C₁₂)alkylsulfinyl or (C₃-C₆)cycloalkyl(C₁-C₁₂)alkylsulfonyl;
R₁₄ represents H, OH (with the proviso that the OH group must be at least 2 carbon atoms away from any heteroatom in the B ring/ring system), (C₁-C₁₂)alkyl (optionally interrupted by oxygen and/or optionally substituted by one or more of OH, COOH and COOR^{e} (wherein R^{e} represents aryl, cycloalkyl, heterocyclyl or (C₁-C₁₂)alkyl (optionally substituted by one or more of halogen atoms, OH, aryl, cycloalkyl and heterocyclyl))) aryl, heterocyclyl, one or more halogen atoms, (C₃-C₆)cycloalkyl, hydroxy(C₁-C₁₂)alkyl, (C₁-C₁₂)alkoxy, (C₃-C₆)cycloalkoxy, (C₁-C₁₂)alkylsulfinyl, (C₁-C₁₂)alkylsulfonyl, (C₁-C₁₂)alkylthio, (C₃-C₆)cycloalkylthio, arylsulfinyl, arylsulfonyl, arylthio, aryl(C₁-C₁₂)alkylthio, aryl(C₁-C₁₂)alkylsulfinyl, aryl(C₁-C₁₂)alkylsulfonyl, heterocyclyl(C₁-C₁₂)alkylthio, heterocyclyl(C₁-C₁₂)alkylsulfinyl, heterocyclyl(C₁-C₁₂)alkylsulfonyl, (C₃-C₆)cycloalkyl(C₁-C₁₂)alkylthio, (C₃-C₆)cycloalkyl(C₁-C₁₂)alkylsulfinyl or (C₃-C₆)cycloalkyl(C₁-C₁₂)alkylsulfonyl, a group of formula NR^{a(14)}R^{b(14)} in which R^{a(14)} and R^{b(14)} independently represent H, (C₁-C₁₂)alkyl, (C₁-C₁₂)alkylC(O), (C₁-C₁₂)alkoxyC(O) or R^{a(14)} and R^{b(14)} together with the nitrogen atom represent piperidine, pyrrolidine, azetidine or aziridine;
R₁₅ represents H, OH (with the proviso that the OH group must be at least 2 carbon atoms away from any heteroatom in the B ring/ring system), (C₁-C₁₂)alkyl (optionally interrupted by oxygen and/or optionally substituted by one or more of OH, COOH and COOR^{e} (wherein R^{e} represents aryl, cycloalkyl, heterocyclyl or (C₁-C₁₂)alkyl (optionally substituted by one or more of halogen atoms, OH, aryl, cycloalkyl and heterocyclyl))), aryl, heterocyclyl, one or more halogen atoms, (C₃-C₆)cycloalkyl, hydroxy(C₁-C₁₂)alkyl, (C₁-C₁₂)alkoxy, (C₃-C₆)cycloalkoxy, (C₁-C₁₂)alkylsulfinyl, (C₁-C₁₂)alkylsulfonyl, (C₁-C₁₂)alkylthio, (C₃-C₆)cycloalkylthio, arylsulfinyl, arylsulfonyl, arylthio, aryl(C₁-C₁₂)alkylthio, aryl(C₁-C₁₂)alkylsulfinyl, aryl(C₁-C₁₂)alkylsulfonyl, heterocyclyl(C₁-C₁₂)alkylthio, heterocyclyl(C₁-C₁₂)alkylsulfinyl, heterocyclyl(C₁-C₁₂)alkylsulfonyl, (C₃-C₆)cycloalkyl(C₁-C₁₂)alkylthio, (C₃-C₆)cycloalkyl(C₁-C₁₂)alkylsulfinyl, (C₃-C₆)cycloalkyl(C₁-C₁₂)alkylsulfonyl or a group of formula NR^{a(15)}R^{b(15)} in which R^{a(15)} and R^{b(15)} independently represent H, (C₁-C₁₂)alkyl, (C₁-C₁₂)alkylC(O), (C₁-C₁₂)alkoxyC(O) or R^{a(15)} and R^{b(15)} together with the nitrogen atom represent piperidine, pyrrolidine, azetidine or aziridine;
R₁₆ represents (C₁-C₁₂)alkyl (optionally interrupted by oxygen and/or optionally substituted by OH, aryl, cycloalkyl, heterocyclyl or one or more halogen atoms) (C₃-C₆)cycloalkyl, hydroxy(C₂-C₁₂)alkyl, (C₁-C₁₂)alkoxy, (C₃-C₆)cycloalkoxy, aryl or heterocyclyl;
R₁₇ represents (C₁-C₁₂)alkyl (optionally interrupted by oxygen and/or optionally substituted by OH, aryl, cycloalkyl, heterocyclyl or one or more halogen atoms) (C₃-C₆)cycloalkyl, hydroxy(C₁-C₁₂)alkyl, (C₁-C₁₂)alkoxy, (C₃-C₆)cycloalkoxy, aryl or heterocyclyl;
R₁₈ represents (C₁-C₁₂)alkyl (optionally interrupted by oxygen and/or optionally substituted by OH, aryl, cycloalkyl, heterocyclyl or one or more halogen atoms) (C₃-C₆)cycloalkyl, hydroxy(C₁-C₁₂)alkyl, (C₁-C₁₂)alkoxy, (C₃-C₆)cycloalkoxy, aryl or heterocyclyl;
R^{c} represents an unsubstituted or monosubstituted or polysubstituted (C₁-C₄)alkylene group, (C₁-C₄)oxoalkylene group, (C₁-C₄)alkyleneoxy or oxy-(C₁-C₄)alkylene group, wherein any substituents each individually and independently are selected from (C₁-C₄)alkyl, (C₁-C₄)alkoxyl, oxy-(C₁-C₄)alkyl, (C₂-C₄)alkenyl, (C₂-C₄)alkynyl, (C₃-C₆)cycloalkyl, carboxyl, carboxy-(C₁-C₄)alkyl, aryl, heterocyclyl, nitro, cyano, halogeno, hydroxyl, NR^{a(Rc)}R^{b(Rc)} (in which R^{a(Rc)} and R^{b(Rc)} individually and independently from each other represents hydrogen, (C₁-C₄)alkyl or R^{a(Rc)} and R^{b(Rc)} together with the nitrogen atom represent piperidine, pyrrolidine, azetidine or aziridine) imino (—NH—), N-substituted imino (—NR₁₉—), (C₁-C₄)alkyleneimino or N-substituted (C₁-C₄)alkyleneimino (—N(R₁₉)—((C₁-C₄)alkylene) wherein the mentioned alkylene groups are unsubstituted or monosubstituted or polysubstituted with any substituents according to above;
R₁₉ represents H or (C₁-C₄)alkyl;
R^{d} represents (C₃-C₈)cycloalkyl, aryl or heterocyclyl, and anyone of these groups optionally substituted with one or more halogen atoms and/or one or more of the following groups, OH, CN, NO₂, (C₁-C₁₂)alkyl, (C₁-C₁₂)alkoxyC(O), (C₁-C₁₂)alkoxy, halogen substituted (C₁-C₁₂)alkyl, (C₃-C₆)cycloalkyl, aryl, heterocyclyl, (C₁-C₁₂)alkylsulfinyl, (C₁-C₁₂)alkylsulfonyl, (C₁-C₁₂)alkylthio, (C₃-C₆)cycloalkylthio, arylsulfinyl, arylsulfonyl, arylthio, aryl(C₁-C₁₂)alkylthio, aryl(C₁-C₁₂)alkylsulfinyl, aryl(C₁-C₁₂)alkylsulfonyl, heterocyclyl(C₁-C₁₂)alkylthio, heterocyclyl(C₁-C₁₂)alkylsulfinyl, heterocyclyl(C₁-C₁₂)alkylsulfonyl, (C₃-C₆)cycloalkyl(C₁-C₁₂)alkylthio, (C₃-C₆)cycloalkyl(C₁-C₁₂)alkylsulfinyl, (C₃-C₆)cycloalkyl(C₁-C₁₂)alkylsulfonyl or a group of formula NR^{a(Rd)}R^{b(Rd)} in which R^{a(Rd)} and R^{b(Rd)} independently represent H, (C₁-C₁₂)alkyl, (C₁-C₁₂)alkylC(O) or R^{a(Rd)} and R^{b(Rd)} together with the nitrogen atom represent piperidine, pyrrolidine, azetidine or aziridine;
X represents a single bond, imino (—NH—), methylene (—CH₂—), iminomethylene (—CH₂—NH—) wherein the carbon is connected to the B-ring/ring system, methyleneimino (—NH—CH₂—) wherein the nitrogen is connected to the B-ring/ring system (and any carbon and/or nitrogen in these groups may optionally be substituted with (C₁-C₆) alkyl), a group (—CH₂—)ₙ wherein n=2-6, which optionally is unsaturated and/or substituted by one or more substituent selected from halogen, hydroxyl and (C₁-C₆)alkyl; and
B is a monocyclic or bicyclic, 4 to 11-membered heterocyclic ring/ring system comprising one or more nitrogen and optionally one or more atoms selected from oxygen or sulphur, which nitrogen is connected to the pyridine-ring (according to formula I) and further the B-ring/ring system is connected to X in another of its positions; the substituents R₁₄ and R₁₅ are connected to the B ring/ring system in such a way that no quarternary ammonium compounds are formed (by these connections).

2. A compound according to claim 1 wherein:
R₂ represents H, CN, NO₂, (C₁-C₆)alkyl (optionally interrupted by oxygen and/or optionally substituted by OH, aryl, cycloalkyl, heterocyclyl or one or more halogen atoms), (C₁-C₆)alkoxy (optionally substituted by one or more halogen atoms), (C₃-C₆)cycloalkyl, hydroxy(C₁-C₆)alkyl, (C₁-C₆)alkylC(O), (C₁-C₆)alkylthioC(O), (C₁-C₆)alkylC(S), (C₁-C₆)alkoxyC(O), (C₃-C₆)cycloalkoxy, aryl, arylC(O), aryl(C₁-C₆)alkylC(O), heterocyclyl, heterocyclylC(O), heterocyclyl(C₁-C₆)alkylC(O), (C₁-C₆)alkylsulfinyl, (C₁-C₆)alkylsulfonyl, (C₁-C₆)alkylthio, (C₃-C₆)cycloalkylthio, arylsulfinyl, arylsulfonyl, arylthio, aryl(C₁-C₆)alkylthio, aryl(C₁-C₆)alkylsulfinyl, aryl(C₁-C₆)alkylsulfonyl, heterocyclyl(C₁-C₆)alkylthio, heterocyclyl(C₁-C₆)alkylsulfinyl, heterocyclyl(C₁-C₆)alkylsulfonyl, (C₃-C₆)cycloalkyl(C₁-C₆)alkylthio, (C₃-C₆)cycloalkyl(C₁-C₆)alkylsulfinyl, (C₃-C₆)cycloalkyl(C₁-C₆)alkylsulfonyl or a group of formula NR^{a(2)}R^{b(2)} in which R^{a(2)} and R^{b(2)} independently represent H, (C₁-C₆)alkyl, (C₁-C₆)alkylC(O) or R^{a(2)} and R^{b(2)} together with the nitrogen atom represent piperidine, pyrrolidine, azetidine or aziridine; or
R₁ and R₂ together (with two carbons from the pyridine ring) form a 5-membered or 6-membered cyclic lactone;
R₃ represents H, CN, NO₂, halogen, (C₁-C₆)alkyl (optionally interrupted by oxygen and/or optionally substituted by OH, aryl, cycloalkyl, heterocyclyl or one or more halogen atoms), (C₁-C₆)alkoxy (optionally substituted by one or more halogen atoms), (C₃-C₆)cycloalkyl, hydroxy(C₁-C₆)alkyl, (C₁-C₆)alkylC(O), (C₁-C₆)alkylthioC(O), (C₁-C₆)alkylC(S), (C₁-C₆)alkoxyC(O), (C₃-C₆)cycloalkoxy, aryl, arylC(O), aryl(C₁-C₆)alkylC(O), heterocyclyl, heterocyclylC(O), heterocyclyl(C₁-C₆)alkylC(O), (C₁-C₆)alkylsulfinyl, (C₁-C₆)alkylsulfonyl, (C₁-C₆)alkylthio, (C₃-C₆)cycloalkylthio, arylsulfinyl, arylsulfonyl, arylthio, aryl(C₁-C₆)alkylthio, aryl(C₁-C₆)alkylsulfinyl, aryl(C₁-C₆)alkylsulfonyl, heterocyclyl(C₁-C₆)alkylthio, heterocyclyl(C₁-C₆)alkylsulfinyl, heterocyclyl(C₁-C₆)alkylsulfonyl, (C₃-C₆)cycloalkyl(C₁-C₆)alkylthio, (C₃-C₆)cycloalkyl(C₁-C₆)alkylsulfinyl, (C₃-C₆)cycloalkyl(C₁-C₆)alkylsulfonyl or a group of formula NR^{a(3)}R^{b(3)} in which R^{a(3)} and R^{b(3)} independently represent H, (C₁-C₆)alkyl, (C₁-C₆)alkylC(O) or R^{a(3)} and R^{b(3)} together with the nitrogen atom represent piperidine, pyrrolidine, azetidine or aziridine;
R₄ represents H, CN, NO₂, halogen, (C₁-C₆)alkyl (optionally interrupted by oxygen and/or optionally substituted by OH, COOH, (C₁-C₆)alkoxycarbonyl, aryl, cycloalkyl, heterocyclyl or one or more halogen atoms), (C₃-C₆)cycloalkyl, hydroxy(C₁-C₆)alkyl, (C₁-C₆)alkylC(O), (C₁-C₆)alkoxy (wherein the alkoxy group may optionally be substituted by one or more halogen atoms, OH and/or COOH and/or (C₁-C₃)alkoxycarbonyl), (C₁-C₆)alkylthioC(O), (C₁-C₆)alkylC(S), (C₁-C₆)alkoxyC(O), (C₃-C₆)cycloalkoxy, aryl, arylC(O), aryl(C₁-C₆)alkylC(O), heterocyclyl, heterocyclylC(O), heterocyclyl(C₁-C₆)alkylC(O), (C₁-C₆)alkylsulfinyl, (C₁-C₆)alkylsulfonyl, (C₁-C₆)alkylthio, (C₃-C₆)cycloalkylthio, arylsulfinyl, arylsulfonyl, arylthio, aryl(C₁-C₆)alkylthio, aryl(C₁-C₆)alkylsulfinyl, aryl(C₁-C₆)alkylsulfonyl, heterocyclyl(C₁-C₆)alkylthio, heterocyclyl(C₁-C₆)alkylsulfinyl, heterocyclyl(C₁-C₆)alkylsulfonyl, (C₃-C₆)cycloalkyl(C₁-C₆)alkylthio, (C₃-C₆)cycloalkyl(C₁-C₆)alkylsulfinyl, (C₃-C₆)cycloalkyl(C₁-C₆)alkylsulfonyl or a group of formula NR^{a(4)}R^{b(4)} in which R^{a(4)} and R^{b(4)} independently represent H, (C₁-C₆)alkyl, (C₁-C₆)alkylC(O) or R^{a(4)} and R^{b(4)} together with the nitrogen atom represent piperidine, pyrrolidine, azetidine or aziridine;
R₅ represents H or (C₁-C₆)alkyl;
R₆ represents (C₁-C₆)alkyl (optionally interrupted by oxygen, (with the proviso that any such oxygen must be at least 1 carbon atom away from the ester-oxygen connecting the R₆ group) and/or optionally substituted by OH, aryl, cycloalkyl, heterocyclyl or one or more halogen atoms) (C₃-C₆)cycloalkyl, hydroxy(C₂-C₆)alkyl, aryl or heterocyclyl;
R₇ represents (C₁-C₆)alkyl (optionally interrupted by oxygen, and/or optionally substituted by OH, aryl, cycloalkyl, heterocyclyl or one or more halogen atoms), (C₃-C₆)cycloalkyl, hydroxy(C₁-C₆)alkyl, aryl or heterocyclyl;
R₈ represents H, (C₁-C₆)alkyl (optionally interrupted by oxygen, and/or optionally substituted by aryl, cycloalkyl, heterocyclyl or one or more halogen atoms), (C₃-C₆)cycloalkyl, hydroxy(C₁-C₆)alkyl, (C₁-C₆)alkoxy, (C₃-C₆)cycloalkoxy, aryl, heterocyclyl, (C₁-C₆)alkylsulfinyl, (C₁-C₆)alkylsulfonyl, (C₁-C₆)alkylthio, (C₃-C₆)cycloalkylthio, arylsulfinyl, arylsulfonyl, arylthio, aryl(C₁-C₆)alkylthio, aryl(C₁-C₆)alkylsulfinyl, aryl(C₁-C₆)alkylsulfonyl, heterocyclyl(C₁-C₆)alkylthio, heterocyclyl(C₁-C₆)alkylsulfinyl, heterocyclyl(C₁-C₆)alkylsulfonyl, (C₃-C₆)cycloalkyl(C₁-C₆)alkylthio, (C₃-C₆)cycloalkyl(C₁-C₆)alkylsulfinyl or (C₃-C₆)cycloalkyl(C₁-C₆)alkylsulfonyl;
R₁₄ represents H, OH (with the proviso that the OH group must be at least 2 carbon atoms away from any heteroatom in the B ring/ring system), (C₁-C₆)alkyl (optionally interrupted by oxygen and/or optionally substituted by one or more of OH, COOH and COOR^{e} (wherein R^{e} represents aryl, cycloalkyl, heterocyclyl or (C₁-C₆)alkyl (optionally substituted by one or more of halogen atoms, OH, aryl, cycloalkyl and heterocyclyl))), aryl, heterocyclyl, one or more halogen atoms, (C₃-C₆)cycloalkyl, hydroxy(C₁-C₆)alkyl, (C₁-C₆)alkoxy, (C₃-C₆)cycloalkoxy, (C₁-C₆)alkylsulfinyl, (C₁-C₆)alkylsulfonyl, (C₁-C₆)alkylthio, (C₃-C₆)cycloalkylthio, arylsulfinyl, arylsulfonyl, arylthio, aryl(C₁-C₆)alkylthio, aryl(C₁-C₆)alkylsulfinyl, aryl(C₁-C₆)alkylsulfonyl, heterocyclyl(C₁-C₆)alkylthio, heterocyclyl(C₁-C₆)alkylsulfinyl, heterocyclyl(C₁-C₆)alkylsulfonyl, (C₃-C₆)cycloalkyl(C₁-C₆)alkylthio, (C₃-C₆)cycloalkyl(C₁-C₆)alkylsulfinyl, (C₃-C₆)cycloalkyl(C₁-C₆)alkylsulfonyl or a group of formula NR^{a(14)}R^{b(14)} in which R^{a(14)} and R^{b(14)} independently represent H, (C₁-C₆)alkyl, (C₁-C₆)alkylC(O), (C₁-C₆)alkoxyC(O) or R^{a(14)} and R^{b(14)} together with the nitrogen atom represent piperidine, pyrrolidine, azetidine or aziridine;
R₁₅ represents H, OH (with the proviso that the OH group must be at least 2 carbon atoms away from any heteroatom in the B ring/ring system), (C₁-C₆)alkyl (optionally interrupted by oxygen and/or optionally substituted by one or more of OH, COOH and COOR^{e} (wherein R^{e} represents aryl, cycloalkyl, heterocyclyl or (C₁-C₆)alkyl (optionally substituted by one or more of halogen atoms, OH, aryl, cycloalkyl and heterocyclyl))), aryl, heterocyclyl, one or more halogen atoms, (C₃-C₆)cycloalkyl, hydroxy(C₁-C₆)alkyl, (C₁-C₆)alkoxy, (C₃-C₆)cycloalkoxy, (C₁-C₆)alkylsulfinyl, (C₁-C₆)alkylsulfonyl, (C₁-C₆)alkylthio, (C₃-C₆)cycloalkylthio, arylsulfinyl, arylsulfonyl, arylthio, aryl(C₁-C₆)alkylthio, aryl(C₁-C₆)alkylsulfinyl, aryl(C₁-C₆)alkylsulfonyl, heterocyclyl(C₁-C₆)alkylthio, heterocyclyl(C₁-C₆)alkylsulfinyl, heterocyclyl(C₁-C₆)alkylsulfonyl, (C₃-C₆)cycloalkyl(C₁-C₆)alkylthio, (C₃-C₆)cycloalkyl(C₁-C₆)alkylsulfinyl, (C₃-C₆)cycloalkyl(C₁-C₆)alkylsulfonyl or a group of formula NR^{a(15)}R^{b(15)} in which R^{a(15)} and R^{b(15)} independently represent H, (C₁-C₆)alkyl, (C₁-C₆)alkylC(O), (C₁-C₆)alkoxyC(O) or R^{a(15)} and R^{b(15)} together with the nitrogen atom represent piperidine, pyrrolidine, azetidine or aziridine;
R₁₆ represents (C₁-C₆)alkyl optionally (optionally interrupted by oxygen and/or optionally substituted by OH, aryl, cycloalkyl, heterocyclyl or one or more halogen atoms), (C₃-C₆)cycloalkyl, hydroxy(C₂-C₆)alkyl, (C₁-C₆)alkoxy, (C₃-C₆)cycloalkoxy, aryl, or heterocyclyl;
R₁₇ represents (C₁-C₆)alkyl (optionally interrupted by oxygen and/or optionally substituted by OH, aryl, cycloalkyl, heterocyclyl or one or more halogen atoms), (C₃-C₆)cycloalkyl, hydroxy(C₁-C₆)alkyl, (C₁-C₆)alkoxy, (C₃-C₆)cycloalkoxy, aryl or heterocyclyl;
R₁₈ represents (C₁-C₆)alkyl (optionally interrupted by oxygen and/or optionally substituted by OH, aryl, cycloalkyl, heterocyclyl or one or more halogen atoms), (C₃-C₆)cycloalkyl, hydroxy(C₁-C₆)alkyl, (C₁-C₆)alkoxy, (C₃-C₆)cycloalkoxy, aryl or heterocyclyl; and
R^{d} represents (C₃-C₈)cycloalkyl, aryl or heterocyclyl, and anyone of these groups optionally substituted with one or more halogen atoms and/or one or more of the following groups, OH, CN, NO₂, (C₁-C₆)alkyl, (C₁-C₆)alkoxyC(O), (C₁-C₆)alkoxy, halogen substituted (C₁-C₆)alkyl, (C₃-C₆)cycloalkyl, aryl, heterocyclyl, (C₁-C₆)alkylsulfinyl, (C₁-C₆)alkylsulfonyl, (C₁-C₆)alkylthio, (C₃-C₆)cycloalkylthio, arylsulfinyl, arylsulfonyl, arylthio, aryl(C₁-C₆)alkylthio, aryl(C₁-C₆)alkylsulfinyl, aryl(C₁-C₆)alkylsulfonyl, heterocyclyl(C₁-C₆)alkylthio, heterocyclyl(C₁-C₆)alkylsulfinyl, heterocyclyl(C₁-C₆)alkylsulfonyl, (C₃-C₆)cycloalkyl(C₁-C₆)alkylthio, (C₃-C₆)cycloalkyl(C₁-C₆)alkylsulfinyl, (C₃-C₆)cycloalkyl(C₁-C₆)alkylsulfonyl or a group of formula NR^{a(Rd)}R^{b(Rd)} in which R^{a(Rd)} and R^{b(Rd)} independently represent H, (C₁-C₆)alkyl, (C₁-C₆)alkylC(O) or R^{a(Rd)} and R^{b(Rd)} together with the nitrogen atom represent piperidine, pyrrolidine, azetidine or aziridine.

3. A compound according to claim 2 wherein:
R₁ represents R₆OC(O), R₁₆SC(O), or a group gII, 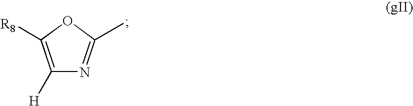
R₂ represents H, CN, NO₂, (C₁-C₆)alkyl (optionally interrupted by oxygen and/or optionally substituted by OH, aryl, cycloalkyl, heterocyclyl or one or more halogen atoms) (C₁-C₆)alkoxy (optionally substituted by one or more halogen atoms), (C₃-C₆)cycloalkyl, hydroxy(C₁-C₆)alkyl, (C₁-C₆)alkylC(O), (C₁-C₆)alkylthioC(O), (C₁-C₆)alkylC(S), (C₁-C₆)alkoxyC(O), (C₃-C₆)cycloalkoxy, aryl, arylC(O), aryl(C₁-C₆)alkylC(O), heterocyclyl, heterocyclylC(O), heterocyclyl(C₁-C₆)alkylC(O) or a group of formula NR^{a(2)}R^{b(2)} in which R^{a(2)} and R^{b(2)} independently represent H, (C₁-C₆)alkyl, (C₁-C₆)alkylC(O) or R^{a(2)} and R^{b(2)} together with the nitrogen atom represent piperidine, pyrrolidine, azetidine or aziridine;
R₃ represents H, CN, NO₂, halogen, (C₁-C₆)alkyl (optionally interrupted by oxygen and/or optionally substituted by OH, aryl, cycloalkyl, heterocyclyl or one or more halogen atoms), (C₁-C₆)alkoxy (optionally substituted by one or more halogen atoms), (C₃-C₆)cycloalkyl, hydroxy(C₁-C₆)alkyl, (C₁-C₆)alkylC(O), (C₁-C₆)alkylthioC(O), (C₁-C₆)alkylC(S), (C₁-C₆)alkoxyC(O), (C₃-C₆)cycloalkoxy, aryl, arylC(O), aryl(C₁-C₆)alkylC(O), heterocyclyl, heterocyclylC(O), heterocyclyl(C₁-C₆)alkylC(O), (C₁-C₆)alkylsulfinyl, or a group of formula NR^{a(3)}R^{b(3)} in which R^{a(3)} and R^{b(3)} independently represent H, (C₁-C₆)alkyl, (C₁-C₆)alkylC(O) or R^{a(3)} and R^{b(3)} together with the nitrogen atom represent piperidine, pyrrolidine, azetidine or aziridine;
R₄ represents H, CN, NO₂, halogen, (C₁-C₆)alkyl (optionally interrupted by oxygen and/or optionally substituted by OH, COOH, aryl, cycloalkyl, heterocyclyl or one or more halogen atoms) (C₃-C₆)cycloalkyl, hydroxy(C₁-C₆)alkyl, (C₁-C₆)alkylC(O), (C₁-C₆)alkoxy (wherein the alkoxy group may optionally be substituted by one or more halogen atoms, OH and/or COOH and/or methoxycarbonyl), (C₁-C₆)alkylthioC(O), (C₁-C₆)alkylC(S), (C₁-C₆)alkoxyC(O), (C₃-C₆)cycloalkoxy, aryl, arylC(O), aryl(C₁-C₆)alkylC(O), heterocyclyl, heterocyclylC(O), heterocyclyl(C₁-C₆)alkylC(O) or a group of formula NR^{a(4)}R^{b(4)} in which R^{a(4)} and R^{b(4)} independently represent H, (C₁-C₆)alkyl, (C₁-C₆)alkylC(O) or R^{a(4)} and R^{b(4)} together with the nitrogen atom represent piperidine, pyrrolidine, azetidine or aziridine;
R₈ represents H, (C₁-C₆)alkyl (optionally interrupted by oxygen, and/or optionally substituted by aryl, cycloalkyl, heterocyclyl or one or more halogen atoms), (C₃-C₆)cycloalkyl, hydroxy(C₁-C₆)alkyl, (C₁-C₆)alkoxy, (C₃-C₆)cycloalkoxy, aryl or heterocyclyl;
R₁₄ represents H, OH (with the proviso that the OH group must be at least 2 carbon atoms away from any heteroatom in the B ring/ring system), (C₁-C₆)alkyl (optionally interrupted by oxygen and/or optionally substituted by one or more of OH, COOH and COOR^{e} (wherein R^{e} represents aryl, cycloalkyl, heterocyclyl or (C₁-C₆)alkyl (optionally substituted by one or more of halogen atoms, OH, aryl, cycloalkyl and heterocyclyl))), aryl, heterocyclyl, one or more halogen atoms, (C₃-C₆)cycloalkyl, hydroxy(C₁-C₆)alkyl, (C₁-C₆)alkoxy, (C₃-C₆)cycloalkoxy, or a group of formula NR^{a(14)}R^{b(14)} in which R^{a(14)} and R^{b(14)} independently represent H, (C₁-C₆)alkyl, (C₁-C₆)alkylC(O), (C₁-C₆)alkoxyC(O) or R^{a(14)} and R^{b(14)} together with the nitrogen atom represent piperidine, pyrrolidine, azetidine or aziridine;
R₁₅ represents H, OH (with the proviso that the OH group must be at least 2 carbon atoms away from any heteroatom in the B ring/ring system), (C₁-C₆)alkyl (optionally interrupted by oxygen and/or optionally substituted by one or more of OH, COOH and COOR^{e} (wherein R^{e} represents aryl, cycloalkyl, heterocyclyl or (C₁-C₆)alkyl (optionally substituted by one or more of halogen atoms, OH, aryl, cycloalkyl and heterocyclyl))), aryl, heterocyclyl, one or more halogen atoms, (C₃-C₆)cycloalkyl, hydroxy(C₁-C₆)alkyl, (C₁-C₆)alkoxy, (C₃-C₆)cycloalkoxy, or a group of formula NR^{a(15)}R^{b(15)} in which R^{a(15)} and R^{b(15)} independently represent H, (C₁-C₆)alkyl, (C₁-C₆)alkylC(O), (C₁-C₆)alkoxyC(O) or R^{a(15)} and R^{b(15)} together with the nitrogen atom represent piperidine, pyrrolidine, azetidine or aziridine;
R₁₆ is ethyl;
R^{c} represents an unsubstituted or monosubstituted or polysubstituted (C₁-C₄)alkylene group, (C₁-C₄)oxoalkylene group, (C₁-C₄)alkyleneoxy or oxy-(C₁-C₄)alkylene group, wherein any substituents each individually and independently are selected from (C₁-C₄)alkyl, (C₁-C₄)alkoxyl, oxy-(C₁-C₄)alkyl, (C₂-C₄)alkenyl, (C₂-C₄)alkynyl, (C₃-C₆)cycloalkyl, carboxyl, carboxy-(C₁-C₄)alkyl, aryl, heterocyclyl, nitro, cyano, halogeno, hydroxyl, NR^{a(Rc)}R^{b(Rc)} (in which R^{a(Rc)} and R^{b(Rc)} individually and independently from each other represents hydrogen, (C₁-C₄)alkyl or R^{a(Rc)} and R^{b(Rc)} together with the nitrogen atom represent piperidine, pyrrolidine, azetidine or aziridine) imino (—NH—), N-substituted imino (—NR₁₉—), (C₁-C₄)alkyleneimino or N-substituted (C₁-C₄)alkyleneimino (—N(R₁₉)—((C₁-C₄)alkylene) wherein the mentioned alkylene groups are unsubstituted or monosubstituted or polysubstituted with any substituents according to above; and
R^{d} represents (C₃-C₈)cycloalkyl, aryl or heterocyclyl, and anyone of these groups optionally substituted with one or more halogen atoms and/or one or more of the following groups, CN, NO₂, (C₁-C₆)alkyl, (C₁-C₆)alkoxy, halosubstituted (C₁-C₆)alkyl, (C₃-C₆)cycloalkyl, aryl, heterocyclyl, (C₁-C₆)alkylsulfinyl, (C₁-C₆)alkylsulfonyl, (C₁-C₆)alkylthio, (C₃-C₆)cycloalkylthio, arylsulfinyl, arylsulfonyl, arylthio, aryl(C₁-C₆)alkylthio, aryl(C₁-C₆)alkylsulfinyl, aryl(C₁-C₆)alkylsulfonyl, heterocyclyl(C₁-C₆)alkylthio, heterocyclyl(C₁-C₆)alkylsulfinyl, heterocyclyl(C₁-C₆)alkylsulfonyl, (C₃-C₆)cycloalkyl(C₁-C₆)alkylthio, (C₃-C₆)cycloalkyl(C₁-C₆)alkylsulfinyl or (C₃-C₆)cycloalkyl(C₁-C₆)alkylsulfonyl.

4. A compound according to claim 1 wherein:
R₁ represents R₆OC(O), R₁₆SC(O) or a group gII 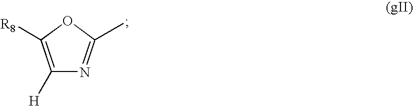
R₂ represents H or (C₁-C₆)alkyl (optionally interrupted by oxygen and/or optionally substituted by OH, aryl, cycloalkyl, heterocyclyl or one or more halogen atoms) a group of formula NR^{a(2)}R^{b(2)} in which R^{a(2)} and R^{b(2)} independently represent H, (C₁-C₆)alkyl, (C₁-C₆)alkylC(O) or R^{a(2)} and R^{b(2)} together with the nitrogen atom represent piperidine, pyrrolidine, azetidine or aziridine;
R₃ represents H or a group of formula NR^{a(3)}R^{b(3)} in which R^{a(3)} and R^{b(3)} independently represent H, (C₁-C₆)alkyl, (C₁-C₆)alkylC(O) or R^{a(3)} and R^{b(3)} together with the nitrogen atom represent piperidine, pyrrolidine, azetidine or aziridine;
R₄ represents CN, halogen (C₁-C₆)alkylC(O), (C₁-C₆)alkoxy wherein the alkoxy group may optionally be substituted by one or more halogen atoms, OH and/or COOH and/or methoxycarbonyl;
R₅ represents H;
R₆ represents (C₁-C₁₂)alkyl (optionally interrupted by oxygen, (with the proviso that any such oxygen must be at least 2 carbon atoms away from the ester-oxygen connecting the R₆ group) and/or optionally substituted by OH, aryl, cycloalkyl, heterocyclyl or one or more halogen atoms) (C₃-C₆)cycloalkyl or hydroxy(C₂-C₁₂)alkyl;
R₈ represents H, (C₁-C₆)alkyl optionally interrupted by oxygen, and/or optionally substituted by aryl, cycloalkyl, heterocyclyl or one or more halogen atoms;
R₁₄ represents H, OH (with the proviso that the OH group must be at least 2 carbon atoms away from any heteroatom in the B ring/ring system), (C₁-C₆)alkyl (optionally interrupted by oxygen and/or optionally substituted by one or more of OH, COOH and COOR^{e} (wherein R^{e} represents aryl, cycloalkyl, heterocyclyl or (C₁-C₆)alkyl (optionally substituted by one or more of halogen atoms, OH, aryl, cycloalkyl and heterocyclyl))), or a group of formula NR^{a(14)}R^{b(14)} in which R^{a(14)} and R^{b(14)} independently represent H, (C₁-C₆)alkyl, (C₁-C₆)alkylC(O), (C₁-C₆)alkoxyC(O) or R^{a(14)} and R^{b(14)} together with the nitrogen atom represent piperidine, pyrrolidine, azetidine or aziridine;
R₁₅ represents H;
R₁₆ is ethyl;
R^{c} represents an unsubstituted or monosubstituted (C₁-C₄)alkylene group, (C₁-C₄)alkyleneoxy or oxy-(C₁-C₄)alkylene group, wherein group (wherein any substituents each individually and independently are selected from (C₁-C₄)alkyl), imino (—NH—), and N-substituted imino (—NR₁₉—);
R₁₉ represents H or methyl;
R^{d} represents (C₃-C₈)cycloalkyl, aryl or heterocyclyl, and anyone of these groups optionally substituted with one or more halogen atoms and/or one or more of the following groups, CN, NO₂, (C₁-C₆)alkyl, (C₁-C₆)alkoxy, halosubstituted (C₁-C₆)alkyl; and
X represents a single bond, imino (—NH—) or methylene (—CH₂—).

5. A compound according to claim 1 wherein:
R₁ is selected from methoxycarbonyl, ethoxycarbonyl, (n-propyl)-oxycarbonyl, (iso-propyl)-oxycarbonyl, (iso-butyl)-oxycarbonyl, (tert-butyl)-oxycarbonyl, (2,2-dimethyl-propyl)-oxycarbonyl, (cyclo-propyl)-oxycarbonyl, (cyclo-butyl)-oxycarbonyl, (cyclo-pentyl)-oxycarbonyl, (2-hydroxyethyl)-oxycarbonyl), (2,2,2-trifluoroethyl)-oxycarbonyl, benzyl-oxycarbonyl, 4-fluorobenzyl-oxycarbonyl, ethylthiocarbonyl, and 5-ethyl-1,3-oxazol-2-yl;
R₂ is selected from H, methyl, ethyl, isopropyl, and dimethylamino;
R₃ is H or amino;
R₄ is selected from methoxy, chloro, cyano, (4-methoxy-4-oxobutoxy), (3-carboxy-propoxy) and methylcarbonyl;
Z represents 0 or is absent;
R₅ is H;
R₆ is selected from methyl, ethyl, 2-hydroxyethyl, (2,2,2-trifluoroethyl), n-propyl, iso-propyl, cyclo-propyl, iso-butyl, tert-butyl, cyclo-butyl, 2,2-dimethylpropyl, cyclo-pentyl, benzyl and 4-fluorobenzyl;
R₈ is ethyl;
R₁₄ is selected from H, methyl, tert-butyloxycarbonyl-imino and amino;
R₁₅ is H;
R₁₆ is ethyl;
R^{c} is selected from methylene (—CH₂—), methylmethylene (—CH(CH₃)—), ethylene (—CH₂CH₂—), oxypropylene (—OCH₂CH₂CH₂—), imino (—NH—) and methylimino (—N(CH₃)—;
R₁₉ is H or methyl;
R^{d} is selected from cyclopentyl, cyclohexyl, 4-methylcyclohexyl, phenyl, 2-methylphenyl, 3-methylphenyl, 4-methylphenyl, 4-ethylphenyl, 2-methoxycarbonyl-phenyl, 3-(trifluoromethyl)phenyl, 4-(trifluoromethyl)phenyl, 2-(trifluoromethyl)phenyl, 2-fluorophenyl, 3-fluorophenyl, 4-fluorophenyl, 2-chlorophenyl, 3-chlorophenyl, 4-chlorophenyl, 3-bromophenyl, 4-cyanophenyl, 4-methoxyphenyl, 2-nitrophenyl, 3-nitrophenyl, 4-nitrophenyl, 3,4-dichlorophenyl, 3,5-dichlorophenyl, 3,4-difluorophenyl, 2,5-dimethylphenyl, 3,5-dimethylphenyl, 4-isopropylphenyl, 3-fluoro-4-methyl-phenyl, 2-pyridyl, 3-pyridyl, 4-pyridyl, N-oxido-2-pyridyl, 6-[3-benzo[d]isoxazol-3-yl] and N-[(1,2-benzisoxazol-3-yl)];
X represents a single bond, imino (—NH—) or methylene (—CH₂—); and
B is selected from 4-piperazin-1-ylene, 4-piperidin-1-ylene, 3-piperidin-1-ylene, 3-azetidin-1-ylene, and the substituents R₁₄ and R₁₅ are connected to the B ring/ring system, in such a way that no quarternary ammonium compounds are formed (by these connections).

6. A compound according to any claim 1 which is of the formula (Ia):

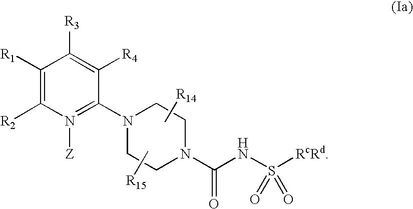

7. A compound according to claim 1 which is of the formula (Ib):

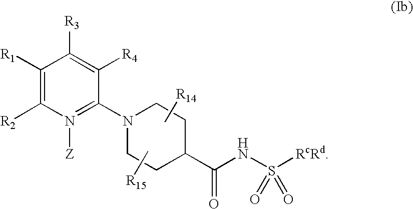

8. A compound according to claim 1 which is of the formula (Ic):

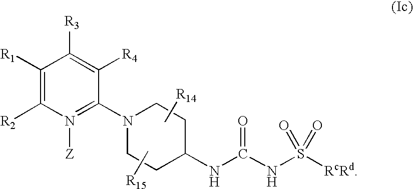

9. A compound according to claim 1 which is of the formula (Id):

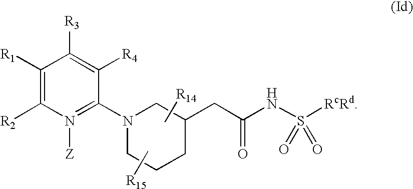

10. A compound according to claim 1 which is of the formula (Ie):

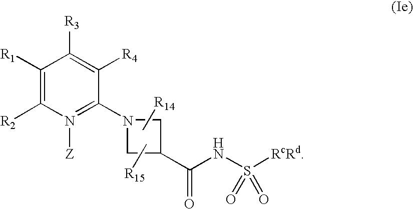

11. A compound according to claim 1 which is of the formula (If):

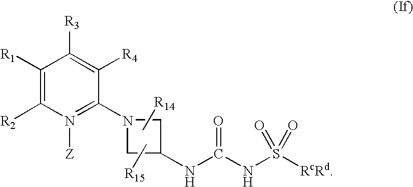

12. A compound according to claim 1 which is of the formula (Ig):

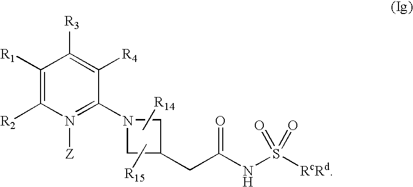

13. A compound according to claim 1 wherein Z is absent.

14. A compound according to claim 1 wherein Z is O.

15. A compound according to claim 1 wherein R₁ represents R₆OC(O).

16. A compound according claim 1 wherein R₁ represents R₁₆SC(O) or a group gII

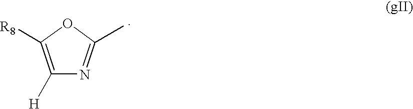

17. A compound according to claim 15 which is of the formula (Iaa):

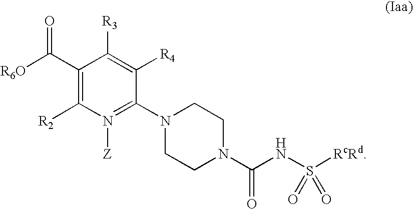

18. A compound according to claim 15 which is of the formula (Ibb):

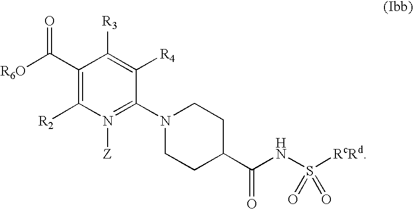

19. A compound according to claim 15 which is of the formula (Ibc):

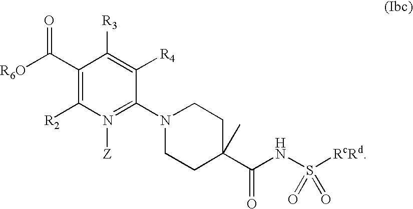

20. A compound according to claim 15 which is of the formula (Ibd):

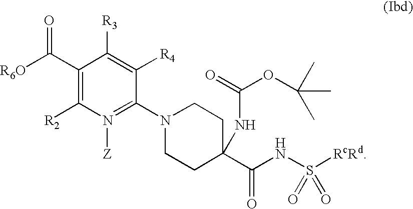

21. A compound according to claim 15 which is of the formula (Ibe):

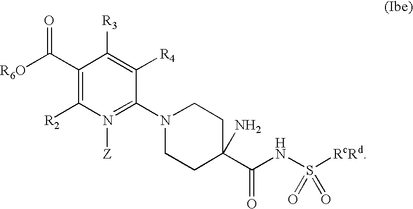

22. A compound according to claim 15 which is of the formula (Icc):

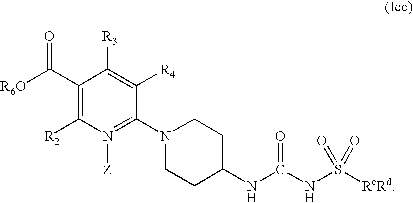

23. A compound according to claim 15 which is of the formula (Idd):

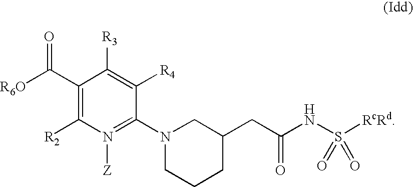

24. A compound according to claim 15 which is of the formula (Iee):

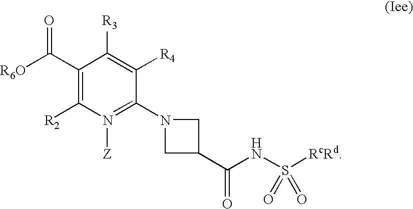

25. A compound according to claim 15 which is of the formula (Iff):

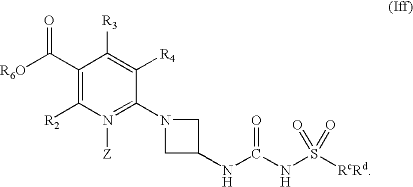

26. A compound according to claim 16 which is of the formula (Igg):

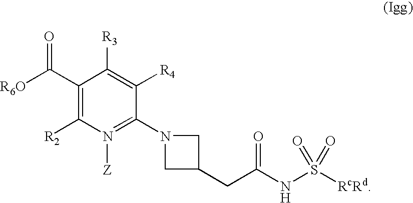

27. A compound according to claim 16 which is of the formula (Ihh):

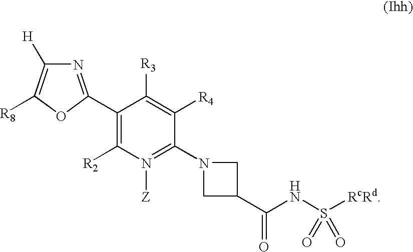

28. A compound according to claim 16 which is of the formula (Iii):

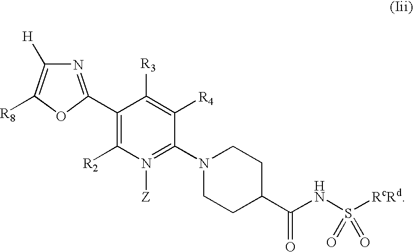

29. A compound according to claim 16 which is of the formula (Ijj):

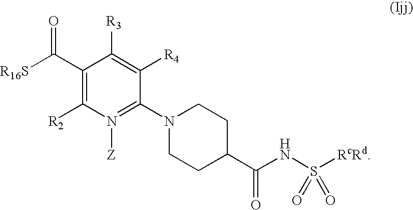

30. A compound according to claim 1 wherein R₁ represents R₆OC(O), R₁₆SC(O) or a group gII

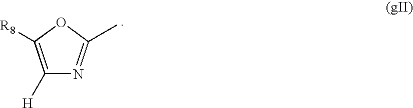

31. A compound according to claim 30 wherein R₁ represents a group gII;

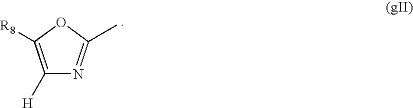

32. A compound according to claim 30 wherein R₁ represents R₁₆SC(O).

33. A compound selected from:
5-Cyano-6-[3-(2-methoxycarbonyl-phenylmethanesulfonylaminocarbonyl)azetidin-1-yl]-2-methyl-nicotinic acid ethyl ester;
6-[3-({[(3-Bromobenzyl)sulfonyl]amino}carbonyl)azetidin-1-yl]-5-cyano-2-methylnicotinic acid ethyl ester
5-Cyano-2-methyl-6-[3-(2-nitro-phenylmethanesulfonylaminocarbonyl)-zetidin-1-yl]-nicotinic acid ethyl ester;
6-[3-(2-Chloro-phenylmethanesulfonylaminocarbonyl)-azetidin-1-yl]-5-cyano-2-methyl-nicotinic acid ethyl ester;
6-[3-(4-Chloro-phenylmethanesulfonylaminocarbonyl)-azetidin-1-yl]-5-cyano-2-methyl-nicotinic acid ethyl ester;
5-Cyano-2-methyl-6-[3-(4-trifluoromethyl-phenylmethanesulfonylaminocarbonyl)azetidin-1-yl]-nicotinic acid ethyl ester;
5-Cyano-6-[3-(3-fluoro-phenylmethanesulfonylaminocarbonyl)-azetidin-1-yl]-2-methyl-nicotinic acid ethyl ester;
5-Cyano-2-methyl-6-[3-(3-trifluoromethyl-phenylmethanesulfonylaminocarbonyl)azetidin-1-yl]-nicotinic acid ethyl ester;
6-[3-(3-Chloro-phenylmethanesulfonylaminocarbonyl)-azetidin-1-yl]-5-cyano-2-methyl-nicotinic acid ethyl ester;
6-{3-[2-(3-Chloro-phenyl)-ethanesulfonylaminocarbonyl]-azetidin-1-yl}-5-cyano-2-methyl-nicotinic acid ethyl ester;
5-Cyano-2-methyl-6-[3-(4-nitro-phenylmethanesulfonylaminocarbonyl)-azetidin-1-yl]-nicotinic acid ethyl ester;
5-Cyano-2-methyl-6-[3-(2-phenyl-ethanesulfonylaminocarbonyl)-azetidin-1-yl]-nicotinic acid ethyl ester;
5-Cyano-2-methyl-6-(3-o-tolylmethanesulfonylaminocarbonyl-azetidin-1-yl)-nicotinic acid ethyl ester;
5-Cyano-2-methyl-6-[3-(3-nitro-phenylmethanesulfonylaminocarbonyl)-azetidin-1-yl]-nicotinic acid ethyl ester;
5-Cyano-6-{3-[2-(4-fluoro-phenyl)-ethanesulfonylaminocarbonyl]-azetidin-1-yl}-2-methyl-nicotinic acid ethyl ester;
5-Cyano-2-methyl-6-[3-(2-trifluoromethyl-phenylmethanesulfonylaminocarbonyl)azetidin-1-yl]-nicotinic acid ethyl ester;
5-Cyano-6-[3-(4-fluoro-phenylmethanesulfonylaminocarbonyl)-azetidin-1-yl]-2-methyl-nicotinic acid ethyl ester;
5-Cyano-6-(3-cyclopentylmethanesulfonylaminocarbonyl-azetidin-1-yl)-2-methyl-nicotinic acid ethyl ester;
5-Cyano-6-{3-[2-(2-fluoro-phenyl)-ethanesulfonylaminocarbonyl]-azetidin-1-yl}-2-methyl-nicotinic acid ethyl ester;
5-Cyano-6-[3-(3,5-dichloro-phenylmethanesulfonylaminocarbonyl)-azetidin-1-yl]-2-methyl-nicotinic acid ethyl ester;
5-Cyano-6-(3-cyclohexylmethanesulfonylaminocarbonyl-azetidin-1-yl)-2-methyl-nicotinic acid ethyl ester;
5-Cyano-6-{3-[2-(3-fluoro-phenyl)-ethanesulfonylaminocarbonyl]-azetidin-1-yl}-2-methyl-nicotinic acid ethyl ester;
6-[3-(Benzo[d]isoxazol-3-ylmethanesulfonylaminocarbonyl)-azetidin-1-yl]-5-cyano-2-methyl-nicotinic acid ethyl ester;
1-[4-Amino-3-chloro-5-(5-ethyl-1,3-oxazol-2-yl)pyridin-2-yl]-N(benzylsulfonyl)piperidine-4-carboxamide;
4-Amino-6-(4-{[(benzylsulfonyl)amino]carbonyl}piperidin-1-yl)-5-chloronicotic acid ethyl ester;
6-[3-({[(Benzylsulfonyl)amino]carbonyl}amino)azetidin-1-yl]-5-cyano-2-methylnicotinic acid isopropyl ester;
6-[3-({[(Benzylsulfonyl)amino]carbonyl}amino)azetidin-1-yl]-5-cyano-2-methylnicotinic acid tert-butyl ester;
6-[3-({[(Benzylsulfonyl)amino]carbonyl}amino)azetidin-1-yl]-5-cyano-2-methylnicotic acid ethyl ester;
6-(3-{2-[(Benzylsulfonyl)amino]-2-oxoethyl}piperidin-1-yl)-5-cyano-2-methylnicotinic acid ethyl ester;
6-(4-{[(Benzylsulfonyl)amino]carbonyl}-4-methylpiperidin-1-yl)-5-cyano-2-methylnicotinic acid ethyl ester;
N-(Benzylsulfonyl)-1-[3-chloro-5-(5-ethyl-1,3-oxazol-2-yl)pyridin-2-yl]piperidine-4-carboxamide;
6-(3-{[(Benzylsulfonyl)amino]carbonyl}azetidin-1-yl)-5-cyano-2-methylnicotinic acid cyclopentyl ester;
6-(4-{[(Benzylsulfonyl)amino]carbonyl}piperidin-1-yl)-5-cyano-2-methylnicotinic acid propyl ester;
6-(4-{[(Benzylsulfonyl)amino]carbonyl}piperidin-1-yl)-5-cyano-2-isopropylnicotinic acid ethyl ester;
6-(4-{[(Benzylsulfonyl)amino]carbonyl}piperidin-1-yl)-5-cyano-2-ethylnicotinic acid ethyl ester;
6-(3-{[(Benzylsulfonyl)amino]carbonyl}azetidin-1-yl)-5-cyano-2-methylnicotinic acid 2,2-dimethylpropyl ester;
N-(Benzylsulfonyl)-1-[3-cyano-5-(5-ethyl-1,3-oxazol-2-yl)-6-methylpyridin-2-yl]piperidine-4-carboxamide;
6-(3-{[(Benzylsulfonyl)amino]carbonyl}azetidin-1-yl)-5-cyano-2-methylnicotinic acid isopropyl ester;
6-(4-{[(Benzylsulfonyl)amino]carbonyl}piperidin-1-yl)-5-cyano-2-methylnicotinic acid isopropyl ester;
5-Cyano-6-[4-({[(4-cyanobenzyl)sulfonyl]amino}carbonyl)piperidin-1-yl]-2-methylnicotinic acid ethyl ester;
6-[4-({[(4-Chlorobenzyl)sulfonyl]amino}carbonyl)piperidin-1-yl]-5-cyano-2-methylnicotinic acid ethyl ester;
6-(4-{[(Benzylsulfonyl)amino]carbonyl}piperidin-1-yl)-5-cyano-2-methylnicotinic acid ethyl ester;
N-[(1,2-Benzisoxazol-3-ylmethyl)sulfonyl]-1-[3-cyano-5-(5-ethyl-1,3-oxazol-2-yl)-6-methylpyridin-2-yl]piperidine-4-carboxamide;
N-(Benzylsulfonyl)-1-[3-cyano-5-(5-ethyl-1,3-oxazol-2-yl)-6-methylpyridin-2-yl]azetidine-3-carboxamide;
N-[(4-Chlorobenzyl)sulfonyl]-1-[3-cyano-5-(5-ethyl-1,3-oxazol-2-yl)-6-methylpyridin-2-yl]piperidine-4-carboxamide;
5-Cyano-2-methyl-6-(3-phenylmethanesulfonylaminocarbonyl-azetidin-1-yl)-nicotinic acid ethyl ester;
ethyl 5-cyano-6-{3-[({[3-(4-methoxyphenoxy)propyl]sulfonyl}amino)carbonyl]azetidin-1-yl}-2-methyhlnicotinate;
ethyl 4-amino-6-(3-{[(benzylsulfonyl)amino]carbonyl}azetidin-1-yl)-5-chloronicotinate;
ethyl 5-cyano-2-methyl-6-[3-({[(3-methylbenzyl)sulfonyl]amino}carbonyl)azetidin-1-yl]nicotinate;
2,2-dimethylpropyl 6-(4-{[(benzylsulfonyl)amino]carbonyl}piperidin-1-yl)-5-cyano-2-methylnicotinate;
ethyl 5-cyano-2-methyl-6-[3-({[(4-methylbenzyl)sulfonyl]amino}carbonyl)azetidin-1-yl]nicotinate;
ethyl 5-cyano-6-[4-({[(4-fluorobenzyl)sulfonyl]amino}carbonyl)piperidin-1-yl]-2-methylnicotinate;
ethyl 6-[4-({[(3-bromobenzyl)sulfonyl]amino}carbonyl)piperidin-1-yl]-5-cyano-2-methylnicotinate;
cyclopropyl 6-(3-{[(benzylsulfonyl)amino]carbonyl}azetidin-1-yl)-5-cyano-2-methylnicotinate;
2,2,2-trifluoroethyl 6-(4-{[(benzylsulfonyl)amino]carbonyl}piperidin-1-yl)-5-cyano-2-methylnicotinate;
2,2,2-trifluoroethyl 6-(3-{[(benzylsulfonyl)amino]carbonyl}azetidin-1-yl)-5-cyano-2-methylnicotinate;
2,2,2-trifluoroethyl 6-[3-({[(4-chlorobenzyl)sulfonyl]amino}carbonyl)azetidin-1-yl]-5-cyano-2-methylnicotinate;
cyclopropyl 6-(4-{[(benzylsulfonyl)amino]carbonyl}piperidin-1-yl)-5-cyano-2-methylnicotinate;
cyclobutyl 6-(3-{[(benzylsulfonyl)amino]carbonyl}azetidin-1-yl)-5-cyano-2-methylnicotinate;
2-hydroxyethyl 6-(3-{[(benzylsulfonyl)amino]carbonyl}azetidin-1-yl)-5-cyano-2-methylnicotinate;
benzyl 6-(3-{[(benzylsulfonyl)amino]carbonyl}azetidin-1-yl)-5-cyano-2-methylnicotinate;
isopropyl 5-cyano-6-[4-({[(3,4-dichlorobenzyl)sulfonyl]amino}carbonyl)piperidin-1-yl]-2-methylnicotinate;
ethyl 5-cyano-6-[3-({[(3,4-dichlorobenzyl)sulfonyl]amino}carbonyl)azetidin-1-yl]-2-methylnicotinate;
ethyl 5-cyano-6-[4-({[(3,4-dichlorobenzyl)sulfonyl]amino}carbonyl)piperidin-1-yl]-2-methylnicotinate;
isopropyl 5-cyano-6-[4-({[(4-cyanobenzyl)sulfonyl]amino}carbonyl)piperidin-1-yl]-2-methylnicotinate;
ethyl 5-cyano-6-[3-({[(4-cyanobenzyl)sulfonyl]amino}carbonyl)azetidin-1-yl]-2-methylnicotinate;
isopropyl 5-cyano-6-[4-({[(4-fluorobenzyl)sulfonyl]amino}carbonyl)piperidin-1-yl]-2-methylnicotinate;
isopropyl 6-[4-({[(4-chlorobenzyl)sulfonyl]amino}carbonyl)piperidin-1-yl]-5-cyano-2-methylnicotinate;
ethyl 6-(3-{[(benzylsulfonyl)amino]carbonyl}azetidin-1-yl)-5-cyano-2-isopropylnicotinate;
ethyl 6-(3-{[(benzylsulfonyl)amino]carbonyl}azetidin-1-yl)-5-cyano-2-ethylnicotinate;
ethyl 5-cyano-2-methyl-6-[3-({[(1-phenylethyl)sulfonyl]amino}carbonyl)azetidin-1-yl]nicotinate;
propyl 6-(3-{[(benzylsulfonyl)amino]carbonyl}azetidin-1-yl)-5-cyano-2-methylnicotinate;
isobutyl 6-(3-{[(benzylsulfonyl)amino]carbonyl}azetidin-1-yl)-5-cyano-2-methylnicotinate;
isopropyl 5-cyano-2-methyl-6-{4-[({[4-(trifluoromethyl)benzyl]sulfonyl}amino) carbonyl]piperidin-1-yl}nicotinate;
isopropyl 5-cyano-2-methyl-6-[4-({[(4-methylbenzyl)sulfonyl]amino}carbonyl)piperidin-1-yl]nicotinate;
isopropyl 5-cyano-2-methyl-6-[4-({[(3-methylbenzyl)sulfonyl]amino}carbonyl)piperidin-1-yl]nicotinate;
isopropyl 5-cyano-6-[4-({[(3-fluorobenzyl)sulfonyl]amino}carbonyl)piperidin-1-yl]-2-methylnicotinate;
isopropyl 5-cyano-6-[4-({[(2-fluorobenzyl)sulfonyl]amino}carbonyl)piperidin-1-yl]-2-methylnicotinate;
isopropyl 6-[4-({[(3-chlorobenzyl)sulfonyl]amino}carbonyl)piperidin-1-yl]-5-cyano-2-methylnicotinate;
isopropyl 6-[4-({[(2-chlorobenzyl)sulfonyl]amino}carbonyl)piperidin-1-yl]-5-cyano-2-methylnicotinate;
ethyl 5-cyano-2-methyl-6-[4-({[(4-methylbenzyl)sulfonyl]amino}carbonyl)piperidin-1-yl]nicotinate;
ethyl 5-cyano-6-{4-[({[2-(methoxycarbonyl)benzyl]sulfonyl}amino)carbonyl]piperidin-1-yl}-2-methylnicotinate;
ethyl 5-cyano-6-[4-({[(3-fluorobenzyl)sulfonyl]amino}carbonyl)piperidin-1-yl]-2-methylnicotinate;
isopropyl 5-cyano-2-methyl-6-{4-[({[2-(2-methylphenyl)ethyl]sulfonyl}amino) carbonyl]piperidin-1-yl}nicotinate;
ethyl 6-(4-{[(benzylsulfonyl)amino]carbonyl}piperidin-1-yl)-5-(4-methoxy-4-oxobutoxy)-2-methylnicotinate;
4-{[2-(4-{[(benzylsulfonyl)amino]carbonyl}piperidin-1-yl)-5-(ethoxycarbonyl)-6-methylpyridin-3-yl]oxy}butanoic acid;
ethyl 6-(3-{[(benzylsulfonyl)amino]carbonyl}azetidin-1-yl)-5-(4-methoxy-4-oxobutoxy)-2-methylnicotinate;
ethyl 6-(4-{[(anilinosulfonyl)amino]carbonyl}piperidin-1-yl)-5-cyano-2-methylnicotinate;
ethyl 5-cyano-2-methyl-6-{4-[({[methyl(phenyl)amino]sulfonyl}amino) carbonyl]piperidin-1-yl}nicotinate;
isopropyl 5-cyano-2-methyl-6-[3-({[(4-methylbenzyl)sulfonyl]amino}carbonyl)azetidin-1-yl]nicotinate;
isopropyl 5-cyano-6-[3-({[(3-fluorobenzyl)sulfonyl]amino}carbonyl)azetidin-1-yl]-2-methylnicotinate;
isopropyl 5-cyano-2-methyl-6-[3-({[(2-phenylethyl)sulfonyl]amino}carbonyl)azetidin-1-yl]nicotinate;
isopropyl 5-cyano-6-[3-({[(cyclopentylmethyl)sulfonyl]amino}carbonyl)azetidin-1-yl]-2-methylnicotinate;
isopropyl 5-cyano-6-{3-[({[2-(methoxycarbonyl)benzyl]sulfonyl}amino)carbonyl]azetidin-1-yl}-nicotinate;
isopropyl 5-cyano-6-[3-({[(2-fluorobenzyl)sulfonyl]amino}carbonyl)azetidin-1-yl]-2-methylnicotinate;
isopropyl 6-[3-({[(4-chlorobenzyl)sulfonyl]amino}carbonyl)azetidin-1-yl]-5-cyano-2-methylnicotinate;
isopropyl 5-cyano-6-[3-({[(4-fluorobenzyl)sulfonyl]amino}carbonyl)azetidin-1-yl]-2-methylnicotinate;
isopropyl 5-cyano-6-[3-({[(4-cyanobenzyl)sulfonyl]amino}carbonyl)azetidin-1-yl]-2-methylnicotinate;
methyl 6-(4-{[(benzylsulfonyl)amino]carbonyl}piperidin-1-yl)-5-cyano-2-methylnicotinate;
methyl 5-cyano-2-methyl-6-[4-({[(4-methylbenzyl)sulfonyl]amino}carbonyl)piperidin-1-yl]nicotinate;
S-ethyl 6-(4-{[(benzylsulfonyl)amino]carbonyl}piperidin-1-yl)-5-cyano-2-methylpyridine-3-carbothioate;
S-ethyl 5-cyano-2-methyl-6-[4-({[(4-methylbenzyl)sulfonyl]amino}carbonyl)piperidin-1-yl]pyridine-3-carbothioate;
S-ethyl 6-[4-({[(4-chlorobenzyl)sulfonyl]amino}carbonyl)piperidin-1-yl]-5-cyano-2-methylpyridine-3-carbothioate;
S-ethyl 5-cyano-6-[4-({[(4-fluorobenzyl)sulfonyl]amino}carbonyl)piperidin-1-yl]-2-methylpyridine-3-carbothioate;
ethyl 6-(3-{[(benzylsulfonyl)amino]carbonyl}azetidin-1-yl)-5-methoxy-2-methylnicotinate;
ethyl 6-[4-({[(benzylsulfonyl)amino]carbonyl}amino)piperidin-1-yl]-5-cyano-2-methylnicotinate;
ethyl 6-(4-{[(benzylsulfonyl)amino]carbonyl}piperazin-1-yl)-5-cyano-2-methylnicotinate;
4-{[2-(3-{[(benzylsulfonyl)amino]carbonyl}azetidin-1-yl)-5-(ethoxycarbonyl)-6-methylpyridin-3-yl]oxy}butanoic acid;
ethyl 5-cyano-2-methyl-6-{3-[({[(1-oxidopyridin-2-yl)methyl]sulfonyl}amino) carbonyl]azetidin-1-yl}nicotinate;
ethyl 5-cyano-2-methyl-6-[3-({[(pyridin-3-ylmethyl)sulfonyl]amino}carbonyl)azetidin-1-yl]nicotinate;
ethyl 5-cyano-2-methyl-6-{4-[({[(1-oxidopyridin-2-yl)methyl]sulfonyl}amino) carbonyl]piperidin-1-yl}nicotinate;
ethyl 5-cyano-2-methyl-6-[4-({[(pyridin-3-ylmethyl)sulfonyl]amino}carbonyl)piperidin-1-yl]nicotinate;
ethyl 6-(4-{[(benzylsulfonyl)amino]carbonyl}piperidin-1-yl)-5-cyano-2-(dimethylamino) nicotinate;
ethyl 5-cyano-2-methyl-6-[4-({[(pyridin-4-ylmethyl)sulfonyl]amino}carbonyl)piperidin-1-yl]nicotinate;
ethyl 5-cyano-2-methyl-6-[3-({[(pyridin-2-ylmethyl)sulfonyl]amino}carbonyl)azetidin-1-yl]nicotinate;
ethyl 5-cyano-6-[3-({[(3,5-dimethylbenzyl)sulfonyl]amino}carbonyl)azetidin-1-yl]-2-methylnicotinate;
isopropyl 5-cyano-6-[4-({[(cyclopentylmethyl)sulfonyl]amino}carbonyl)piperidin-1-yl]-2-methylnicotinate;
ethyl 5-cyano-6-[4-({[(2,5-dimethylbenzyl)sulfonyl]amino}carbonyl)piperidin-1-yl]-2-methylnicotinate;
ethyl 5-cyano-6-[4-({[(4-isopropylbenzyl)sulfonyl]amino}carbonyl)piperidin-1-yl]-2-methylnicotinate;
benzyl 6-(4-{[(benzylsulfonyl)amino]carbonyl}piperidin-1-yl)-5-cyano-2-methylnicotinate;
ethyl 5-cyano-2-methyl-6-{4-[({[(4-methylcyclohexyl)methyl]sulfonyl}amino)carbonyl]piperidin-1-yl}nicotinate;
ethyl 5-cyano-6-[3-({[(4-isopropylbenzyl)sulfonyl]amino}carbonyl)azetidin-1-yl]-2-methylnicotinate;
ethyl 5-cyano-2-methyl-6-[4-({[(2-phenylethyl)sulfonyl]amino}carbonyl)piperidin-1-yl]nicotinate;
ethyl 5-cyano-2-methyl-6-[4-({[(pyridin-2-ylmethyl)sulfonyl]amino}carbonyl)piperidin-1-yl]nicotinate;
ethyl 5-cyano-6-[3-({[(2,5-dimethylbenzyl)sulfonyl]amino}carbonyl)azetidin-1-yl]-2-methylnicotinate;
ethyl 6-(3-{[(benzylsulfonyl)amino]carbonyl}azetidin-1-yl)-5-chloro-2-methylnicotinate;
ethyl 6-(3-{2-[(benzylsulfonyl)amino]-2-oxoethyl}azetidin-1-yl)-5-cyano-2-methylnicotinate;
ethyl 5-cyano-6-[4-({[(cyclopentylmethyl)sulfonyl]amino}carbonyl)piperidin-1-yl]-2-methylnicotinate;
ethyl 5-cyano-6-[3-(2-{[(4-fluorobenzyl)sulfonyl]amino}-2-oxoethyl)azetidin-1-yl]-2-methylnicotinate;
ethyl 5-cyano-6-[4-({[(3-fluoro-4-methylbenzyl)sulfonyl]amino}carbonyl)piperidin-1-yl]-2-methylnicotinate;
ethyl 6-(4-{[(benzylsulfonyl)amino]carbonyl}piperidin-1-yl)-5-chloro-2-methylnicotinate;
4-fluorobenzyl 6-(4-{[(benzylsulfonyl)amino]carbonyl}piperidin-1-yl)-5-cyano-2-methylnicotinate;
ethyl 5-cyano-6-[4-({[(4-ethylbenzyl)sulfonyl]amino}carbonyl)piperidin-1-yl]-2-methylnicotinate;
ethyl 5-cyano-6-[3-({[(3,4-difluorobenzyl)sulfonyl]amino}carbonyl)azetidin-1-yl]-2-methylnicotinate;
ethyl 5-cyano-6-[4-({[(4-methoxybenzyl)sulfonyl]amino}carbonyl)piperidin-1-yl]-2-methylnicotinate;
ethyl 5-cyano-2-methyl-6-[4-({[(3-methylbenzyl)sulfonyl]amino}carbonyl)piperidin-1-yl]nicotinate;
ethyl 5-cyano-6-[3-({[(4-ethylbenzyl)sulfonyl]amino}carbonyl)azetidin-1-yl]-2-methylnicotinate;
ethyl 5-chloro-2-methyl-6-[3-({[(4-methylbenzyl)sulfonyl]amino}carbonyl)azetidin-1-yl]nicotinate;
ethyl 5-cyano-6-[4-({[(3,4-difluorobenzyl)sulfonyl]amino}carbonyl)piperidin-1-yl]-2-methylnicotinate;
ethyl 5-cyano-6-[3-({[(4-methoxybenzyl)sulfonyl]amino}carbonyl)azetidin-1-yl]-2-methylnicotinate;
cyclopropyl 5-cyano-2-methyl-6-[4-({[(4-methylbenzyl)sulfonyl]amino}carbonyl)piperidin-1-yl]nicotinate;
ethyl 5-cyano-2-methyl-6-[3-({[(pyridin-4-ylmethyl)sulfonyl]amino}carbonyl)azetidin-1-yl]nicotinate;
ethyl 6-(3-{[(benzylsulfonyl)amino]carbonyl}azetidin-1-yl)-5-cyano-2-(dimethylamino) nicotinate;
ethyl 6-(4-{[(benzylsulfonyl)amino]carbonyl}piperidin-1-yl)-5-cyano-2-methylnicotinate 1-oxide;
ethyl 5-acetyl-6-(4-{[(benzylsulfonyl)amino]carbonyl}piperidin-1-yl)-2-methylnicotinate;
ethyl 6-{4-{[(benzylsulfonyl)amino]carbonyl}-4-[(tert-butoxycarbonyl)amino]piperidin-1-yl}-5-cyano-2-methylnicotinate;
ethyl 6-(4-amino-4-{[(benzylsulfonyl)amino]carbonyl}piperidin-1-yl)-5-cyano-2-methylnicotinate;
or a pharmaceutically acceptable salt thereof.

34. A process for manufacturing a compound of formula (I) in which R₂, R₃, R₄, B, R₁₄, R₁₅, R^{c} and R^{d} are defined according to claim 1, R₁ is R₆OC(O) wherein R₆ is defined according to claim 1, X is a single bond, Z is absent and R₅ is hydrogen, comprising:
i) reacting a compound of the formula R₁CH₂C(O)R₂, with dimethoxy-N,N-dimethylmethaneamine to form a compound of the formula 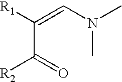
ii) reacting the compound from i) with a compound of the formula R₄CH₂C(O)NH₂ in an inert solvent in the presence of a strong base to give a compound of the formula 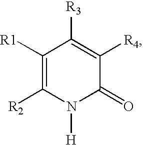
in which R₂, R₃, R₄, are defined claim 1, R₁ is R₆OC(O) wherein R₆ is defined according to claim 1, and Z is
iii) washing the product from ii) with an alkaline water solution, and then washing with water whereafter the washed product is collected,
iv) reacting the compound from iii) with a chlorinating agent in an inert solvent, to give a compound of formula (VII) wherein L is a chlorine,
v) reacting a compound of formula (X) with a compound of formula (III), in which B, R₁₄, R₁₅, R^{c} and R^{d} are defined according to claim 1, X is a single bond and R₅ is a hydrogen, while the compound of formula (III) is having the ring nitrogen protected by t-butyloxycarbonyl, in an inert organic solvent, in the presence of a coupling reagent and optionally an organic base to give a compound of the formula (VIII) after standard deprotection of the t-butyloxycarbonyl, and
vi) reacting the product from v) with the product from iv) in an inert solvent, optionally in the presence of an organic base to give a compound of formula (I) in which R₂, R₃, R₄, B, R₁₄, R₁₅, R^{c} and R^{d} are defined according to claim 1, R₁ is R₆OC(O) and R₆ is defined according to claim 1, X is a single bond, Z is absent and R₅ is hydrogen.

35. The process according to claim 34 wherein iv) comprises adding dimethylformamide to the reaction mixture.

36. The process according to claim 35 wherein the inert solvent in iv) is toluene.

37. The process according to claim 34 wherein the inert organic solvent in v) is THF.

38. The process according to claim 34 wherein the coupling reagent in step v) is TBTU.

39. The process according to claim 34 wherein LiCl is added to the reaction mixture in v).

40. The process according to claim 34 wherein v) comprises isolating the product by adding ammonia dissolved in water.

41. The process according to claim 34 wherein the product from step vi) is purified and isolated by recrystallisation from ethyl acetate.

42. A pharmaceutical composition comprising a compound according to claim 1 in combination with a pharmaceutically acceptable adjuvant, diluent and/or carrier.

43-45. (canceled)

46. A method of treatment of a platelet aggregation disorder comprising administering to a patient suffering from such a disorder a therapeutically effective amount of a compound according to claim 1.

47. A process according to claim 34 wherein:
the inert solvent in ii) is ethanol, and the strong base in ii) is sodium ethoxide;
the alkaline water solution in iii) is a sodium bicarbonate solution;
the chlorinating agent in iv) is thionyl chloride;
the organic base in v) is triethylamine or DIPEA; and
the organic base in vi) is triethylamine.
